(19) United States
(12) Patent Application Publication
(10) Pub. No.: US 2026/0206754 A1
(43) Pub. Date: Jul. 23, 2026

(54) HETEROARYL COMPOUNDS FOR THE CONTROL OF INVERTEBRATE PESTS

(71) Applicant: BASF SE, Ludwigshafen (DE)

(72) Inventors: Julia Pedroni, Ludwigshafen (DE); Matthias Pohlman, Ludwigshafen (DE); Christoph Schissler, Ludwigshafen (DE)

(21) Appl. No.: 19/139,401

(22) PCT Filed: Dec. 11, 2023
(86) PCT No.: PCT/EP2023/085120

§ 371 Date: Jun. 16, 2025

(30) Foreign Application Priority Data
Dec. 21, 2022 (EP) 22215629.1
Dec. 21, 2022 (EP) 22215631.7
Apr. 27, 2023 (EP) 23170420.6
Oct. 19, 2023 (EP) 23204601.1

(51) Int. Cl. A01N 43/40; A01N 43/56; A01N 43/60; A01N 43/647; A01P 7/00; C07D 401/14

(52) U.S. Cl. CPC A01N 43/40; A01N 43/56; A01N 43/60; A01N 43/647; A01P 7/00; C07D 401/14

(57) ABSTRACT

The invention relates to compounds of formula (I) wherein the variables have the meanings as defined in the specification, to compositions comprising them, to active compound combinations comprising them, and to their use for protecting growing plants and animals from attack or infestation by invertebrate pests, furthermore, to seed comprising such compounds.

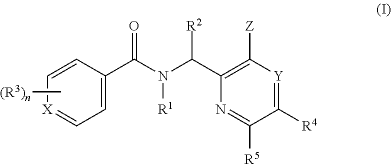

The invention relates to compounds of formula I

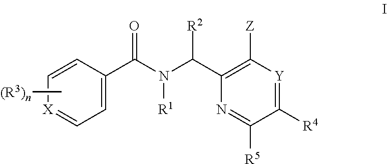

wherein
R¹ is H, OR¹⁰, C₁-C₆-alkyl, C₁-C₆-haloalkyl, C₃-C₆-cycloalkyl, C₃-C₆-halocycloalkyl, C₁-C₅-alkoxy, C₁-C₄-alkyl-C₃-C₆-cycloalkyl, C₁-C₄-alkyl-C₃-C₆-halocycloalkyl, which groups are unsubstituted, or partially or fully substituted with R¹¹;
   or C(═N—R¹¹)R¹², C(O)R^{11a};
   R¹⁰ is H, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halocycloalkyl, or phenyl which is unsubstituted or partially or fully substituted with R^{3a};
   R¹¹ is halogen, CN, NO₂, NR¹²R¹³, C(O)NH₂, C(S)NH₂, C(O)OH, OR¹⁰, Si(CH₃)₃; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, 5- or 6-membered hetaryl, or phenyl, which rings are unsubstituted or substituted with halogen, C₁-C₃-haloalkyl, and/or CN;
   R^{11a} is NR¹²R¹³, C(O)NH₂, C(S)NH₂, C(O)OH, OR¹⁰, Si(CH₃)₃; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, which rings are unsubstituted or substituted with halogen, C₁-C₃-haloalkyl, and/or CN;
   R¹², R¹³ are independently from each other H, C₁-C₄-alkyl, C₁-C₄-alkoxy, C₁-C₄-haloalkoxy, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NH—C₁-C₄-alkyl, C(O)NH—C₁-C₄-haloalkyl, C(O)N(C₁-C₄-alkyl)-C₁-C₄-alkyl, C(O)N(C₁-C₄-haloalkyl)-C₁-C₄-alkyl, C(O)N(C₁-C₄-haloalkyl)-C₁-C₄-haloalkyl, C(O)NH—C₁-C₄-alkoxy, C(O)NH—C₁-C₄-haloalkoxy, C(O)NH—C₁-C₄-alkoxy-C₁-C₄-alkyl, C(O)NH—C₁-C₄-alkoxy-C₁-C₄-haloalkyl; C(O)NH-phenyl, C(O)NH-3-6-membered heterocyclyl or 5- or 6-membered hetaryl, C(O)NH—C₁-C₄-alkyl-phenyl, C(O)NH—C₁-C₄-alkyl-3-6-membered heterocyclyl or 5- or 6-membered hetaryl which rings are unsubstituted or substituted with halogen, C₁-C₃-haloalkyl, and/or CN; S(O)ₘ—C₁-C₄-haloalkyl, S(O)ₘ—C₃-C₄-cycloalkyl, S(O)ₘ—C₃-C₄-halocycloalkyl; 3- to 6-membered heterocyclyl, 5- or 6-membered hetaryl, or phenyl, which rings are unsubstituted or substituted with halogen, C₁-C₃-haloalkyl, and/or CN; or
   or R¹² and R¹³ together with the nitrogen atom to which they are bound, form a 3-, 4-, 5-, 6-, or 7-membered saturated, partially or fully unsaturated heterocycle, which heterocycle may additionally contain 1 or 2 heteroatoms or heteroatom-containing groups selected from N, O, S(O)ₘ, and C(O) as ring members, and which heterocycle is unsubstituted or substituted with one or more R^{3a};
   R¹⁴ is as defined for R¹⁰;
   m is 0, 1, or 2;
R² is H, CN, C₁-C₃-alkyl, C₁-C₃-haloalkyl, C₂-C₃-alkynyl;
R³ is halogen, CN, NO₂;
   C₁-C₄-alkyl, C₃-C₆-cycloalkyl, C₁-C₆-haloalkyl, C₁-C₆-halocycloalkyl, C₁-C₆-alkenyl, C₁-C₆-alkynyl, C₃-C₆-cycloalkyl-C₁-C₆-alkyl, C₁-C₆-alkyl-C₃-C₆-cycloalkyl, which are unsubstituted or substituted with R^{3a};
   OR¹⁴, NR¹²R¹³, C(O)NR¹²R¹³, C(O)OR¹⁴, C(O)R¹⁵, S(O)ₘ—R¹⁵, C(═N—OR¹⁰)R¹⁴, C(═N—OR¹⁰)NR¹²R¹³, or C(═N—NR¹²R¹³)R¹⁴;
   R^{3a} is halogen, CN, NO₂, OH, C₁-C₄-alkyl, C₁-C₄-haloalkyl, C₁-C₄-alkoxy-C₁-C₄-alkyl, C₁-C₄-alkoxy, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl;
   R¹⁵ is H, C₁-C₄-alkyl, or C₁-C₄-haloalkyl, C₃-C₆-cycloalkyl, C₁-C₆-halocycloalkyl, which carbon chains are unsubstituted or partially or fully substituted with R¹¹; or 3- to 6-membered heterocyclyl, 5- or 6-membered hetaryl, or phenyl, which rings are unsubstituted or substituted with R^{3a};
n is 0, 1, 2, or 3;
R⁴ and R⁵ are, independently from each other, H, halogen, CN, C₁-C₆-alkyl, C₁-C₃-haloalkyl, C₃-C₆-cycloalkyl, C₃-C₆-cyclohaloalkyl, OR¹⁰, NR¹²R¹³, 3- to 6-membered heterocyclyl, 5- or 6-membered hetaryl, or phenyl, which rings are unsubstituted or partially or fully substituted with R³;
X, Y are independently from each other N, CH, or CR^{3b};
R^{3b} is as defined for R³;
Z is Q1, Q2, Q3, or Q4, wherein # indicates the bond to the adjacent 6-membered ring

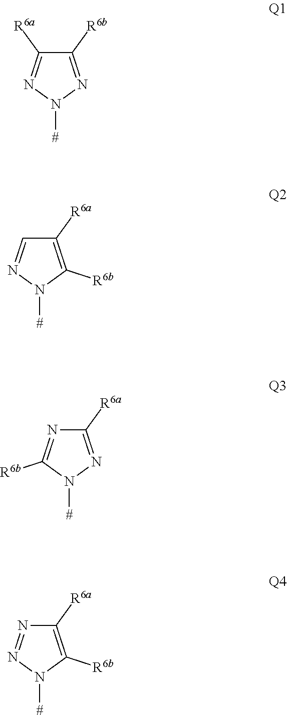

R^{6a} is S(O)ₘ—C₁-C₄-alkyl, S(O)ₘ—C₁-C₄-haloalkyl, S(O)ₘ—C₃-C₆-cycloalkyl, S(O)ₘ—C₃-C₄-halocycloalkyl, NR¹²R¹³, C(O)NR¹²R¹³, C(O)OR¹⁵, 3- to 6-membered heterocyclyl, 5- or 6-membered hetaryl, or phenyl, which rings are unsubstituted or substituted with R³; and
R^{6b} is H, halogen, CN, C₁-C₃-alkyl, C₁-C₃-haloalkyl, C₃-C₄-cycloalkyl, C₃-C₄-halocycloalkyl, NR¹²R¹³, or OR¹⁰, which rings are unsubstituted or substituted with R³;
or, provided that Y is not N,
   R^{6a}, R^{6b} are, independently from each other, H, halogen, CN, C₁-C₃-alkyl, C₁-C₃-haloalkyl, C₃-C₄-cycloalkyl, C₃-C₄-halocycloalkyl, OR¹⁰, S(O)ₘ—C₁-C₄-alkyl, S(O)ₘ—C₁-C₄-haloalkyl, S(O)ₘ—C₃-C₆-cycloalkyl, S(O)ₘ—C₃-C₄-halocycloalkyl, NR¹²R¹³, C(O)NR¹²R¹³, C(O)OR¹⁵, 3- to 6-membered heterocyclyl, 5- or 6-membered hetaryl, or phenyl, which rings are unsubstituted or substituted with R³;
and the N-oxides, stereoisomers, and agriculturally or veterinarily acceptable salts thereof.

The invention also provides agricultural compositions comprising at least one compound of formula I, a stereoisomer thereof and/or an agriculturally acceptable salt thereof and at least one liquid and/or solid carrier, especially at least one inert liquid and/or solid agriculturally acceptable carrier.

The invention also provides a veterinary composition comprising at least one compound of formula I, a stereoisomer thereof and/or a veterinarily acceptable salt thereof and at least one liquid and/or solid carrier, especially at least one inert veterinarily liquid and/or solid acceptable carrier.

The invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The invention also relates to plant propagation material, in particular seed, comprising at least one compound of formula I and/or an agriculturally acceptable salt thereof.

The invention further relates to a method for treating or protecting an animal from infestation or infection by parasites which comprises bringing the animal in contact with a parasiticidally effective amount of a compound of formula I or a veterinarily acceptable salt thereof. Bringing the animal in contact with the compound I, its salt or the veterinary composition of the invention means applying or administering it to the animal.

WO2021/037614, WO2021/122645, and WO2021/259997 describe structurally closely related active compounds. These compounds are mentioned to be useful for combating invertebrate pests.

Nevertheless, there remains a need for highly effective and versatile agents for combating invertebrate pests. It is therefore an object of the invention to provide compounds having a good pesticidal activity and showing a broad activity spectrum against a large number of different invertebrate pests, especially against difficult to control pests, such as insects.

It has been found that these objects can be achieved by compounds of formula I as depicted and defined below, and by their stereoisomers, salts, tautomers and N-oxides, in particular their agriculturally acceptable salts.

Compounds I can be prepared by reaction of corresponding amines II with a carboxylic acid Ill (e.g., A. EI-Fayam et al., “Peptide Coupling Reagents, More than a Letter Soup” Chem Rev 2011, 6557):

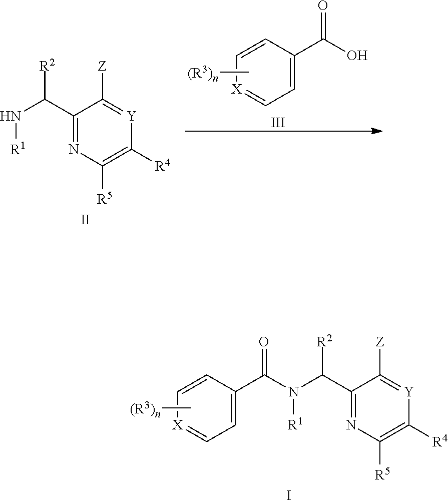

Carboxylic acids Ill are either commercially available or can be prepared following procedures described in the literature (e.g., WO2019/197468, WO2021/037614).

Amines II with Y═N can be prepared from 1-(3-chloropyrazin-2-yl)ethanone as described in WO2021/037614 employing suitably substituted NH-pyrazoles or NH-triazoles Z—H as precursors.

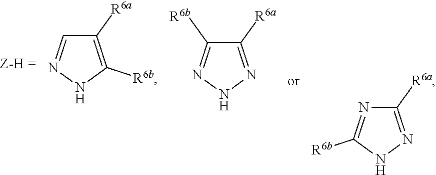

The latter are either commercially available (e.g. CAS 19959-71-8, 23195-62-2, 120241-79-4) or can be prepared by known methods (e.g., S. Fustero et al., “Recent Advances in the Synthesis of Pyrazoles. A Review” Org Prep Proceed Int 2009, 41, 253-290; T. Opsomer et al., “Metal-free syntheses of N-functionalized and NH-1,2,3-triazoles: an update on recent developments” Chem. Commun. 2021, 57, 1568-1590; A. Abdelli et al., “Recent advances in the chemistry of 1,2,4-triazoles: Synthesis, reactivity and biological activities” Tetrahedron Lett 2021, 86, 153518; and references therein).

Amines II with Y═CH or CR^{3b} can be prepared from the corresponding 3-bromo-2-pyridinecarboxylic acids VII in 4 steps: (1) Ullmann-type nucleophilic aromatic substitution of bromopyridine VII with the appropriate reagent Z—H in the presence of a copper catalyst, such as CuI, and a base, such as K₂CO₃ or Cs₂CO₃, and optionally a ligand, such as BINOL or a chelating diamine (e.g. D. Zhu et al., “Efficient copper-catalyzed amination of aryl halides with amines and N—H heterocycles using rac-BINOL as a ligand” J Mol Catal A Chem 2006, 256, 256-260; WO2016/100157); (2) reaction with oxalyl chloride and dimethylformamide (DMF) with subsequent addition of N,O-dimethylhydroxylamine to generate Weinreb amide V (e.g. WO2020/070049) or Weinreb amide preparation by known amide coupling reactions; (3) synthesis of the ketone IV by addition of an organometallic reagent, such as R²Li or R²MgBr, to the Weinreb amide intermediate V (e.g. WO2020/070049); and (4) reductive amination of ketone IV to deliver amine II (e.g. WO2020/070049; WO2021/037614).

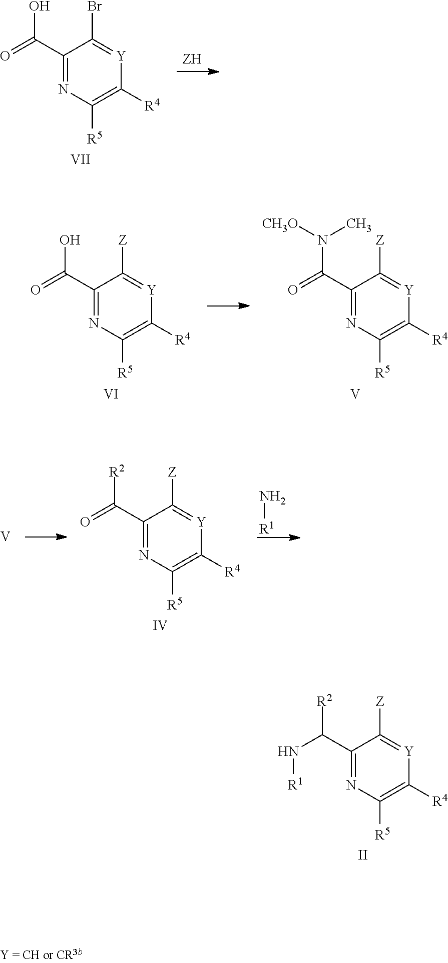

Alternatively, compounds I with Y═N can be prepared from the chloropyrazine precursors VIII by C—N coupling reaction with the corresponding NH-pyrazole or NH-triazole Z—H as described in WO2021/037614:

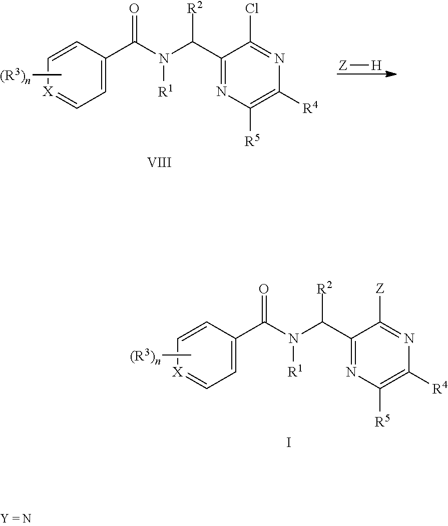

Compounds VIII are known from WO2021/037614 and WO2021/170881.

The reaction mixtures are worked up in a customary manner, for example by mixing with water, extracting with an appropriate organic solvent, separating the phases and, if appropriate, chromatographic purification of the crude products. Some of the intermediates and end products are obtained in the form of colourless or slightly brownish viscous oils which are purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

However, if the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the pest to be controlled.

The organic moieties groups mentioned in the above definitions of the variables are—like the term halogen-collective terms for individual listings of the individual group members. The prefix Cₙ-Cₘ indicates in each case the possible number of carbon atoms in the group.

The term “partially or fully substituted” by a radical means that in general the group is substituted with same or different radicals.

The term “halogen” denotes in each case fluorine, bromine, chlorine, or iodine, in particular fluorine, chlorine, or bromine.

The term “alkyl” as used herein and in the alkyl moieties of alkylamino, alkylcarbonyl, alkylthio, alkylsulfinyl, alkylsulfonyl and alkoxyalkyl denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl (Me), ethyl (Et), n-propyl (n-Pr), iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein and in the haloalkyl moieties of haloalkylcarbonyl, haloalkoxycarbonyl, haloalkylthio, haloalkylsulfonyl, haloalkylsulfinyl, haloalkoxy and haloalkoxyalkyl, denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “alkoxy” as used herein denotes in each case a straight-chain or branched alkyl group which is bonded via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-propoxy, n-butyloxy, 2-butyloxy, iso-butyloxy, tert.-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “haloalkoxy” as used herein denotes in each case a straight-chain or branched alkoxy group having from 1 to 10 carbon atoms, frequently from 1 to 6 carbon atoms, preferably 1 to 4 carbon atoms, wherein the hydrogen atoms of this group are partially or totally replaced with halogen atoms, in particular fluorine atoms. Preferred haloalkoxy moieties include C₁-C₄-haloalkoxy, in particular C₁-C₂-fluoroalkoxy, such as fluoromethoxy, difluoromethoxy, trifluoro-methoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoro-ethoxy, 2,2dichloro-2-fluorethoxy, 2,2,2-trichloroethoxy, penta-fluoroethoxy and the like.

The term “alkylthio “(alkylsulfanyl: S-alkyl)” as used herein refers to a straight-chain or branched saturated alkyl group having 1 to 10 carbon atoms, preferably 1 to 4 carbon atoms (═C₁-C₄-alkylthio), more preferably 1 to 3 carbon atoms, which is attached via a sulfur atom.

The term “haloalkylthio” as used herein refers to an alkylthio group as mentioned above wherein the hydrogen atoms are partially or fully substituted by fluorine, chlorine, bromine and/or iodine.

The term “alkylsulfinyl” (alkylsulfoxyl: S(═O)-alkyl), as used herein refers to a straight-chain or branched saturated alkyl group (as mentioned above) having 1 to 10 carbon atoms, preferably 1 to 4 carbon atoms (═C₁-C₄-alkylsulfinyl), more preferably 1 to 3 carbon atoms bonded through the sulfur atom of the sulfinyl group at any position in the alkyl group.

The term “haloalkylsulfinyl” as used herein refers to an alkylsulfinyl group as mentioned above wherein the hydrogen atoms are partially or fully substituted by fluorine, chlorine, bromine and/or iodine.

The term “alkylsulfonyl” (S(═O)₂-alkyl) as used herein refers to a straight-chain or branched saturated alkyl group having 1 to 10 carbon atoms, preferably 1 to 4 carbon atoms (═C₁-C₄-alkylsulfonyl), preferably 1 to 3 carbon atoms, which is bonded via the sulfur atom of the sulfonyl group at any position in the alkyl group.

The term “haloalkylsulfonyl” as used herein refers to an alkylsulfonyl group as mentioned above wherein the hydrogen atoms are partially or fully substituted by fluorine, chlorine, bromine and/or iodine.

The term “alkylcarbonyl” refers to an alkyl group as defined above, which is bonded via the carbon atom of a carbonyl group (C═O) to the remainder of the molecule.

The term “haloalkylcarbonyl” refers to an alkylcarbonyl group as mentioned above, wherein the hydrogen atoms are partially or fully substituted by fluorine, chlorine, bromine and/or iodine.

The term “alkoxycarbonyl” refers to an alkylcarbonyl group as defined above, which is bonded via an oxygen atom to the remainder of the molecule.

The term “haloalkoxycarbonyl” refers to an alkoxycarbonyl group as mentioned above, wherein the hydrogen atoms are partially or fully substituted by fluorine, chlorine, bromine and/or iodine.

The term “alkenyl” as used herein denotes in each case a singly unsaturated hydrocarbon radical having usually 2 to 10, frequently 2 to 6, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkynyl” as used herein denotes in each case a singly unsaturated hydrocarbon radical having usually 2 to 10, frequently 2 to 6, preferably 2 to 4 carbon atoms, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “cycloalkyl” as used herein and in the cycloalkyl moieties of cycloalkoxy and cycloalkylthio denotes in each case a monocyclic cycloaliphatic radical having usually from 3 to 10 or from 3 to 6 carbon atoms, such as cyclopropyl (cC₃H₅), cyclobutyl (cC₄H₇), cyclopentyl (cC₅H₉), cyclohexyl (cC₆H₁₁), cycloheptyl, cyclooctyl, cyclononyl and cyclodecyl or cyclopropyl, cyclobutyl, cyclopentyl and cyclohexyl.

The term “halocycloalkyl” as used herein and in the halocycloalkyl moieties of halocycloalkoxy and halocycloalkylthio denotes in each case a monocyclic cycloaliphatic radical having usually from 3 to 10 C atoms or 3 to 6 C atoms, wherein at least one, e.g. 1, 2, 3, 4 or 5 of the hydrogen atoms, are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichloro-cyclopropyl, 2,2,3,3-tetrachlorocyclpropyl, 1-,2- and 3-fluorocyclopentyl, 1,2-, 2,2-, 2,3-, 3,3-, 3,4-, 2,5-difluorocyclopentyl, 1-,2- and 3-chlorocyclopentyl, 1,2-, 2,2-, 2,3-, 3,3-, 3,4-, 2,5-dichlorocyclopentyl and the like.

The term “halocycloalkenyl” as used herein and in the halocycloalkenyl moieties of halocycloalkenyloxy and halocycloalkenylthio denotes in each case a monocyclic singly unsaturated non-aromatic radical having usually from 3 to 10, e.g. 3 or 4 or from 5 to 10 carbon atoms, preferably from 3- to 8 carbon atoms, wherein at least one, e.g. 1, 2, 3, 4 or 5 of the hydrogen atoms, are replaced by halogen, in particular by fluorine or chlorine. Examples are 3,3-difluorocyclopropen-1-yl and 3,3-dichlorocyclopropen-1-yl.

The term “cycloalkenylalkyl” refers to a cycloalkenyl group as defined above which is bonded via an alkyl group, such as a C₁-C₅-alkyl group or a C₁-C₄-alkyl group, in particular a methyl group (=cycloalkenylmethyl), to the remainder of the molecule.

The term “carbocycle” or “carbocyclyl” includes in general a 3- to 12-membered, preferably a 3- to 8-membered or a 5- to 8-membered, more preferably a 5- or 6-membered monocyclic, non-aromatic ring comprising 3 to 12, preferably 3 to 8 or 5 to 8, more preferably 5 or 6 carbon atoms. Preferably, the term “carbocycle” covers cycloalkyl and cycloalkenyl groups as defined above.

The term “heterocycle” or “heterocyclyl” includes in general 3- to 12-membered, preferably 3-to 6-membered, in particular 6-membered monocyclic heterocyclic non-aromatic radicals. The heterocyclic non-aromatic radicals usually comprise 1, 2, 3, 4 or 5, preferably 1, 2 or 3 heteroatoms selected from N, O, and S as ring members, wherein S-atoms as ring members may be present as S, SO, or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S. oxothiopyranyl, S-dioxothiopyranyl, dihydrothiopyranyl, S-oxodihydrothiopyranyl, S-dioxodihydrothiopyranyl, tetrahydrothiopyranyl, S-oxotetrahydrothiopyranyl, S-dioxotetrahydrothiopyranyl, morpholinyl, thiomorpholinyl, S-oxothiomorpholinyl, S-dioxothio-morpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-only, and the like.

The term “hetaryl” includes monocyclic 5- or 6-membered heteroaromatic radicals comprising as ring members 1, 2, 3 or 4 heteroatoms selected from N, O, and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “hetaryl” also includes bicyclic 8 to 10-membered heteroaromatic radicals comprising as ring members 1, 2 or 3 heteroatoms selected from N, O, and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzo-thienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety.

The terms “heterocyclylalkyl” and “hetarylalkyl” refer to heterocyclyl or hetaryl, respectively, as defined above which are bonded via a C₁-C₅-alkyl group or a C₁-C₄-alkyl group, in particular a methyl group (=heterocyclylmethyl or hetarylmethyl, respectively), to the remainder of the molecule.

The term “arylalkyl” and “phenylalkyl” refer to aryl as defined above and phenyl, respectively, which are bonded via C₁-C₅-alkyl group or a C₁-C₄-alkyl group, in particular a methyl group (=arylmethyl or phenylmethyl), to the remainder of the molecule, examples including benzyl, 1-phenylethyl, 2-phenylethyl, 2-phenoxyethyl etc.

The terms “alkylene”, “cycloalkylene”, “heterocycloalkylene”, “alkenylene”, “cycloalkenylene”, “heterocycloalkenylene” and “alkynylene” refer to alkyl, cycloalkyl, heterocycloalkyl, alkenyl, cycloalkenyl, heterocycloalkenyl and alkynyl as defined above, respectively, which are bonded to the remainder of the molecule, via two atoms, preferably via two carbon atoms, of the respective group, so that they represent a linker between two moieties of the molecule.

In a particular embodiment, the variables of the compounds of the formula I have the following meanings, these meanings, both on their own and in combination with one another, being particular embodiments of the compounds of the formula I.

Embodiments and preferred compounds of the invention for use in pesticidal methods and for insecticidal application purposes are outlined in the following paragraphs.

With respect to the variables, the particularly preferred embodiments of the intermediates correspond to those of the compounds of the formula I.

In a preferred embodiment, the compounds I are present in form of a mixture of compounds I.S and I.R, wherein compound I.S with S-configuration of the carbon atom neighboring the nitrogen is present in an amount of more than 50% by weight, in particular of at least 70% by weight, more particularly of at least 85% by weight, specifically of at least 90% by weight, based on the total weight of compounds I.S and I.R.

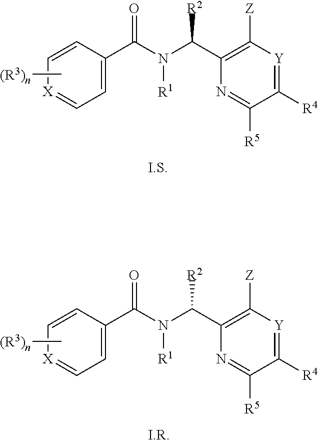

In one particularly preferred embodiment of the invention, the method comprises the step of contacting the plant, parts of it, its propagation material, the pests, their food supply, habitat or breeding grounds with a pesticidally effective amount of a compound of formula I.S.

Preferably R¹ is H, C₁-C₆-alkyl, C₃-C₆-alkynyl, C₃-C₆-cycloalkyl, or C₁-C₄-alkyl-C₃-C₆-cycloalkyl, particularly selected from H, CH₃, C₂H₅, and CH₂cC₃H₅.

Preferably R² is CH₃.

X is preferably CH or CR³, particularly CH. Such compounds correspond to Formula I.1

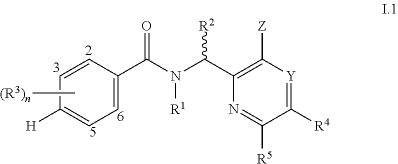

In another embodiment X is N. Such compounds correspond to formula I.2.

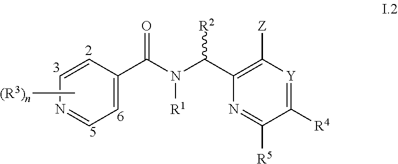

In a preferred embodiment R³ is halogen, CN, NO₂; C₁-C₄-alkyl, C₃-C₆-cycloalkyl, C₁-C₆-haloalkyl, C₁-C₆-halocycloalkyl, C₁-C₆-alkenyl, C₁-C₆-alkynyl, C₃-C₆-cycloalkyl-C₁-C₆-alkyl, C₁-C₆-alkyl-C₃-C₆-cycloalkyl, which are unsubstituted or substituted with R^{3a};
or OR¹⁴, NR¹²R¹³, C(O)NR¹²R¹³, C(O)OR¹⁴, C(O)R¹⁵, S(O)ₘ—R¹⁵.

In another preferred embodiment R³ is halogen, CN, NO₂; C₁-C₄-alkyl, C₃-C₆-cycloalkyl, C₁-C₆-haloalkyl, C₁-C₆-halocycloalkyl, which are unsubstituted or substituted with R^{3a}; OR¹⁰, S(O)ₘ—R¹⁵.

In a preferred embodiment R^{3a} is halogen, CN, NO₂, OH, C₁-C₄-alkyl, C₁-C₄-haloalkyl, C₁-C₄-alkoxy-C₁-C₄-alkyl, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl.

R³ is preferably halogen, CN, C₁-C₄-haloalkyl, C₁-C₄-haloalkoxy, C₃-C₄-cycloalkyl unsubstituted or substituted with one or more CN or halogen, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, S(O)ₘ-(substituted phenyl); particularly selected from F, C, Br, I, CN, CF₃, CHF₂, OCH₃, OC₂H₅, OcC₃H₅, O(4-F—C₆H₄), OCF₃, OCHF₂, 1-CN-cC₃H₄, 2,2-F₂-cC₃H₃, 2,2-Cl₂-cC₃H₃, SO₂CH₃, SO₂C₂H₅, SO₂CH(CH₃)₂, SO₂CF₃, SO₂(4-F—CeH₄), and C(CH₃)₂CN.

Index m in R³ is preferably 2. Index n is preferably 2.

R³ groups stand preferably in positions 3 and 5.

In another embodiment R³ is preferably halogen, CN, C₁-C₄-haloalkyl, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, or S(O)ₘ—R¹⁴, wherein R¹⁴ is phenyl, which is partially substituted with R^{3a}.

In another preferred embodiment at least one R³ is C(═N—OR¹⁰)R¹⁴, C(═N—OR¹⁰)NR¹²R¹³ or C(═N—NR¹²R¹³)R¹⁴.

Preferred R¹⁰ are H, C₁-C₆-alkyl, and C₃-C₆-cycloalkyl, particularly H, C₁-C₄-alkyl, and C₃-cycloalkyl.

In group C(═N—OR¹⁰)R¹⁴ only one of R¹⁰ and R¹⁴ is C₁-C₄-alkyl, and R¹⁴ is preferably not H.

Preferred R¹²/R¹³ are H, C₁-C₆-alkyl, C₃-C₆-cycloalkyl, C(O)NH—C₁-C₄-alkyl, and C(O)NH—C₆H₅.

Preferred R¹⁴ are H, C₁-C₄-alkyl, C₃-C₆-cycloalkyl, or C₃-C₆-halocycloalkyl, particularly H, CH₃ or cyclopropyl.

R^{3b} is preferably halogen, C₁-C₄-alkyl, or C₃-C₆-cycloalkyl, particularly F, Cl, CH₃, or cC₃H₅.

R⁴ is preferably H, halogen, or C₁-C₄-alkyl, particularly H, F, Cl, or CH₃. In another preferred embodiment R⁴ is H.

R⁵ is preferably H, halogen, C₁-C₄-alkyl, or C₃-C₆-cycloalkyl, particularly H, F, Cl, CH₃, or cC₃H₅.

In a preferred embodiment only one of R⁴ and R⁵, preferably R⁵, is different from H. In another embodiment R⁴ and R⁵ are both H.

In a preferred embodiment Y is N. Such compounds correspond to Formula I.A

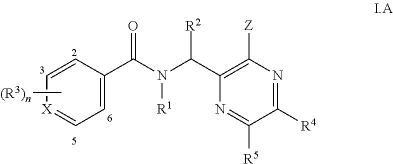

In a preferred embodiment of compounds I.A R^{6a} is S(O)ₘ—C₁-C₄-alkyl, S(O)ₘ—C₁-C₄-haloalkyl, S(O)ₘ—C₃-C₆-cycloalkyl, S(O)ₘ—C₃-C₄-halocycloalkyl, NR¹²R¹³, C(O)NR¹²R¹³, C(O)OR¹⁵, 3- to 6-membered heterocyclyl, 5- or 6-membered hetaryl, or phenyl, which rings are unsubstituted or substituted with R³; and R^{6b} is H, halogen, CN, C₁-C₃-alkyl, C₁-C₃-haloalkyl, C₃-C₄-cycloalkyl, C₃-C₄-halocycloalkyl, OR¹⁰, which rings are unsubstituted or substituted with R³. In a particularly preferred embodiment of compounds I.A R^{6b} is H.

In another particularly preferred embodiment of compounds I.A X is N.

In another embodiment Y is CH. Such compounds correspond to Formula I.B

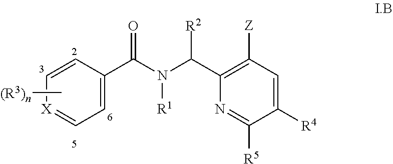

In compounds I.B R^{6a} and R^{6b} are, independently from each other, H, halogen, CN, C₁-C₃-alkyl, C₁-C₃-haloalkyl, C₃-C₄-cycloalkyl, C₃-C₄-halocycloalkyl, OR¹⁰, S(O)ₘ—C₁-C₄-alkyl, S(O)ₘ—C₁-C₄-haloalkyl, S(O)ₘ—C₃-C₆-cycloalkyl, S(O)ₘ—C₃-C₄-halocycloalkyl, NR¹²R¹³, C(O)NR¹²R¹³, C(O)OR¹⁵, 3- to 6-membered heterocyclyl, 5- or 6-membered hetaryl, or phenyl, which rings are unsubstituted or substituted with R³. In a preferred embodiment of compounds I.B R^{6a} and R^{6b} are H.

In another embodiment Y is CR^{3b}. Such compounds correspond to Formula I.C

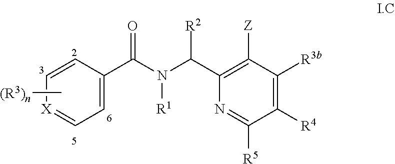

In a preferred embodiment of compounds I.C R^{3b} is halogen.

In compounds I.C R^{6a} and R^{6b} are, independently from each other, H, halogen, CN, C₁-C₃-alkyl, C₁-C₃-haloalkyl, C₃-C₄-cycloalkyl, C₃-C₄-halocycloalkyl, OR¹⁰, S(O)ₘ—C₁-C₄-alkyl, S(O)ₘ—C₁-C₄-haloalkyl, S(O)ₘ—C₃-C₆-cycloalkyl, S(O)ₘ—C₃-C₄-halocycloalkyl, NR¹²R¹³, C(O)NR¹²R¹³, C(O)OR¹⁵, 3- to 6-membered heterocyclyl, 5- or 6-membered hetaryl, or phenyl, which rings are unsubstituted or substituted with R³. In a preferred embodiment of compounds I.C R^{6a} and R^{6b} are H.

In a preferred embodiment Z is Q1 or Q2, particularly Q1.

Particularly preferred compounds I correspond to formulae I.AQ1, I.AQ2, and I.BQ1.

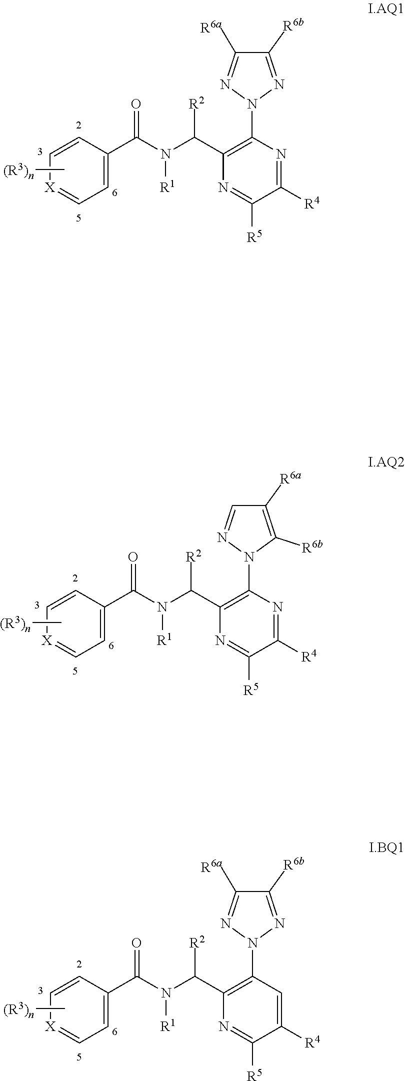

Another preferred embodiment relates to compounds of formulae I.AQ1, I.BQ1, and I.BQ2.

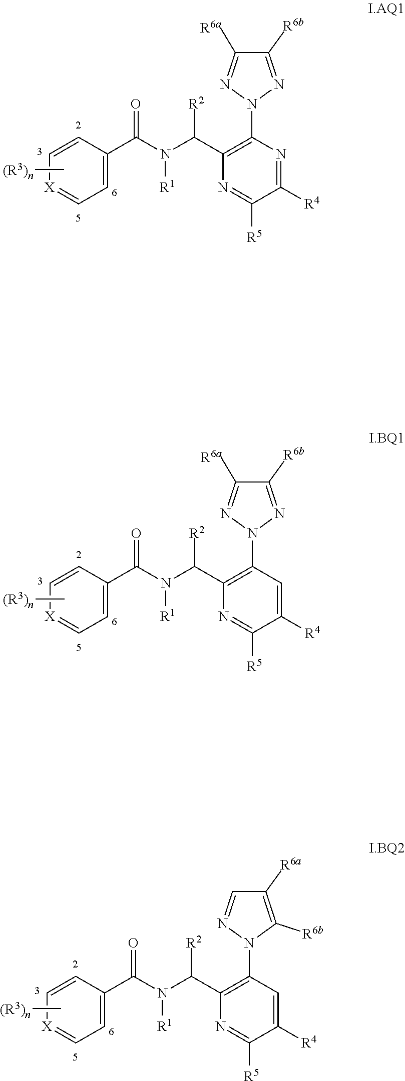

Another preferred embodiment relates to compounds of formulae I.CQ1 and I.CQ2.

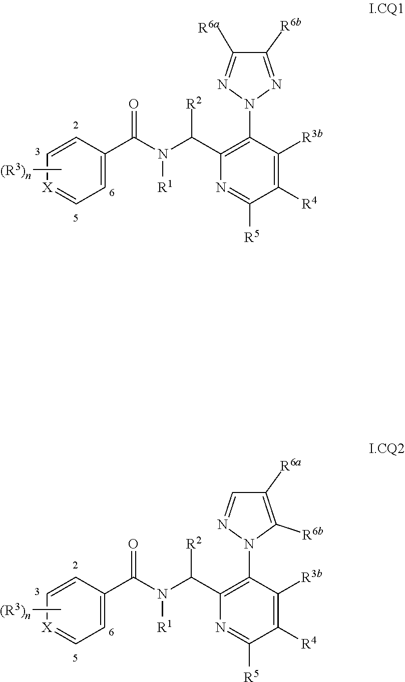

R^{6a} is preferably 6-membered hetaryl with 1 or 2 N atoms as ring atoms, unsubstituted or substituted with 1 or 2 substituents R³, particularly 2-, 3- or 4-pyridine, unsubstituted or substituted with R³. Preferably hetaryl is unsubstituted.

A particularly preferred embodiment relates to compounds I.AQ1, wherein R^{6a} is hetaryl or phenyl, which rings are unsubstituted or substituted with C(O)NHR¹², C(O)NR¹²R¹³, C(O)OR¹², NR¹²R¹³, C(═N—OR¹⁰)R¹⁴, C(═N—OR¹⁰)NR¹²R¹³, or C(═N—NR¹²R¹³)R¹⁴, S(O)ₘ—C₃-C₆-cycloalkyl, or S(O)ₘR¹⁵, wherein R¹⁵ is preferably 5- or 6-membered hetaryl, or phenyl, which rings are unsubstituted or substituted with R^{3a}.

Another particularly preferred embodiment relates to Q2, wherein R^{6a} is 3- to 6-membered heterocyclyl, 5- or 6-membered hetaryl, or phenyl, which rings are unsubstituted or substituted with C(O)NHR¹², C(O)NR¹²R¹³, C(O)OR¹², NR¹²R¹³, C(═N—OR¹⁰)R¹⁴, C(═N—OR¹⁰)NR¹²R¹³, or C(═N—NR¹²R¹³)R¹⁴, S(O)ₘ—C₃-C₆-cycloalkyl, or S(O)ₘR¹⁵, wherein R¹⁵ is preferably 5- or 6-membered hetaryl, or phenyl, which rings are unsubstituted or substituted with R^{3a}.

In another embodiment R^{6a} is S(O)ₘ—C₁-C₄-alkyl, S(O)ₘ—C₁-C₄-haloalkyl, S(O)ₘ—C₃-C₆-cycloalkyl, S(O)ₘ—C₃-C₄-halocycloalkyl, NR¹²R¹³, C(O)NR¹²R¹³, C(O)OR¹⁵; preferably S(O)ₘ—C₁-C₄-alkyl, S(O)ₘ—C₁-C₄-haloalkyl, S(O)ₘ—C₃-C₆-cycloalkyl, S(O)ₘ—C₃-C₄-halocycloalkyl.

In another embodiment of compounds I.B and I.C R^{6a} is H.

R^{6b} is preferably H, halogen, C₁-C₄-alkyl, or C₃-C₆-cycloalkyl, preferably H, F, Cl, CH₃, or cC₃H₅, particularly R^{6b} is H.

One embodiment relates to compounds I wherein Y is N and Z is Q1 or Q4.

Another embodiment relates to compounds I wherein Y is N and Z is Q2.

Another embodiment relates to compounds I wherein Y is N and Z is Q3.

Another embodiment relates to compounds I wherein Y is CH or CR^{3b}.

In particular with a view to their use, preference is given to the compounds of formula I compiled in the tables below. Each of the groups mentioned for a substituent in the tables is furthermore per se, independently of the combination in which it is mentioned, a particularly preferred aspect of the substituent in question.

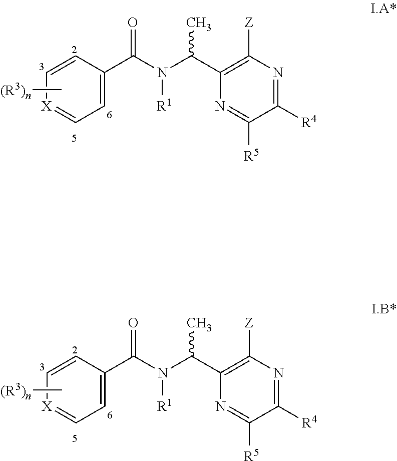

Table 1: Compounds of formula I.A* in which R⁴ and R⁵ are H, Z is Q1, R^{6a} is 2-pyridyl, R^{6b} is H, and the combination of R¹, X, and (R³)ₙ for a compound corresponds in each case to one row of Table A

Table 2: Compounds of formula I.A* in which R⁴ is H, R⁵ is F, Z is Q1, R^{6a} is 2-pyridyl, R^{6b} is H, and the combination of R¹, X, and (R³)ₙ for a compound corresponds in each case to one row of Table A

Table 3: Compounds of formula I.A* in which R⁴ is H, R⁵ is Cl, Z is Q1, R^{6a} is 2-pyridyl, R^{6b} is H, and the combination of R¹, X, and (R³)ₙ for a compound corresponds in each case to one row of Table A

Table 4: Compounds of formula I.A* in which R⁴ is H, R⁵ is CH₃, Z is Q1, R^{6a} is 2-pyridyl, R^{6b} is H, and the combination of R¹, X, and (R³)ₙ for a compound corresponds in each case to one row of Table A

Table 5: Compounds of formula I.A* in which R⁴ is H, R⁵ is cC₃H₅, Z is Q1, R^{6a} is 2-pyridyl, R^{6b} is H, and the combination of R¹, X, and (R³)ₙ for a compound corresponds in each case to one row of Table A

Table 6: Compounds of formula I.A* in which R⁴ and R⁵ are H, Z is Q2, R^{6a} is 2-pyridyl, R^{6b} is H, and the combination of R¹, X, and (R³)ₙ for a compound corresponds in each case to one row of Table A

Table 7: Compounds of formula I.A* in which R⁴ is H, R⁵ is F, Z is Q2, R^{6a} is 2-pyridyl, R^{6b} is H, and the combination of R¹, X, and (R³)ₙ for a compound corresponds in each case to one row of Table A

Table 8: Compounds of formula I.A* in which R⁴ is H, R⁵ is Cl, Z is Q2, R^{6a} is 2-pyridyl, R^{6b} is H, and the combination of R¹, X, and (R³)ₙ for a compound corresponds in each case to one row of Table A

Table 9: Compounds of formula I.A* in which R⁴ is H, R⁵ is CH₃, Z is Q2, R^{6a} is 2-pyridyl, R^{6b} is H, and the combination of R¹, X, and (R³)ₙ for a compound corresponds in each case to one row of Table A

Table 10: Compounds of formula I.A* in which R⁴ is H, R⁵ is cC₃H₅, Z is Q2, R^{6a} is 2-pyridyl, R^{6b} is H, and the combination of R¹, X, and (R³)ₙ for a compound corresponds in each case to one row of Table A

Table 11: Compounds of formula I.A* in which R⁴ and R⁵ are H, Z is Q3, R^{6a} is 2-pyridyl, R^{6b} is H, and the combination of R¹, X, and (R³)ₙ for a compound corresponds in each case to one row of Table A

Table 12: Compounds of formula I.A* in which R⁴ is H, R⁵ is F, Z is Q3, R^{6a} is 2-pyridyl, R^{6b} is H, and the combination of R¹, X, and (R³)ₙ for a compound corresponds in each case to one row of Table A

Table 13: Compounds of formula I.A* in which R⁴ is H, R⁵ is Cl, Z is Q3, R^{6a} is 2-pyridyl, R^{6b} is H, and the combination of R¹, X, and (R³)ₙ for a compound corresponds in each case to one row of Table A

Table 14: Compounds of formula I.A* in which R⁴ is H, R⁵ is CH₃, Z is Q3, R^{6a} is 2-pyridyl, R^{6b} is H, and the combination of R¹, X, and (R³)ₙ for a compound corresponds in each case to one row of Table A

Table 15: Compounds of formula I.A* in which R⁴ is H, R⁵ is cC₃H₅, Z is Q3, R^{6a} is 2-pyridyl, R^{6b} is H, and the combination of R¹, X, and (R³)ₙ for a compound corresponds in each case to one row of Table A

Table 16: Compounds of formula I.A* in which R⁴ and R⁵ are H, Z is Q4, R^{6a} is 2-pyridyl, R^{6b} is H, and the combination of R¹, X, and (R³)ₙ for a compound corresponds in each case to one row of Table A

Table 17: Compounds of formula I.A* in which R⁴ is H, R⁵ is F, Z is Q4, R^{6a} is 2-pyridyl, R^{6b} is H, and the combination of R¹, X, and (R³)ₙ for a compound corresponds in each case to one row of Table A

Table 18: Compounds of formula I.A* in which R⁴ is H, R⁵ is Cl, Z is Q4, R^{6a} is 2-pyridyl, R^{6b} is H, and the combination of R¹, X, and (R³)ₙ for a compound corresponds in each case to one row of Table A

Table 19: Compounds of formula I.A* in which R⁴ is H, R⁵ is CH₃, Z is Q4, R^{6a} is 2-pyridyl, R^{6b} is H, and the combination of R¹, X, and (R³)ₙ for a compound corresponds in each case to one row of Table A

Table 20: Compounds of formula I.A* in which R⁴ is H, R⁵ is cC₃H₅, Z is Q4, R^{6a} is 2-pyridyl, R^{6b} is H, and the combination of R¹, X, and (R³)ₙ for a compound corresponds in each case to one row of Table A

Table 21: Compounds of formula I.A* in which R⁴ and R⁵ are H, Z is Q1, R^{6a} is 3-pyridyl, R^{6b} is H, and the combination of R¹, X, and (R³)ₙ for a compound corresponds in each case to one row of Table A

Table 22: Compounds of formula I.A* in which R⁴ is H, R⁵ is F, Z is Q1, R^{6a} is 3-pyridyl, R^{6b} is H, and the combination of R¹, X, and (R³)ₙ for a compound corresponds in each case to one row of Table A

Table 23: Compounds of formula I.A* in which R⁴ is H, R⁵ is Cl, Z is Q1, R^{6a} is 3-pyridyl, R^{6b} is H, and the combination of R¹, X, and (R³)ₙ for a compound corresponds in each case to one row of Table A

Table 24: Compounds of formula I.A* in which R⁴ is H, R⁵ is CH₃, Z is Q1, R^{6a} is 3-pyridyl, R^{6b} is H, and the combination of R¹, X, and (R³)ₙ for a compound corresponds in each case to one row of Table A

Table 25: Compounds of formula I.A* in which R⁴ is H, R⁵ is cC₃H₅, Z is Q1, R^{6a} is 3-pyridyl, R^{6b} is H, and the combination of R¹, X, and (R³)ₙ for a compound corresponds in each case to one row of Table A

Table 26: Compounds of formula I.A* in which R⁴ and R⁵ are H, Z is Q2, R^{6a} is 3-pyridyl, R^{6b} is H, and the combination of R¹, X, and (R³)ₙ for a compound corresponds in each case to one row of Table A

Table 27: Compounds of formula I.A* in which R⁴ is H, R⁵ is F, Z is Q2, R^{6a} is 3-pyridyl, R^{6b} is H, and the combination of R¹, X, and (R³)ₙ for a compound corresponds in each case to one row of Table A

Table 28: Compounds of formula I.A* in which R⁴ is H, R⁵ is Cl, Z is Q2, R^{6a} is 3-pyridyl, R^{6b} is H, and the combination of R¹, X, and (R³)ₙ for a compound corresponds in each case to one row of Table A

Table 29: Compounds of formula I.A* in which R⁴ is H, R⁵ is CH₃, Z is Q2, R^{6a} is 3-pyridyl, R^{6b} is H, and the combination of R¹, X, and (R³)ₙ for a compound corresponds in each case to one row of Table A

Table 30: Compounds of formula I.A* in which R⁴ is H, R⁵ is cC₃H₅, Z is Q2, R^{6a} is 3-pyridyl, R^{6b} is H, and the combination of R¹, X, and (R³)ₙ for a compound corresponds in each case to one row of Table A

Table 31: Compounds of formula I.A* in which R⁴ and R⁵ are H, Z is Q3, R^{6a} is 3-pyridyl, R^{6b} is H, and the combination of R¹, X, and (R³)ₙ for a compound corresponds in each case to one row of Table A

Table 32: Compounds of formula I.A* in which R⁴ is H, R⁵ is F, Z is Q3, R^{6a} is 3-pyridyl, R^{6b} is H, and the combination of R¹, X, and (R³)ₙ for a compound corresponds in each case to one row of Table A

Table 33: Compounds of formula I.A* in which R⁴ is H, R⁵ is Cl, Z is Q3, R^{6a} is 3-pyridyl, R^{6b} is H, and the combination of R¹, X, and (R³)ₙ for a compound corresponds in each case to one row of Table A

Table 34: Compounds of formula I.A* in which R⁴ is H, R⁵ is CH₃, Z is Q3, R^{6a} is 3-pyridyl, R^{6b} is H, and the combination of R¹, X, and (R³)ₙ for a compound corresponds in each case to one row of Table A

Table 35: Compounds of formula I.A* in which R⁴ is H, R⁵ is cC₃H₅, Z is Q3, R^{6a} is 3-pyridyl, R^{6b} is H, and the combination of R¹, X, and (R³)ₙ for a compound corresponds in each case to one row of Table A

Table 36: Compounds of formula I.A* in which R⁴ and R⁵ are H, Z is Q4, R^{6a} is 3-pyridyl, R^{6b} is H, and the combination of R¹, X, and (R³)ₙ for a compound corresponds in each case to one row of Table A

Table 37: Compounds of formula I.A* in which R⁴ is H, R⁵ is F, Z is Q4, R^{6a} is 3-pyridyl, R^{6b} is H, and the combination of R¹, X, and (R³)ₙ for a compound corresponds in each case to one row of Table A

Table 38: Compounds of formula I.A* in which R⁴ is H, R⁵ is Cl, Z is Q4, R^{6a} is 3-pyridyl, R^{6b} is H, and the combination of R¹, X, and (R³)ₙ for a compound corresponds in each case to one row of Table A

Table 39: Compounds of formula I.A* in which R⁴ is H, R⁵ is CH₃, Z is Q4, R^{6a} is 3-pyridyl, R^{6b} is H, and the combination of R¹, X, and (R³)ₙ for a compound corresponds in each case to one row of Table A

Table 40: Compounds of formula I.A* in which R⁴ is H, R⁵ is cC₃H₅, Z is Q4, R^{6a} is 3-pyridyl, R^{6b} is H, and the combination of R¹, X, and (R³)ₙ for a compound corresponds in each case to one row of Table A

Table 41: Compounds of formula I.A* in which R⁴ is F, R⁵ is H, Z is Q1, R^{6a} is 2-pyridyl, R^{6b} is H, and the combination of R¹, X, and (R³)ₙ for a compound corresponds in each case to one row of Table A

Table 42: Compounds of formula I.A* in which R⁴ is F, R⁵ is F, Z is Q1, R^{6a} is 2-pyridyl, R^{6b} is H, and the combination of R¹, X, and (R³)ₙ for a compound corresponds in each case to one row of Table A

Table 43: Compounds of formula I.A* in which R⁴ is F, R⁵ is Cl, Z is Q1, R^{6a} is 2-pyridyl, R^{6b} is H, and the combination of R¹, X, and (R³)ₙ for a compound corresponds in each case to one row of Table A

Table 44: Compounds of formula I.A* in which R⁴ is F, R⁵ is CH₃, Z is Q1, R^{6a} is 2-pyridyl, R^{6b} is H, and the combination of R¹, X, and (R³)ₙ for a compound corresponds in each case to one row of Table A

Table 45: Compounds of formula I.A* in which R⁴ is F, R⁵ is cC₃H₅, Z is Q1, R^{6a} is 2-pyridyl, R^{6b} is H, and the combination of R¹, X, and (R³)ₙ for a compound corresponds in each case to one row of Table A

Table 46: Compounds of formula I.A* in which R⁴ is F, R⁵ is H, Z is Q2, R^{6a} is 2-pyridyl, R^{6b} is H, and the combination of R¹, X, and (R³)ₙ for a compound corresponds in each case to one row of Table A

Table 47: Compounds of formula I.A* in which R⁴ is F, R⁵ is F, Z is Q2, R^{6a} is 2-pyridyl, R^{6b} is H, and the combination of R¹, X, and (R³)ₙ for a compound corresponds in each case to one row of Table A

Table 48: Compounds of formula I.A* in which R⁴ is F, R⁵ is Cl, Z is Q2, R^{6a} is 2-pyridyl, R^{6b} is H, and the combination of R¹, X, and (R³)ₙ for a compound corresponds in each case to one row of Table A

Table 49: Compounds of formula I.A* in which R⁴ is F, R⁵ is CH₃, Z is Q2, R^{6a} is 2-pyridyl, R^{6b} is H, and the combination of R¹, X, and (R³)ₙ for a compound corresponds in each case to one row of Table A

Table 50: Compounds of formula I.A* in which R⁴ is F, R⁵ is cC₃H₅, Z is Q2, R^{6a} is 2-pyridyl, R^{6b} is H, and the combination of R¹, X, and (R³)ₙ for a compound corresponds in each case to one row of Table A

Table 51: Compounds of formula I.A* in which R⁴ is F, R⁵ is H, Z is Q3, R^{6a} is 2-pyridyl, R^{6b} is H, and the combination of R¹, X, and (R³)ₙ for a compound corresponds in each case to one row of Table A

Table 52: Compounds of formula I.A* in which R⁴ is F, R⁵ is F, Z is Q3, R^{6a} is 2-pyridyl, R^{6b} is H, and the combination of R¹, X, and (R³)ₙ for a compound corresponds in each case to one row of Table A

Table 53: Compounds of formula I.A* in which R⁴ is F, R⁵ is Cl, Z is Q3, R^{6a} is 2-pyridyl, R^{6b} is H, and the combination of R¹, X, and (R³)ₙ for a compound corresponds in each case to one row of Table A

Table 54: Compounds of formula I.A* in which R⁴ is F, R⁵ is CH₃, Z is Q3, R^{6a} is 2-pyridyl, R^{6b} is H, and the combination of R¹, X, and (R³)ₙ for a compound corresponds in each case to one row of Table A

Table 55: Compounds of formula I.A* in which R⁴ is F, R⁵ is cC₃H₅, Z is Q3, R^{6a} is 2-pyridyl, R^{6b} is H, and the combination of R¹, X, and (R³)ₙ for a compound corresponds in each case to one row of Table A

Table 56: Compounds of formula I.A* in which R⁴ is F, R⁵ is H, Z is Q4, R^{6a} is 2-pyridyl, R^{6b} is H, and the combination of R¹, X, and (R³)ₙ for a compound corresponds in each case to one row of Table A

Table 57: Compounds of formula I.A* in which R⁴ is F, R⁵ is F, Z is Q4, R^{6a} is 2-pyridyl, R^{6b} is H, and the combination of R¹, X, and (R³)ₙ for a compound corresponds in each case to one row of Table A

Table 58: Compounds of formula I.A* in which R⁴ is F, R⁵ is Cl, Z is Q4, R^{6a} is 2-pyridyl, R^{6b} is H, and the combination of R¹, X, and (R³)ₙ for a compound corresponds in each case to one row of Table A

Table 59: Compounds of formula I.A* in which R⁴ is F, R⁵ is CH₃, Z is Q4, R^{6a} is 2-pyridyl, R^{6b} is H, and the combination of R¹, X, and (R³)ₙ for a compound corresponds in each case to one row of Table A

Table 60: Compounds of formula I.A* in which R⁴ is F, R⁵ is cC₃H₅, Z is Q4, R^{6a} is 2-pyridyl, R^{6b} is H, and the combination of R¹, X, and (R³)ₙ for a compound corresponds in each case to one row of Table A

Table 61: Compounds of formula I.A* in which R⁴ is F, R⁵ is H, Z is Q1, R^{6a} is 3-pyridyl, R^{6b} is H, and the combination of R¹, X, and (R³)ₙ for a compound corresponds in each case to one row of Table A

Table 62: Compounds of formula I.A* in which R⁴ is F, R⁵ is F, Z is Q1, R^{6a} is 3-pyridyl, R^{6b} is H, and the combination of R¹, X, and (R³)ₙ for a compound corresponds in each case to one row of Table A

Table 63: Compounds of formula I.A* in which R⁴ is F, R⁵ is Cl, Z is Q1, R^{6a} is 3-pyridyl, R^{6b} is H, and the combination of R¹, X, and (R³)ₙ for a compound corresponds in each case to one row of Table A

Table 64: Compounds of formula I.A* in which R⁴ is F, R⁵ is CH₃, Z is Q1, R^{6a} is 3-pyridyl, R^{6b} is H, and the combination of R¹, X, and (R³)ₙ for a compound corresponds in each case to one row of Table A

Table 65: Compounds of formula I.A* in which R⁴ is F, R⁵ is cC₃H₅, Z is Q1, R^{6a} is 3-pyridyl, R^{6b} is H, and the combination of R¹, X, and (R³)ₙ for a compound corresponds in each case to one row of Table A

Table 66: Compounds of formula I.A* in which R⁴ is F, R⁵ is H, Z is Q2, R^{6a} is 3-pyridyl, R^{6b} is H, and the combination of R¹, X, and (R³)ₙ for a compound corresponds in each case to one row of Table A

Table 67: Compounds of formula I.A* in which R⁴ is F, R⁵ is F, Z is Q2, R^{6a} is 3-pyridyl, R^{6b} is H, and the combination of R¹, X, and (R³)ₙ for a compound corresponds in each case to one row of Table A

Table 68: Compounds of formula I.A* in which R⁴ is F, R⁵ is Cl, Z is Q2, R^{6a} is 3-pyridyl, R^{6b} is H, and the combination of R¹, X, and (R³)ₙ for a compound corresponds in each case to one row of Table A

Table 69: Compounds of formula I.A* in which R⁴ is F, R⁵ is CH₃, Z is Q2, R^{6a} is 3-pyridyl, R^{6b} is H, and the combination of R¹, X, and (R³)ₙ for a compound corresponds in each case to one row of Table A

Table 70: Compounds of formula I.A* in which R⁴ is F, R⁵ is cC₃H₅, Z is Q2, R^{6a} is 3-pyridyl, R^{6b} is H, and the combination of R¹, X, and (R³)ₙ for a compound corresponds in each case to one row of Table A

Table 71: Compounds of formula I.A* in which R⁴ is F, R⁵ is H, Z is Q3, R^{6a} is 3-pyridyl, R^{6b} is H, and the combination of R¹, X, and (R³)ₙ for a compound corresponds in each case to one row of Table A

Table 72: Compounds of formula I.A* in which R⁴ is F, R⁵ is F, Z is Q3, R^{6a} is 3-pyridyl, R^{6b} is H, and the combination of R¹, X, and (R³)ₙ for a compound corresponds in each case to one row of Table A

Table 73: Compounds of formula I.A* in which R⁴ is F, R⁵ is Cl, Z is Q3, R^{6a} is 3-pyridyl, R^{6b} is H, and the combination of R¹, X, and (R³)ₙ for a compound corresponds in each case to one row of Table A

Table 74: Compounds of formula I.A* in which R⁴ is F, R⁵ is CH₃, Z is Q3, R^{6a} is 3-pyridyl, R^{6b} is H, and the combination of R¹, X, and (R³)ₙ for a compound corresponds in each case to one row of Table A

Table 75: Compounds of formula I.A* in which R⁴ is F, R⁵ is cC₃H₅, Z is Q3, R^{6a} is 3-pyridyl, R^{6b} is H, and the combination of R¹, X, and (R³)ₙ for a compound corresponds in each case to one row of Table A

Table 76: Compounds of formula I.A* in which R⁴ is F, R⁵ is H, Z is Q4, R^{6a} is 3-pyridyl, R^{6b} is H, and the combination of R¹, X, and (R³)ₙ for a compound corresponds in each case to one row of Table A

Table 77: Compounds of formula I.A* in which R⁴ is F, R⁵ is F, Z is Q4, R^{6a} is 3-pyridyl, R^{6b} is H, and the combination of R¹, X, and (R³)ₙ for a compound corresponds in each case to one row of Table A

Table 78: Compounds of formula I.A* in which R⁴ is F, R⁵ is Cl, Z is Q4, R^{6a} is 3-pyridyl, R^{6b} is H, and the combination of R¹, X, and (R³)ₙ for a compound corresponds in each case to one row of Table A

Table 79: Compounds of formula I.A* in which R⁴ is F, R⁵ is CH₃, Z is Q4, R^{6a} is 3-pyridyl, R^{6b} is H, and the combination of R¹, X, and (R³)ₙ for a compound corresponds in each case to one row of Table A

Table 80: Compounds of formula I.A* in which R⁴ is F, R⁵ is cC₃H₅, Z is Q4, R^{6a} is 3-pyridyl, R^{6b} is H, and the combination of R¹, X, and (R³)ₙ for a compound corresponds in each case to one row of Table A

Table 81: Compounds of formula I.A* in which R⁴ is Cl, R⁵ is H, Z is Q1, R^{6a} is 2-pyridyl, R^{6b} is H, and the combination of R¹, X, and (R³)ₙ for a compound corresponds in each case to one row of Table A

Table 82: Compounds of formula I.A* in which R⁴ is Cl, R⁵ is F, Z is Q1, R^{6a} is 2-pyridyl, R^{6b} is H, and the combination of R¹, X, and (R³)ₙ for a compound corresponds in each case to one row of Table A

Table 83: Compounds of formula I.A* in which R⁴ is Cl, R⁵ is Cl, Z is Q1, R^{6a} is 2-pyridyl, R^{6b} is H, and the combination of R¹, X, and (R³)ₙ for a compound corresponds in each case to one row of Table A

Table 84: Compounds of formula I.A* in which R⁴ is Cl, R⁵ is CH₃, Z is Q1, R^{6a} is 2-pyridyl, R^{6b} is H, and the combination of R¹, X, and (R³)ₙ for a compound corresponds in each case to one row of Table A

Table 85: Compounds of formula I.A* in which R⁴ is Cl, R⁵ is cC₃H₅, Z is Q1, R^{6a} is 2-pyridyl, R^{6b} is H, and the combination of R¹, X, and (R³)ₙ for a compound corresponds in each case to one row of Table A

Table 86: Compounds of formula I.A* in which R⁴ is Cl, R⁵ is H, Z is Q2, R^{6a} is 2-pyridyl, R^{6b} is H, and the combination of R¹, X, and (R³)ₙ for a compound corresponds in each case to one row of Table A

Table 87: Compounds of formula I.A* in which R⁴ is Cl, R⁵ is F, Z is Q2, R^{6a} is 2-pyridyl, R^{6b} is H, and the combination of R¹, X, and (R³)ₙ for a compound corresponds in each case to one row of Table A

Table 88: Compounds of formula I.A* in which R⁴ is Cl, R⁵ is Cl, Z is Q2, R^{6a} is 2-pyridyl, R^{6b} is H, and the combination of R¹, X, and (R³)ₙ for a compound corresponds in each case to one row of Table A

Table 89: Compounds of formula I.A* in which R⁴ is Cl, R⁵ is CH₃, Z is Q2, R^{6a} is 2-pyridyl, R^{6b} is H, and the combination of R¹, X, and (R³)ₙ for a compound corresponds in each case to one row of Table A

Table 90: Compounds of formula I.A* in which R⁴ is Cl, R⁵ is cC₃H₅, Z is Q2, R^{6a} is 2-pyridyl, R^{6b} is H, and the combination of R¹, X, and (R³)ₙ for a compound corresponds in each case to one row of Table A

Table 91: Compounds of formula I.A* in which R⁴ is Cl, R⁵ is H, Z is Q3, R^{6a} is 2-pyridyl, R^{6b} is H, and the combination of R¹, X, and (R³)ₙ for a compound corresponds in each case to one row of Table A

Table 92: Compounds of formula I.A* in which R⁴ is Cl, R⁵ is F, Z is Q3, R^{6a} is 2-pyridyl, R^{6b} is H, and the combination of R¹, X, and (R³)ₙ for a compound corresponds in each case to one row of Table A

Table 93: Compounds of formula I.A* in which R⁴ is Cl, R⁵ is Cl, Z is Q3, R^{6a} is 2-pyridyl, R^{6b} is H, and the combination of R¹, X, and (R³)ₙ for a compound corresponds in each case to one row of Table A

Table 94: Compounds of formula I.A* in which R⁴ is Cl, R⁵ is CH₃, Z is Q3, R^{6a} is 2-pyridyl, R^{6b} is H, and the combination of R¹, X, and (R³)ₙ for a compound corresponds in each case to one row of Table A

Table 95: Compounds of formula I.A* in which R⁴ is Cl, R⁵ is cC₃H₅, Z is Q3, R^{6a} is 2-pyridyl, R^{6b} is H, and the combination of R¹, X, and (R³)ₙ for a compound corresponds in each case to one row of Table A

Table 96: Compounds of formula I.A* in which R⁴ is Cl, R⁵ is H, Z is Q4, R^{6a} is 2-pyridyl, R^{6b} is H, and the combination of R¹, X, and (R³)ₙ for a compound corresponds in each case to one row of Table A

Table 97: Compounds of formula I.A* in which R⁴ is Cl, R⁵ is F, Z is Q4, R^{6a} is 2-pyridyl, R^{6b} is H, and the combination of R¹, X, and (R³)ₙ for a compound corresponds in each case to one row of Table A

Table 98: Compounds of formula I.A* in which R⁴ is Cl, R⁵ is Cl, Z is Q4, R^{6a} is 2-pyridyl, R^{6b} is H, and the combination of R¹, X, and (R³)ₙ for a compound corresponds in each case to one row of Table A

Table 99: Compounds of formula I.A* in which R⁴ is Cl, R⁵ is CH₃, Z is Q4, R^{6a} is 2-pyridyl, R^{6b} is H, and the combination of R¹, X, and (R³)ₙ for a compound corresponds in each case to one row of Table A

Table 100: Compounds of formula I.A* in which R⁴ is Cl, R⁵ is cC₃H₅, Z is Q4, R^{6a} is 2-pyridyl, R^{6b} is H, and the combination of R¹, X, and (R³)ₙ for a compound corresponds in each case to one row of Table A

Table 101: Compounds of formula I.A* in which R⁴ is Cl, R⁵ is H, Z is Q1, R^{6a} is 3-pyridyl, R^{6b} is H, and the combination of R¹, X, and (R³)ₙ for a compound corresponds in each case to one row of Table A

Table 102: Compounds of formula I.A* in which R⁴ is Cl, R⁵ is F, Z is Q1, R^{6a} is 3-pyridyl, R^{6b} is H, and the combination of R¹, X, and (R³)ₙ for a compound corresponds in each case to one row of Table A

Table 103: Compounds of formula I.A* in which R⁴ is Cl, R⁵ is Cl, Z is Q1, R^{6a} is 3-pyridyl, R^{6b} is H, and the combination of R¹, X, and (R³)ₙ for a compound corresponds in each case to one row of Table A

Table 104: Compounds of formula I.A* in which R⁴ is Cl, R⁵ is CH₃, Z is Q1, R^{6a} is 3-pyridyl, R^{6b} is H, and the combination of R¹, X, and (R³)ₙ for a compound corresponds in each case to one row of Table A

Table 105: Compounds of formula I.A* in which R⁴ is Cl, R⁵ is cC₃H₅, Z is Q1, R^{6a} is 3-pyridyl, R^{6b} is H, and the combination of R¹, X, and (R³)ₙ for a compound corresponds in each case to one row of Table A

Table 106: Compounds of formula I.A* in which R⁴ is Cl, R⁵ is H, Z is Q2, R^{6a} is 3-pyridyl, R^{6b} is H, and the combination of R¹, X, and (R³)ₙ for a compound corresponds in each case to one row of Table A

Table 107: Compounds of formula I.A* in which R⁴ is Cl, R⁵ is F, Z is Q2, R^{6a} is 3-pyridyl, R^{6b} is H, and the combination of R¹, X, and (R³)ₙ for a compound corresponds in each case to one row of Table A

Table 108: Compounds of formula I.A* in which R⁴ is Cl, R⁵ is Cl, Z is Q2, R^{6a} is 3-pyridyl, R^{6b} is H, and the combination of R¹, X, and (R³)ₙ for a compound corresponds in each case to one row of Table A

Table 109: Compounds of formula I.A* in which R⁴ is Cl, R⁵ is CH₃, Z is Q2, R^{6a} is 3-pyridyl, R^{6b} is H, and the combination of R¹, X, and (R³)ₙ for a compound corresponds in each case to one row of Table A

Table 110: Compounds of formula I.A* in which R⁴ is Cl, R⁵ is cC₃H₅, Z is Q2, R^{6a} is 3-pyridyl, R^{6b} is H, and the combination of R¹, X, and (R³)ₙ for a compound corresponds in each case to one row of Table A

Table 111: Compounds of formula I.A* in which R⁴ is Cl, R⁵ is H, Z is Q3, R^{6a} is 3-pyridyl, R^{6b} is H, and the combination of R¹, X, and (R³)ₙ for a compound corresponds in each case to one row of Table A

Table 112: Compounds of formula I.A* in which R⁴ is Cl, R⁵ is F, Z is Q3, R^{6a} is 3-pyridyl, R^{6b} is H, and the combination of R¹, X, and (R³)ₙ for a compound corresponds in each case to one row of Table A

Table 113: Compounds of formula I.A* in which R⁴ is Cl, R⁵ is Cl, Z is Q3, R^{6a} is 3-pyridyl, R^{6b} is H, and the combination of R¹, X, and (R³)ₙ for a compound corresponds in each case to one row of Table A

Table 114: Compounds of formula I.A* in which R⁴ is Cl, R⁵ is CH₃, Z is Q3, R^{6a} is 3-pyridyl, R^{6b} is H, and the combination of R¹, X, and (R³)ₙ for a compound corresponds in each case to one row of Table A

Table 115: Compounds of formula I.A* in which R⁴ is Cl, R⁵ is cC₃H₅, Z is Q3, R^{6a} is 3-pyridyl, R^{6b} is H, and the combination of R¹, X, and (R³)ₙ for a compound corresponds in each case to one row of Table A

Table 116: Compounds of formula I.A* in which R⁴ is Cl, R⁵ is H, Z is Q4, R^{6a} is 3-pyridyl, R^{6b} is H, and the combination of R¹, X, and (R³)ₙ for a compound corresponds in each case to one row of Table A

Table 117: Compounds of formula I.A* in which R⁴ is Cl, R⁵ is F, Z is Q4, R^{6a} is 3-pyridyl, R^{6b} is H, and the combination of R¹, X, and (R³)ₙ for a compound corresponds in each case to one row of Table A

Table 118: Compounds of formula I.A* in which R⁴ is Cl, R⁵ is Cl, Z is Q4, R^{6a} is 3-pyridyl, R^{6b} is H, and the combination of R¹, X, and (R³)ₙ for a compound corresponds in each case to one row of Table A

Table 119: Compounds of formula I.A* in which R⁴ is Cl, R⁵ is CH₃, Z is Q4, R^{6a} is 3-pyridyl, R^{6b} is H, and the combination of R¹, X, and (R³)ₙ for a compound corresponds in each case to one row of Table A

Table 120: Compounds of formula I.A* in which R⁴ is Cl, R⁵ is cC₃H₅, Z is Q4, R^{6a} is 3-pyridyl, R^{6b} is H, and the combination of R¹, X, and (R³)ₙ for a compound corresponds in each case to one row of Table A

Table 121: Compounds of formula I.A* in which R⁴ is CH₃, R⁵ is H, Z is Q1, R^{6a} is 2-pyridyl, R^{6b} is H, and the combination of R¹, X, and (R³)ₙ for a compound corresponds in each case to one row of Table A

Table 122: Compounds of formula I.A* in which R⁴ is CH₃, R⁵ is F, Z is Q1, R^{6a} is 2-pyridyl, R^{6b} is H, and the combination of R¹, X, and (R³)ₙ for a compound corresponds in each case to one row of Table A

Table 123: Compounds of formula I.A* in which R⁴ is CH₃, R⁵ is Cl, Z is Q1, R^{6a} is 2-pyridyl, R^{6b} is H, and the combination of R¹, X, and (R³)ₙ for a compound corresponds in each case to one row of Table A

Table 124: Compounds of formula I.A* in which R⁴ is CH₃, R⁵ is CH₃, Z is Q1, R^{6a} is 2-pyridyl, R^{6b} is H, and the combination of R¹, X, and (R³)ₙ for a compound corresponds in each case to one row of Table A

Table 125: Compounds of formula I.A* in which R⁴ is CH₃, R⁵ is cC₃H₅, Z is Q1, R^{6a} is 2-pyridyl, R^{6b} is H, and the combination of R¹, X, and (R³)ₙ for a compound corresponds in each case to one row of Table A

Table 126: Compounds of formula I.A* in which R⁴ is CH₃, R⁵ is H, Z is Q2, R^{6a} is 2-pyridyl, R^{6b} is H, and the combination of R¹, X, and (R³)ₙ for a compound corresponds in each case to one row of Table A

Table 127: Compounds of formula I.A* in which R⁴ is CH₃, R⁵ is F, Z is Q2, R^{6a} is 2-pyridyl, R^{6b} is H, and the combination of R¹, X, and (R³)ₙ for a compound corresponds in each case to one row of Table A

Table 128: Compounds of formula I.A* in which R⁴ is CH₃, R⁵ is Cl, Z is Q2, R^{6a} is 2-pyridyl, R^{6b} is H, and the combination of R¹, X, and (R³)ₙ for a compound corresponds in each case to one row of Table A

Table 129: Compounds of formula I.A* in which R⁴ is CH₃, R⁵ is CH₃, Z is Q2, R^{6a} is 2-pyridyl, R^{6b} is H, and the combination of R¹, X, and (R³)ₙ for a compound corresponds in each case to one row of Table A

Table 130: Compounds of formula I.A* in which R⁴ is CH₃, R⁵ is cC₃H₅, Z is Q2, R^{6a} is 2-pyridyl, R^{6b} is H, and the combination of R¹, X, and (R³)ₙ for a compound corresponds in each case to one row of Table A

Table 131: Compounds of formula I.A* in which R⁴ is CH₃, R⁵ is H, Z is Q3, R^{6a} is 2-pyridyl, R^{6b} is H, and the combination of R¹, X, and (R³)ₙ for a compound corresponds in each case to one row of Table A

Table 132: Compounds of formula I.A* in which R⁴ is CH₃, R⁵ is F, Z is Q3, R^{6a} is 2-pyridyl, R^{6b} is H, and the combination of R¹, X, and (R³)ₙ for a compound corresponds in each case to one row of Table A

Table 133: Compounds of formula I.A* in which R⁴ is CH₃, R⁵ is Cl, Z is Q3, R^{6a} is 2-pyridyl, R^{6b} is H, and the combination of R¹, X, and (R³)ₙ for a compound corresponds in each case to one row of Table A

Table 134: Compounds of formula I.A* in which R⁴ is CH₃, R⁵ is CH₃, Z is Q3, R^{6a} is 2-pyridyl, R^{6b} is H, and the combination of R¹, X, and (R³)ₙ for a compound corresponds in each case to one row of Table A

Table 135: Compounds of formula I.A* in which R⁴ is CH₃, R⁵ is cC₃H₅, Z is Q3, R^{6a} is 2-pyridyl, R^{6b} is H, and the combination of R¹, X, and (R³)ₙ for a compound corresponds in each case to one row of Table A

Table 136: Compounds of formula I.A* in which R⁴ is CH₃, R⁵ is H, Z is Q4, R^{6a} is 2-pyridyl, R^{6b} is H, and the combination of R¹, X, and (R³)ₙ for a compound corresponds in each case to one row of Table A

Table 137: Compounds of formula I.A* in which R⁴ is CH₃, R⁵ is F, Z is Q4, R^{6a} is 2-pyridyl, R^{6b} is H, and the combination of R¹, X, and (R³)ₙ for a compound corresponds in each case to one row of Table A

Table 138: Compounds of formula I.A* in which R⁴ is CH₃, R⁵ is Cl, Z is Q4, R^{6a} is 2-pyridyl, R^{6b} is H, and the combination of R¹, X, and (R³)ₙ for a compound corresponds in each case to one row of Table A

Table 139: Compounds of formula I.A* in which R⁴ is CH₃, R⁵ is CH₃, Z is Q4, R^{6a} is 2-pyridyl, R^{6b} is H, and the combination of R¹, X, and (R³)ₙ for a compound corresponds in each case to one row of Table A

Table 140: Compounds of formula I.A* in which R⁴ is CH₃, R⁵ is cC₃H₅, Z is Q4, R^{6a} is 2-pyridyl, R^{6b} is H, and the combination of R¹, X, and (R³)ₙ for a compound corresponds in each case to one row of Table A

Table 141: Compounds of formula I.A* in which R⁴ is CH₃, R⁵ is H, Z is Q1, R^{6a} is 3-pyridyl, R^{6b} is H, and the combination of R¹, X, and (R³)ₙ for a compound corresponds in each case to one row of Table A

Table 142: Compounds of formula I.A* in which R⁴ is CH₃, R⁵ is F, Z is Q1, R^{6a} is 3-pyridyl, R^{6b} is H, and the combination of R¹, X, and (R³)ₙ for a compound corresponds in each case to one row of Table A

Table 143: Compounds of formula I.A* in which R⁴ is CH₃, R⁵ is Cl, Z is Q1, R^{6a} is 3-pyridyl, R^{6b} is H, and the combination of R¹, X, and (R³)ₙ for a compound corresponds in each case to one row of Table A

Table 144: Compounds of formula I.A* in which R⁴ is CH₃, R⁵ is CH₃, Z is Q1, R^{6a} is 3-pyridyl, R^{6b} is H, and the combination of R¹, X, and (R³)ₙ for a compound corresponds in each case to one row of Table A

Table 145: Compounds of formula I.A* in which R⁴ is CH₃, R⁵ is cC₃H₅, Z is Q1, R^{6a} is 3-pyridyl, R^{6b} is H, and the combination of R¹, X, and (R³)ₙ for a compound corresponds in each case to one row of Table A

Table 146: Compounds of formula I.A* in which R⁴ is CH₃, R⁵ is H, Z is Q2, R^{6a} is 3-pyridyl, R^{6b} is H, and the combination of R¹, X, and (R³)ₙ for a compound corresponds in each case to one row of Table A

Table 147: Compounds of formula I.A* in which R⁴ is CH₃, R⁵ is F, Z is Q2, R^{6a} is 3-pyridyl, R^{6b} is H, and the combination of R¹, X, and (R³)ₙ for a compound corresponds in each case to one row of Table A

Table 148: Compounds of formula I.A* in which R⁴ is CH₃, R⁵ is Cl, Z is Q2, R^{6a} is 3-pyridyl, R^{6b} is H, and the combination of R¹, X, and (R³)ₙ for a compound corresponds in each case to one row of Table A

Table 149: Compounds of formula I.A* in which R⁴ is CH₃, R⁵ is CH₃, Z is Q2, R^{6a} is 3-pyridyl, R^{6b} is H, and the combination of R¹, X, and (R³)ₙ for a compound corresponds in each case to one row of Table A

Table 150: Compounds of formula I.A* in which R⁴ is CH₃, R⁵ is cC₃H₅, Z is Q2, R^{6a} is 3-pyridyl, R^{6b} is H, and the combination of R¹, X, and (R³)ₙ for a compound corresponds in each case to one row of Table A

Table 151: Compounds of formula I.A* in which R⁴ is CH₃, R⁵ is H, Z is Q3, R^{6a} is 3-pyridyl, R^{6b} is H, and the combination of R¹, X, and (R³)ₙ for a compound corresponds in each case to one row of Table A

Table 152: Compounds of formula I.A* in which R⁴ is CH₃, R⁵ is F, Z is Q3, R^{6a} is 3-pyridyl, R^{6b} is H, and the combination of R¹, X, and (R³)ₙ for a compound corresponds in each case to one row of Table A

Table 153: Compounds of formula I.A* in which R⁴ is CH₃, R⁵ is Cl, Z is Q3, R^{6a} is 3-pyridyl, R^{6b} is H, and the combination of R¹, X, and (R³)ₙ for a compound corresponds in each case to one row of Table A

Table 154: Compounds of formula I.A* in which R⁴ is CH₃, R⁵ is CH₃, Z is Q3, R^{6a} is 3-pyridyl, R^{6b} is H, and the combination of R¹, X, and (R³)ₙ for a compound corresponds in each case to one row of Table A

Table 155: Compounds of formula I.A* in which R⁴ is CH₃, R⁵ is cC₃H₅, Z is Q3, R^{6a} is 3-pyridyl, R^{6b} is H, and the combination of R¹, X, and (R³)ₙ for a compound corresponds in each case to one row of Table A

Table 156: Compounds of formula I.A* in which R⁴ is CH₃, R⁵ is H, Z is Q4, R^{6a} is 3-pyridyl, R^{6b} is H, and the combination of R¹, X, and (R³)ₙ for a compound corresponds in each case to one row of Table A

Table 157: Compounds of formula I.A* in which R⁴ is CH₃, R⁵ is F, Z is Q4, R^{6a} is 3-pyridyl, R^{6b} is H, and the combination of R¹, X, and (R³)ₙ for a compound corresponds in each case to one row of Table A

Table 158: Compounds of formula I.A* in which R⁴ is CH₃, R⁵ is Cl, Z is Q4, R^{6a} is 3-pyridyl, R^{6b} is H, and the combination of R¹, X, and (R³)ₙ for a compound corresponds in each case to one row of Table A

Table 159: Compounds of formula I.A* in which R⁴ is CH₃, R⁵ is CH₃, Z is Q4, R^{6a} is 3-pyridyl, R^{6b} is H, and the combination of R¹, X, and (R³)ₙ for a compound corresponds in each case to one row of Table A

Table 160: Compounds of formula I.A* in which R⁴ is CH₃, R⁵ is cC₃H₅, Z is Q4, R^{6a} is 3-pyridyl, R^{6b} is H, and the combination of R¹, X, and (R³)ₙ for a compound corresponds in each case to one row of Table A

Table 161: Compounds of formula I.A* in which R⁴ and R⁵ are H, Z is Q1, R^{6a} is 2-pyridyl, R^{6b} is F, and the combination of R¹, X, and (R³)ₙ for a compound corresponds in each case to one row of Table A

Table 162: Compounds of formula I.A* in which R⁴ is H, R⁵ is F, Z is Q1, R^{6a} is 2-pyridyl, R^{6b} is F, and the combination of R¹, X, and (R³)ₙ for a compound corresponds in each case to one row of Table A

Table 163: Compounds of formula I.A* in which R⁴ is H, R⁵ is Cl, Z is Q1, R^{6a} is 2-pyridyl, R^{6b} is F, and the combination of R¹, X, and (R³)ₙ for a compound corresponds in each case to one row of Table A

Table 164: Compounds of formula I.A* in which R⁴ is H, R⁵ is CH₃, Z is Q1, R^{6a} is 2-pyridyl, R^{6b} is F, and the combination of R¹, X, and (R³)ₙ for a compound corresponds in each case to one row of Table A

Table 165: Compounds of formula I.A* in which R⁴ is H, R⁵ is cC₃H₅, Z is Q1, R^{6a} is 2-pyridyl, R^{6b} is F, and the combination of R¹, X, and (R³)ₙ for a compound corresponds in each case to one row of Table A

Table 166: Compounds of formula I.A* in which R⁴ and R⁵ are H, Z is Q2, R^{6a} is 2-pyridyl, R^{6b} is F, and the combination of R¹, X, and (R³)ₙ for a compound corresponds in each case to one row of Table A

Table 167: Compounds of formula I.A* in which R⁴ is H, R⁵ is F, Z is Q2, R^{6a} is 2-pyridyl, R^{6b} is F, and the combination of R¹, X, and (R³)ₙ for a compound corresponds in each case to one row of Table A

Table 168: Compounds of formula I.A* in which R⁴ is H, R⁵ is Cl, Z is Q2, R^{6a} is 2-pyridyl, R^{6b} is F, and the combination of R¹, X, and (R³)ₙ for a compound corresponds in each case to one row of Table A

Table 169: Compounds of formula I.A* in which R⁴ is H, R⁵ is CH₃, Z is Q2, R^{6a} is 2-pyridyl, R^{6b} is F, and the combination of R¹, X, and (R³)ₙ for a compound corresponds in each case to one row of Table A

Table 170: Compounds of formula I.A* in which R⁴ is H, R⁵ is cC₃H₅, Z is Q2, R^{6a} is 2-pyridyl, R^{6b} is F, and the combination of R¹, X, and (R³)ₙ for a compound corresponds in each case to one row of Table A

Table 171: Compounds of formula I.A* in which R⁴ and R⁵ are H, Z is Q3, R^{6a} is 2-pyridyl, R^{6b} is F, and the combination of R¹, X, and (R³)ₙ for a compound corresponds in each case to one row of Table A

Table 172: Compounds of formula I.A* in which R⁴ is H, R⁵ is F, Z is Q3, R^{6a} is 2-pyridyl, R^{6b} is F, and the combination of R¹, X, and (R³)ₙ for a compound corresponds in each case to one row of Table A

Table 173: Compounds of formula I.A* in which R⁴ is H, R⁵ is Cl, Z is Q3, R^{6a} is 2-pyridyl, R^{6b} is F, and the combination of R¹, X, and (R³)ₙ for a compound corresponds in each case to one row of Table A

Table 174: Compounds of formula I.A* in which R⁴ is H, R⁵ is CH₃, Z is Q3, R^{6a} is 2-pyridyl, R^{6b} is F, and the combination of R¹, X, and (R³)ₙ for a compound corresponds in each case to one row of Table A

Table 175: Compounds of formula I.A* in which R⁴ is H, R⁵ is cC₃H₅, Z is Q3, R^{6a} is 2-pyridyl, R^{6b} is F, and the combination of R¹, X, and (R³)ₙ for a compound corresponds in each case to one row of Table A

Table 176: Compounds of formula I.A* in which R⁴ and R⁵ are H, Z is Q4, R^{6a} is 2-pyridyl, R^{6b} is F, and the combination of R¹, X, and (R³)ₙ for a compound corresponds in each case to one row of Table A

Table 177: Compounds of formula I.A* in which R⁴ is H, R⁵ is F, Z is Q4, R^{6a} is 2-pyridyl, R^{6b} is F, and the combination of R¹, X, and (R³)ₙ for a compound corresponds in each case to one row of Table A

Table 178: Compounds of formula I.A* in which R⁴ is H, R⁵ is Cl, Z is Q4, R^{6a} is 2-pyridyl, R^{6b} is F, and the combination of R¹, X, and (R³)ₙ for a compound corresponds in each case to one row of Table A

Table 179: Compounds of formula I.A* in which R⁴ is H, R⁵ is CH₃, Z is Q4, R^{6a} is 2-pyridyl, R^{6b} is F, and the combination of R¹, X, and (R³)ₙ for a compound corresponds in each case to one row of Table A

Table 180: Compounds of formula I.A* in which R⁴ is H, R⁵ is cC₃H₅, Z is Q4, R^{6a} is 2-pyridyl, R^{6b} is F, and the combination of R¹, X, and (R³)ₙ for a compound corresponds in each case to one row of Table A

Table 181: Compounds of formula I.A* in which R⁴ and R⁵ are H, Z is Q1, R^{6a} is 3-pyridyl, R^{6b} is F, and the combination of R¹, X, and (R³)ₙ for a compound corresponds in each case to one row of Table A

Table 182: Compounds of formula I.A* in which R⁴ is H, R⁵ is F, Z is Q1, R^{6a} is 3-pyridyl, R^{6b} is F, and the combination of R¹, X, and (R³)ₙ for a compound corresponds in each case to one row of Table A

Table 183: Compounds of formula I.A* in which R⁴ is H, R⁵ is Cl, Z is Q1, R^{6a} is 3-pyridyl, R^{6b} is F, and the combination of R¹, X, and (R³)ₙ for a compound corresponds in each case to one row of Table A

Table 184: Compounds of formula I.A* in which R⁴ is H, R⁵ is CH₃, Z is Q1, R^{6a} is 3-pyridyl, R^{6b} is F, and the combination of R¹, X, and (R³)ₙ for a compound corresponds in each case to one row of Table A

Table 185: Compounds of formula I.A* in which R⁴ is H, R⁵ is cC₃H₅, Z is Q1, R^{6a} is 3-pyridyl, R^{6b} is F, and the combination of R¹, X, and (R³)ₙ for a compound corresponds in each case to one row of Table A

Table 186: Compounds of formula I.A* in which R⁴ and R⁵ are H, Z is Q2, R^{6a} is 3-pyridyl, R^{6b} is F, and the combination of R¹, X, and (R³)ₙ for a compound corresponds in each case to one row of Table A

Table 187: Compounds of formula I.A* in which R⁴ is H, R⁵ is F, Z is Q2, R^{6a} is 3-pyridyl, R^{6b} is F, and the combination of R¹, X, and (R³)ₙ for a compound corresponds in each case to one row of Table A

Table 188: Compounds of formula I.A* in which R⁴ is H, R⁵ is Cl, Z is Q2, R^{6a} is 3-pyridyl, R^{6b} is F, and the combination of R¹, X, and (R³)ₙ for a compound corresponds in each case to one row of Table A

Table 189: Compounds of formula I.A* in which R⁴ is H, R⁵ is CH₃, Z is Q2, R^{6a} is 3-pyridyl, R^{6b} is F, and the combination of R¹, X, and (R³)ₙ for a compound corresponds in each case to one row of Table A

Table 190: Compounds of formula I.A* in which R⁴ is H, R⁵ is cC₃H₅, Z is Q2, R^{6a} is 3-pyridyl, R^{6b} is F, and the combination of R¹, X, and (R³)ₙ for a compound corresponds in each case to one row of Table A

Table 191: Compounds of formula I.A* in which R⁴ and R⁵ are H, Z is Q3, R^{6a} is 3-pyridyl, R^{6b} is F, and the combination of R¹, X, and (R³)ₙ for a compound corresponds in each case to one row of Table A

Table 192: Compounds of formula I.A* in which R⁴ is H, R⁵ is F, Z is Q3, R^{6a} is 3-pyridyl, R^{6b} is F, and the combination of R R¹, X, ¹ and (R³)ₙ for a compound corresponds in each case to one row of Table A

Table 193: Compounds of formula I.A* in which R⁴ is H, R⁵ is Cl, Z is Q3, R^{6a} is 3-pyridyl, R^{6b} is F, and the combination of R¹, X, and (R³)ₙ for a compound corresponds in each case to one row of Table A

Table 194: Compounds of formula I.A* in which R⁴ is H, R⁵ is CH₃, Z is Q3, R^{6a} is 3-pyridyl, R^{6b} is F, and the combination of R¹, X, and (R³)ₙ for a compound corresponds in each case to one row of Table A

Table 195: Compounds of formula I.A* in which R⁴ is H, R⁵ is cC₃H₅, Z is Q3, R^{6a} is 3-pyridyl, R^{6b} is F, and the combination of R¹, X, and (R³)ₙ for a compound corresponds in each case to one row of Table A

Table 196: Compounds of formula I.A* in which R⁴ and R⁵ are H, Z is Q4, R^{6a} is 3-pyridyl, R^{6b} is F, and the combination of R¹, X, and (R³)ₙ for a compound corresponds in each case to one row of Table A

Table 197: Compounds of formula I.A* in which R⁴ is H, R⁵ is F, Z is Q4, R^{6a} is 3-pyridyl, R^{6b} is F, and the combination of R¹, X, and (R³)ₙ for a compound corresponds in each case to one row of Table A

Table 198: Compounds of formula I.A* in which R⁴ is H, R⁵ is Cl, Z is Q4, R^{6a} is 3-pyridyl, R^{6b} is F, and the combination of R¹, X, and (R³)ₙ for a compound corresponds in each case to one row of Table A

Table 199: Compounds of formula I.A* in which R⁴ is H, R⁵ is CH₃, Z is Q4, R^{6a} is 3-pyridyl, R^{6b} is F, and the combination of R¹, X, and (R³)ₙ for a compound corresponds in each case to one row of Table A

Table 200: Compounds of formula I.A* in which R⁴ is H, R⁵ is cC₃H₅, Z is Q4, R^{6a} is 3-pyridyl, R^{6b} is F, and the combination of R¹, X, and (R³)ₙ for a compound corresponds in each case to one row of Table A

Table 201: Compounds of formula I.A* in which R⁴ is F, R⁵ is H, Z is Q1, R^{6a} is 2-pyridyl, R^{6b} is F, and the combination of R¹, X, and (R³)ₙ for a compound corresponds in each case to one row of Table A

Table 202: Compounds of formula I.A* in which R⁴ is F, R⁵ is F, Z is Q1, R^{6a} is 2-pyridyl, R^{6b} is F, and the combination of R¹, X, and (R³)ₙ for a compound corresponds in each case to one row of Table A

Table 203: Compounds of formula I.A* in which R⁴ is F, R⁵ is Cl, Z is Q1, R^{6a} is 2-pyridyl, R^{6b} is F, and the combination of R¹, X, and (R³)ₙ for a compound corresponds in each case to one row of Table A

Table 204: Compounds of formula I.A* in which R⁴ is F, R⁵ is CH₃, Z is Q1, R^{6a} is 2-pyridyl, R^{6b} is F, and the combination of R¹, X, and (R³)ₙ for a compound corresponds in each case to one row of Table A

Table 205: Compounds of formula I.A* in which R⁴ is F, R⁵ is cC₃H₅, Z is Q1, R^{6a} is 2-pyridyl, R^{6b} is F, and the combination of R¹, X, and (R³)ₙ for a compound corresponds in each case to one row of Table A

Table 206: Compounds of formula I.A* in which R⁴ is F, R⁵ is H, Z is Q2, R^{6a} is 2-pyridyl, R^{6b} is F, and the combination of R¹, X, and (R³)ₙ for a compound corresponds in each case to one row of Table A

Table 207: Compounds of formula I.A* in which R⁴ is F, R⁵ is F, Z is Q2, R^{6a} is 2-pyridyl, R^{6b} is F, and the combination of R¹, X, and (R³)ₙ for a compound corresponds in each case to one row of Table A

Table 208: Compounds of formula I.A* in which R⁴ is F, R⁵ is Cl, Z is Q2, R^{6a} is 2-pyridyl, R^{6b} is F, and the combination of R¹, X, and (R³)ₙ for a compound corresponds in each case to one row of Table A

Table 209: Compounds of formula I.A* in which R⁴ is F, R⁵ is CH₃, Z is Q2, R^{6a} is 2-pyridyl, R^{6b} is F, and the combination of R¹, X, and (R³)ₙ for a compound corresponds in each case to one row of Table A

Table 210: Compounds of formula I.A* in which R⁴ is F, R⁵ is cC₃H₅, Z is Q2, R^{6a} is 2-pyridyl, R^{6b} is F, and the combination of R¹, X, and (R³)ₙ for a compound corresponds in each case to one row of Table A

Table 211: Compounds of formula I.A* in which R⁴ is F, R⁵ is H, Z is Q3, R^{6a} is 2-pyridyl, R^{6b} is F, and the combination of R¹, X, and (R³)ₙ for a compound corresponds in each case to one row of Table A

Table 212: Compounds of formula I.A* in which R⁴ is F, R⁵ is F, Z is Q3, R^{6a} is 2-pyridyl, R^{6b} is F, and the combination of R¹, X, and (R³)ₙ for a compound corresponds in each case to one row of Table A

Table 213: Compounds of formula I.A* in which R⁴ is F, R⁵ is Cl, Z is Q3, R^{6a} is 2-pyridyl, R^{6b} is F, and the combination of R¹, X, and (R³)ₙ for a compound corresponds in each case to one row of Table A

Table 214: Compounds of formula I.A* in which R⁴ is F, R⁵ is CH₃, Z is Q3, R^{6a} is 2-pyridyl, R^{6b} is F, and the combination of R¹, X, and (R³)ₙ for a compound corresponds in each case to one row of Table A

Table 215: Compounds of formula I.A* in which R⁴ is F, R⁵ is cC₃H₅, Z is Q3, R^{6a} is 2-pyridyl, R^{6b} is F, and the combination of R¹, X, and (R³)ₙ for a compound corresponds in each case to one row of Table A

Table 216: Compounds of formula I.A* in which R⁴ is F, R⁵ is H, Z is Q4, R^{6a} is 2-pyridyl, R^{6b} is F, and the combination of R¹, X, and (R³)ₙ for a compound corresponds in each case to one row of Table A

Table 217: Compounds of formula I.A* in which R⁴ is F, R⁵ is F, Z is Q4, R^{6a} is 2-pyridyl, R^{6b} is F, and the combination of R¹, X, and (R³)ₙ for a compound corresponds in each case to one row of Table A

Table 218: Compounds of formula I.A* in which R⁴ is F, R⁵ is Cl, Z is Q4, R^{6a} is 2-pyridyl, R^{6b} is F, and the combination of R¹, X, and (R³)ₙ for a compound corresponds in each case to one row of Table A

Table 219: Compounds of formula I.A* in which R⁴ is F, R⁵ is CH₃, Z is Q4, R^{6a} is 2-pyridyl, R^{6b} is F, and the combination of R¹, X, and (R³)ₙ for a compound corresponds in each case to one row of Table A

Table 220: Compounds of formula I.A* in which R⁴ is F, R⁵ is cC₃H₅, Z is Q4, R^{6a} is 2-pyridyl, R^{6b} is F, and the combination of R¹, X, and (R³)ₙ for a compound corresponds in each case to one row of Table A

Table 221: Compounds of formula I.A* in which R⁴ is F, R⁵ is H, Z is Q1, R^{6a} is 3-pyridyl, R^{6b} is F, and the combination of R¹, X, and (R³)ₙ for a compound corresponds in each case to one row of Table A

Table 222: Compounds of formula I.A* in which R⁴ is F, R⁵ is F, Z is Q1, R^{6a} is 3-pyridyl, R^{6b} is F, and the combination of R¹, X, and (R³)ₙ for a compound corresponds in each case to one row of Table A

Table 223: Compounds of formula I.A* in which R⁴ is F, R⁵ is Cl, Z is Q1, R^{6a} is 3-pyridyl, R^{6b} is F, and the combination of R¹, X, and (R³)ₙ for a compound corresponds in each case to one row of Table A

Table 224: Compounds of formula I.A* in which R⁴ is F, R⁵ is CH₃, Z is Q1, R^{6a} is 3-pyridyl, R^{6b} is F, and the combination of R¹, X, and (R³)ₙ for a compound corresponds in each case to one row of Table A

Table 225: Compounds of formula I.A* in which R⁴ is F, R⁵ is cC₃H₅, Z is Q1, R^{6a} is 3-pyridyl, R^{6b} is F, and the combination of R¹, X, and (R³)ₙ for a compound corresponds in each case to one row of Table A

Table 226: Compounds of formula I.A* in which R⁴ is F, R⁵ is H, Z is Q2, R^{6a} is 3-pyridyl, R^{6b} is F, and the combination of R¹, X, and (R³)ₙ for a compound corresponds in each case to one row of Table A

Table 227: Compounds of formula I.A* in which R⁴ is F, R⁵ is F, Z is Q2, R^{6a} is 3-pyridyl, R^{6b} is F, and the combination of R¹, X, and (R³)ₙ for a compound corresponds in each case to one row of Table A

Table 228: Compounds of formula I.A* in which R⁴ is F, R⁵ is Cl, Z is Q2, R^{6a} is 3-pyridyl, R^{6b} is F, and the combination of R¹, X, and (R³)ₙ for a compound corresponds in each case to one row of Table A

Table 229: Compounds of formula I.A* in which R⁴ is F, R⁵ is CH₃, Z is Q2, R^{6a} is 3-pyridyl, R^{6b} is F, and the combination of R¹, X, and (R³)ₙ for a compound corresponds in each case to one row of Table A

Table 230: Compounds of formula I.A* in which R⁴ is F, R⁵ is cC₃H₅, Z is Q2, R^{6a} is 3-pyridyl, R^{6b} is F, and the combination of R¹, X, and (R³)ₙ for a compound corresponds in each case to one row of Table A

Table 231: Compounds of formula I.A* in which R⁴ is F, R⁵ is H, Z is Q3, R^{6a} is 3-pyridyl, R^{6b} is F, and the combination of R¹, X, and (R³)ₙ for a compound corresponds in each case to one row of Table A

Table 232: Compounds of formula I.A* in which R⁴ is F, R⁵ is F, Z is Q3, R^{6a} is 3-pyridyl, R^{6b} is F, and the combination of R¹, X, and (R³)ₙ for a compound corresponds in each case to one row of Table A

Table 233: Compounds of formula I.A* in which R⁴ is F, R⁵ is Cl, Z is Q3, R^{6a} is 3-pyridyl, R^{6b} is F, and the combination of R¹, X, and (R³)ₙ for a compound corresponds in each case to one row of Table A

Table 234: Compounds of formula I.A* in which R⁴ is F, R⁵ is CH₃, Z is Q3, R^{6a} is 3-pyridyl, R^{6b} is F, and the combination of R¹, X, and (R³)ₙ for a compound corresponds in each case to one row of Table A

Table 235: Compounds of formula I.A* in which R⁴ is F, R⁵ is cC₃H₅, Z is Q3, R^{6a} is 3-pyridyl, R^{6b} is F, and the combination of R¹, X, and (R³)ₙ for a compound corresponds in each case to one row of Table A

Table 236: Compounds of formula I.A* in which R⁴ is F, R⁵ is H, Z is Q4, R^{6a} is 3-pyridyl, R^{6b} is F, and the combination of R¹, X, and (R³)ₙ for a compound corresponds in each case to one row of Table A

Table 237: Compounds of formula I.A* in which R⁴ is F, R⁵ is F, Z is Q4, R^{6a} is 3-pyridyl, R^{6b} is F, and the combination of R¹, X, and (R³)ₙ for a compound corresponds in each case to one row of Table A

Table 238: Compounds of formula I.A* in which R⁴ is F, R⁵ is Cl, Z is Q4, R^{6a} is 3-pyridyl, R^{6b} is F, and the combination of R¹, X, and (R³)ₙ for a compound corresponds in each case to one row of Table A

Table 239: Compounds of formula I.A* in which R⁴ is F, R⁵ is CH₃, Z is Q4, R^{6a} is 3-pyridyl, R^{6b} is F, and the combination of R¹, X, and (R³)ₙ for a compound corresponds in each case to one row of Table A

Table 240: Compounds of formula I.A* in which R⁴ is F, R⁵ is cC₃H₅, Z is Q4, R^{6a} is 3-pyridyl, R^{6b} is F, and the combination of R¹, X, and (R³)ₙ for a compound corresponds in each case to one row of Table A

Table 241: Compounds of formula I.A* in which R⁴ is Cl, R⁵ is H, Z is Q1, R^{6a} is 2-pyridyl, R^{6b} is F, and the combination of R¹, X, and (R³)ₙ for a compound corresponds in each case to one row of Table A

Table 242: Compounds of formula I.A* in which R⁴ is Cl, R⁵ is F, Z is Q1, R^{6a} is 2-pyridyl, R^{6b} is F, and the combination of R¹, X, and (R³)ₙ for a compound corresponds in each case to one row of Table A

Table 243: Compounds of formula I.A* in which R⁴ is Cl, R⁵ is Cl, Z is Q1, R^{6a} is 2-pyridyl, R^{6b} is F, and the combination of R¹, X, and (R³)ₙ for a compound corresponds in each case to one row of Table A

Table 244: Compounds of formula I.A* in which R⁴ is Cl, R⁵ is CH₃, Z is Q1, R^{6a} is 2-pyridyl, R^{6b} is F, and the combination of R¹, X, and (R³)ₙ for a compound corresponds in each case to one row of Table A

Table 245: Compounds of formula I.A* in which R⁴ is Cl, R⁵ is cC₃H₅, Z is Q1, R^{6a} is 2-pyridyl, R^{6b} is F, and the combination of R¹, X, and (R³)ₙ for a compound corresponds in each case to one row of Table A

Table 246: Compounds of formula I.A* in which R⁴ is Cl, R⁵ is H, Z is Q2, R^{6a} is 2-pyridyl, R^{6b} is F, and the combination of R¹, X, and (R³)ₙ for a compound corresponds in each case to one row of Table A

Table 247: Compounds of formula I.A* in which R⁴ is Cl, R⁵ is F, Z is Q2, R^{6a} is 2-pyridyl, R^{6b} is F, and the combination of R¹, X, and (R³)ₙ for a compound corresponds in each case to one row of Table A

Table 248: Compounds of formula I.A* in which R⁴ is Cl, R⁵ is Cl, Z is Q2, R^{6a} is 2-pyridyl, R^{6b} is F, and the combination of R¹, X, and (R³)ₙ for a compound corresponds in each case to one row of Table A

Table 249: Compounds of formula I.A* in which R⁴ is Cl, R⁵ is CH₃, Z is Q2, R^{6a} is 2-pyridyl, R^{6b} is F, and the combination of R¹, X, and (R³)ₙ for a compound corresponds in each case to one row of Table A

Table 250: Compounds of formula I.A* in which R⁴ is Cl, R⁵ is cC₃H₅, Z is Q2, R^{6a} is 2-pyridyl, R^{6b} is F, and the combination of R¹, X, and (R³)ₙ for a compound corresponds in each case to one row of Table A

Table 251: Compounds of formula I.A* in which R⁴ is Cl, R⁵ is H, Z is Q3, R^{6a} is 2-pyridyl, R^{6b} is F, and the combination of R¹, X, and (R³)ₙ for a compound corresponds in each case to one row of Table A

Table 252: Compounds of formula I.A* in which R⁴ is Cl, R⁵ is F, Z is Q3, R^{6a} is 2-pyridyl, R^{6b} is F, and the combination of R¹, X, and (R³)ₙ for a compound corresponds in each case to one row of Table A

Table 253: Compounds of formula I.A* in which R⁴ is Cl, R⁵ is Cl, Z is Q3, R^{6a} is 2-pyridyl, R^{6b} is F, and the combination of R¹, X, and (R³)ₙ for a compound corresponds in each case to one row of Table A

Table 254: Compounds of formula I.A* in which R⁴ is Cl, R⁵ is CH₃, Z is Q3, R^{6a} is 2-pyridyl, R^{6b} is F, and the combination of R¹, X, and (R³)ₙ for a compound corresponds in each case to one row of Table A

Table 255: Compounds of formula I.A* in which R⁴ is Cl, R⁵ is cC₃H₅, Z is Q3, R^{6a} is 2-pyridyl, R^{6b} is F, and the combination of R¹, X, and (R³)ₙ for a compound corresponds in each case to one row of Table A

Table 256: Compounds of formula I.A* in which R⁴ is Cl, R⁵ is H, Z is Q4, R^{6a} is 2-pyridyl, R^{6b} is F, and the combination of R¹, X, and (R³)ₙ for a compound corresponds in each case to one row of Table A

Table 257: Compounds of formula I.A* in which R⁴ is Cl, R⁵ is F, Z is Q4, R^{6a} is 2-pyridyl, R^{6b} is F, and the combination of R¹, X, and (R³)ₙ for a compound corresponds in each case to one row of Table A

Table 258: Compounds of formula I.A* in which R⁴ is Cl, R⁵ is Cl, Z is Q4, R^{6a} is 2-pyridyl, R^{6b} is F, and the combination of R¹, X, and (R³)ₙ for a compound corresponds in each case to one row of Table A

Table 259: Compounds of formula I.A* in which R⁴ is Cl, R⁵ is CH₃, Z is Q4, R^{6a} is 2-pyridyl, R^{6b} is F, and the combination of R¹, X, and (R³)ₙ for a compound corresponds in each case to one row of Table A

Table 260: Compounds of formula I.A* in which R⁴ is Cl, R⁵ is cC₃H₅, Z is Q4, R^{6a} is 2-pyridyl, R^{6b} is F, and the combination of R¹, X, and (R³)ₙ for a compound corresponds in each case to one row of Table A

Table 261: Compounds of formula I.A* in which R⁴ is Cl, R⁵ is H, Z is Q1, R^{6a} is 3-pyridyl, R^{6b} is F, and the combination of R¹, X, and (R³)ₙ for a compound corresponds in each case to one row of Table A

Table 262: Compounds of formula I.A* in which R⁴ is Cl, R⁵ is F, Z is Q1, R^{6a} is 3-pyridyl, R^{6b} is F, and the combination of R¹, X, and (R³)ₙ for a compound corresponds in each case to one row of Table A

Table 263: Compounds of formula I.A* in which R⁴ is Cl, R⁵ is Cl, Z is Q1, R^{6a} is 3-pyridyl, R^{6b} is F, and the combination of R¹, X, and (R³)ₙ for a compound corresponds in each case to one row of Table A

Table 264: Compounds of formula I.A* in which R⁴ is Cl, R⁵ is CH₃, Z is Q1, R^{6a} is 3-pyridyl, R^{6b} is F, and the combination of R¹, X, and (R³)ₙ for a compound corresponds in each case to one row of Table A

Table 265: Compounds of formula I.A* in which R⁴ is Cl, R⁵ is cC₃H₅, Z is Q1, R^{6a} is 3-pyridyl, R^{6b} is F, and the combination of R¹, X, and (R³)ₙ for a compound corresponds in each case to one row of Table A

Table 266: Compounds of formula I.A* in which R⁴ is Cl, R⁵ is H, Z is Q2, R^{6a} is 3-pyridyl, R^{6b} is F, and the combination of R¹, X, and (R³)ₙ for a compound corresponds in each case to one row of Table A

Table 267: Compounds of formula I.A* in which R⁴ is Cl, R⁵ is F, Z is Q2, R^{6a} is 3-pyridyl, R^{6b} is F, and the combination of R¹, X, and (R³)ₙ for a compound corresponds in each case to one row of Table A

Table 268: Compounds of formula I.A* in which R⁴ is Cl, R⁵ is Cl, Z is Q2, R^{6a} is 3-pyridyl, R^{6b} is F, and the combination of R¹, X, and (R³)ₙ for a compound corresponds in each case to one row of Table A

Table 269: Compounds of formula I.A* in which R⁴ is Cl, R⁵ is CH₃, Z is Q2, R^{6a} is 3-pyridyl, R^{6b} is F, and the combination of R¹, X, and (R³)ₙ for a compound corresponds in each case to one row of Table A

Table 270: Compounds of formula I.A* in which R⁴ is Cl, R⁵ is cC₃H₅, Z is Q2, R^{6a} is 3-pyridyl, R^{6b} is F, and the combination of R¹, X, and (R³)ₙ for a compound corresponds in each case to one row of Table A

Table 271: Compounds of formula I.A* in which R⁴ is Cl, R⁵ is H, Z is Q3, R^{6a} is 3-pyridyl, R^{6b} is F, and the combination of R¹, X, and (R³)ₙ for a compound corresponds in each case to one row of Table A

Table 272: Compounds of formula I.A* in which R⁴ is Cl, R⁵ is F, Z is Q3, R^{6a} is 3-pyridyl, R^{6b} is F, and the combination of R¹, X, and (R³)ₙ for a compound corresponds in each case to one row of Table A

Table 273: Compounds of formula I.A* in which R⁴ is Cl, R⁵ is Cl, Z is Q3, R^{6a} is 3-pyridyl, R^{6b} is F, and the combination of R¹, X, and (R³)ₙ for a compound corresponds in each case to one row of Table A

Table 274: Compounds of formula I.A* in which R⁴ is Cl, R⁵ is CH₃, Z is Q3, R^{6a} is 3-pyridyl, R^{6b} is F, and the combination of R¹, X, and (R³)ₙ for a compound corresponds in each case to one row of Table A

Table 275: Compounds of formula I.A* in which R⁴ is Cl, R⁵ is cC₃H₅, Z is Q3, R^{6a} is 3-pyridyl, R^{6b} is F, and the combination of R¹, X, and (R³)ₙ for a compound corresponds in each case to one row of Table A

Table 276: Compounds of formula I.A* in which R⁴ is Cl, R⁵ is H, Z is Q4, R^{6a} is 3-pyridyl, R^{6b} is F, and the combination of R¹, X, and (R³)ₙ for a compound corresponds in each case to one row of Table A

Table 277: Compounds of formula I.A* in which R⁴ is Cl, R⁵ is F, Z is Q4, R^{6a} is 3-pyridyl, R^{6b} is F, and the combination of R¹, X, and (R³)ₙ for a compound corresponds in each case to one row of Table A

Table 278: Compounds of formula I.A* in which R⁴ is Cl, R⁵ is Cl, Z is Q4, R^{6a} is 3-pyridyl, R^{6b} is F, and the combination of R¹, X, and (R³)ₙ for a compound corresponds in each case to one row of Table A

Table 279: Compounds of formula I.A* in which R⁴ is Cl, R⁵ is CH₃, Z is Q4, R^{6a} is 3-pyridyl, R^{6b} is F, and the combination of R¹, X, and (R³)ₙ for a compound corresponds in each case to one row of Table A

Table 280: Compounds of formula I.A* in which R⁴ is Cl, R⁵ is cC₃H₅, Z is Q4, R^{6a} is 3-pyridyl, R^{6b} is F, and the combination of R¹, X, and (R³)ₙ for a compound corresponds in each case to one row of Table A

Table 281: Compounds of formula I.A* in which R⁴ is CH₃, R⁵ is H, Z is Q1, R^{6a} is 2-pyridyl, R^{6b} is F, and the combination of R¹, X, and (R³)ₙ for a compound corresponds in each case to one row of Table A

Table 282: Compounds of formula I.A* in which R⁴ is CH₃, R⁵ is F, Z is Q1, R^{6a} is 2-pyridyl, R^{6b} is F, and the combination of R¹, X, and (R³)ₙ for a compound corresponds in each case to one row of Table A

Table 283: Compounds of formula I.A* in which R⁴ is CH₃, R⁵ is Cl, Z is Q1, R^{6a} is 2-pyridyl, R^{6b} is F, and the combination of R¹, X, and (R³)ₙ for a compound corresponds in each case to one row of Table A

Table 284: Compounds of formula I.A* in which R⁴ is CH₃, R⁵ is CH₃, Z is Q1, R^{6a} is 2-pyridyl, R^{6b} is F, and the combination of R¹, X, and (R³)ₙ for a compound corresponds in each case to one row of Table A

Table 285: Compounds of formula I.A* in which R⁴ is CH₃, R⁵ is cC₃H₅, Z is Q1, R^{6a} is 2-pyridyl, R^{6b} is F, and the combination of R¹, X, and (R³)ₙ for a compound corresponds in each case to one row of Table A

Table 286: Compounds of formula I.A* in which R⁴ is CH₃, R⁵ is H, Z is Q2, R^{6a} is 2-pyridyl, R^{6b} is F, and the combination of R¹, X, and (R³)ₙ for a compound corresponds in each case to one row of Table A

Table 287: Compounds of formula I.A* in which R⁴ is CH₃, R⁵ is F, Z is Q2, R^{6a} is 2-pyridyl, R^{6b} is F, and the combination of R¹, X, and (R³)ₙ for a compound corresponds in each case to one row of Table A

Table 288: Compounds of formula I.A* in which R⁴ is CH₃, R⁵ is Cl, Z is Q2, R^{6a} is 2-pyridyl, R^{6b} is F, and the combination of R¹, X, and (R³)ₙ for a compound corresponds in each case to one row of Table A

Table 289: Compounds of formula I.A* in which R⁴ is CH₃, R⁵ is CH₃, Z is Q2, R^{6a} is 2-pyridyl, R^{6b} is F, and the combination of R¹, X, and (R³)ₙ for a compound corresponds in each case to one row of Table A

Table 290: Compounds of formula I.A* in which R⁴ is CH₃, R⁵ is cC₃H₅, Z is Q2, R^{6a} is 2-pyridyl, R^{6b} is F, and the combination of R¹, X, and (R³)ₙ for a compound corresponds in each case to one row of Table A

Table 291: Compounds of formula I.A* in which R⁴ is CH₃, R⁵ is H, Z is Q3, R^{6a} is 2-pyridyl, R^{6b} is F, and the combination of R¹, X, and (R³)ₙ for a compound corresponds in each case to one row of Table A

Table 292: Compounds of formula I.A* in which R⁴ is CH₃, R⁵ is F, Z is Q3, R^{6a} is 2-pyridyl, R^{6b} is F, and the combination of R¹, X, and (R³)ₙ for a compound corresponds in each case to one row of Table A

Table 293: Compounds of formula I.A* in which R⁴ is CH₃, R⁵ is Cl, Z is Q3, R^{6a} is 2-pyridyl, R^{6b} is F, and the combination of R¹, X, and (R³)ₙ for a compound corresponds in each case to one row of Table A

Table 294: Compounds of formula I.A* in which R⁴ is CH₃, R⁵ is CH₃, Z is Q3, R^{6a} is 2-pyridyl, R^{6b} is F, and the combination of R¹, X, and (R³)ₙ for a compound corresponds in each case to one row of Table A

Table 295: Compounds of formula I.A* in which R⁴ is CH₃, R⁵ is cC₃H₅, Z is Q3, R^{6a} is 2-pyridyl, R^{6b} is F, and the combination of R¹, X, and (R³)ₙ for a compound corresponds in each case to one row of Table A

Table 296: Compounds of formula I.A* in which R⁴ is CH₃, R⁵ is H, Z is Q4, R^{6a} is 2-pyridyl, R^{6b} is F, and the combination of R¹, X, and (R³)ₙ for a compound corresponds in each case to one row of Table A

Table 297: Compounds of formula I.A* in which R⁴ is CH₃, R⁵ is F, Z is Q4, R^{6a} is 2-pyridyl, R^{6b} is F, and the combination of R¹, X, and (R³)ₙ for a compound corresponds in each case to one row of Table A

Table 298: Compounds of formula I.A* in which R⁴ is CH₃, R⁵ is Cl, Z is Q4, R^{6a} is 2-pyridyl, R^{6b} is F, and the combination of R¹, X, and (R³)ₙ for a compound corresponds in each case to one row of Table A

Table 299: Compounds of formula I.A* in which R⁴ is CH₃, R⁵ is CH₃, Z is Q4, R^{6a} is 2-pyridyl, R^{6b} is F, and the combination of R¹, X, and (R³)ₙ for a compound corresponds in each case to one row of Table A

Table 300: Compounds of formula I.A* in which R⁴ is CH₃, R⁵ is cC₃H₅, Z is Q4, R^{6a} is 2-pyridyl, R^{6b} is F, and the combination of R¹, X, and (R³)ₙ for a compound corresponds in each case to one row of Table A

Table 301: Compounds of formula I.A* in which R⁴ is CH₃, R⁵ is H, Z is Q1, R^{6a} is 3-pyridyl, R^{6b} is F, and the combination of R¹, X, and (R³)ₙ for a compound corresponds in each case to one row of Table A

Table 302: Compounds of formula I.A* in which R⁴ is CH₃, R⁵ is F, Z is Q1, R^{6a} is 3-pyridyl, R^{6b} is F, and the combination of R¹, X, and (R³)ₙ for a compound corresponds in each case to one row of Table A

Table 303: Compounds of formula I.A* in which R⁴ is CH₃, R⁵ is Cl, Z is Q1, R^{6a} is 3-pyridyl, R^{6b} is F, and the combination of R¹, X, and (R³)ₙ for a compound corresponds in each case to one row of Table A

Table 304: Compounds of formula I.A* in which R⁴ is CH₃, R⁵ is CH₃, Z is Q1, R^{6a} is 3-pyridyl, R^{6b} is F, and the combination of R¹, X, and (R³)ₙ for a compound corresponds in each case to one row of Table A

Table 305: Compounds of formula I.A* in which R⁴ is CH₃, R⁵ is cC₃H₅, Z is Q1, R^{6a} is 3-pyridyl, R^{6b} is F, and the combination of R¹, X, and (R³)ₙ for a compound corresponds in each case to one row of Table A

Table 306: Compounds of formula I.A* in which R⁴ is CH₃, R⁵ is H, Z is Q2, R^{6a} is 3-pyridyl, R^{6b} is F, and the combination of R¹, X, and (R³)ₙ for a compound corresponds in each case to one row of Table A

Table 307: Compounds of formula I.A* in which R⁴ is CH₃, R⁵ is F, Z is Q2, R^{6a} is 3-pyridyl, R^{6b} is F, and the combination of R¹, X, and (R³)ₙ for a compound corresponds in each case to one row of Table A

Table 308: Compounds of formula I.A* in which R⁴ is CH₃, R⁵ is Cl, Z is Q2, R^{6a} is 3-pyridyl, R^{6b} is F, and the combination of R¹, X, and (R³)ₙ for a compound corresponds in each case to one row of Table A

Table 309: Compounds of formula I.A* in which R⁴ is CH₃, R⁵ is CH₃, Z is Q2, R^{6a} is 3-pyridyl, R^{6b} is F, and the combination of R¹, X, and (R³)ₙ for a compound corresponds in each case to one row of Table A

Table 310: Compounds of formula I.A* in which R⁴ is CH₃, R⁵ is cC₃H₅, Z is Q2, R^{6a} is 3-pyridyl, R^{6b} is F, and the combination of R¹, X, and (R³)ₙ for a compound corresponds in each case to one row of Table A

Table 311: Compounds of formula I.A* in which R⁴ is CH₃, R⁵ is H, Z is Q3, R^{6a} is 3-pyridyl, R^{6b} is F, and the combination of R¹, X, and (R³)ₙ for a compound corresponds in each case to one row of Table A

Table 312: Compounds of formula I.A* in which R⁴ is CH₃, R⁵ is F, Z is Q3, R^{6a} is 3-pyridyl, R^{6b} is F, and the combination of R¹, X, and (R³)ₙ for a compound corresponds in each case to one row of Table A

Table 313: Compounds of formula I.A* in which R⁴ is CH₃, R⁵ is Cl, Z is Q3, R^{6a} is 3-pyridyl, R^{6b} is F, and the combination of R¹, X, and (R³)ₙ for a compound corresponds in each case to one row of Table A

Table 314: Compounds of formula I.A* in which R⁴ is CH₃, R⁵ is CH₃, Z is Q3, R^{6a} is 3-pyridyl, R^{6b} is F, and the combination of R¹, X, and (R³)ₙ for a compound corresponds in each case to one row of Table A

Table 315: Compounds of formula I.A* in which R⁴ is CH₃, R⁵ is cC₃H₅, Z is Q3, R^{6a} is 3-pyridyl, R^{6b} is F, and the combination of R¹, X, and (R³)ₙ for a compound corresponds in each case to one row of Table A

Table 316: Compounds of formula I.A* in which R⁴ is CH₃, R⁵ is H, Z is Q4, R^{6a} is 3-pyridyl, R^{6b} is F, and the combination of R¹, X, and (R³)ₙ for a compound corresponds in each case to one row of Table A

Table 317: Compounds of formula I.A* in which R⁴ is CH₃, R⁵ is F, Z is Q4, R^{6a} is 3-pyridyl, R^{6b} is F, and the combination of R¹, X, and (R³)ₙ for a compound corresponds in each case to one row of Table A

Table 318: Compounds of formula I.A* in which R⁴ is CH₃, R⁵ is Cl, Z is Q4, R^{6a} is 3-pyridyl, R^{6b} is F, and the combination of R¹, X, and (R³)ₙ for a compound corresponds in each case to one row of Table A

Table 319: Compounds of formula I.A* in which R⁴ is CH₃, R⁵ is CH₃, Z is Q4, R^{6a} is 3-pyridyl, R^{6b} is F, and the combination of R¹, X, and (R³)ₙ for a compound corresponds in each case to one row of Table A

Table 320: Compounds of formula I.A* in which R⁴ is CH₃, R⁵ is cC₃H₅, Z is Q4, R^{6a} is 3-pyridyl, R^{6b} is F, and the combination of R¹, X, and (R³)ₙ for a compound corresponds in each case to one row of Table A

Table 321: Compounds of formula I.A* in which R⁴ and R⁵ are H, Z is Q1, R^{6a} is 2-pyridyl, R^{6b} is Cl, and the combination of R¹, X, and (R³)ₙ for a compound corresponds in each case to one row of Table A

Table 322: Compounds of formula I.A* in which R⁴ is H, R⁵ is F, Z is Q1, R^{6a} is 2-pyridyl, R^{6b} is Cl, and the combination of R¹, X, and (R³)ₙ for a compound corresponds in each case to one row of Table A

Table 323: Compounds of formula I.A* in which R⁴ is H, R⁵ is Cl, Z is Q1, R^{6a} is 2-pyridyl, R^{6b} is Cl, and the combination of R¹, X, and (R³)ₙ for a compound corresponds in each case to one row of Table A

Table 324: Compounds of formula I.A* in which R⁴ is H, R⁵ is CH₃, Z is Q1, R^{6a} is 2-pyridyl, R^{6b} is Cl, and the combination of R¹, X, and (R³)ₙ for a compound corresponds in each case to one row of Table A

Table 325: Compounds of formula I.A* in which R⁴ is H, R⁵ is cC₃H₅, Z is Q1, R^{6a} is 2-pyridyl, R^{6b} is Cl, and the combination of R¹, X, and (R³)ₙ for a compound corresponds in each case to one row of Table A

Table 326: Compounds of formula I.A* in which R⁴ and R⁵ are H, Z is Q2, R^{6a} is 2-pyridyl, R^{6b} is Cl, and the combination of R¹, X, and (R³)ₙ for a compound corresponds in each case to one row of Table A

Table 327: Compounds of formula I.A* in which R⁴ is H, R⁵ is F, Z is Q2, R^{6a} is 2-pyridyl, R^{6b} is Cl, and the combination of R¹, X, and (R³)ₙ for a compound corresponds in each case to one row of Table A

Table 328: Compounds of formula I.A* in which R⁴ is H, R⁵ is Cl, Z is Q2, R^{6a} is 2-pyridyl, R^{6b} is Cl, and the combination of R¹, X, and (R³)ₙ for a compound corresponds in each case to one row of Table A

Table 329: Compounds of formula I.A* in which R⁴ is H, R⁵ is CH₃, Z is Q2, R^{6a} is 2-pyridyl, R^{6b} is Cl, and the combination of R¹, X, and (R³)ₙ for a compound corresponds in each case to one row of Table A

Table 330: Compounds of formula I.A* in which R⁴ is H, R⁵ is cC₃H₅, Z is Q2, R^{6a} is 2-pyridyl, R^{6b} is Cl, and the combination of R¹, X, and (R³)ₙ for a compound corresponds in each case to one row of Table A

Table 331: Compounds of formula I.A* in which R⁴ and R⁵ are H, Z is Q3, R^{6a} is 2-pyridyl, R^{6b} is Cl, and the combination of R¹, X, and (R³)ₙ for a compound corresponds in each case to one row of Table A

Table 332: Compounds of formula I.A* in which R⁴ is H, R⁵ is F, Z is Q3, R^{6a} is 2-pyridyl, R^{6b} is Cl, and the combination of R¹, X, and (R³)ₙ for a compound corresponds in each case to one row of Table A

Table 333: Compounds of formula I.A* in which R⁴ is H, R⁵ is Cl, Z is Q3, R^{6a} is 2-pyridyl, R^{6b} is Cl, and the combination of R¹, X, and (R³)ₙ for a compound corresponds in each case to one row of Table A

Table 334: Compounds of formula I.A* in which R⁴ is H, R⁵ is CH₃, Z is Q3, R^{6a} is 2-pyridyl, R^{6b} is Cl, and the combination of R¹, X, and (R³)ₙ for a compound corresponds in each case to one row of Table A

Table 335: Compounds of formula I.A* in which R⁴ is H, R⁵ is cC₃H₅, Z is Q3, R^{6a} is 2-pyridyl, R^{6b} is Cl, and the combination of R¹, X, and (R³)ₙ for a compound corresponds in each case to one row of Table A

Table 336: Compounds of formula I.A* in which R⁴ and R⁵ are H, Z is Q4, R^{6a} is 2-pyridyl, R^{6b} is Cl, and the combination of R¹, X, and (R³)ₙ for a compound corresponds in each case to one row of Table A

Table 337: Compounds of formula I.A* in which R⁴ is H, R⁵ is F, Z is Q4, R^{6a} is 2-pyridyl, R^{6b} is Cl, and the combination of R¹, X, and (R³)ₙ for a compound corresponds in each case to one row of Table A

Table 338: Compounds of formula I.A* in which R⁴ is H, R⁵ is Cl, Z is Q4, R^{6a} is 2-pyridyl, R^{6b} is Cl, and the combination of R¹, X, and (R³)ₙ for a compound corresponds in each case to one row of Table A

Table 339: Compounds of formula I.A* in which R⁴ is H, R⁵ is CH₃, Z is Q4, R^{6a} is 2-pyridyl, R^{6b} is Cl, and the combination of R¹, X, and (R³)ₙ for a compound corresponds in each case to one row of Table A

Table 340: Compounds of formula I.A* in which R⁴ is H, R⁵ is cC₃H₅, Z is Q4, R^{6a} is 2-pyridyl, R^{6b} is Cl, and the combination of R¹, X, and (R³)ₙ for a compound corresponds in each case to one row of Table A

Table 341: Compounds of formula I.A* in which R⁴ and R⁵ are H, Z is Q1, R^{6a} is 3-pyridyl, R^{6b} is Cl, and the combination of R¹, X, and (R³)ₙ for a compound corresponds in each case to one row of Table A

Table 342: Compounds of formula I.A* in which R⁴ is H, R⁵ is F, Z is Q1, R^{6a} is 3-pyridyl, R^{6b} is Cl, and the combination of R¹, X, and (R³)ₙ for a compound corresponds in each case to one row of Table A

Table 343: Compounds of formula I.A* in which R⁴ is H, R⁵ is Cl, Z is Q1, R^{6a} is 3-pyridyl, R^{6b} is Cl, and the combination of R¹, X, and (R³)ₙ for a compound corresponds in each case to one row of Table A

Table 344: Compounds of formula I.A* in which R⁴ is H, R⁵ is CH₃, Z is Q1, R^{6a} is 3-pyridyl, R^{6b} is Cl, and the combination of R¹, X, and (R³)ₙ for a compound corresponds in each case to one row of Table A

Table 345: Compounds of formula I.A* in which R⁴ is H, R⁵ is cC₃H₅, Z is Q1, R^{6a} is 3-pyridyl, R^{6b} is Cl, and the combination of R¹, X, and (R³)ₙ for a compound corresponds in each case to one row of Table A

Table 346: Compounds of formula I.A* in which R⁴ and R⁵ are H, Z is Q2, R^{6a} is 3-pyridyl, R^{6b} is Cl, and the combination of R¹, X, and (R³)ₙ for a compound corresponds in each case to one row of Table A

Table 347: Compounds of formula I.A* in which R⁴ is H, R⁵ is F, Z is Q2, R^{6a} is 3-pyridyl, R^{6b} is Cl, and the combination of R¹, X, and (R³)ₙ for a compound corresponds in each case to one row of Table A

Table 348: Compounds of formula I.A* in which R⁴ is H, R⁵ is Cl, Z is Q2, R^{6a} is 3-pyridyl, R^{6b} is Cl, and the combination of R¹, X, and (R³)ₙ for a compound corresponds in each case to one row of Table A

Table 349: Compounds of formula I.A* in which R⁴ is H, R⁵ is CH₃, Z is Q2, R^{6a} is 3-pyridyl, R^{6b} is Cl, and the combination of R¹, X, and (R³)ₙ for a compound corresponds in each case to one row of Table A

Table 350: Compounds of formula I.A* in which R⁴ is H, R⁵ is cC₃H₅, Z is Q2, R^{6a} is 3-pyridyl, R^{6b} is Cl, and the combination of R¹, X, and (R³)ₙ for a compound corresponds in each case to one row of Table A

Table 351: Compounds of formula I.A* in which R⁴ and R⁵ are H, Z is Q3, R^{6a} is 3-pyridyl, R^{6b} is Cl, and the combination of R¹, X, and (R³)ₙ for a compound corresponds in each case to one row of Table A

Table 352: Compounds of formula I.A* in which R⁴ is H, R⁵ is F, Z is Q3, R^{6a} is 3-pyridyl, R^{6b} is Cl, and the combination of R¹, X, and (R³)ₙ for a compound corresponds in each case to one row of Table A

Table 353: Compounds of formula I.A* in which R⁴ is H, R⁵ is Cl, Z is Q3, R^{6a} is 3-pyridyl, R^{6b} is Cl, and the combination of R¹, X, and (R³)ₙ for a compound corresponds in each case to one row of Table A

Table 354: Compounds of formula I.A* in which R⁴ is H, R⁵ is CH₃, Z is Q3, R^{6a} is 3-pyridyl, R^{6b} is Cl, and the combination of R¹, X, and (R³)ₙ for a compound corresponds in each case to one row of Table A

Table 355: Compounds of formula I.A* in which R⁴ is H, R⁵ is cC₃H₅, Z is Q3, R^{6a} is 3-pyridyl, R^{6b} is Cl, and the combination of R¹, X, and (R³)ₙ for a compound corresponds in each case to one row of Table A

Table 356: Compounds of formula I.A* in which R⁴ and R⁵ are H, Z is Q4, R^{6a} is 3-pyridyl, R^{6b} is Cl, and the combination of R¹, X, and (R³)ₙ for a compound corresponds in each case to one row of Table A

Table 357: Compounds of formula I.A* in which R⁴ is H, R⁵ is F, Z is Q4, R^{6a} is 3-pyridyl, R^{6b} is Cl, and the combination of R¹, X, and (R³)ₙ for a compound corresponds in each case to one row of Table A

Table 358: Compounds of formula I.A* in which R⁴ is H, R⁵ is Cl, Z is Q4, R^{6a} is 3-pyridyl, R^{6b} is Cl, and the combination of R¹, X, and (R³)ₙ for a compound corresponds in each case to one row of Table A

Table 359: Compounds of formula I.A* in which R⁴ is H, R⁵ is CH₃, Z is Q4, R^{6a} is 3-pyridyl, R^{6b} is Cl, and the combination of R¹, X, and (R³)ₙ for a compound corresponds in each case to one row of Table A

Table 360: Compounds of formula I.A* in which R⁴ is H, R⁵ is cC₃H₅, Z is Q4, R^{6a} is 3-pyridyl, R^{6b} is Cl, and the combination of R¹, X, and (R³)ₙ for a compound corresponds in each case to one row of Table A

Table 361: Compounds of formula I.A* in which R⁴ is F, R⁵ is H, Z is Q1, R^{6a} is 2-pyridyl, R^{6b} is Cl, and the combination of R¹, X, and (R³)ₙ for a compound corresponds in each case to one row of Table A

Table 362: Compounds of formula I.A* in which R⁴ is F, R⁵ is F, Z is Q1, R^{6a} is 2-pyridyl, R^{6b} is Cl, and the combination of R¹, X, and (R³)ₙ for a compound corresponds in each case to one row of Table A

Table 363: Compounds of formula I.A* in which R⁴ is F, R⁵ is Cl, Z is Q1, R^{6a} is 2-pyridyl, R^{6b} is Cl, and the combination of R¹, X, and (R³)ₙ for a compound corresponds in each case to one row of Table A

Table 364: Compounds of formula I.A* in which R⁴ is F, R⁵ is CH₃, Z is Q1, R^{6a} is 2-pyridyl, R^{6b} is Cl, and the combination of R¹, X, and (R³)ₙ for a compound corresponds in each case to one row of Table A

Table 365: Compounds of formula I.A* in which R⁴ is F, R⁵ is cC₃H₅, Z is Q1, R^{6a} is 2-pyridyl, R^{6b} is Cl, and the combination of R¹, X, and (R³)ₙ for a compound corresponds in each case to one row of Table A

Table 366: Compounds of formula I.A* in which R⁴ is F, R⁵ is H, Z is Q2, R^{6a} is 2-pyridyl, R^{6b} is Cl, and the combination of R¹, X, and (R³)ₙ for a compound corresponds in each case to one row of Table A

Table 367: Compounds of formula I.A* in which R⁴ is F, R⁵ is F, Z is Q2, R^{6a} is 2-pyridyl, R^{6b} is Cl, and the combination of R¹, X, and (R³)ₙ for a compound corresponds in each case to one row of Table A

Table 368: Compounds of formula I.A* in which R⁴ is F, R⁵ is Cl, Z is Q2, R^{6a} is 2-pyridyl, R^{6b} is Cl, and the combination of R¹, X, and (R³)ₙ for a compound corresponds in each case to one row of Table A

Table 369: Compounds of formula I.A* in which R⁴ is F, R⁵ is CH₃, Z is Q2, R^{6a} is 2-pyridyl, R^{6b} is Cl, and the combination of R¹, X, and (R³)ₙ for a compound corresponds in each case to one row of Table A

Table 370: Compounds of formula I.A* in which R⁴ is F, R⁵ is cC₃H₅, Z is Q2, R^{6a} is 2-pyridyl, R^{6b} is Cl, and the combination of R¹, X, and (R³)ₙ for a compound corresponds in each case to one row of Table A

Table 371: Compounds of formula I.A* in which R⁴ is F, R⁵ is H, Z is Q3, R^{6a} is 2-pyridyl, R^{6b} is Cl, and the combination of R¹, X, and (R³)ₙ for a compound corresponds in each case to one row of Table A

Table 372: Compounds of formula I.A* in which R⁴ is F, R⁵ is F, Z is Q3, R^{6a} is 2-pyridyl, R^{6b} is Cl, and the combination of R¹, X, and (R³)ₙ for a compound corresponds in each case to one row of Table A

Table 373: Compounds of formula I.A* in which R⁴ is F, R⁵ is Cl, Z is Q3, R^{6a} is 2-pyridyl, R^{6b} is Cl, and the combination of R¹, X, and (R³)ₙ for a compound corresponds in each case to one row of Table A

Table 374: Compounds of formula I.A* in which R⁴ is F, R⁵ is CH₃, Z is Q3, R^{6a} is 2-pyridyl, R^{6b} is Cl, and the combination of R¹, X, and (R³)ₙ for a compound corresponds in each case to one row of Table A

Table 375: Compounds of formula I.A* in which R⁴ is F, R⁵ is cC₃H₅, Z is Q3, R^{6a} is 2-pyridyl, R^{6b} is Cl, and the combination of R¹, X, and (R³)ₙ for a compound corresponds in each case to one row of Table A

Table 376: Compounds of formula I.A* in which R⁴ is F, R⁵ is H, Z is Q4, R^{6a} is 2-pyridyl, R^{6b} is Cl, and the combination of R¹, X, and (R³)ₙ for a compound corresponds in each case to one row of Table A

Table 377: Compounds of formula I.A* in which R⁴ is F, R⁵ is F, Z is Q4, R^{6a} is 2-pyridyl, R^{6b} is Cl, and the combination of R¹, X, and (R³)ₙ for a compound corresponds in each case to one row of Table A

Table 378: Compounds of formula I.A* in which R⁴ is F, R⁵ is Cl, Z is Q4, R^{6a} is 2-pyridyl, R^{6b} is Cl, and the combination of R¹, X, and (R³)ₙ for a compound corresponds in each case to one row of Table A

Table 379: Compounds of formula I.A* in which R⁴ is F, R⁵ is CH₃, Z is Q4, R^{6a} is 2-pyridyl, R^{6b} is Cl, and the combination of R¹, X, and (R³)ₙ for a compound corresponds in each case to one row of Table A

Table 380: Compounds of formula I.A* in which R⁴ is F, R⁵ is cC₃H₅, Z is Q4, R^{6a} is 2-pyridyl, R^{6b} is Cl, and the combination of R¹, X, and (R³)ₙ for a compound corresponds in each case to one row of Table A

Table 381: Compounds of formula I.A* in which R⁴ is F, R⁵ is H, Z is Q1, R^{6a} is 3-pyridyl, R^{6b} is Cl, and the combination of R¹, X, and (R³)ₙ for a compound corresponds in each case to one row of Table A

Table 382: Compounds of formula I.A* in which R⁴ is F, R⁵ is F, Z is Q1, R^{6a} is 3-pyridyl, R^{6b} is Cl, and the combination of R¹, X, and (R³)ₙ for a compound corresponds in each case to one row of Table A

Table 383: Compounds of formula I.A* in which R⁴ is F, R⁵ is Cl, Z is Q1, R^{6a} is 3-pyridyl, R^{6b} is Cl, and the combination of R¹, X, and (R³)ₙ for a compound corresponds in each case to one row of Table A

Table 384: Compounds of formula I.A* in which R⁴ is F, R⁵ is CH₃, Z is Q1, R^{6a} is 3-pyridyl, R^{6b} is Cl, and the combination of R¹, X, and (R³)ₙ for a compound corresponds in each case to one row of Table A

Table 385: Compounds of formula I.A* in which R⁴ is F, R⁵ is cC₃H₅, Z is Q1, R^{6a} is 3-pyridyl, R^{6b} is Cl, and the combination of R¹, X, and (R³)ₙ for a compound corresponds in each case to one row of Table A

Table 386: Compounds of formula I.A* in which R⁴ is F, R⁵ is H, Z is Q2, R^{6a} is 3-pyridyl, R^{6b} is Cl, and the combination of R¹, X, and (R³)ₙ for a compound corresponds in each case to one row of Table A

Table 387: Compounds of formula I.A* in which R⁴ is F, R⁵ is F, Z is Q2, R^{6a} is 3-pyridyl, R^{6b} is Cl, and the combination of R¹, X, and (R³)ₙ for a compound corresponds in each case to one row of Table A

Table 388: Compounds of formula I.A* in which R⁴ is F, R⁵ is Cl, Z is Q2, R^{6a} is 3-pyridyl, R^{6b} is Cl, and the combination of R¹, X, and (R³)ₙ for a compound corresponds in each case to one row of Table A

Table 389: Compounds of formula I.A* in which R⁴ is F, R⁵ is CH₃, Z is Q2, R^{6a} is 3-pyridyl, R^{6b} is Cl, and the combination of R¹, X, and (R³)ₙ for a compound corresponds in each case to one row of Table A

Table 390: Compounds of formula I.A* in which R⁴ is F, R⁵ is cC₃H₅, Z is Q2, R^{6a} is 3-pyridyl, R^{6b} is Cl, and the combination of R¹, X, and (R³)ₙ for a compound corresponds in each case to one row of Table A

Table 391: Compounds of formula I.A* in which R⁴ is F, R⁵ is H, Z is Q3, R^{6a} is 3-pyridyl, R^{6b} is Cl, and the combination of R¹, X, and (R³)ₙ for a compound corresponds in each case to one row of Table A

Table 392: Compounds of formula I.A* in which R⁴ is F, R⁵ is F, Z is Q3, R^{6a} is 3-pyridyl, R^{6b} is Cl, and the combination of R¹, X, and (R³)ₙ for a compound corresponds in each case to one row of Table A

Table 393: Compounds of formula I.A* in which R⁴ is F, R⁵ is Cl, Z is Q3, R^{6a} is 3-pyridyl, R^{6b} is Cl, and the combination of R¹, X, and (R³)ₙ for a compound corresponds in each case to one row of Table A

Table 394: Compounds of formula I.A* in which R⁴ is F, R⁵ is CH₃, Z is Q3, R^{6a} is 3-pyridyl, R^{6b} is Cl, and the combination of R¹, X, and (R³)ₙ for a compound corresponds in each case to one row of Table A

Table 395: Compounds of formula I.A* in which R⁴ is F, R⁵ is cC₃H₅, Z is Q3, R^{6a} is 3-pyridyl, R^{6b} is Cl, and the combination of R¹, X, and (R³)ₙ for a compound corresponds in each case to one row of Table A

Table 396: Compounds of formula I.A* in which R⁴ is F, R⁵ is H, Z is Q4, R^{6a} is 3-pyridyl, R^{6b} is Cl, and the combination of R¹, X, and (R³)ₙ for a compound corresponds in each case to one row of Table A

Table 397: Compounds of formula I.A* in which R⁴ is F, R⁵ is F, Z is Q4, R^{6a} is 3-pyridyl, R^{6b} is Cl, and the combination of R¹, X, and (R³)ₙ for a compound corresponds in each case to one row of Table A

Table 398: Compounds of formula I.A* in which R⁴ is F, R⁵ is Cl, Z is Q4, R^{6a} is 3-pyridyl, R^{6b} is Cl, and the combination of R¹, X, and (R³)ₙ for a compound corresponds in each case to one row of Table A

Table 399: Compounds of formula I.A* in which R⁴ is F, R⁵ is CH₃, Z is Q4, R^{6a} is 3-pyridyl, R^{6b} is Cl, and the combination of R¹, X, and (R³)ₙ for a compound corresponds in each case to one row of Table A

Table 400: Compounds of formula I.A* in which R⁴ is F, R⁵ is cC₃H₅, Z is Q4, R^{6a} is 3-pyridyl, R^{6b} is Cl, and the combination of R¹, X, and (R³)ₙ for a compound corresponds in each case to one row of Table A

Table 401: Compounds of formula I.A* in which R⁴ is Cl, R⁵ is H, Z is Q1, R^{6a} is 2-pyridyl, R^{6b} is Cl, and the combination of R¹, X, and (R³)ₙ for a compound corresponds in each case to one row of Table A

Table 402: Compounds of formula I.A* in which R⁴ is Cl, R⁵ is F, Z is Q1, R^{6a} is 2-pyridyl, R^{6b} is Cl, and the combination of R¹, X, and (R³)ₙ for a compound corresponds in each case to one row of Table A

Table 403: Compounds of formula I.A* in which R⁴ is Cl, R⁵ is Cl, Z is Q1, R^{6a} is 2-pyridyl, R^{6b} is Cl, and the combination of R¹, X, and (R³)ₙ for a compound corresponds in each case to one row of Table A

Table 404: Compounds of formula I.A* in which R⁴ is Cl, R⁵ is CH₃, Z is Q1, R^{6a} is 2-pyridyl, R^{6b} is Cl, and the combination of R¹, X, and (R³)ₙ for a compound corresponds in each case to one row of Table A

Table 405: Compounds of formula I.A* in which R⁴ is Cl, R⁵ is cC₃H₅, Z is Q1, R^{6a} is 2-pyridyl, R^{6b} is Cl, and the combination of R¹, X, and (R³)ₙ for a compound corresponds in each case to one row of Table A

Table 406: Compounds of formula I.A* in which R⁴ is Cl, R⁵ is H, Z is Q2, R^{6a} is 2-pyridyl, R^{6b} is Cl, and the combination of R¹, X, and (R³)ₙ for a compound corresponds in each case to one row of Table A

Table 407: Compounds of formula I.A* in which R⁴ is Cl, R⁵ is F, Z is Q2, R^{6a} is 2-pyridyl, R^{6b} is Cl, and the combination of R¹, X, and (R³)ₙ for a compound corresponds in each case to one row of Table A

Table 408: Compounds of formula I.A* in which R⁴ is Cl, R⁵ is Cl, Z is Q2, R^{6a} is 2-pyridyl, R^{6b} is Cl, and the combination of R¹, X, and (R³)ₙ for a compound corresponds in each case to one row of Table A

Table 409: Compounds of formula I.A* in which R⁴ is Cl, R⁵ is CH₃, Z is Q2, R^{6a} is 2-pyridyl, R^{6b} is Cl, and the combination of R¹, X, and (R³)ₙ for a compound corresponds in each case to one row of Table A

Table 410: Compounds of formula I.A* in which R⁴ is Cl, R⁵ is cC₃H₅, Z is Q2, R^{6a} is 2-pyridyl, R^{6b} is Cl, and the combination of R¹, X, and (R³)ₙ for a compound corresponds in each case to one row of Table A

Table 411: Compounds of formula I.A* in which R⁴ is Cl, R⁵ is H, Z is Q3, R^{6a} is 2-pyridyl, R^{6b} is Cl, and the combination of R¹, X, and (R³)ₙ for a compound corresponds in each case to one row of Table A

Table 412: Compounds of formula I.A* in which R⁴ is Cl, R⁵ is F, Z is Q3, R^{6a} is 2-pyridyl, R^{6b} is Cl, and the combination of R¹, X, and (R³)ₙ for a compound corresponds in each case to one row of Table A

Table 413: Compounds of formula I.A* in which R⁴ is Cl, R⁵ is Cl, Z is Q3, R^{6a} is 2-pyridyl, R^{6b} is Cl, and the combination of R¹, X, and (R³)ₙ for a compound corresponds in each case to one row of Table A

Table 414: Compounds of formula I.A* in which R⁴ is Cl, R⁵ is CH₃, Z is Q3, R^{6a} is 2-pyridyl, R^{6b} is Cl, and the combination of R¹, X, and (R³)ₙ for a compound corresponds in each case to one row of Table A

Table 415: Compounds of formula I.A* in which R⁴ is Cl, R⁵ is cC₃H₅, Z is Q3, R^{6a} is 2-pyridyl, R^{6b} is Cl, and the combination of R¹, X, and (R³)ₙ for a compound corresponds in each case to one row of Table A

Table 416: Compounds of formula I.A* in which R⁴ is Cl, R⁵ is H, Z is Q4, R^{6a} is 2-pyridyl, R^{6b} is Cl, and the combination of R¹, X, and (R³)ₙ for a compound corresponds in each case to one row of Table A

Table 417: Compounds of formula I.A* in which R⁴ is Cl, R⁵ is F, Z is Q4, R^{6a} is 2-pyridyl, R^{6b} is Cl, and the combination of R¹, X, and (R³)ₙ for a compound corresponds in each case to one row of Table A

Table 418: Compounds of formula I.A* in which R⁴ is Cl, R⁵ is Cl, Z is Q4, R^{6a} is 2-pyridyl, R^{6b} is Cl, and the combination of R¹, X, and (R³)ₙ for a compound corresponds in each case to one row of Table A

Table 419: Compounds of formula I.A* in which R⁴ is Cl, R⁵ is CH₃, Z is Q4, R^{6a} is 2-pyridyl, R^{6b} is Cl, and the combination of R¹, X, and (R³)ₙ for a compound corresponds in each case to one row of Table A

Table 420: Compounds of formula I.A* in which R⁴ is Cl, R⁵ is cC₃H₅, Z is Q4, R^{6a} is 2-pyridyl, R^{6b} is Cl, and the combination of R¹, X, and (R³)ₙ for a compound corresponds in each case to one row of Table A

Table 421: Compounds of formula I.A* in which R⁴ is Cl, R⁵ is H, Z is Q1, R^{6a} is 3-pyridyl, R^{6b} is Cl, and the combination of R¹, X, and (R³)ₙ for a compound corresponds in each case to one row of Table A

Table 422: Compounds of formula I.A* in which R⁴ is Cl, R⁵ is F, Z is Q1, R^{6a} is 3-pyridyl, R^{6b} is Cl, and the combination of R¹, X, and (R³)ₙ for a compound corresponds in each case to one row of Table A

Table 423: Compounds of formula I.A* in which R⁴ is Cl, R⁵ is Cl, Z is Q1, R^{6a} is 3-pyridyl, R^{6b} is Cl, and the combination of R¹, X, and (R³)ₙ for a compound corresponds in each case to one row of Table A

Table 424: Compounds of formula I.A* in which R⁴ is Cl, R⁵ is CH₃, Z is Q1, R^{6a} is 3-pyridyl, R^{6b} is Cl, and the combination of R¹, X, and (R³)ₙ for a compound corresponds in each case to one row of Table A

Table 425: Compounds of formula I.A* in which R⁴ is Cl, R⁵ is cC₃H₅, Z is Q1, R^{6a} is 3-pyridyl, R^{6b} is Cl, and the combination of R¹, X, and (R³)ₙ for a compound corresponds in each case to one row of Table A

Table 426: Compounds of formula I.A* in which R⁴ is Cl, R⁵ is H, Z is Q2, R^{6a} is 3-pyridyl, R^{6b} is Cl, and the combination of R¹, X, and (R³)ₙ for a compound corresponds in each case to one row of Table A

Table 427: Compounds of formula I.A* in which R⁴ is Cl, R⁵ is F, Z is Q2, R^{6a} is 3-pyridyl, R^{6b} is Cl, and the combination of R¹, X, and (R³)ₙ for a compound corresponds in each case to one row of Table A

Table 428: Compounds of formula I.A* in which R⁴ is Cl, R⁵ is Cl, Z is Q2, R^{6a} is 3-pyridyl, R^{6b} is Cl, and the combination of R¹, X, and (R³)ₙ for a compound corresponds in each case to one row of Table A

Table 429: Compounds of formula I.A* in which R⁴ is Cl, R⁵ is CH₃, Z is Q2, R^{6a} is 3-pyridyl, R^{6b} is Cl, and the combination of R¹, X, and (R³)ₙ for a compound corresponds in each case to one row of Table A

Table 430: Compounds of formula I.A* in which R⁴ is Cl, R⁵ is cC₃H₅, Z is Q2, R^{6a} is 3-pyridyl, R^{6b} is Cl, and the combination of R¹, X, and (R³)ₙ for a compound corresponds in each case to one row of Table A

Table 431: Compounds of formula I.A* in which R⁴ is Cl, R⁵ is H, Z is Q3, R^{6a} is 3-pyridyl, R^{6b} is Cl, and the combination of R¹, X, and (R³)ₙ for a compound corresponds in each case to one row of Table A

Table 432: Compounds of formula I.A* in which R⁴ is Cl, R⁵ is F, Z is Q3, R^{6a} is 3-pyridyl, R^{6b} is Cl, and the combination of R¹, X, and (R³)ₙ for a compound corresponds in each case to one row of Table A

Table 433: Compounds of formula I.A* in which R⁴ is Cl, R⁵ is Cl, Z is Q3, R^{6a} is 3-pyridyl, R^{6b} is Cl, and the combination of R¹, X, and (R³)ₙ for a compound corresponds in each case to one row of Table A

Table 434: Compounds of formula I.A* in which R⁴ is Cl, R⁵ is CH₃, Z is Q3, R^{6a} is 3-pyridyl, R^{6b} is Cl, and the combination of R¹, X, and (R³)ₙ for a compound corresponds in each case to one row of Table A

Table 435: Compounds of formula I.A* in which R⁴ is Cl, R⁵ is cC₃H₅, Z is Q3, R^{6a} is 3-pyridyl, R^{6b} is Cl, and the combination of R¹, X, and (R³)ₙ for a compound corresponds in each case to one row of Table A

Table 436: Compounds of formula I.A* in which R⁴ is Cl, R⁵ is H, Z is Q4, R^{6a} is 3-pyridyl, R^{6b} is Cl, and the combination of R¹, X, and (R³)ₙ for a compound corresponds in each case to one row of Table A

Table 437: Compounds of formula I.A* in which R⁴ is Cl, R⁵ is F, Z is Q4, R^{6a} is 3-pyridyl, R^{6b} is Cl, and the combination of R¹, X, and (R³)ₙ for a compound corresponds in each case to one row of Table A

Table 438: Compounds of formula I.A* in which R⁴ is Cl, R⁵ is Cl, Z is Q4, R^{6a} is 3-pyridyl, R^{6b} is Cl, and the combination of R¹, X, and (R³)ₙ for a compound corresponds in each case to one row of Table A

Table 439: Compounds of formula I.A* in which R⁴ is Cl, R⁵ is CH₃, Z is Q4, R^{6a} is 3-pyridyl, R^{6b} is Cl, and the combination of R¹, X, and (R³)ₙ for a compound corresponds in each case to one row of Table A

Table 440: Compounds of formula I.A* in which R⁴ is Cl, R⁵ is cC₃H₅, Z is Q4, R^{6a} is 3-pyridyl, R^{6b} is Cl, and the combination of R¹, X, and (R³)ₙ for a compound corresponds in each case to one row of Table A

Table 441: Compounds of formula I.A* in which R⁴ is CH₃, R⁵ is H, Z is Q1, R^{6a} is 2-pyridyl, R^{6b} is Cl, and the combination of R¹, X, and (R³)ₙ for a compound corresponds in each case to one row of Table A

Table 442: Compounds of formula I.A* in which R⁴ is CH₃, R⁵ is F, Z is Q1, R^{6a} is 2-pyridyl, R^{6b} is Cl, and the combination of R¹, X, and (R³)ₙ for a compound corresponds in each case to one row of Table A

Table 443: Compounds of formula I.A* in which R⁴ is CH₃, R⁵ is Cl, Z is Q1, R^{6a} is 2-pyridyl, R^{6b} is Cl, and the combination of R¹, X, and (R³)ₙ for a compound corresponds in each case to one row of Table A

Table 444: Compounds of formula I.A* in which R⁴ is CH₃, R⁵ is CH₃, Z is Q1, R^{6a} is 2-pyridyl, R^{6b} is Cl, and the combination of R¹, X, and (R³)ₙ for a compound corresponds in each case to one row of Table A

Table 445: Compounds of formula I.A* in which R⁴ is CH₃, R⁵ is cC₃H₅, Z is Q1, R^{6a} is 2-pyridyl, R^{6b} is Cl, and the combination of R¹, X, and (R³)ₙ for a compound corresponds in each case to one row of Table A

Table 446: Compounds of formula I.A* in which R⁴ is CH₃, R⁵ is H, Z is Q2, R^{6a} is 2-pyridyl, R^{6b} is Cl, and the combination of R¹, X, and (R³)ₙ for a compound corresponds in each case to one row of Table A

Table 447: Compounds of formula I.A* in which R⁴ is CH₃, R⁵ is F, Z is Q2, R^{6a} is 2-pyridyl, R^{6b} is Cl, and the combination of R¹, X, and (R³)ₙ for a compound corresponds in each case to one row of Table A

Table 448: Compounds of formula I.A* in which R⁴ is CH₃, R⁵ is Cl, Z is Q2, R^{6a} is 2-pyridyl, R^{6b} is Cl, and the combination of R¹, X, and (R³)ₙ for a compound corresponds in each case to one row of Table A

Table 449: Compounds of formula I.A* in which R⁴ is CH₃, R⁵ is CH₃, Z is Q2, R^{6a} is 2-pyridyl, R^{6b} is Cl, and the combination of R¹, X, and (R³)ₙ for a compound corresponds in each case to one row of Table A

Table 450: Compounds of formula I.A* in which R⁴ is CH₃, R⁵ is cC₃H₅, Z is Q2, R^{6a} is 2-pyridyl, R^{6b} is Cl, and the combination of R¹, X, and (R³)ₙ for a compound corresponds in each case to one row of Table A

Table 451: Compounds of formula I.A* in which R⁴ is CH₃, R⁵ is H, Z is Q3, R^{6a} is 2-pyridyl, R^{6b} is Cl, and the combination of R¹, X, and (R³)ₙ for a compound corresponds in each case to one row of Table A

Table 452: Compounds of formula I.A* in which R⁴ is CH₃, R⁵ is F, Z is Q3, R^{6a} is 2-pyridyl, R^{6b} is Cl, and the combination of R¹, X, and (R³)ₙ for a compound corresponds in each case to one row of Table A

Table 453: Compounds of formula I.A* in which R⁴ is CH₃, R⁵ is Cl, Z is Q3, R^{6a} is 2-pyridyl, R^{6b} is Cl, and the combination of R¹, X, and (R³)ₙ for a compound corresponds in each case to one row of Table A

Table 454: Compounds of formula I.A* in which R⁴ is CH₃, R⁵ is CH₃, Z is Q3, R^{6a} is 2-pyridyl, R^{6b} is Cl, and the combination of R¹, X, and (R³)ₙ for a compound corresponds in each case to one row of Table A

Table 455: Compounds of formula I.A* in which R⁴ is CH₃, R⁵ is cC₃H₅, Z is Q3, R^{6a} is 2-pyridyl, R^{6b} is Cl, and the combination of R¹, X, and (R³)ₙ for a compound corresponds in each case to one row of Table A

Table 456: Compounds of formula I.A* in which R⁴ is CH₃, R⁵ is H, Z is Q4, R^{6a} is 2-pyridyl, R^{6b} is Cl, and the combination of R¹, X, and (R³)ₙ for a compound corresponds in each case to one row of Table A

Table 457: Compounds of formula I.A* in which R⁴ is CH₃, R⁵ is F, Z is Q4, R^{6a} is 2-pyridyl, R^{6b} is Cl, and the combination of R¹, X, and (R³)ₙ for a compound corresponds in each case to one row of Table A

Table 458: Compounds of formula I.A* in which R⁴ is CH₃, R⁵ is Cl, Z is Q4, R^{6a} is 2-pyridyl, R^{6b} is Cl, and the combination of R¹, X, and (R³)ₙ for a compound corresponds in each case to one row of Table A

Table 459: Compounds of formula I.A* in which R⁴ is CH₃, R⁵ is CH₃, Z is Q4, R^{6a} is 2-pyridyl, R^{6b} is Cl, and the combination of R¹, X, and (R³)ₙ for a compound corresponds in each case to one row of Table A

Table 460: Compounds of formula I.A* in which R⁴ is CH₃, R⁵ is cC₃H₅, Z is Q4, R^{6a} is 2-pyridyl, R^{6b} is Cl, and the combination of R¹, X, and (R³)ₙ for a compound corresponds in each case to one row of Table A

Table 461: Compounds of formula I.A* in which R⁴ is CH₃, R⁵ is H, Z is Q1, R^{6a} is 3-pyridyl, R^{6b} is Cl, and the combination of R¹, X, and (R³)ₙ for a compound corresponds in each case to one row of Table A

Table 462: Compounds of formula I.A* in which R⁴ is CH₃, R⁵ is F, Z is Q1, R^{6a} is 3-pyridyl, R^{6b} is Cl, and the combination of R¹, X, and (R³)ₙ for a compound corresponds in each case to one row of Table A

Table 463: Compounds of formula I.A* in which R⁴ is CH₃, R⁵ is Cl, Z is Q1, R^{6a} is 3-pyridyl, R^{6b} is Cl, and the combination of R¹, X, and (R³)ₙ for a compound corresponds in each case to one row of Table A

Table 464: Compounds of formula I.A* in which R⁴ is CH₃, R⁵ is CH₃, Z is Q1, R^{6a} is 3-pyridyl, R^{6b} is Cl, and the combination of R¹, X, and (R³)ₙ for a compound corresponds in each case to one row of Table A

Table 465: Compounds of formula I.A* in which R⁴ is CH₃, R⁵ is cC₃H₅, Z is Q1, R^{6a} is 3-pyridyl, R^{6b} is Cl, and the combination of R¹, X, and (R³)ₙ for a compound corresponds in each case to one row of Table A

Table 466: Compounds of formula I.A* in which R⁴ is CH₃, R⁵ is H, Z is Q2, R^{6a} is 3-pyridyl, R^{6b} is Cl, and the combination of R¹, X, and (R³)ₙ for a compound corresponds in each case to one row of Table A

Table 467: Compounds of formula I.A* in which R⁴ is CH₃, R⁵ is F, Z is Q2, R^{6a} is 3-pyridyl, R^{6b} is Cl, and the combination of R¹, X, and (R³)ₙ for a compound corresponds in each case to one row of Table A

Table 468: Compounds of formula I.A* in which R⁴ is CH₃, R⁵ is Cl, Z is Q2, R^{6a} is 3-pyridyl, R^{6b} is Cl, and the combination of R¹, X, and (R³)ₙ for a compound corresponds in each case to one row of Table A

Table 469: Compounds of formula I.A* in which R⁴ is CH₃, R⁵ is CH₃, Z is Q2, R^{6a} is 3-pyridyl, R^{6b} is Cl, and the combination of R¹, X, and (R³)ₙ for a compound corresponds in each case to one row of Table A

Table 470: Compounds of formula I.A* in which R⁴ is CH₃, R⁵ is cC₃H₅, Z is Q2, R^{6a} is 3-pyridyl, R^{6b} is Cl, and the combination of R¹, X, and (R³)ₙ for a compound corresponds in each case to one row of Table A

Table 471: Compounds of formula I.A* in which R⁴ is CH₃, R⁵ is H, Z is Q3, R^{6a} is 3-pyridyl, R^{6b} is Cl, and the combination of R¹, X, and (R³)ₙ for a compound corresponds in each case to one row of Table A

Table 472: Compounds of formula I.A* in which R⁴ is CH₃, R⁵ is F, Z is Q3, R^{6a} is 3-pyridyl, R^{6b} is Cl, and the combination of R¹, X, and (R³)ₙ for a compound corresponds in each case to one row of Table A

Table 473: Compounds of formula I.A* in which R⁴ is CH₃, R⁵ is Cl, Z is Q3, R^{6a} is 3-pyridyl, R^{6b} is Cl, and the combination of R¹, X, and (R³)ₙ for a compound corresponds in each case to one row of Table A

Table 474: Compounds of formula I.A* in which R⁴ is CH₃, R⁵ is CH₃, Z is Q3, R^{6a} is 3-pyridyl, R^{6b} is Cl, and the combination of R¹, X, and (R³)ₙ for a compound corresponds in each case to one row of Table A

Table 475: Compounds of formula I.A* in which R⁴ is CH₃, R⁵ is cC₃H₅, Z is Q3, R^{6a} is 3-pyridyl, R^{6b} is Cl, and the combination of R¹, X, and (R³)ₙ for a compound corresponds in each case to one row of Table A

Table 476: Compounds of formula I.A* in which R⁴ is CH₃, R⁵ is H, Z is Q4, R^{6a} is 3-pyridyl, R^{6b} is Cl, and the combination of R¹, X, and (R³)ₙ for a compound corresponds in each case to one row of Table A

Table 477: Compounds of formula I.A* in which R⁴ is CH₃, R⁵ is F, Z is Q4, R^{6a} is 3-pyridyl, R^{6b} is Cl, and the combination of R¹, X, and (R³)ₙ for a compound corresponds in each case to one row of Table A

Table 478: Compounds of formula I.A* in which R⁴ is CH₃, R⁵ is Cl, Z is Q4, R^{6a} is 3-pyridyl, R^{6b} is Cl, and the combination of R¹, X, and (R³)ₙ for a compound corresponds in each case to one row of Table A

Table 479: Compounds of formula I.A* in which R⁴ is CH₃, R⁵ is CH₃, Z is Q4, R^{6a} is 3-pyridyl, R^{6b} is Cl, and the combination of R¹, X, and (R³)ₙ for a compound corresponds in each case to one row of Table A

Table 480: Compounds of formula I.A* in which R⁴ is CH₃, R⁵ is cC₃H₅, Z is Q4, R^{6a} is 3-pyridyl, R^{6b} is Cl, and the combination of R¹, X, and (R³)ₙ for a compound corresponds in each case to one row of Table A

Table 481: Compounds of formula I.A* in which R⁴ and R⁵ are H, Z is Q1, R^{6a} is 2-pyridyl, R^{6b} is methyl, and the combination of R¹, X, and (R³)ₙ for a compound corresponds in each case to one row of Table A

Table 482: Compounds of formula I.A* in which R⁴ is H, R⁵ is F, Z is Q1, R^{6a} is 2-pyridyl, R^{6b} is methyl, and the combination of R¹, X, and (R³)ₙ for a compound corresponds in each case to one row of Table A

Table 483: Compounds of formula I.A* in which R⁴ is H, R⁵ is Cl, Z is Q1, R^{6a} is 2-pyridyl, R^{6b} is methyl, and the combination of R¹, X, and (R³)ₙ for a compound corresponds in each case to one row of Table A

Table 484: Compounds of formula I.A* in which R⁴ is H, R⁵ is CH₃, Z is Q1, R^{6a} is 2-pyridyl, R^{6b} is methyl, and the combination of R¹, X, and (R³)ₙ for a compound corresponds in each case to one row of Table A

Table 485: Compounds of formula I.A* in which R⁴ is H, R⁵ is cC₃H₅, Z is Q1, R^{6a} is 2-pyridyl, R^{6b} is methyl, and the combination of R¹, X, and (R³)ₙ for a compound corresponds in each case to one row of Table A

Table 486: Compounds of formula I.A* in which R⁴ and R⁵ are H, Z is Q2, R^{6a} is 2-pyridyl, R^{6b} is methyl, and the combination of R¹, X, and (R³)ₙ for a compound corresponds in each case to one row of Table A

Table 487: Compounds of formula I.A* in which R⁴ is H, R⁵ is F, Z is Q2, R^{6a} is 2-pyridyl, R^{6b} is methyl, and the combination of R¹, X, and (R³)ₙ for a compound corresponds in each case to one row of Table A

Table 488: Compounds of formula I.A* in which R⁴ is H, R⁵ is Cl, Z is Q2, R^{6a} is 2-pyridyl, R^{6b} is methyl, and the combination of R¹, X, and (R³)ₙ for a compound corresponds in each case to one row of Table A

Table 489: Compounds of formula I.A* in which R⁴ is H, R⁵ is CH₃, Z is Q2, R^{6a} is 2-pyridyl, R^{6b} is methyl, and the combination of R¹, X, and (R³)ₙ for a compound corresponds in each case to one row of Table A

Table 490: Compounds of formula I.A* in which R⁴ is H, R⁵ is cC₃H₅, Z is Q2, R^{6a} is 2-pyridyl, R^{6b} is methyl, and the combination of R¹, X, and (R³)ₙ for a compound corresponds in each case to one row of Table A

Table 491: Compounds of formula I.A* in which R⁴ and R⁵ are H, Z is Q3, R^{6a} is 2-pyridyl, R^{6b} is methyl, and the combination of R¹, X, and (R³)ₙ for a compound corresponds in each case to one row of Table A

Table 492: Compounds of formula I.A* in which R⁴ is H, R⁵ is F, Z is Q3, R^{6a} is 2-pyridyl, R^{6b} is methyl, and the combination of R¹, X, and (R³)ₙ for a compound corresponds in each case to one row of Table A

Table 493: Compounds of formula I.A* in which R⁴ is H, R⁵ is Cl, Z is Q3, R^{6a} is 2-pyridyl, R^{6b} is methyl, and the combination of R¹, X, and (R³)ₙ for a compound corresponds in each case to one row of Table A

Table 494: Compounds of formula I.A* in which R⁴ is H, R⁵ is CH₃, Z is Q3, R^{6a} is 2-pyridyl, R^{6b} is methyl, and the combination of R¹, X, and (R³)ₙ for a compound corresponds in each case to one row of Table A

Table 495: Compounds of formula I.A* in which R⁴ is H, R⁵ is cC₃H₅, Z is Q3, R^{6a} is 2-pyridyl, R^{6b} is methyl, and the combination of R¹, X, and (R³)ₙ for a compound corresponds in each case to one row of Table A

Table 496: Compounds of formula I.A* in which R⁴ and R⁵ are H, Z is Q4, R^{6a} is 2-pyridyl, R^{6b} is methyl, and the combination of R¹, X, and (R³)ₙ for a compound corresponds in each case to one row of Table A

Table 497: Compounds of formula I.A* in which R⁴ is H, R⁵ is F, Z is Q4, R^{6a} is 2-pyridyl, R^{6b} is methyl, and the combination of R¹, X, and (R³)ₙ for a compound corresponds in each case to one row of Table A

Table 498: Compounds of formula I.A* in which R⁴ is H, R⁵ is Cl, Z is Q4, R^{6a} is 2-pyridyl, R^{6b} is methyl, and the combination of R¹, X, and (R³)ₙ for a compound corresponds in each case to one row of Table A

Table 499: Compounds of formula I.A* in which R⁴ is H, R⁵ is CH₃, Z is Q4, R^{6a} is 2-pyridyl, R^{6b} is methyl, and the combination of R¹, X, and (R³)ₙ for a compound corresponds in each case to one row of Table A

Table 500: Compounds of formula I.A* in which R⁴ is H, R⁵ is cC₃H₅, Z is Q4, R^{6a} is 2-pyridyl, R^{6b} is methyl, and the combination of R¹, X, and (R³)ₙ for a compound corresponds in each case to one row of Table A

Table 501: Compounds of formula I.A* in which R⁴ and R⁵ are H, Z is Q1, R^{6a} is 3-pyridyl, R^{6b} is methyl, and the combination of R¹, X, and (R³)ₙ for a compound corresponds in each case to one row of Table A

Table 502: Compounds of formula I.A* in which R⁴ is H, R⁵ is F, Z is Q1, R^{6a} is 3-pyridyl, R^{6b} is methyl, and the combination of R¹, X, and (R³)ₙ for a compound corresponds in each case to one row of Table A

Table 503: Compounds of formula I.A* in which R⁴ is H, R⁵ is Cl, Z is Q1, R^{6a} is 3-pyridyl, R^{6b} is methyl, and the combination of R¹, X, and (R³)ₙ for a compound corresponds in each case to one row of Table A

Table 504: Compounds of formula I.A* in which R⁴ is H, R⁵ is CH₃, Z is Q1, R^{6a} is 3-pyridyl, R^{6b} is methyl, and the combination of R¹, X, and (R³)ₙ for a compound corresponds in each case to one row of Table A

Table 505: Compounds of formula I.A* in which R⁴ is H, R⁵ is cC₃H₅, Z is Q1, R^{6a} is 3-pyridyl, R^{6b} is methyl, and the combination of R¹, X, and (R³)ₙ for a compound corresponds in each case to one row of Table A

Table 506: Compounds of formula I.A* in which R⁴ and R⁵ are H, Z is Q2, R^{6a} is 3-pyridyl, R^{6b} is methyl, and the combination of R¹, X, and (R³)ₙ for a compound corresponds in each case to one row of Table A

Table 507: Compounds of formula I.A* in which R⁴ is H, R⁵ is F, Z is Q2, R^{6a} is 3-pyridyl, R^{6b} is methyl, and the combination of R¹, X, and (R³)ₙ for a compound corresponds in each case to one row of Table A

Table 508: Compounds of formula I.A* in which R⁴ is H, R⁵ is Cl, Z is Q2, R^{6a} is 3-pyridyl, R^{6b} is methyl, and the combination of R¹, X, and (R³)ₙ for a compound corresponds in each case to one row of Table A

Table 509: Compounds of formula I.A* in which R⁴ is H, R⁵ is CH₃, Z is Q2, R^{6a} is 3-pyridyl, R^{6b} is methyl, and the combination of R¹, X, and (R³)ₙ for a compound corresponds in each case to one row of Table A

Table 510: Compounds of formula I.A* in which R⁴ is H, R⁵ is cC₃H₅, Z is Q2, R^{6a} is 3-pyridyl, R^{6b} is methyl, and the combination of R¹, X, and (R³)ₙ for a compound corresponds in each case to one row of Table A

Table 511: Compounds of formula I.A* in which R⁴ and R⁵ are H, Z is Q3, R^{6a} is 3-pyridyl, R^{6b} is methyl, and the combination of R¹, X, and (R³)ₙ for a compound corresponds in each case to one row of Table A

Table 512: Compounds of formula I.A* in which R⁴ is H, R⁵ is F, Z is Q3, R^{6a} is 3-pyridyl, R^{6b} is methyl, and the combination of R¹, X, and (R³)ₙ for a compound corresponds in each case to one row of Table A

Table 513: Compounds of formula I.A* in which R⁴ is H, R⁵ is Cl, Z is Q3, R^{6a} is 3-pyridyl, R^{6b} is methyl, and the combination of R¹, X, and (R³)ₙ for a compound corresponds in each case to one row of Table A

Table 514: Compounds of formula I.A* in which R⁴ is H, R⁵ is CH₃, Z is Q3, R^{6a} is 3-pyridyl, R^{6b} is methyl, and the combination of R¹, X, and (R³)ₙ for a compound corresponds in each case to one row of Table A

Table 515: Compounds of formula I.A* in which R⁴ is H, R⁵ is cC₃H₅, Z is Q3, R^{6a} is 3-pyridyl, R^{6b} is methyl, and the combination of R¹, X, and (R³)ₙ for a compound corresponds in each case to one row of Table A

Table 516: Compounds of formula I.A* in which R⁴ and R⁵ are H, Z is Q4, R^{6a} is 3-pyridyl, R^{6b} is methyl, and the combination of R¹, X, and (R³)ₙ for a compound corresponds in each case to one row of Table A

Table 517: Compounds of formula I.A* in which R⁴ is H, R⁵ is F, Z is Q4, R^{6a} is 3-pyridyl, R^{6b} is methyl, and the combination of R¹, X, and (R³)ₙ for a compound corresponds in each case to one row of Table A

Table 518: Compounds of formula I.A* in which R⁴ is H, R⁵ is Cl, Z is Q4, R^{6a} is 3-pyridyl, R^{6b} is methyl, and the combination of R¹, X, and (R³)ₙ for a compound corresponds in each case to one row of Table A

Table 519: Compounds of formula I.A* in which R⁴ is H, R⁵ is CH₃, Z is Q4, R^{6a} is 3-pyridyl, R^{6b} is methyl, and the combination of R¹, X, and (R³)ₙ for a compound corresponds in each case to one row of Table A

Table 520: Compounds of formula I.A* in which R⁴ is H, R⁵ is cC₃H₅, Z is Q4, R^{6a} is 3-pyridyl, R^{6b} is methyl, and the combination of R¹, X, and (R³)ₙ for a compound corresponds in each case to one row of Table A

Table 521: Compounds of formula I.A* in which R⁴ is F, R⁵ is H, Z is Q1, R^{6a} is 2-pyridyl, R^{6b} is methyl, and the combination of R¹, X, and (R³)ₙ for a compound corresponds in each case to one row of Table A

Table 522: Compounds of formula I.A* in which R⁴ is F, R⁵ is F, Z is Q1, R^{6a} is 2-pyridyl, R^{6b} is methyl, and the combination of R¹, X, and (R³)ₙ for a compound corresponds in each case to one row of Table A

Table 523: Compounds of formula I.A* in which R⁴ is F, R⁵ is Cl, Z is Q1, R^{6a} is 2-pyridyl, R^{6b} is methyl, and the combination of R¹, X, and (R³)ₙ for a compound corresponds in each case to one row of Table A

Table 524: Compounds of formula I.A* in which R⁴ is F, R⁵ is CH₃, Z is Q1, R^{6a} is 2-pyridyl, R^{6b} is methyl, and the combination of R¹, X, and (R³)ₙ for a compound corresponds in each case to one row of Table A

Table 525: Compounds of formula I.A* in which R⁴ is F, R⁵ is cC₃H₅, Z is Q1, R^{6a} is 2-pyridyl, R^{6b} is methyl, and the combination of R¹, X, and (R³)ₙ for a compound corresponds in each case to one row of Table A

Table 526: Compounds of formula I.A* in which R⁴ is F, R⁵ is H, Z is Q2, R^{6a} is 2-pyridyl, R^{6b} is methyl, and the combination of R¹, X, and (R³)ₙ for a compound corresponds in each case to one row of Table A

Table 527: Compounds of formula I.A* in which R⁴ is F, R⁵ is F, Z is Q2, R^{6a} is 2-pyridyl, R^{6b} is methyl, and the combination of R¹, X, and (R³)ₙ for a compound corresponds in each case to one row of Table A

Table 528: Compounds of formula I.A* in which R⁴ is F, R⁵ is Cl, Z is Q2, R^{6a} is 2-pyridyl, R^{6b} is methyl, and the combination of R¹, X, and (R³)ₙ for a compound corresponds in each case to one row of Table A

Table 529: Compounds of formula I.A* in which R⁴ is F, R⁵ is CH₃, Z is Q2, R^{6a} is 2-pyridyl, R^{6b} is methyl, and the combination of R¹, X, and (R³)ₙ for a compound corresponds in each case to one row of Table A

Table 530: Compounds of formula I.A* in which R⁴ is F, R⁵ is cC₃H₅, Z is Q2, R^{6a} is 2-pyridyl, R^{6b} is methyl, and the combination of R¹, X, and (R³)ₙ for a compound corresponds in each case to one row of Table A

Table 531: Compounds of formula I.A* in which R⁴ is F, R⁵ is H, Z is Q3, R^{6a} is 2-pyridyl, R^{6b} is methyl, and the combination of R¹, X, and (R³)ₙ for a compound corresponds in each case to one row of Table A

Table 532: Compounds of formula I.A* in which R⁴ is F, R⁵ is F, Z is Q3, R^{6a} is 2-pyridyl, R^{6b} is methyl, and the combination of R¹, X, and (R³)ₙ for a compound corresponds in each case to one row of Table A

Table 533: Compounds of formula I.A* in which R⁴ is F, R⁵ is Cl, Z is Q3, R^{6a} is 2-pyridyl, R^{6b} is methyl, and the combination of R¹, X, and (R³)ₙ for a compound corresponds in each case to one row of Table A

Table 534: Compounds of formula I.A* in which R⁴ is F, R⁵ is CH₃, Z is Q3, R^{6a} is 2-pyridyl, R^{6b} is methyl, and the combination of R¹, X, and (R³)ₙ for a compound corresponds in each case to one row of Table A

Table 535: Compounds of formula I.A* in which R⁴ is F, R⁵ is cC₃H₅, Z is Q3, R^{6a} is 2-pyridyl, R^{6b} is methyl, and the combination of R¹, X, and (R³)ₙ for a compound corresponds in each case to one row of Table A

Table 536: Compounds of formula I.A* in which R⁴ is F, R⁵ is H, Z is Q4, R^{6a} is 2-pyridyl, R^{6b} is methyl, and the combination of R¹, X, and (R³)ₙ for a compound corresponds in each case to one row of Table A

Table 537: Compounds of formula I.A* in which R⁴ is F, R⁵ is F, Z is Q4, R^{6a} is 2-pyridyl, R^{6b} is methyl, and the combination of R¹, X, and (R³)ₙ for a compound corresponds in each case to one row of Table A

Table 538: Compounds of formula I.A* in which R⁴ is F, R⁵ is Cl, Z is Q4, R^{6a} is 2-pyridyl, R^{6b} is methyl, and the combination of R¹, X, and (R³)ₙ for a compound corresponds in each case to one row of Table A

Table 539: Compounds of formula I.A* in which R⁴ is F, R⁵ is CH₃, Z is Q4, R^{6a} is 2-pyridyl, R^{6b} is methyl, and the combination of R¹, X, and (R³)ₙ for a compound corresponds in each case to one row of Table A

Table 540: Compounds of formula I.A* in which R⁴ is F, R⁵ is cC₃H₅, Z is Q4, R^{6a} is 2-pyridyl, R^{6b} is methyl, and the combination of R¹, X, and (R³)ₙ for a compound corresponds in each case to one row of Table A

Table 541: Compounds of formula I.A* in which R⁴ is F, R⁵ is H, Z is Q1, R^{6a} is 3-pyridyl, R^{6b} is methyl, and the combination of R¹, X, and (R³)ₙ for a compound corresponds in each case to one row of Table A

Table 542: Compounds of formula I.A* in which R⁴ is F, R⁵ is F, Z is Q1, R^{6a} is 3-pyridyl, R^{6b} is methyl, and the combination of R¹, X, and (R³)ₙ for a compound corresponds in each case to one row of Table A

Table 543: Compounds of formula I.A* in which R⁴ is F, R⁵ is Cl, Z is Q1, R^{6a} is 3-pyridyl, R^{6b} is methyl, and the combination of R¹, X, and (R³)ₙ for a compound corresponds in each case to one row of Table A

Table 544: Compounds of formula I.A* in which R⁴ is F, R⁵ is CH₃, Z is Q1, R^{6a} is 3-pyridyl, R^{6b} is methyl, and the combination of R¹, X, and (R³)ₙ for a compound corresponds in each case to one row of Table A

Table 545: Compounds of formula I.A* in which R⁴ is F, R⁵ is cC₃H₅, Z is Q1, R^{6a} is 3-pyridyl, R^{6b} is methyl, and the combination of R¹, X, and (R³)ₙ for a compound corresponds in each case to one row of Table A

Table 546: Compounds of formula I.A* in which R⁴ is F, R⁵ is H, Z is Q2, R^{6a} is 3-pyridyl, R^{6b} is methyl, and the combination of R¹, X, and (R³)ₙ for a compound corresponds in each case to one row of Table A

Table 547: Compounds of formula I.A* in which R⁴ is F, R⁵ is F, Z is Q2, R^{6a} is 3-pyridyl, R^{6b} is methyl, and the combination of R¹, X, and (R³)ₙ for a compound corresponds in each case to one row of Table A

Table 548: Compounds of formula I.A* in which R⁴ is F, R⁵ is Cl, Z is Q2, R^{6a} is 3-pyridyl, R^{6b} is methyl, and the combination of R¹, X, and (R³)ₙ for a compound corresponds in each case to one row of Table A

Table 549: Compounds of formula I.A* in which R⁴ is F, R⁵ is CH₃, Z is Q2, R^{6a} is 3-pyridyl, R^{6b} is methyl, and the combination of R¹, X, and (R³)ₙ for a compound corresponds in each case to one row of Table A

Table 550: Compounds of formula I.A* in which R⁴ is F, R⁵ is cC₃H₅, Z is Q2, R^{6a} is 3-pyridyl, R^{6b} is methyl, and the combination of R¹, X, and (R³)ₙ for a compound corresponds in each case to one row of Table A

Table 551: Compounds of formula I.A* in which R⁴ is F, R⁵ is H, Z is Q3, R^{6a} is 3-pyridyl, R^{6b} is methyl, and the combination of R¹, X, and (R³)ₙ for a compound corresponds in each case to one row of Table A

Table 552: Compounds of formula I.A* in which R⁴ is F, R⁵ is F, Z is Q3, R^{6a} is 3-pyridyl, R^{6b} is methyl, and the combination of R¹, X, and (R³)ₙ for a compound corresponds in each case to one row of Table A

Table 553: Compounds of formula I.A* in which R⁴ is F, R⁵ is Cl, Z is Q3, R^{6a} is 3-pyridyl, R^{6b} is methyl, and the combination of R¹, X, and (R³)ₙ for a compound corresponds in each case to one row of Table A

Table 554: Compounds of formula I.A* in which R⁴ is F, R⁵ is CH₃, Z is Q3, R^{6a} is 3-pyridyl, R^{6b} is methyl, and the combination of R¹, X, and (R³)ₙ for a compound corresponds in each case to one row of Table A

Table 555: Compounds of formula I.A* in which R⁴ is F, R⁵ is cC₃H₅, Z is Q3, R^{6a} is 3-pyridyl, R^{6b} is methyl, and the combination of R¹, X, and (R³)ₙ for a compound corresponds in each case to one row of Table A

Table 556: Compounds of formula I.A* in which R⁴ is F, R⁵ is H, Z is Q4, R^{6a} is 3-pyridyl, R^{6b} is methyl, and the combination of R¹, X, and (R³)ₙ for a compound corresponds in each case to one row of Table A

Table 557: Compounds of formula I.A* in which R⁴ is F, R⁵ is F, Z is Q4, R^{6a} is 3-pyridyl, R^{6b} is methyl, and the combination of R¹, X, and (R³)ₙ for a compound corresponds in each case to one row of Table A

Table 558: Compounds of formula I.A* in which R⁴ is F, R⁵ is Cl, Z is Q4, R^{6a} is 3-pyridyl, R^{6b} is methyl, and the combination of R¹, X, and (R³)ₙ for a compound corresponds in each case to one row of Table A

Table 559: Compounds of formula I.A* in which R⁴ is F, R⁵ is CH₃, Z is Q4, R^{6a} is 3-pyridyl, R^{6b} is methyl, and the combination of R¹, X, and (R³)ₙ for a compound corresponds in each case to one row of Table A

Table 560: Compounds of formula I.A* in which R⁴ is F, R⁵ is cC₃H₅, Z is Q4, R^{6a} is 3-pyridyl, R^{6b} is methyl, and the combination of R¹, X, and (R³)ₙ for a compound corresponds in each case to one row of Table A

Table 561: Compounds of formula I.A* in which R⁴ is Cl, R⁵ is H, Z is Q1, R^{6a} is 2-pyridyl, R^{6b} is methyl, and the combination of R¹, X, and (R³)ₙ for a compound corresponds in each case to one row of Table A

Table 562: Compounds of formula I.A* in which R⁴ is Cl, R⁵ is F, Z is Q1, R^{6a} is 2-pyridyl, R^{6b} is methyl, and the combination of R¹, X, and (R³)ₙ for a compound corresponds in each case to one row of Table A

Table 563: Compounds of formula I.A* in which R⁴ is Cl, R⁵ is Cl, Z is Q1, R^{6a} is 2-pyridyl, R^{6b} is methyl, and the combination of R¹, X, and (R³)ₙ for a compound corresponds in each case to one row of Table A

Table 564: Compounds of formula I.A* in which R⁴ is Cl, R⁵ is CH₃, Z is Q1, R^{6a} is 2-pyridyl, R^{6b} is methyl, and the combination of R¹, X, and (R³)ₙ for a compound corresponds in each case to one row of Table A

Table 565: Compounds of formula I.A* in which R⁴ is Cl, R⁵ is cC₃H₅, Z is Q1, R^{6a} is 2-pyridyl, R^{6b} is methyl, and the combination of R¹, X, and (R³)ₙ for a compound corresponds in each case to one row of Table A

Table 566: Compounds of formula I.A* in which R⁴ is Cl, R⁵ is H, Z is Q2, R^{6a} is 2-pyridyl, R^{6b} is methyl, and the combination of R¹, X, and (R³)ₙ for a compound corresponds in each case to one row of Table A

Table 567: Compounds of formula I.A* in which R⁴ is Cl, R⁵ is F, Z is Q2, R^{6a} is 2-pyridyl, R^{6b} is methyl, and the combination of R¹, X, and (R³)ₙ for a compound corresponds in each case to one row of Table A

Table 568: Compounds of formula I.A* in which R⁴ is Cl, R⁵ is Cl, Z is Q2, R^{6a} is 2-pyridyl, R^{6b} is methyl, and the combination of R¹, X, and (R³)ₙ for a compound corresponds in each case to one row of Table A

Table 569: Compounds of formula I.A* in which R⁴ is Cl, R⁵ is CH₃, Z is Q2, R^{6a} is 2-pyridyl, R^{6b} is methyl, and the combination of R¹, X, and (R³)ₙ for a compound corresponds in each case to one row of Table A

Table 570: Compounds of formula I.A* in which R⁴ is Cl, R⁵ is cC₃H₅, Z is Q2, R^{6a} is 2-pyridyl, R^{6b} is methyl, and the combination of R¹, X, and (R³)ₙ for a compound corresponds in each case to one row of Table A

Table 571: Compounds of formula I.A* in which R⁴ is Cl, R⁵ is H, Z is Q3, R^{6a} is 2-pyridyl, R^{6b} is methyl, and the combination of R¹, X, and (R³)ₙ for a compound corresponds in each case to one row of Table A

Table 572: Compounds of formula I.A* in which R⁴ is Cl, R⁵ is F, Z is Q3, R^{6a} is 2-pyridyl, R^{6b} is methyl, and the combination of R¹, X, and (R³)ₙ for a compound corresponds in each case to one row of Table A

Table 573: Compounds of formula I.A* in which R⁴ is Cl, R⁵ is Cl, Z is Q3, R^{6a} is 2-pyridyl, R^{6b} is methyl, and the combination of R¹, X, and (R³)ₙ for a compound corresponds in each case to one row of Table A

Table 574: Compounds of formula I.A* in which R⁴ is Cl, R⁵ is CH₃, Z is Q3, R^{6a} is 2-pyridyl, R^{6b} is methyl, and the combination of R¹, X, and (R³)ₙ for a compound corresponds in each case to one row of Table A

Table 575: Compounds of formula I.A* in which R⁴ is Cl, R⁵ is cC₃H₅, Z is Q3, R^{6a} is 2-pyridyl, R^{6b} is methyl, and the combination of R¹, X, and (R³)ₙ for a compound corresponds in each case to one row of Table A

Table 576: Compounds of formula I.A* in which R⁴ is Cl, R⁵ is H, Z is Q4, R^{6a} is 2-pyridyl, R^{6b} is methyl, and the combination of R¹, X, and (R³)ₙ for a compound corresponds in each case to one row of Table A

Table 577: Compounds of formula I.A* in which R⁴ is Cl, R⁵ is F, Z is Q4, R^{6a} is 2-pyridyl, R^{6b} is methyl, and the combination of R¹, X, and (R³)ₙ for a compound corresponds in each case to one row of Table A

Table 578: Compounds of formula I.A* in which R⁴ is Cl, R⁵ is Cl, Z is Q4, R^{6a} is 2-pyridyl, R^{6b} is methyl, and the combination of R¹, X, and (R³)ₙ for a compound corresponds in each case to one row of Table A

Table 579: Compounds of formula I.A* in which R⁴ is Cl, R⁵ is CH₃, Z is Q4, R^{6a} is 2-pyridyl, R^{6b} is methyl, and the combination of R¹, X, and (R³)ₙ for a compound corresponds in each case to one row of Table A

Table 580: Compounds of formula I.A* in which R⁴ is Cl, R⁵ is cC₃H₅, Z is Q4, R^{6a} is 2-pyridyl, R^{6b} is methyl, and the combination of R¹, X, and (R³)ₙ for a compound corresponds in each case to one row of Table A

Table 581: Compounds of formula I.A* in which R⁴ is Cl, R⁵ is H, Z is Q1, R^{6a} is 3-pyridyl, R^{6b} is methyl, and the combination of R¹, X, and (R³)ₙ for a compound corresponds in each case to one row of Table A

Table 582: Compounds of formula I.A* in which R⁴ is Cl, R⁵ is F, Z is Q1, R^{6a} is 3-pyridyl, R^{6b} is methyl, and the combination of R¹, X, and (R³)ₙ for a compound corresponds in each case to one row of Table A

Table 583: Compounds of formula I.A* in which R⁴ is Cl, R⁵ is Cl, Z is Q1, R^{6a} is 3-pyridyl, R^{6b} is methyl, and the combination of R¹, X, and (R³)ₙ for a compound corresponds in each case to one row of Table A

Table 584: Compounds of formula I.A* in which R⁴ is Cl, R⁵ is CH₃, Z is Q1, R^{6a} is 3-pyridyl, R^{6b} is methyl, and the combination of R¹, X, and (R³)ₙ for a compound corresponds in each case to one row of Table A

Table 585: Compounds of formula I.A* in which R⁴ is Cl, R⁵ is cC₃H₅, Z is Q1, R^{6a} is 3-pyridyl, R^{6b} is methyl, and the combination of R¹, X, and (R³)ₙ for a compound corresponds in each case to one row of Table A

Table 586: Compounds of formula I.A* in which R⁴ is Cl, R⁵ is H, Z is Q2, R^{6a} is 3-pyridyl, R^{6b} is methyl, and the combination of R¹, X, and (R³)ₙ for a compound corresponds in each case to one row of Table A

Table 587: Compounds of formula I.A* in which R⁴ is Cl, R⁵ is F, Z is Q2, R^{6a} is 3-pyridyl, R^{6b} is methyl, and the combination of R¹, X, and (R³)ₙ for a compound corresponds in each case to one row of Table A

Table 588: Compounds of formula I.A* in which R⁴ is Cl, R⁵ is Cl, Z is Q2, R^{6a} is 3-pyridyl, R^{6b} is methyl, and the combination of R¹, X, and (R³)ₙ for a compound corresponds in each case to one row of Table A

Table 589: Compounds of formula I.A* in which R⁴ is Cl, R⁵ is CH₃, Z is Q2, R^{6a} is 3-pyridyl, R^{6b} is methyl, and the combination of R¹, X, and (R³)ₙ for a compound corresponds in each case to one row of Table A

Table 590: Compounds of formula I.A* in which R⁴ is Cl, R⁵ is cC₃H₅, Z is Q2, R^{6a} is 3-pyridyl, R^{6b} is methyl, and the combination of R¹, X, and (R³)ₙ for a compound corresponds in each case to one row of Table A

Table 591: Compounds of formula I.A* in which R⁴ is Cl, R⁵ is H, Z is Q3, R^{6a} is 3-pyridyl, R^{6b} is methyl, and the combination of R¹, X, and (R³)ₙ for a compound corresponds in each case to one row of Table A

Table 592: Compounds of formula I.A* in which R⁴ is Cl, R⁵ is F, Z is Q3, R^{6a} is 3-pyridyl, R^{6b} is methyl, and the combination of R¹, X, and (R³)ₙ for a compound corresponds in each case to one row of Table A

Table 593: Compounds of formula I.A* in which R⁴ is Cl, R⁵ is Cl, Z is Q3, R^{6a} is 3-pyridyl, R^{6b} is methyl, and the combination of R¹, X, and (R³)ₙ for a compound corresponds in each case to one row of Table A

Table 594: Compounds of formula I.A* in which R⁴ is Cl, R⁵ is CH₃, Z is Q3, R^{6a} is 3-pyridyl, R^{6b} is methyl, and the combination of R¹, X, and (R³)ₙ for a compound corresponds in each case to one row of Table A

Table 595: Compounds of formula I.A* in which R⁴ is Cl, R⁵ is cC₃H₅, Z is Q3, R^{6a} is 3-pyridyl, R^{6b} is methyl, and the combination of R¹, X, and (R³)ₙ for a compound corresponds in each case to one row of Table A

Table 596: Compounds of formula I.A* in which R⁴ is Cl, R⁵ is H, Z is Q4, R^{6a} is 3-pyridyl, R^{6b} is methyl, and the combination of R¹, X, and (R³)ₙ for a compound corresponds in each case to one row of Table A

Table 597: Compounds of formula I.A* in which R⁴ is Cl, R⁵ is F, Z is Q4, R^{6a} is 3-pyridyl, R^{6b} is methyl, and the combination of R¹, X, and (R³)ₙ for a compound corresponds in each case to one row of Table A

Table 598: Compounds of formula I.A* in which R⁴ is Cl, R⁵ is Cl, Z is Q4, R^{6a} is 3-pyridyl, R^{6b} is methyl, and the combination of R¹, X, and (R³)ₙ for a compound corresponds in each case to one row of Table A

Table 599: Compounds of formula I.A* in which R⁴ is Cl, R⁵ is CH₃, Z is Q4, R^{6a} is 3-pyridyl, R^{6b} is methyl, and the combination of R¹, X, and (R³)ₙ for a compound corresponds in each case to one row of Table A

Table 600: Compounds of formula I.A* in which R⁴ is Cl, R⁵ is cC₃H₅, Z is Q4, R^{6a} is 3-pyridyl, R^{6b} is methyl, and the combination of R¹, X, and (R³)ₙ for a compound corresponds in each case to one row of Table A

Table 601: Compounds of formula I.A* in which R⁴ is CH₃, R⁵ is H, Z is Q1, R^{6a} is 2-pyridyl, R^{6b} is methyl, and the combination of R¹, X, and (R³)ₙ for a compound corresponds in each case to one row of Table A

Table 602: Compounds of formula I.A* in which R⁴ is CH₃, R⁵ is F, Z is Q1, R^{6a} is 2-pyridyl, R^{6b} is methyl, and the combination of R¹, X, and (R³)ₙ for a compound corresponds in each case to one row of Table A

Table 603: Compounds of formula I.A* in which R⁴ is CH₃, R⁵ is Cl, Z is Q1, R^{6a} is 2-pyridyl, R^{6b} is methyl, and the combination of R¹, X, and (R³)ₙ for a compound corresponds in each case to one row of Table A

Table 604: Compounds of formula I.A* in which R⁴ is CH₃, R⁵ is CH₃, Z is Q1, R^{6a} is 2-pyridyl, R^{6b} is methyl, and the combination of R¹, X, and (R³)ₙ for a compound corresponds in each case to one row of Table A

Table 605: Compounds of formula I.A* in which R⁴ is CH₃, R⁵ is cC₃H₅, Z is Q1, R^{6a} is 2-pyridyl, R^{6b} is methyl, and the combination of R¹, X, and (R³)ₙ for a compound corresponds in each case to one row of Table A

Table 606: Compounds of formula I.A* in which R⁴ is CH₃, R⁵ is H, Z is Q2, R^{6a} is 2-pyridyl, R^{6b} is methyl, and the combination of R¹, X, and (R³)ₙ for a compound corresponds in each case to one row of Table A

Table 607: Compounds of formula I.A* in which R⁴ is CH₃, R⁵ is F, Z is Q2, R^{6a} is 2-pyridyl, R^{6b} is methyl, and the combination of R¹, X, and (R³)ₙ for a compound corresponds in each case to one row of Table A

Table 608: Compounds of formula I.A* in which R⁴ is CH₃, R⁵ is Cl, Z is Q2, R^{6a} is 2-pyridyl, R^{6b} is methyl, and the combination of R¹, X, and (R³)ₙ for a compound corresponds in each case to one row of Table A

Table 609: Compounds of formula I.A* in which R⁴ is CH₃, R⁵ is CH₃, Z is Q2, R^{6a} is 2-pyridyl, R^{6b} is methyl, and the combination of R¹, X, and (R³)ₙ for a compound corresponds in each case to one row of Table A

Table 610: Compounds of formula I.A* in which R⁴ is CH₃, R⁵ is cC₃H₅, Z is Q2, R^{6a} is 2-pyridyl, R^{6b} is methyl, and the combination of R¹, X, and (R³)ₙ for a compound corresponds in each case to one row of Table A

Table 611: Compounds of formula I.A* in which R⁴ is CH₃, R⁵ is H, Z is Q3, R^{6a} is 2-pyridyl, R^{6b} is methyl, and the combination of R¹, X, and (R³)ₙ for a compound corresponds in each case to one row of Table A

Table 612: Compounds of formula I.A* in which R⁴ is CH₃, R⁵ is F, Z is Q3, R^{6a} is 2-pyridyl, R^{6b} is methyl, and the combination of R¹, X, and (R³)ₙ for a compound corresponds in each case to one row of Table A

Table 613: Compounds of formula I.A* in which R⁴ is CH₃, R⁵ is Cl, Z is Q3, R^{6a} is 2-pyridyl, R^{6b} is methyl, and the combination of R¹, X, and (R³)ₙ for a compound corresponds in each case to one row of Table A

Table 614: Compounds of formula I.A* in which R⁴ is CH₃, R⁵ is CH₃, Z is Q3, R^{6a} is 2-pyridyl, R^{6b} is methyl, and the combination of R¹, X, and (R³)ₙ for a compound corresponds in each case to one row of Table A

Table 615: Compounds of formula I.A* in which R⁴ is CH₃, R⁵ is cC₃H₅, Z is Q3, R^{6a} is 2-pyridyl, R^{6b} is methyl, and the combination of R¹, X, and (R³)ₙ for a compound corresponds in each case to one row of Table A

Table 616: Compounds of formula I.A* in which R⁴ is CH₃, R⁵ is H, Z is Q4, R^{6a} is 2-pyridyl, R^{6b} is methyl, and the combination of R¹, X, and (R³)ₙ for a compound corresponds in each case to one row of Table A

Table 617: Compounds of formula I.A* in which R⁴ is CH₃, R⁵ is F, Z is Q4, R^{6a} is 2-pyridyl, R^{6b} is methyl, and the combination of R¹, X, and (R³)ₙ for a compound corresponds in each case to one row of Table A

Table 618: Compounds of formula I.A* in which R⁴ is CH₃, R⁵ is Cl, Z is Q4, R^{6a} is 2-pyridyl, R^{6b} is methyl, and the combination of R¹, X, and (R³)ₙ for a compound corresponds in each case to one row of Table A

Table 619: Compounds of formula I.A* in which R⁴ is CH₃, R⁵ is CH₃, Z is Q4, R^{6a} is 2-pyridyl, R^{6b} is methyl, and the combination of R¹, X, and (R³)ₙ for a compound corresponds in each case to one row of Table A

Table 620: Compounds of formula I.A* in which R⁴ is CH₃, R⁵ is cC₃H₅, Z is Q4, R^{6a} is 2-pyridyl, R^{6b} is methyl, and the combination of R¹, X, and (R³)ₙ for a compound corresponds in each case to one row of Table A

Table 621: Compounds of formula I.A* in which R⁴ is CH₃, R⁵ is H, Z is Q1, R^{6a} is 3-pyridyl, R^{6b} is methyl, and the combination of R¹, X, and (R³)ₙ for a compound corresponds in each case to one row of Table A

Table 622: Compounds of formula I.A* in which R⁴ is CH₃, R⁵ is F, Z is Q1, R^{6a} is 3-pyridyl, R^{6b} is methyl, and the combination of R¹, X, and (R³)ₙ for a compound corresponds in each case to one row of Table A

Table 623: Compounds of formula I.A* in which R⁴ is CH₃, R⁵ is Cl, Z is Q1, R^{6a} is 3-pyridyl, R^{6b} is methyl, and the combination of R¹, X, and (R³)ₙ for a compound corresponds in each case to one row of Table A

Table 624: Compounds of formula I.A* in which R⁴ is CH₃, R⁵ is CH₃, Z is Q1, R^{6a} is 3-pyridyl, R^{6b} is methyl, and the combination of R¹, X, and (R³)ₙ for a compound corresponds in each case to one row of Table A

Table 625: Compounds of formula I.A* in which R⁴ is CH₃, R⁵ is cC₃H₅, Z is Q1, R^{6a} is 3-pyridyl, R^{6b} is methyl, and the combination of R¹, X, and (R³)ₙ for a compound corresponds in each case to one row of Table A

Table 626: Compounds of formula I.A* in which R⁴ is CH₃, R⁵ is H, Z is Q2, R^{6a} is 3-pyridyl, R^{6b} is methyl, and the combination of R¹, X, and (R³)ₙ for a compound corresponds in each case to one row of Table A

Table 627: Compounds of formula I.A* in which R⁴ is CH₃, R⁵ is F, Z is Q2, R^{6a} is 3-pyridyl, R^{6b} is methyl, and the combination of R¹, X, and (R³)ₙ for a compound corresponds in each case to one row of Table A

Table 628: Compounds of formula I.A* in which R⁴ is CH₃, R⁵ is Cl, Z is Q2, R^{6a} is 3-pyridyl, R^{6b} is methyl, and the combination of R¹, X, and (R³)ₙ for a compound corresponds in each case to one row of Table A

Table 629: Compounds of formula I.A* in which R⁴ is CH₃, R⁵ is CH₃, Z is Q2, R^{6a} is 3-pyridyl, R^{6b} is methyl, and the combination of R¹, X, and (R³)ₙ for a compound corresponds in each case to one row of Table A

Table 630: Compounds of formula I.A* in which R⁴ is CH₃, R⁵ is cC₃H₅, Z is Q2, R^{6a} is 3-pyridyl, R^{6b} is methyl, and the combination of R¹, X, and (R³)ₙ for a compound corresponds in each case to one row of Table A

Table 631: Compounds of formula I.A* in which R⁴ is CH₃, R⁵ is H, Z is Q3, R^{6a} is 3-pyridyl, R^{6b} is methyl, and the combination of R¹, X, and (R³)ₙ for a compound corresponds in each case to one row of Table A

Table 632: Compounds of formula I.A* in which R⁴ is CH₃, R⁵ is F, Z is Q3, R^{6a} is 3-pyridyl, R^{6b} is methyl, and the combination of R¹, X, and (R³)ₙ for a compound corresponds in each case to one row of Table A

Table 633: Compounds of formula I.A* in which R⁴ is CH₃, R⁵ is Cl, Z is Q3, R^{6a} is 3-pyridyl, R^{6b} is methyl, and the combination of R¹, X, and (R³)ₙ for a compound corresponds in each case to one row of Table A

Table 634: Compounds of formula I.A* in which R⁴ is CH₃, R⁵ is CH₃, Z is Q3, R^{6a} is 3-pyridyl, R^{6b} is methyl, and the combination of R¹, X, and (R³)ₙ for a compound corresponds in each case to one row of Table A

Table 635: Compounds of formula I.A* in which R⁴ is CH₃, R⁵ is cC₃H₅, Z is Q3, R^{6a} is 3-pyridyl, R^{6b} is methyl, and the combination of R¹, X, and (R³)ₙ for a compound corresponds in each case to one row of Table A

Table 636: Compounds of formula I.A* in which R⁴ is CH₃, R⁵ is H, Z is Q4, R^{6a} is 3-pyridyl, R^{6b} is methyl, and the combination of R¹, X, and (R³)ₙ for a compound corresponds in each case to one row of Table A

Table 637: Compounds of formula I.A* in which R⁴ is CH₃, R⁵ is F, Z is Q4, R^{6a} is 3-pyridyl, R^{6b} is methyl, and the combination of R¹, X, and (R³)ₙ for a compound corresponds in each case to one row of Table A

Table 638: Compounds of formula I.A* in which R⁴ is CH₃, R⁵ is Cl, Z is Q4, R^{6a} is 3-pyridyl, R^{6b} is methyl, and the combination of R¹, X, and (R³)ₙ for a compound corresponds in each case to one row of Table A

Table 639: Compounds of formula I.A* in which R⁴ is CH₃, R⁵ is CH₃, Z is Q4, R^{6a} is 3-pyridyl, R^{6b} is methyl, and the combination of R¹, X, and (R³)ₙ for a compound corresponds in each case to one row of Table A

Table 640: Compounds of formula I.A* in which R⁴ is CH₃, R⁵ is cC₃H₅, Z is Q4, R^{6a} is 3-pyridyl, R^{6b} is methyl, and the combination of R¹, X, and (R³)ₙ for a compound corresponds in each case to one row of Table A

Table 641: Compounds of formula I.A* in which R⁴ and R⁵ are H, Z is Q1, R^{6a} is 2-pyridyl, R^{6b} is cyclopropyl, and the combination of R¹, X, and (R³)ₙ for a compound corresponds in each case to one row of Table A

Table 642: Compounds of formula I.A* in which R⁴ is H, R⁵ is F, Z is Q1, R^{6a} is 2-pyridyl, R^{6b} is cyclopropyl, and the combination of R¹, X, and (R³)ₙ for a compound corresponds in each case to one row of Table A

Table 643: Compounds of formula I.A* in which R⁴ is H, R⁵ is Cl, Z is Q1, R^{6a} is 2-pyridyl, R^{6b} is cyclopropyl, and the combination of R¹, X, and (R³)ₙ for a compound corresponds in each case to one row of Table A

Table 644: Compounds of formula I.A* in which R⁴ is H, R⁵ is CH₃, Z is Q1, R^{6a} is 2-pyridyl, R^{6b} is cyclopropyl, and the combination of R¹, X, and (R³)ₙ for a compound corresponds in each case to one row of Table A

Table 645: Compounds of formula I.A* in which R⁴ is H, R⁵ is cC₃H₅, Z is Q1, R^{6a} is 2-pyridyl, R^{6b} is cyclopropyl, and the combination of R¹, X, and (R³)ₙ for a compound corresponds in each case to one row of Table A

Table 646: Compounds of formula I.A* in which R⁴ and R⁵ are H, Z is Q2, R^{6a} is 2-pyridyl, R^{6b} is cyclopropyl, and the combination of R¹, X, and (R³)ₙ for a compound corresponds in each case to one row of Table A

Table 647: Compounds of formula I.A* in which R⁴ is H, R⁵ is F, Z is Q2, R^{6a} is 2-pyridyl, R^{6b} is cyclopropyl, and the combination of R¹, X, and (R³)ₙ for a compound corresponds in each case to one row of Table A

Table 648: Compounds of formula I.A* in which R⁴ is H, R⁵ is Cl, Z is Q2, R^{6a} is 2-pyridyl, R^{6b} is cyclopropyl, and the combination of R¹, X, and (R³)ₙ for a compound corresponds in each case to one row of Table A

Table 649: Compounds of formula I.A* in which R⁴ is H, R⁵ is CH₃, Z is Q2, R^{6a} is 2-pyridyl, R^{6b} is cyclopropyl, and the combination of R¹, X, and (R³)ₙ for a compound corresponds in each case to one row of Table A

Table 650: Compounds of formula I.A* in which R⁴ is H, R⁵ is cC₃H₅, Z is Q2, R^{6a} is 2-pyridyl, R^{6b} is cyclopropyl, and the combination of R¹, X, and (R³)ₙ for a compound corresponds in each case to one row of Table A

Table 651: Compounds of formula I.A* in which R⁴ and R⁵ are H, Z is Q3, R^{6a} is 2-pyridyl, R^{6b} is cyclopropyl, and the combination of R¹, X, and (R³)ₙ for a compound corresponds in each case to one row of Table A

Table 652: Compounds of formula I.A* in which R⁴ is H, R⁵ is F, Z is Q3, R^{6a} is 2-pyridyl, R^{6b} is cyclopropyl, and the combination of R¹, X, and (R³)ₙ for a compound corresponds in each case to one row of Table A

Table 653: Compounds of formula I.A* in which R⁴ is H, R⁵ is Cl, Z is Q3, R^{6a} is 2-pyridyl, R^{6b} is cyclopropyl, and the combination of R¹, X, and (R³)ₙ for a compound corresponds in each case to one row of Table A

Table 654: Compounds of formula I.A* in which R⁴ is H, R⁵ is CH₃, Z is Q3, R^{6a} is 2-pyridyl, R^{6b} is cyclopropyl, and the combination of R¹, X, and (R³)ₙ for a compound corresponds in each case to one row of Table A

Table 655: Compounds of formula I.A* in which R⁴ is H, R⁵ is cC₃H₅, Z is Q3, R^{6a} is 2-pyridyl, R^{6b} is cyclopropyl, and the combination of R¹, X, and (R³)ₙ for a compound corresponds in each case to one row of Table A

Table 656: Compounds of formula I.A* in which R⁴ and R⁵ are H, Z is Q4, R^{6a} is 2-pyridyl, R^{6b} is cyclopropyl, and the combination of R¹, X, and (R³)ₙ for a compound corresponds in each case to one row of Table A

Table 657: Compounds of formula I.A* in which R⁴ is H, R⁵ is F, Z is Q4, R^{6a} is 2-pyridyl, R^{6b} is cyclopropyl, and the combination of R¹, X, and (R³)ₙ for a compound corresponds in each case to one row of Table A

Table 658: Compounds of formula I.A* in which R⁴ is H, R⁵ is Cl, Z is Q4, R^{6a} is 2-pyridyl, R^{6b} is cyclopropyl, and the combination of R¹, X, and (R³)ₙ for a compound corresponds in each case to one row of Table A

Table 659: Compounds of formula I.A* in which R⁴ is H, R⁵ is CH₃, Z is Q4, R^{6a} is 2-pyridyl, R^{6b} is cyclopropyl, and the combination of R¹, X, and (R³)ₙ for a compound corresponds in each case to one row of Table A

Table 660: Compounds of formula I.A* in which R⁴ is H, R⁵ is cC₃H₅, Z is Q4, R^{6a} is 2-pyridyl, R^{6b} is cyclopropyl, and the combination of R¹, X, and (R³)ₙ for a compound corresponds in each case to one row of Table A

Table 661: Compounds of formula I.A* in which R⁴ and R⁵ are H, Z is Q1, R^{6a} is 3-pyridyl, R^{6b} is cyclopropyl, and the combination of R¹, X, and (R³)ₙ for a compound corresponds in each case to one row of Table A

Table 662: Compounds of formula I.A* in which R⁴ is H, R⁵ is F, Z is Q1, R^{6a} is 3-pyridyl, R^{6b} is cyclopropyl, and the combination of R¹, X, and (R³)ₙ for a compound corresponds in each case to one row of Table A

Table 663: Compounds of formula I.A* in which R⁴ is H, R⁵ is Cl, Z is Q1, R^{6a} is 3-pyridyl, R^{6b} is cyclopropyl, and the combination of R¹, X, and (R³)ₙ for a compound corresponds in each case to one row of Table A

Table 664: Compounds of formula I.A* in which R⁴ is H, R⁵ is CH₃, Z is Q1, R^{6a} is 3-pyridyl, R^{6b} is cyclopropyl, and the combination of R¹, X, and (R³)ₙ for a compound corresponds in each case to one row of Table A

Table 665: Compounds of formula I.A* in which R⁴ is H, R⁵ is cC₃H₅, Z is Q1, R^{6a} is 3-pyridyl, R^{6b} is cyclopropyl, and the combination of R¹, X, and (R³)ₙ for a compound corresponds in each case to one row of Table A

Table 666: Compounds of formula I.A* in which R⁴ and R⁵ are H, Z is Q2, R^{6a} is 3-pyridyl, R^{6b} is cyclopropyl, and the combination of R¹, X, and (R³)ₙ for a compound corresponds in each case to one row of Table A

Table 667: Compounds of formula I.A* in which R⁴ is H, R⁵ is F, Z is Q2, R^{6a} is 3-pyridyl, R^{6b} is cyclopropyl, and the combination of R¹, X, and (R³)ₙ for a compound corresponds in each case to one row of Table A

Table 668: Compounds of formula I.A* in which R⁴ is H, R⁵ is Cl, Z is Q2, R^{6a} is 3-pyridyl, R^{6b} is cyclopropyl, and the combination of R¹, X, and (R³)ₙ for a compound corresponds in each case to one row of Table A

Table 669: Compounds of formula I.A* in which R⁴ is H, R⁵ is CH₃, Z is Q2, R^{6a} is 3-pyridyl, R^{6b} is cyclopropyl, and the combination of R¹, X, and (R³)ₙ for a compound corresponds in each case to one row of Table A

Table 670: Compounds of formula I.A* in which R⁴ is H, R⁵ is cC₃H₅, Z is Q2, R^{6a} is 3-pyridyl, R^{6b} is cyclopropyl, and the combination of R¹, X, and (R³)ₙ for a compound corresponds in each case to one row of Table A

Table 671: Compounds of formula I.A* in which R⁴ and R⁵ are H, Z is Q3, R^{6a} is 3-pyridyl, R^{6b} is cyclopropyl, and the combination of R¹, X, and (R³)ₙ for a compound corresponds in each case to one row of Table A

Table 672: Compounds of formula I.A* in which R⁴ is H, R⁵ is F, Z is Q3, R^{6a} is 3-pyridyl, R^{6b} is cyclopropyl, and the combination of R¹, X, and (R³)ₙ for a compound corresponds in each case to one row of Table A

Table 673: Compounds of formula I.A* in which R⁴ is H, R⁵ is Cl, Z is Q3, R^{6a} is 3-pyridyl, R^{6b} is cyclopropyl, and the combination of R¹, X, and (R³)ₙ for a compound corresponds in each case to one row of Table A

Table 674: Compounds of formula I.A* in which R⁴ is H, R⁵ is CH₃, Z is Q3, R^{6a} is 3-pyridyl, R^{6b} is cyclopropyl, and the combination of R¹, X, and (R³)ₙ for a compound corresponds in each case to one row of Table A

Table 675: Compounds of formula I.A* in which R⁴ is H, R⁵ is cC₃H₅, Z is Q3, R^{6a} is 3-pyridyl, R^{6b} is cyclopropyl, and the combination of R¹, X, and (R³)ₙ for a compound corresponds in each case to one row of Table A

Table 676: Compounds of formula I.A* in which R⁴ and R⁵ are H, Z is Q4, R^{6a} is 3-pyridyl, R^{6b} is cyclopropyl, and the combination of R¹, X, and (R³)ₙ for a compound corresponds in each case to one row of Table A

Table 677: Compounds of formula I.A* in which R⁴ is H, R⁵ is F, Z is Q4, R^{6a} is 3-pyridyl, R^{6b} is cyclopropyl, and the combination of R¹, X, and (R³)ₙ for a compound corresponds in each case to one row of Table A

Table 678: Compounds of formula I.A* in which R⁴ is H, R⁵ is Cl, Z is Q4, R^{6a} is 3-pyridyl, R^{6b} is cyclopropyl, and the combination of R¹, X, and (R³)ₙ for a compound corresponds in each case to one row of Table A

Table 679: Compounds of formula I.A* in which R⁴ is H, R⁵ is CH₃, Z is Q4, R^{6a} is 3-pyridyl, R^{6b} is cyclopropyl, and the combination of R¹, X, and (R³)ₙ for a compound corresponds in each case to one row of Table A

Table 680: Compounds of formula I.A* in which R⁴ is H, R⁵ is cC₃H₅, Z is Q4, R^{6a} is 3-pyridyl, R^{6b} is cyclopropyl, and the combination of R¹, X, and (R³)ₙ for a compound corresponds in each case to one row of Table A

Table 681: Compounds of formula I.A* in which R⁴ is F, R⁵ is H, Z is Q1, R^{6a} is 2-pyridyl, R^{6b} is cyclopropyl, and the combination of R¹, X, and (R³)ₙ for a compound corresponds in each case to one row of Table A

Table 682: Compounds of formula I.A* in which R⁴ is F, R⁵ is F, Z is Q1, R^{6a} is 2-pyridyl, R^{6b} is cyclopropyl, and the combination of R¹, X, and (R³)ₙ for a compound corresponds in each case to one row of Table A

Table 683: Compounds of formula I.A* in which R⁴ is F, R⁵ is Cl, Z is Q1, R^{6a} is 2-pyridyl, R^{6b} is cyclopropyl, and the combination of R¹, X, and (R³)ₙ for a compound corresponds in each case to one row of Table A

Table 684: Compounds of formula I.A* in which R⁴ is F, R⁵ is CH₃, Z is Q1, R^{6a} is 2-pyridyl, R^{6b} is cyclopropyl, and the combination of R¹, X, and (R³)ₙ for a compound corresponds in each case to one row of Table A

Table 685: Compounds of formula I.A* in which R⁴ is F, R⁵ is cC₃H₅, Z is Q1, R^{6a} is 2-pyridyl, R^{6b} is cyclopropyl, and the combination of R¹, X, and (R³)ₙ for a compound corresponds in each case to one row of Table A

Table 686: Compounds of formula I.A* in which R⁴ is F, R⁵ is H, Z is Q2, R^{6a} is 2-pyridyl, R^{6b} is cyclopropyl, and the combination of R¹, X, and (R³)ₙ for a compound corresponds in each case to one row of Table A

Table 687: Compounds of formula I.A* in which R⁴ is F, R⁵ is F, Z is Q2, R^{6a} is 2-pyridyl, R^{6b} is cyclopropyl, and the combination of R¹, X, and (R³)ₙ for a compound corresponds in each case to one row of Table A

Table 688: Compounds of formula I.A* in which R⁴ is F, R⁵ is Cl, Z is Q2, R^{6a} is 2-pyridyl, R^{6b} is cyclopropyl, and the combination of R¹, X, and (R³)ₙ for a compound corresponds in each case to one row of Table A

Table 689: Compounds of formula I.A* in which R⁴ is F, R⁵ is CH₃, Z is Q2, R^{6a} is 2-pyridyl, R^{6b} is cyclopropyl, and the combination of R¹, X, and (R³)ₙ for a compound corresponds in each case to one row of Table A

Table 690: Compounds of formula I.A* in which R⁴ is F, R⁵ is cC₃H₅, Z is Q2, R^{6a} is 2-pyridyl, R^{6b} is cyclopropyl, and the combination of R¹, X, and (R³)ₙ for a compound corresponds in each case to one row of Table A

Table 691: Compounds of formula I.A* in which R⁴ is F, R⁵ is H, Z is Q3, R^{6a} is 2-pyridyl, R^{6b} is cyclopropyl, and the combination of R¹, X, and (R³)ₙ for a compound corresponds in each case to one row of Table A

Table 692: Compounds of formula I.A* in which R⁴ is F, R⁵ is F, Z is Q3, R^{6a} is 2-pyridyl, R^{6b} is cyclopropyl, and the combination of R¹, X, and (R³)ₙ for a compound corresponds in each case to one row of Table A

Table 693: Compounds of formula I.A* in which R⁴ is F, R⁵ is Cl, Z is Q3, R^{6a} is 2-pyridyl, R^{6b} is cyclopropyl, and the combination of R¹, X, and (R³)ₙ for a compound corresponds in each case to one row of Table A

Table 694: Compounds of formula I.A* in which R⁴ is F, R⁵ is CH₃, Z is Q3, R^{6a} is 2-pyridyl, R^{6b} is cyclopropyl, and the combination of R¹, X, and (R³)ₙ for a compound corresponds in each case to one row of Table A

Table 695: Compounds of formula I.A* in which R⁴ is F, R⁵ is cC₃H₅, Z is Q3, R^{6a} is 2-pyridyl, R^{6b} is cyclopropyl, and the combination of R¹, X, and (R³)ₙ for a compound corresponds in each case to one row of Table A

Table 696: Compounds of formula I.A* in which R⁴ is F, R⁵ is H, Z is Q4, R^{6a} is 2-pyridyl, R^{6b} is cyclopropyl, and the combination of R¹, X, and (R³)ₙ for a compound corresponds in each case to one row of Table A

Table 697: Compounds of formula I.A* in which R⁴ is F, R⁵ is F, Z is Q4, R^{6a} is 2-pyridyl, R^{6b} is cyclopropyl, and the combination of R¹, X, and (R³)ₙ for a compound corresponds in each case to one row of Table A

Table 698: Compounds of formula I.A* in which R⁴ is F, R⁵ is Cl, Z is Q4, R^{6a} is 2-pyridyl, R^{6b} is cyclopropyl, and the combination of R¹, X, and (R³)ₙ for a compound corresponds in each case to one row of Table A

Table 699: Compounds of formula I.A* in which R⁴ is F, R⁵ is CH₃, Z is Q4, R^{6a} is 2-pyridyl, R^{6b} is cyclopropyl, and the combination of R¹, X, and (R³)ₙ for a compound corresponds in each case to one row of Table A

Table 700: Compounds of formula I.A* in which R⁴ is F, R⁵ is cC₃H₅, Z is Q4, R^{6a} is 2-pyridyl, R^{6b} is cyclopropyl, and the combination of R¹, X, and (R³)ₙ for a compound corresponds in each case to one row of Table A

Table 701: Compounds of formula I.A* in which R⁴ is F, R⁵ is H, Z is Q1, R^{6a} is 3-pyridyl, R^{6b} is cyclopropyl, and the combination of R¹, X, and (R³)ₙ for a compound corresponds in each case to one row of Table A

Table 702: Compounds of formula I.A* in which R⁴ is F, R⁵ is F, Z is Q1, R^{6a} is 3-pyridyl, R^{6b} is cyclopropyl, and the combination of R¹, X, and (R³)ₙ for a compound corresponds in each case to one row of Table A

Table 703: Compounds of formula I.A* in which R⁴ is F, R⁵ is Cl, Z is Q1, R^{6a} is 3-pyridyl, R^{6b} is cyclopropyl, and the combination of R¹, X, and (R³)ₙ for a compound corresponds in each case to one row of Table A

Table 704: Compounds of formula I.A* in which R⁴ is F, R⁵ is CH₃, Z is Q1, R^{6a} is 3-pyridyl, R^{6b} is cyclopropyl, and the combination of R¹, X, and (R³)ₙ for a compound corresponds in each case to one row of Table A

Table 705: Compounds of formula I.A* in which R⁴ is F, R⁵ is cC₃H₅, Z is Q1, R^{6a} is 3-pyridyl, R^{6b} is cyclopropyl, and the combination of R¹, X, and (R³)ₙ for a compound corresponds in each case to one row of Table A

Table 706: Compounds of formula I.A* in which R⁴ is F, R⁵ is H, Z is Q2, R^{6a} is 3-pyridyl, R^{6b} is cyclopropyl, and the combination of R¹, X, and (R³)ₙ for a compound corresponds in each case to one row of Table A

Table 707: Compounds of formula I.A* in which R⁴ is F, R⁵ is F, Z is Q2, R^{6a} is 3-pyridyl, R^{6b} is cyclopropyl, and the combination of R¹, X, and (R³)ₙ for a compound corresponds in each case to one row of Table A

Table 708: Compounds of formula I.A* in which R⁴ is F, R⁵ is Cl, Z is Q2, R^{6a} is 3-pyridyl, R^{6b} is cyclopropyl, and the combination of R¹, X, and (R³)ₙ for a compound corresponds in each case to one row of Table A

Table 709: Compounds of formula I.A* in which R⁴ is F, R⁵ is CH₃, Z is Q2, R^{6a} is 3-pyridyl, R^{6b} is cyclopropyl, and the combination of R¹, X, and (R³)ₙ for a compound corresponds in each case to one row of Table A

Table 710: Compounds of formula I.A* in which R⁴ is F, R⁵ is cC₃H₅, Z is Q2, R^{6a} is 3-pyridyl, R^{6b} is cyclopropyl, and the combination of R¹, X, and (R³)ₙ for a compound corresponds in each case to one row of Table A

Table 711: Compounds of formula I.A* in which R⁴ is F, R⁵ is H, Z is Q3, R^{6a} is 3-pyridyl, R^{6b} is cyclopropyl, and the combination of R¹, X, and (R³)ₙ for a compound corresponds in each case to one row of Table A

Table 712: Compounds of formula I.A* in which R⁴ is F, R⁵ is F, Z is Q3, R^{6a} is 3-pyridyl, R^{6b} is cyclopropyl, and the combination of R¹, X, and (R³)ₙ for a compound corresponds in each case to one row of Table A

Table 713: Compounds of formula I.A* in which R⁴ is F, R⁵ is Cl, Z is Q3, R^{6a} is 3-pyridyl, R^{6b} is cyclopropyl, and the combination of R¹, X, and (R³)ₙ for a compound corresponds in each case to one row of Table A

Table 714: Compounds of formula I.A* in which R⁴ is F, R⁵ is CH₃, Z is Q3, R^{6a} is 3-pyridyl, R^{6b} is cyclopropyl, and the combination of R¹, X, and (R³)ₙ for a compound corresponds in each case to one row of Table A

Table 715: Compounds of formula I.A* in which R⁴ is F, R⁵ is cC₃H₅, Z is Q3, R^{6a} is 3-pyridyl, R^{6b} is cyclopropyl, and the combination of R¹, X, and (R³)ₙ for a compound corresponds in each case to one row of Table A

Table 716: Compounds of formula I.A* in which R⁴ is F, R⁵ is H, Z is Q4, R^{6a} is 3-pyridyl, R^{6b} is cyclopropyl, and the combination of R¹, X, and (R³)ₙ for a compound corresponds in each case to one row of Table A

Table 717: Compounds of formula I.A* in which R⁴ is F, R⁵ is F, Z is Q4, R^{6a} is 3-pyridyl, R^{6b} is cyclopropyl, and the combination of R¹, X, and (R³)ₙ for a compound corresponds in each case to one row of Table A

Table 718: Compounds of formula I.A* in which R⁴ is F, R⁵ is Cl, Z is Q4, R^{6a} is 3-pyridyl, R^{6b} is cyclopropyl, and the combination of R¹, X, and (R³)ₙ for a compound corresponds in each case to one row of Table A

Table 719: Compounds of formula I.A* in which R⁴ is F, R⁵ is CH₃, Z is Q4, R^{6a} is 3-pyridyl, R^{6b} is cyclopropyl, and the combination of R¹, X, and (R³)ₙ for a compound corresponds in each case to one row of Table A

Table 720: Compounds of formula I.A* in which R⁴ is F, R⁵ is cC₃H₅, Z is Q4, R^{6a} is 3-pyridyl, R^{6b} is cyclopropyl, and the combination of R¹, X, and (R³)ₙ for a compound corresponds in each case to one row of Table A

Table 721: Compounds of formula I.A* in which R⁴ is Cl, R⁵ is H, Z is Q1, R^{6a} is 2-pyridyl, R^{6b} is cyclopropyl, and the combination of R¹, X, and (R³)ₙ for a compound corresponds in each case to one row of Table A

Table 722: Compounds of formula I.A* in which R⁴ is Cl, R⁵ is F, Z is Q1, R^{6a} is 2-pyridyl, R^{6b} is cyclopropyl, and the combination of R¹, X, and (R³)ₙ for a compound corresponds in each case to one row of Table A

Table 723: Compounds of formula I.A* in which R⁴ is Cl, R⁵ is Cl, Z is Q1, R^{6a} is 2-pyridyl, R^{6b} is cyclopropyl, and the combination of R¹, X, and (R³)ₙ for a compound corresponds in each case to one row of Table A

Table 724: Compounds of formula I.A* in which R⁴ is Cl, R⁵ is CH₃, Z is Q1, R^{6a} is 2-pyridyl, R^{6b} is cyclopropyl, and the combination of R¹, X, and (R³)ₙ for a compound corresponds in each case to one row of Table A

Table 725: Compounds of formula I.A* in which R⁴ is Cl, R⁵ is cC₃H₅, Z is Q1, R^{6a} is 2-pyridyl, R^{6b} is cyclopropyl, and the combination of R¹, X, and (R³)ₙ for a compound corresponds in each case to one row of Table A

Table 726: Compounds of formula I.A* in which R⁴ is Cl, R⁵ is H, Z is Q2, R^{6a} is 2-pyridyl, R^{6b} is cyclopropyl, and the combination of R¹, X, and (R³)ₙ for a compound corresponds in each case to one row of Table A

Table 727: Compounds of formula I.A* in which R⁴ is Cl, R⁵ is F, Z is Q2, R^{6a} is 2-pyridyl, R^{6b} is cyclopropyl, and the combination of R¹, X, and (R³)ₙ for a compound corresponds in each case to one row of Table A

Table 728: Compounds of formula I.A* in which R⁴ is Cl, R⁵ is Cl, Z is Q2, R^{6a} is 2-pyridyl, R^{6b} is cyclopropyl, and the combination of R¹, X, and (R³)ₙ for a compound corresponds in each case to one row of Table A

Table 729: Compounds of formula I.A* in which R⁴ is Cl, R⁵ is CH₃, Z is Q2, R^{6a} is 2-pyridyl, R^{6b} is cyclopropyl, and the combination of R¹, X, and (R³)ₙ for a compound corresponds in each case to one row of Table A

Table 730: Compounds of formula I.A* in which R⁴ is Cl, R⁵ is cC₃H₅, Z is Q2, R^{6a} is 2-pyridyl, R^{6b} is cyclopropyl, and the combination of R¹, X, and (R³)ₙ for a compound corresponds in each case to one row of Table A

Table 731: Compounds of formula I.A* in which R⁴ is Cl, R⁵ is H, Z is Q3, R^{6a} is 2-pyridyl, R^{6b} is cyclopropyl, and the combination of R¹, X, and (R³)ₙ for a compound corresponds in each case to one row of Table A

Table 732: Compounds of formula I.A* in which R⁴ is Cl, R⁵ is F, Z is Q3, R^{6a} is 2-pyridyl, R^{6b} is cyclopropyl, and the combination of R¹, X, and (R³)ₙ for a compound corresponds in each case to one row of Table A

Table 733: Compounds of formula I.A* in which R⁴ is Cl, R⁵ is Cl, Z is Q3, R^{6a} is 2-pyridyl, R^{6b} is cyclopropyl, and the combination of R¹, X, and (R³)ₙ for a compound corresponds in each case to one row of Table A

Table 734: Compounds of formula I.A* in which R⁴ is Cl, R⁵ is CH₃, Z is Q3, R^{6a} is 2-pyridyl, R^{6b} is cyclopropyl, and the combination of R¹, X, and (R³)ₙ for a compound corresponds in each case to one row of Table A

Table 735: Compounds of formula I.A* in which R⁴ is Cl, R⁵ is cC₃H₅, Z is Q3, R^{6a} is 2-pyridyl, R^{6b} is cyclopropyl, and the combination of R¹, X, and (R³)ₙ for a compound corresponds in each case to one row of Table A

Table 736: Compounds of formula I.A* in which R⁴ is Cl, R⁵ is H, Z is Q4, R^{6a} is 2-pyridyl, R^{6b} is cyclopropyl, and the combination of R¹, X, and (R³)ₙ for a compound corresponds in each case to one row of Table A

Table 737: Compounds of formula I.A* in which R⁴ is Cl, R⁵ is F, Z is Q4, R^{6a} is 2-pyridyl, R^{6b} is cyclopropyl, and the combination of R¹, X, and (R³)ₙ for a compound corresponds in each case to one row of Table A

Table 738: Compounds of formula I.A* in which R⁴ is Cl, R⁵ is Cl, Z is Q4, R^{6a} is 2-pyridyl, R^{6b} is cyclopropyl, and the combination of R¹, X, and (R³)ₙ for a compound corresponds in each case to one row of Table A

Table 739: Compounds of formula I.A* in which R⁴ is Cl, R⁵ is CH₃, Z is Q4, R^{6a} is 2-pyridyl, R^{6b} is cyclopropyl, and the combination of R¹, X, and (R³)ₙ for a compound corresponds in each case to one row of Table A

Table 740: Compounds of formula I.A* in which R⁴ is Cl, R⁵ is cC₃H₅, Z is Q4, R^{6a} is 2-pyridyl, R^{6b} is cyclopropyl, and the combination of R¹, X, and (R³)ₙ for a compound corresponds in each case to one row of Table A

Table 741: Compounds of formula I.A* in which R⁴ is Cl, R⁵ is H, Z is Q1, R^{6a} is 3-pyridyl, R^{6b} is cyclopropyl, and the combination of R¹, X, and (R³)ₙ for a compound corresponds in each case to one row of Table A

Table 742: Compounds of formula I.A* in which R⁴ is Cl, R⁵ is F, Z is Q1, R^{6a} is 3-pyridyl, R^{6b} is cyclopropyl, and the combination of R¹, X, and (R³)ₙ for a compound corresponds in each case to one row of Table A

Table 743: Compounds of formula I.A* in which R⁴ is Cl, R⁵ is Cl, Z is Q1, R^{6a} is 3-pyridyl, R^{6b} is cyclopropyl, and the combination of R¹, X, and (R³)ₙ for a compound corresponds in each case to one row of Table A

Table 744: Compounds of formula I.A* in which R⁴ is Cl, R⁵ is CH₃, Z is Q1, R^{6a} is 3-pyridyl, R^{6b} is cyclopropyl, and the combination of R¹, X, and (R³)ₙ for a compound corresponds in each case to one row of Table A

Table 745: Compounds of formula I.A* in which R⁴ is Cl, R⁵ is cC₃H₅, Z is Q1, R^{6a} is 3-pyridyl, R^{6b} is cyclopropyl, and the combination of R¹, X, and (R³)ₙ for a compound corresponds in each case to one row of Table A

Table 746: Compounds of formula I.A* in which R⁴ is Cl, R⁵ is H, Z is Q2, R^{6a} is 3-pyridyl, R^{6b} is cyclopropyl, and the combination of R¹, X, and (R³)ₙ for a compound corresponds in each case to one row of Table A

Table 747: Compounds of formula I.A* in which R⁴ is Cl, R⁵ is F, Z is Q2, R^{6a} is 3-pyridyl, R^{6b} is cyclopropyl, and the combination of R¹, X, and (R³)ₙ for a compound corresponds in each case to one row of Table A

Table 748: Compounds of formula I.A* in which R⁴ is Cl, R⁵ is Cl, Z is Q2, R^{6a} is 3-pyridyl, R^{6b} is cyclopropyl, and the combination of R¹, X, and (R³)ₙ for a compound corresponds in each case to one row of Table A

Table 749: Compounds of formula I.A* in which R⁴ is Cl, R⁵ is CH₃, Z is Q2, R^{6a} is 3-pyridyl, R^{6b} is cyclopropyl, and the combination of R¹, X, and (R³)ₙ for a compound corresponds in each case to one row of Table A

Table 750: Compounds of formula I.A* in which R⁴ is Cl, R⁵ is cC₃H₅, Z is Q2, R^{6a} is 3-pyridyl, R^{6b} is cyclopropyl, and the combination of R¹, X, and (R³)ₙ for a compound corresponds in each case to one row of Table A

Table 751: Compounds of formula I.A* in which R⁴ is Cl, R⁵ is H, Z is Q3, R^{6a} is 3-pyridyl, R^{6b} is cyclopropyl, and the combination of R¹, X, and (R³)ₙ for a compound corresponds in each case to one row of Table A

Table 752: Compounds of formula I.A* in which R⁴ is Cl, R⁵ is F, Z is Q3, R^{6a} is 3-pyridyl, R^{6b} is cyclopropyl, and the combination of R¹, X, and (R³)ₙ for a compound corresponds in each case to one row of Table A

Table 753: Compounds of formula I.A* in which R⁴ is Cl, R⁵ is Cl, Z is Q3, R^{6a} is 3-pyridyl, R^{6b} is cyclopropyl, and the combination of R¹, X, and (R³)ₙ for a compound corresponds in each case to one row of Table A

Table 754: Compounds of formula I.A* in which R⁴ is Cl, R⁵ is CH₃, Z is Q3, R^{6a} is 3-pyridyl, R^{6b} is cyclopropyl, and the combination of R¹, X, and (R³)ₙ for a compound corresponds in each case to one row of Table A

Table 755: Compounds of formula I.A* in which R⁴ is Cl, R⁵ is cC₃H₅, Z is Q3, R^{6a} is 3-pyridyl, R^{6b} is cyclopropyl, and the combination of R¹, X, and (R³)ₙ for a compound corresponds in each case to one row of Table A

Table 756: Compounds of formula I.A* in which R⁴ is Cl, R⁵ is H, Z is Q4, R^{6a} is 3-pyridyl, R^{6b} is cyclopropyl, and the combination of R¹, X, and (R³)ₙ for a compound corresponds in each case to one row of Table A

Table 757: Compounds of formula I.A* in which R⁴ is Cl, R⁵ is F, Z is Q4, R^{6a} is 3-pyridyl, R^{6b} is cyclopropyl, and the combination of R¹, X, and (R³)ₙ for a compound corresponds in each case to one row of Table A

Table 758: Compounds of formula I.A* in which R⁴ is Cl, R⁵ is Cl, Z is Q4, R^{6a} is 3-pyridyl, R^{6b} is cyclopropyl, and the combination of R¹, X, and (R³)ₙ for a compound corresponds in each case to one row of Table A

Table 759: Compounds of formula I.A* in which R⁴ is Cl, R⁵ is CH₃, Z is Q4, R^{6a} is 3-pyridyl, R^{6b} is cyclopropyl, and the combination of R¹, X, and (R³)ₙ for a compound corresponds in each case to one row of Table A

Table 760: Compounds of formula I.A* in which R⁴ is Cl, R⁵ is cC₃H₅, Z is Q4, R^{6a} is 3-pyridyl, R^{6b} is cyclopropyl, and the combination of R¹, X, and (R³)ₙ for a compound corresponds in each case to one row of Table A

Table 761: Compounds of formula I.A* in which R⁴ is CH₃, R⁵ is H, Z is Q1, R^{6a} is 2-pyridyl, R^{6b} is cyclopropyl, and the combination of R¹, X, and (R³)ₙ for a compound corresponds in each case to one row of Table A

Table 762: Compounds of formula I.A* in which R⁴ is CH₃, R⁵ is F, Z is Q1, R^{6a} is 2-pyridyl, R^{6b} is cyclopropyl, and the combination of R¹, X, and (R³)ₙ for a compound corresponds in each case to one row of Table A

Table 763: Compounds of formula I.A* in which R⁴ is CH₃, R⁵ is Cl, Z is Q1, R^{6a} is 2-pyridyl, R^{6b} is cyclopropyl, and the combination of R¹, X, and (R³)ₙ for a compound corresponds in each case to one row of Table A

Table 764: Compounds of formula I.A* in which R⁴ is CH₃, R⁵ is CH₃, Z is Q1, R^{6a} is 2-pyridyl, R^{6b} is cyclopropyl, and the combination of R¹, X, and (R³)ₙ for a compound corresponds in each case to one row of Table A

Table 765: Compounds of formula I.A* in which R⁴ is CH₃, R⁵ is cC₃H₅, Z is Q1, R^{6a} is 2-pyridyl, R^{6b} is cyclopropyl, and the combination of R¹, X, and (R³)ₙ for a compound corresponds in each case to one row of Table A

Table 766: Compounds of formula I.A* in which R⁴ is CH₃, R⁵ is H, Z is Q2, R^{6a} is 2-pyridyl, R^{6b} is cyclopropyl, and the combination of R¹, X, and (R³)ₙ for a compound corresponds in each case to one row of Table A

Table 767: Compounds of formula I.A* in which R⁴ is CH₃, R⁵ is F, Z is Q2, R^{6a} is 2-pyridyl, R^{6b} is cyclopropyl, and the combination of R¹, X, and (R³)ₙ for a compound corresponds in each case to one row of Table A

Table 768: Compounds of formula I.A* in which R⁴ is CH₃, R⁵ is Cl, Z is Q2, R^{6a} is 2-pyridyl, R^{6b} is cyclopropyl, and the combination of R¹, X, and (R³)ₙ for a compound corresponds in each case to one row of Table A

Table 769: Compounds of formula I.A* in which R⁴ is CH₃, R⁵ is CH₃, Z is Q2, R^{6a} is 2-pyridyl, R^{6b} is cyclopropyl, and the combination of R¹, X, and (R³)ₙ for a compound corresponds in each case to one row of Table A

Table 770: Compounds of formula I.A* in which R⁴ is CH₃, R⁵ is cC₃H₅, Z is Q2, R^{6a} is 2-pyridyl, R^{6b} is cyclopropyl, and the combination of R¹, X, and (R³)ₙ for a compound corresponds in each case to one row of Table A

Table 771: Compounds of formula I.A* in which R⁴ is CH₃, R⁵ is H, Z is Q3, R^{6a} is 2-pyridyl, R^{6b} is cyclopropyl, and the combination of R¹, X, and (R³)ₙ for a compound corresponds in each case to one row of Table A

Table 772: Compounds of formula I.A* in which R⁴ is CH₃, R⁵ is F, Z is Q3, R^{6a} is 2-pyridyl, R^{6b} is cyclopropyl, and the combination of R¹, X, and (R³)ₙ for a compound corresponds in each case to one row of Table A

Table 773: Compounds of formula I.A* in which R⁴ is CH₃, R⁵ is Cl, Z is Q3, R^{6a} is 2-pyridyl, R^{6b} is cyclopropyl, and the combination of R¹, X, and (R³)ₙ for a compound corresponds in each case to one row of Table A

Table 774: Compounds of formula I.A* in which R⁴ is CH₃, R⁵ is CH₃, Z is Q3, R^{6a} is 2-pyridyl, R^{6b} is cyclopropyl, and the combination of R¹, X, and (R³)ₙ for a compound corresponds in each case to one row of Table A

Table 775: Compounds of formula I.A* in which R⁴ is CH₃, R⁵ is cC₃H₅, Z is Q3, R^{6a} is 2-pyridyl, R^{6b} is cyclopropyl, and the combination of R¹, X, and (R³)ₙ for a compound corresponds in each case to one row of Table A

Table 776: Compounds of formula I.A* in which R⁴ is CH₃, R⁵ is H, Z is Q4, R^{6a} is 2-pyridyl, R^{6b} is cyclopropyl, and the combination of R¹, X, and (R³)ₙ for a compound corresponds in each case to one row of Table A

Table 777: Compounds of formula I.A* in which R⁴ is CH₃, R⁵ is F, Z is Q4, R^{6a} is 2-pyridyl, R^{6b} is cyclopropyl, and the combination of R¹, X, and (R³)ₙ for a compound corresponds in each case to one row of Table A

Table 778: Compounds of formula I.A* in which R⁴ is CH₃, R⁵ is Cl, Z is Q4, R^{6a} is 2-pyridyl, R^{6b} is cyclopropyl, and the combination of R¹, X, and (R³)ₙ for a compound corresponds in each case to one row of Table A

Table 779: Compounds of formula I.A* in which R⁴ is CH₃, R⁵ is CH₃, Z is Q4, R^{6a} is 2-pyridyl, R^{6b} is cyclopropyl, and the combination of R¹, X, and (R³)ₙ for a compound corresponds in each case to one row of Table A

Table 780: Compounds of formula I.A* in which R⁴ is CH₃, R⁵ is cC₃H₅, Z is Q4, R^{6a} is 2-pyridyl, R^{6b} is cyclopropyl, and the combination of R¹, X, and (R³)ₙ for a compound corresponds in each case to one row of Table A

Table 781: Compounds of formula I.A* in which R⁴ is CH₃, R⁵ is H, Z is Q1, R^{6a} is 3-pyridyl, R^{6b} is cyclopropyl, and the combination of R¹, X, and (R³)ₙ for a compound corresponds in each case to one row of Table A

Table 782: Compounds of formula I.A* in which R⁴ is CH₃, R⁵ is F, Z is Q1, R^{6a} is 3-pyridyl, R^{6b} is cyclopropyl, and the combination of R¹, X, and (R³)ₙ for a compound corresponds in each case to one row of Table A

Table 783: Compounds of formula I.A* in which R⁴ is CH₃, R⁵ is Cl, Z is Q1, R^{6a} is 3-pyridyl, R^{6b} is cyclopropyl, and the combination of R¹, X, and (R³)ₙ for a compound corresponds in each case to one row of Table A

Table 784: Compounds of formula I.A* in which R⁴ is CH₃, R⁵ is CH₃, Z is Q1, R^{6a} is 3-pyridyl, R^{6b} is cyclopropyl, and the combination of R¹, X, and (R³)ₙ for a compound corresponds in each case to one row of Table A

Table 785: Compounds of formula I.A* in which R⁴ is CH₃, R⁵ is cC₃H₅, Z is Q1, R^{6a} is 3-pyridyl, R^{6b} is cyclopropyl, and the combination of R¹, X, and (R³)ₙ for a compound corresponds in each case to one row of Table A

Table 786: Compounds of formula I.A* in which R⁴ is CH₃, R⁵ is H, Z is Q2, R^{6a} is 3-pyridyl, R^{6b} is cyclopropyl, and the combination of R¹, X, and (R³)ₙ for a compound corresponds in each case to one row of Table A

Table 787: Compounds of formula I.A* in which R⁴ is CH₃, R⁵ is F, Z is Q2, R^{6a} is 3-pyridyl, R^{6b} is cyclopropyl, and the combination of R¹, X, and (R³)ₙ for a compound corresponds in each case to one row of Table A

Table 788: Compounds of formula I.A* in which R⁴ is CH₃, R⁵ is Cl, Z is Q2, R^{6a} is 3-pyridyl, R^{6b} is cyclopropyl, and the combination of R¹, X, and (R³)ₙ for a compound corresponds in each case to one row of Table A

Table 789: Compounds of formula I.A* in which R⁴ is CH₃, R⁵ is CH₃, Z is Q2, R^{6a} is 3-pyridyl, R^{6b} is cyclopropyl, and the combination of R¹, X, and (R³)ₙ for a compound corresponds in each case to one row of Table A

Table 790: Compounds of formula I.A* in which R⁴ is CH₃, R⁵ is cC₃H₅, Z is Q2, R^{6a} is 3-pyridyl, R^{6b} is cyclopropyl, and the combination of R¹, X, and (R³)ₙ for a compound corresponds in each case to one row of Table A

Table 791: Compounds of formula I.A* in which R⁴ is CH₃, R⁵ is H, Z is Q3, R^{6a} is 3-pyridyl, R^{6b} is cyclopropyl, and the combination of R¹, X, and (R³)ₙ for a compound corresponds in each case to one row of Table A

Table 792: Compounds of formula I.A* in which R⁴ is CH₃, R⁵ is F, Z is Q3, R^{6a} is 3-pyridyl, R^{6b} is cyclopropyl, and the combination of R¹, X, and (R³)ₙ for a compound corresponds in each case to one row of Table A

Table 793: Compounds of formula I.A* in which R⁴ is CH₃, R⁵ is Cl, Z is Q3, R^{6a} is 3-pyridyl, R^{6b} is cyclopropyl, and the combination of R¹, X, and (R³)ₙ for a compound corresponds in each case to one row of Table A

Table 794: Compounds of formula I.A* in which R⁴ is CH₃, R⁵ is CH₃, Z is Q3, R^{6a} is 3-pyridyl, R^{6b} is cyclopropyl, and the combination of R¹, X, and (R³)ₙ for a compound corresponds in each case to one row of Table A

Table 795: Compounds of formula I.A* in which R⁴ is CH₃, R⁵ is cC₃H₅, Z is Q3, R^{6a} is 3-pyridyl, R^{6b} is cyclopropyl, and the combination of R¹, X, and (R³)ₙ for a compound corresponds in each case to one row of Table A

Table 796: Compounds of formula I.A* in which R⁴ is CH₃, R⁵ is H, Z is Q4, R^{6a} is 3-pyridyl, R^{6b} is cyclopropyl, and the combination of R¹, X, and (R³)ₙ for a compound corresponds in each case to one row of Table A

Table 797: Compounds of formula I.A* in which R⁴ is CH₃, R⁵ is F, Z is Q4, R^{6a} is 3-pyridyl, R^{6b} is cyclopropyl, and the combination of R¹, X, and (R³)ₙ for a compound corresponds in each case to one row of Table A

Table 798: Compounds of formula I.A* in which R⁴ is CH₃, R⁵ is Cl, Z is Q4, R^{6a} is 3-pyridyl, R^{6b} is cyclopropyl, and the combination of R¹, X, and (R³)ₙ for a compound corresponds in each case to one row of Table A

Table 799: Compounds of formula I.A* in which R⁴ is CH₃, R⁵ is CH₃, Z is Q4, R^{6a} is 3-pyridyl, R^{6b} is cyclopropyl, and the combination of R¹, X, and (R³)ₙ for a compound corresponds in each case to one row of Table A

Table 800: Compounds of formula I.A* in which R⁴ is CH₃, R⁵ is cC₃H₅, Z is Q4, R^{6a} is 3-pyridyl, R^{6b} is cyclopropyl, and the combination of R¹, X, and (R³)ₙ for a compound corresponds in each case to one row of Table A

Table 801: Compounds of formula I.B* in which R⁴ is H, R⁵ is H, Z is Q1, R^{6a} is H, R^{6b} is H, and the combination of R¹, X, and (R³)ₙ for a compound corresponds in each case to one row of Table A

Table 802: Compounds of formula I.B* in which R⁴ is H, R⁵ is Cl, Z is Q1, R^{6a} is H, R^{6b} is H, and the combination of R¹, X, and (R³)ₙ for a compound corresponds in each case to one row of Table A

Table 803: Compounds of formula I.B* in which R⁴ is H, R⁵ is Br, Z is Q1, R^{6a} is H, R^{6b} is H, and the combination of R¹, X, and (R³)ₙ for a compound corresponds in each case to one row of Table A

Table 804: Compounds of formula I.B* in which R⁴ is H, R⁵ is Cl, Z is Q1, R^{6a} is 2-pyridyl, R^{6b} is H, and the combination of R¹, X, and (R³)ₙ for a compound corresponds in each case to one row of Table A

Table 805: Compounds of formula I.B* in which R⁴ is H, R⁵ is Br, Z is Q1, R^{6a} is 2-pyridyl, R^{6b} is H, and the combination of R¹, X, and (R³)ₙ for a compound corresponds in each case to one row of Table A

Table 806: Compounds of formula I.B* in which R⁴ is H, R⁵ is Cl, Z is Q1, R^{6a} is 3-pyridyl, R^{6b} is H, and the combination of R¹, X, and (R³)ₙ for a compound corresponds in each case to one row of Table A

Table 807: Compounds of formula I.B* in which R⁴ is H, R⁵ is Br, Z is Q1, R^{6a} is 3-pyridyl, R^{6b} is H, and the combination of R¹, X, and (R³)ₙ for a compound corresponds in each case to one row of of Table A

Table 808: Compounds of formula I.B* in which R⁴ is H, R⁵ is H, Z is Q2, R^{6a} is CN, R^{6b} is H, and the combination of R¹, X, and (R³)ₙ for a compound corresponds in each case to one row of Table A

Table 809: Compounds of formula I.B* in which R⁴ is H, R⁵ is Cl, Z is Q2, R^{6a} is CN, R^{6b} is H, and the combination of R¹, X, and (R³)ₙ for a compound corresponds in each case to one row of Table A

Table 810: Compounds of formula I.B* in which R⁴ is H, R⁵ is Br, Z is Q2, R^{6a} is CN, R^{6b} is H, and the combination of R¹, X, and (R³)ₙ for a compound corresponds in each case to one row of Table A

TABLE A

| No. | R¹ | X | (R³)ₙ |
|---|---|---|---|
| A-1 | H | CH | 3,5-F₂ |
| A-2 | H | CH | 3,5-Cl₂ |
| A-3 | H | CH | 3,5-Br₂ |
| A-4 | H | CH | 3,5-I₂ |
| A-5 | H | CH | 3,5-(CF₃)₂ |
| A-6 | H | CH | 3-Cl,5-F |
| A-7 | H | CH | 3-Cl,5-Br |
| A-8 | H | CH | 3-Cl,5-I |
| A-9 | H | CH | 3-F,5-CN |
| A-10 | H | CH | 3-Cl,5-CN |
| A-11 | H | CH | 3-CF₃,5-CN |
| A-12 | H | CH | 3-F,5-CF₃ |
| A-13 | H | CH | 3-Cl,5-CF₃ |
| A-14 | H | CH | 3-Br,5-CF₃ |
| A-15 | H | CH | 3-I,5-CF₃ |
| A-16 | H | CH | 3-Cl,5-SO₂CH₃ |
| A-17 | H | CH | 3-Cl,5-SO₂CF₃ |
| A-18 | H | CH | 3-Cl,5-cC₃H₅ |
| A-19 | H | CH | 3-Cl,5-[2,2-Cl₂-cC₃H₃] |
| A-20 | H | CH | 3-Cl,5-OCF₃ |
| A-21 | H | CH | 3-Br,5-OCF₃ |
| A-22 | H | CH | 3-F,5-cC₃H₅ |
| A-23 | H | CH | 3-Cl,5-CH₂CN |
| A-24 | H | CH | 3-Cl,5-C(CH₃)₂CN |
| A-25 | H | CH | 3-CF₃,5-CH₂CN |
| A-26 | H | CH | 3-CF₃,5-C(CH₃)₂CN |
| A-27 | H | CH | 3-CF₃,5-SO₂CH₃ |
| A-28 | H | CH | 3-CF₃,5-SO₂CF₃ |
| A-29 | H | CH | 3-CF₃,5-OCF₃ |
| A-30 | H | CH | 3-CF₃,5-cC₃H₅ |
| A-31 | H | CH | 3-Cl,5-[(1-CN)cC₃H₄] |
| A-32 | H | CH | 3-CF₃,5-[(1-CN)cC₃H₄] |
| A-33 | H | CH | 3-CF₃,5-[(2,2-Cl₂)-cC₃H₃] |
| A-34 | H | CH | 3-OCF₃,5-cC₃H₅ |
| A-35 | H | CH | 3,5-SO₂CH₃ |
| A-36 | H | CH | 3,5-SO₂CF₃ |
| A-37 | H | CH | 3-Cl,5-OC₆H₅ |
| A-38 | H | CH | 3-CF₃,5-OC₆H₅ |
| A-39 | H | CH | 3-CF₃,5-[O-(4-Cl—C₆H₄)] |
| A-40 | CH₃ | CH | 3,5-F₂ |
| A-41 | CH₃ | CH | 3,5-Cl₂ |
| A-42 | CH₃ | CH | 3,5-Br₂ |
| A-43 | CH₃ | CH | 3,5-I₂ |
| A-44 | CH₃ | CH | 3,5-(CF₃)₂ |
| A-45 | CH₃ | CH | 3-Cl,5-F |
| A-46 | CH₃ | CH | 3-Cl,5-Br |
| A-47 | CH₃ | CH | 3-Cl,5-I |
| A-48 | CH₃ | CH | 3-F,5-CN |
| A-49 | CH₃ | CH | 3-Cl,5-CN |
| A-50 | CH₃ | CH | 3-CF₃,5-CN |
| A-51 | CH₃ | CH | 3-F,5-CF₃ |
| A-52 | CH₃ | CH | 3-Cl,5-CF₃ |
| A-53 | CH₃ | CH | 3-Br,5-CF₃ |
| A-54 | CH₃ | CH | 3-I,5-CF₃ |
| A-55 | CH₃ | CH | 3-Cl,5-SO₂CH₃ |
| A-56 | CH₃ | CH | 3-Cl,5-SO₂CF₃ |
| A-57 | CH₃ | CH | 3-Cl,5-cC₃H₅ |
| A-58 | CH₃ | CH | 3-Cl,5-[2,2-Cl₂-cC₃H₃] |
| A-59 | CH₃ | CH | 3-Cl,5-OCF₃ |
| A-60 | CH₃ | CH | 3-Br,5-OCF₃ |
| A-61 | CH₃ | CH | 3-F,5-cC₃H₅ |
| A-62 | CH₃ | CH | 3-Cl,5-CH₂CN |
| A-63 | CH₃ | CH | 3-Cl,5-C(CH₃)₂CN |
| A-64 | CH₃ | CH | 3-CF₃,5-CH₂CN |
| A-65 | CH₃ | CH | 3-CF₃,5-C(CH₃)₂CN |
| A-66 | CH₃ | CH | 3-CF₃,5-SO₂CH₃ |
| A-67 | CH₃ | CH | 3-CF₃,5-SO₂CF₃ |
| A-68 | CH₃ | CH | 3-CF₃,5-OCF₃ |
| A-69 | CH₃ | CH | 3-CF₃,5-cC₃H₅ |
| A-70 | CH₃ | CH | 3-Cl,5-[(1-CN)cC₃H₄] |
| A-71 | CH₃ | CH | 3-CF₃,5-[(1-CN)cC₃H₄] |
| A-72 | CH₃ | CH | 3-CF₃,5-[(2,2-Cl₂)-cC₃H₃] |
| A-73 | CH₃ | CH | 3-OCF₃,5-cC₃H₅ |
| A-74 | CH₃ | CH | 3,5-SO₂CH₃ |
| A-75 | CH₃ | CH | 3,5-SO₂CF₃ |
| A-76 | CH₃ | CH | 3-Cl,5-OC₆H₅ |
| A-77 | CH₃ | CH | 3-CF₃,5-OC₆H₅ |
| A-78 | CH₃ | CH | 3-CF₃,5-[O-(4-Cl—C₆H₄)] |
| A-79 | C₂H₅ | CH | 3,5-F₂ |
| A-80 | C₂H₅ | CH | 3,5-Cl₂ |
| A-81 | C₂H₅ | CH | 3,5-Br₂ |
| A-82 | C₂H₅ | CH | 3,5-I₂ |
| A-83 | C₂H₅ | CH | 3,5-(CF₃)₂ |
| A-84 | C₂H₅ | CH | 3-Cl,5-F |
| A-85 | C₂H₅ | CH | 3-Cl,5-Br |
| A-86 | C₂H₅ | CH | 3-Cl,5-I |
| A-87 | C₂H₅ | CH | 3-F,5-CN |
| A-88 | C₂H₅ | CH | 3-Cl,5-CN |
| A-89 | C₂H₅ | CH | 3-CF₃,5-CN |
| A-90 | C₂H₅ | CH | 3-F,5-CF₃ |
| A-91 | C₂H₅ | CH | 3-Cl,5-CF₃ |
| A-92 | C₂H₅ | CH | 3-Br,5-CF₃ |
| A-93 | C₂H₅ | CH | 3-I,5-CF₃ |
| A-94 | C₂H₅ | CH | 3-Cl,5-SO₂CH₃ |
| A-95 | C₂H₅ | CH | 3-Cl,5-SO₂CF₃ |
| A-96 | C₂H₅ | CH | 3-Cl,5-cC₃H₅ |
| A-97 | C₂H₅ | CH | 3-Cl,5-[2,2-Cl₂-cC₃H₃] |
| A-98 | C₂H₅ | CH | 3-Cl,5-OCF₃ |
| A-99 | C₂H₅ | CH | 3-Br,5-OCF₃ |
| A-100 | C₂H₅ | CH | 3-F,5-cC₃H₅ |
| A-101 | C₂H₅ | CH | 3-Cl,5-CH₂CN |
| A-102 | C₂H₅ | CH | 3-Cl,5-C(CH₃)₂CN |
| A-103 | C₂H₅ | CH | 3-CF₃,5-CH₂CN |
| A-104 | C₂H₅ | CH | 3-CF₃,5-C(CH₃)₂CN |
| A-105 | C₂H₅ | CH | 3-CF₃,5-SO₂CH₃ |
| A-106 | C₂H₅ | CH | 3-CF₃,5-SO₂CF₃ |
| A-107 | C₂H₅ | CH | 3-CF₃,5-OCF₃ |
| A-108 | C₂H₅ | CH | 3-CF₃,5-cC₃H₅ |
| A-109 | C₂H₅ | CH | 3-Cl,5-[(1-CN)cC₃H₄] |
| A-110 | C₂H₅ | CH | 3-CF₃,5-[(1-CN)cC₃H₄] |
| A-111 | C₂H₅ | CH | 3-CF₃,5-[(2,2-Cl₂)-cC₃H₃] |
| A-112 | C₂H₅ | CH | 3-OCF₃,5-cC₃H₅ |
| A-113 | C₂H₅ | CH | 3,5-SO₂CH₃ |
| A-114 | C₂H₅ | CH | 3,5-SO₂CF₃ |
| A-115 | C₂H₅ | CH | 3-Cl,5-OC₆H₅ |
| A-116 | C₂H₅ | CH | 3-CF₃,5-OC₆H₅ |
| A-117 | C₂H₅ | CH | 3-CF₃,5-[O-(4-Cl—C₆H₄)] |
| A-118 | CH₂-cC₃H₅ | CH | 3,5-F₂ |
| A-119 | CH₂-cC₃H₅ | CH | 3,5-Cl₂ |
| A-120 | CH₂-cC₃H₅ | CH | 3,5-Br₂ |
| A-121 | CH₂-cC₃H₅ | CH | 3,5-I₂ |
| A-122 | CH₂-cC₃H₅ | CH | 3,5-(CF₃)₂ |
| A-123 | CH₂-cC₃H₅ | CH | 3-Cl,5-F |
| A-124 | CH₂-cC₃H₅ | CH | 3-Cl,5-Br |
| A-125 | CH₂-cC₃H₅ | CH | 3-Cl,5-I |
| A-126 | CH₂-cC₃H₅ | CH | 3-F,5-CN |
| A-127 | CH₂-cC₃H₅ | CH | 3-Cl,5-CN |
| A-128 | CH₂-cC₃H₅ | CH | 3-CF₃,5-CN |
| A-129 | CH₂-cC₃H₅ | CH | 3-F,5-CF₃ |
| A-130 | CH₂-cC₃H₅ | CH | 3-Cl,5-CF₃ |
| A-131 | CH₂-cC₃H₅ | CH | 3-Br,5-CF₃ |
| A-132 | CH₂-cC₃H₅ | CH | 3-I,5-CF₃ |
| A-133 | CH₂-cC₃H₅ | CH | 3-Cl,5-SO₂CH₃ |
| A-134 | CH₂-cC₃H₅ | CH | 3-Cl,5-SO₂CF₃ |
| A-135 | CH₂-cC₃H₅ | CH | 3-Cl,5-cC₃H₅ |
| A-136 | CH₂-cC₃H₅ | CH | 3-Cl,5-[2,2-Cl₂-cC₃H₃] |
| A-137 | CH₂-cC₃H₅ | CH | 3-Cl,5-OCF₃ |
| A-138 | CH₂-cC₃H₅ | CH | 3-Br,5-OCF₃ |
| A-139 | CH₂-cC₃H₅ | CH | 3-F,5-cC₃H₅ |
| A-140 | CH₂-cC₃H₅ | CH | 3-Cl,5-CH₂CN |
| A-141 | CH₂-cC₃H₅ | CH | 3-Cl,5-C(CH₃)₂CN |
| A-142 | CH₂-cC₃H₅ | CH | 3-CF₃,5-CH₂CN |
| A-143 | CH₂-cC₃H₅ | CH | 3-CF₃,5-C(CH₃)₂CN |
| A-144 | CH₂-cC₃H₅ | CH | 3-CF₃,5-SO₂CH₃ |
| A-145 | CH₂-cC₃H₅ | CH | 3-CF₃,5-SO₂CF₃ |
| A-146 | CH₂-cC₃H₅ | CH | 3-CF₃,5-OCF₃ |
| A-147 | CH₂-cC₃H₅ | CH | 3-CF₃,5-cC₃H₅ |
| A-148 | CH₂-cC₃H₅ | CH | 3-Cl,5-[(1-CN)cC₃H₄] |
| A-149 | CH₂-cC₃H₅ | CH | 3-CF₃,5-[(1-CN)cC₃H₄] |
| A-150 | CH₂-cC₃H₅ | CH | 3-CF₃,5-[(2,2-Cl₂)-cC₃H₃] |
| A-151 | CH₂-cC₃H₅ | CH | 3-OCF₃,5-cC₃H₅ |
| A-152 | CH₂-cC₃H₅ | CH | 3,5-SO₂CH₃ |
| A-153 | CH₂-cC₃H₅ | CH | 3,5-SO₂CF₃ |
| A-154 | CH₂-cC₃H₅ | CH | 3-Cl,5-OC₆H₅ |
| A-155 | CH₂-cC₃H₅ | CH | 3-CF₃,5-OC₆H₅ |
| A-156 | CH₂-cC₃H₅ | CH | 3-CF₃,5-[O-(4-Cl—C₆H₄)] |
| A-157 | H | N | 3,5-F₂ |
| A-158 | H | N | 3,5-Cl₂ |
| A-159 | H | N | 3,5-Br₂ |
| A-160 | H | N | 3,5-I₂ |
| A-161 | H | N | 3,5-(CF₃)₂ |
| A-162 | H | N | 3-Cl,5-F |
| A-163 | H | N | 3-Cl,5-Br |
| A-164 | H | N | 3-Cl,5-I |
| A-165 | H | N | 3-F,5-CN |
| A-166 | H | N | 3-Cl,5-CN |
| A-167 | H | N | 3-CF₃,5-CN |
| A-168 | H | N | 3-F,5-CF₃ |
| A-169 | H | N | 3-Cl,5-CF₃ |
| A-170 | H | N | 3-Br,5-CF₃ |
| A-171 | H | N | 3-I,5-CF₃ |
| A-172 | H | N | 3-Cl,5-SO₂CH₃ |
| A-173 | H | N | 3-Cl,5-SO₂CF₃ |
| A-174 | H | N | 3-Cl,5-cC₃H₅ |
| A-175 | H | N | 3-Cl,5-[2,2-Cl₂-cC₃H₃] |
| A-176 | H | N | 3-Cl,5-OCF₃ |
| A-177 | H | N | 3-Br,5-OCF₃ |
| A-178 | H | N | 3-F,5-cC₃H₅ |
| A-179 | H | N | 3-Cl,5-CH₂CN |
| A-180 | H | N | 3-Cl,5-C(CH₃)₂CN |
| A-181 | H | N | 3-CF₃,5-CH₂CN |
| A-182 | H | N | 3-CF₃,5-C(CH₃)₂CN |
| A-183 | H | N | 3-CF₃,5-SO₂CH₃ |
| A-184 | H | N | 3-CF₃,5-SO₂CF₃ |
| A-185 | H | N | 3-CF₃,5-OCF₃ |
| A-186 | H | N | 3-CF₃,5-cC₃H₅ |
| A-187 | H | N | 3-Cl,5-[(1-CN)cC₃H₄] |
| A-188 | H | N | 3-CF₃,5-[(1-CN)cC₃H₄] |
| A-189 | H | N | 3-CF₃,5-[(2,2-Cl₂)-cC₃H₃] |
| A-190 | H | N | 3-OCF₃,5-cC₃H₅ |
| A-191 | H | N | 3,5-SO₂CH₃ |
| A-192 | H | N | 3,5-SO₂CF₃ |
| A-193 | H | N | 3-Cl,5-OC₆H₅ |
| A-194 | H | N | 3-CF₃,5-OC₆H₅ |
| A-195 | H | N | 3-CF₃,5-[O-(4-Cl—C₆H₄)] |
| A-196 | CH₃ | N | 3,5-F₂ |
| A-197 | CH₃ | N | 3,5-Cl₂ |
| A-198 | CH₃ | N | 3,5-Br₂ |
| A-199 | CH₃ | N | 3,5-I₂ |
| A-200 | CH₃ | N | 3,5-(CF₃)₂ |
| A-201 | CH₃ | N | 3-Cl,5-F |
| A-202 | CH₃ | N | 3-Cl,5-Br |
| A-203 | CH₃ | N | 3-Cl,5-I |
| A-204 | CH₃ | N | 3-F,5-CN |
| A-205 | CH₃ | N | 3-Cl,5-CN |
| A-206 | CH₃ | N | 3-CF₃,5-CN |
| A-207 | CH₃ | N | 3-F,5-CF₃ |
| A-208 | CH₃ | N | 3-Cl,5-CF₃ |
| A-209 | CH₃ | N | 3-Br,5-CF₃ |
| A-210 | CH₃ | N | 3-I,5-CF₃ |
| A-211 | CH₃ | N | 3-Cl,5-SO₂CH₃ |
| A-212 | CH₃ | N | 3-Cl,5-SO₂CF₃ |
| A-213 | CH₃ | N | 3-Cl,5-cC₃H₅ |
| A-214 | CH₃ | N | 3-Cl,5-[2,2-Cl₂-cC₃H₃] |
| A-215 | CH₃ | N | 3-Cl,5-OCF₃ |
| A-216 | CH₃ | N | 3-Br,5-OCF₃ |
| A-217 | CH₃ | N | 3-F,5-cC₃H₅ |
| A-218 | CH₃ | N | 3-Cl,5-CH₂CN |
| A-219 | CH₃ | N | 3-Cl,5-C(CH₃)₂CN |
| A-220 | CH₃ | N | 3-CF₃,5-CH₂CN |
| A-221 | CH₃ | N | 3-CF₃,5-C(CH₃)₂CN |
| A-222 | CH₃ | N | 3-CF₃,5-SO₂CH₃ |
| A-223 | CH₃ | N | 3-CF₃,5-SO₂CF₃ |
| A-224 | CH₃ | N | 3-CF₃,5-OCF₃ |
| A-225 | CH₃ | N | 3-CF₃,5-cC₃H₅ |
| A-226 | CH₃ | N | 3-Cl,5-[(1-CN)cC₃H₄] |
| A-227 | CH₃ | N | 3-CF₃,5-[(1-CN)cC₃H₄] |
| A-228 | CH₃ | N | 3-CF₃,5-[(2,2-Cl₂)-cC₃H₃] |
| A-229 | CH₃ | N | 3-OCF₃,5-cC₃H₅ |
| A-230 | CH₃ | N | 3,5-SO₂CH₃ |
| A-231 | CH₃ | N | 3,5-SO₂CF₃ |
| A-232 | CH₃ | N | 3-Cl,5-OC₆H₅ |
| A-233 | CH₃ | N | 3-CF₃,5-OC₆H₅ |
| A-234 | CH₃ | N | 3-CF₃,5-[O-(4-Cl—C₆H₄)] |
| A-235 | C₂H₅ | N | 3,5-F₂ |
| A-236 | C₂H₅ | N | 3,5-Cl₂ |
| A-237 | C₂H₅ | N | 3,5-Br₂ |
| A-238 | C₂H₅ | N | 3,5-I₂ |
| A-239 | C₂H₅ | N | 3,5-(CF₃)₂ |
| A-240 | C₂H₅ | N | 3-Cl,5-F |
| A-241 | C₂H₅ | N | 3-Cl,5-Br |
| A-242 | C₂H₅ | N | 3-Cl,5-I |
| A-243 | C₂H₅ | N | 3-F,5-CN |
| A-244 | C₂H₅ | N | 3-Cl,5-CN |
| A-245 | C₂H₅ | N | 3-CF₃,5-CN |
| A-246 | C₂H₅ | N | 3-F,5-CF₃ |
| A-247 | C₂H₅ | N | 3-Cl,5-CF₃ |
| A-248 | C₂H₅ | N | 3-Br,5-CF₃ |
| A-249 | C₂H₅ | N | 3-I,5-CF₃ |
| A-250 | C₂H₅ | N | 3-Cl,5-SO₂CH₃ |
| A-251 | C₂H₅ | N | 3-Cl,5-SO₂CF₃ |
| A-252 | C₂H₅ | N | 3-Cl,5-cC₃H₅ |
| A-253 | C₂H₅ | N | 3-Cl,5-[2,2-Cl₂-cC₃H₃] |
| A-254 | C₂H₅ | N | 3-Cl,5-OCF₃ |
| A-255 | C₂H₅ | N | 3-Br,5-OCF₃ |
| A-256 | C₂H₅ | N | 3-F,5-cC₃H₅ |
| A-257 | C₂H₅ | N | 3-Cl,5-CH₂CN |
| A-258 | C₂H₅ | N | 3-Cl,5-C(CH₃)₂CN |
| A-259 | C₂H₅ | N | 3-CF₃,5-CH₂CN |
| A-260 | C₂H₅ | N | 3-CF₃,5-C(CH₃)₂CN |
| A-261 | C₂H₅ | N | 3-CF₃,5-SO₂CH₃ |
| A-262 | C₂H₅ | N | 3-CF₃,5-SO₂CF₃ |
| A-263 | C₂H₅ | N | 3-CF₃,5-OCF₃ |
| A-264 | C₂H₅ | N | 3-CF₃,5-cC₃H₅ |
| A-265 | C₂H₅ | N | 3-Cl,5-[(1-CN)cC₃H₄] |
| A-266 | C₂H₅ | N | 3-CF₃,5-[(1-CN)cC₃H₄] |
| A-267 | C₂H₅ | N | 3-CF₃,5-[(2,2-Cl₂)-cC₃H₃] |
| A-268 | C₂H₅ | N | 3-OCF₃,5-cC₃H₅ |
| A-269 | C₂H₅ | N | 3,5-SO₂CH₃ |
| A-270 | C₂H₅ | N | 3,5-SO₂CF₃ |
| A-271 | C₂H₅ | N | 3-Cl,5-OC₆H₅ |
| A-272 | C₂H₅ | N | 3-CF₃,5-OC₆H₅ |
| A-273 | C₂H₅ | N | 3-CF₃,5-[O-(4-Cl—C₆H₄)] |
| A-274 | CH₂-cC₃H₅ | N | 3,5-F₂ |
| A-275 | CH₂-cC₃H₅ | N | 3,5-Cl₂ |
| A-276 | CH₂-cC₃H₅ | N | 3,5-Br₂ |
| A-277 | CH₂-cC₃H₅ | N | 3,5-I₂ |
| A-278 | CH₂-cC₃H₅ | N | 3,5-(CF₃)₂ |
| A-279 | CH₂-cC₃H₅ | N | 3-Cl,5-F |
| A-280 | CH₂-cC₃H₅ | N | 3-Cl,5-Br |
| A-281 | CH₂-cC₃H₅ | N | 3-Cl,5-I |
| A-282 | CH₂-cC₃H₅ | N | 3-F,5-CN |
| A-283 | CH₂-cC₃H₅ | N | 3-Cl,5-CN |
| A-284 | CH₂-cC₃H₅ | N | 3-CF₃,5-CN |
| A-285 | CH₂-cC₃H₅ | N | 3-F,5-CF₃ |
| A-286 | CH₂-cC₃H₅ | N | 3-Cl,5-CF₃ |
| A-287 | CH₂-cC₃H₅ | N | 3-Br,5-CF₃ |
| A-288 | CH₂-cC₃H₅ | N | 3-I,5-CF₃ |
| A-289 | CH₂-cC₃H₅ | N | 3-Cl,5-SO₂CH₃ |
| A-290 | CH₂-cC₃H₅ | N | 3-Cl,5-SO₂CF₃ |
| A-291 | CH₂-cC₃H₅ | N | 3-Cl,5-cC₃H₅ |
| A-292 | CH₂-cC₃H₅ | N | 3-Cl,5-[2,2-Cl₂-cC₃H₃] |
| A-293 | CH₂-cC₃H₅ | N | 3-Cl,5-OCF₃ |
| A-294 | CH₂-cC₃H₅ | N | 3-Br,5-OCF₃ |
| A-295 | CH₂-cC₃H₅ | N | 3-F,5-cC₃H₅ |
| A-296 | CH₂-cC₃H₅ | N | 3-Cl,5-CH₂CN |
| A-297 | CH₂-cC₃H₅ | N | 3-Cl,5-C(CH₃)₂CN |
| A-298 | CH₂-cC₃H₅ | N | 3-CF₃,5-CH₂CN |
| A-299 | CH₂-cC₃H₅ | N | 3-CF₃,5-C(CH₃)₂CN |
| A-300 | CH₂-cC₃H₅ | N | 3-CF₃,5-SO₂CH₃ |
| A-301 | CH₂-cC₃H₅ | N | 3-CF₃,5-SO₂CF₃ |
| A-302 | CH₂-cC₃H₅ | N | 3-CF₃,5-OCF₃ |
| A-303 | CH₂-cC₃H₅ | N | 3-CF₃,5-cC₃H₅ |
| A-304 | CH₂-cC₃H₅ | N | 3-Cl,5-[(1-CN)cC₃H₄] |
| A-305 | CH₂-cC₃H₅ | N | 3-CF₃,5-[(1-CN)cC₃H₄] |
| A-306 | CH₂-cC₃H₅ | N | 3-CF₃,5-[(2,2-Cl₂)-cC₃H₃] |
| A-307 | CH₂-cC₃H₅ | N | 3-OCF₃,5-cC₃H₅ |
| A-308 | CH₂-cC₃H₅ | N | 3,5-SO₂CH₃ |
| A-309 | CH₂-cC₃H₅ | N | 3,5-SO₂CF₃ |
| A-310 | CH₂-cC₃H₅ | N | 3-Cl,5-OC₆H₅ |
| A-311 | CH₂-cC₃H₅ | N | 3-CF₃,5-OC₆H₅ |
| A-312 | CH₂-cC₃H₅ | N | 3-CF₃,5-[O-(4-Cl—C₆H₄)] |

The term “compound(s) of the invention” refers to compound(s) of formula I, or “compound(s) I”, and includes their salts, tautomers, stereoisomers, and N-oxides.

The invention also relates to agrochemical compositions comprising an auxiliary and at least one compound I.

An agrochemical composition comprises a pesticidally effective amount of a compound I.

The compounds I can be converted into customary types of agro-chemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials e.g. seeds (e.g. GF). These and further compositions types are defined in the “Catalogue of pesticide formulation types and international coding system”, Technical Monograph No. 2, 6th Ed. May 2008, CropLife International. The compositions are prepared in a known manner, e.g. described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents. Suitable solid carriers or fillers are mineral earths.

Suitable surfactants are surface-active compounds, e.g. anionic, cationic, nonionic, and amphoteric surfactants, block polymers, polyelectrolytes. Such surfactants can be used as emusifier, dispersant, solubilizer, wetter, penetration enhancer, protective colloid, or adjuvant. Surfactants are listed in McCutcheon's, Vol. 1: Emulsifiers & Detergents, McCutcheon's Directories, Glen Rock, USA, 2008 (International or North American Ed.). Suitable anionic surfactants are alkali, alkaline earth, or ammonium salts of sulfonates, sulfates, phosphates, carboxylates. Suitable nonionic surfactants are alkoxylates, N-subsituted fatty acid amides, amine oxides, esters, sugar-based surfactants, polymeric surfactants. Suitable cationic surfactants are qua-ternary surfactants.

The agrochemical compositions generally comprise between 0.01 and 95%, preferably between 0.1 and 90%, and most preferably between 0.5 and 75%, by weight of active substance. The active substances are employed in a purity of from 90% to 100%, preferably from 95% to 100%.

Various types of oils, wetters, adjuvants, or fertilizer may be added to the active substances or the compositions comprising them as premix or, if appropriate not until immediately prior to use (tank mix). These agents can be admixed with the compositions according to the invention in a weight ratio of 1:100 to 100:1.

The user applies the composition according to the invention usually from a predosage device, a knapsack sprayer, a spray tank, a spray plane, or an irrigation system. Usually, the agro-chemical composition is made up with water, buffer, and/or further auxiliaries to the desired application concentration and the ready-to-use spray liquor or the agrochemical composition according to the invention is thus obtained. Usually, 20 to 2000 liters, of the ready-to-use spray liquor are applied per hectare of agricultural useful area.

The compounds I are suitable for use in protecting crops, plants, plant propagation materials, e.g. seeds, or soil or water, in which the plants are growing, from attack or infestation by animal pests. Therefore, the invention also relates to a plant protection method, which comprises contacting crops, plants, plant propagation materials, e.g. seeds, or soil or water, in which the plants are growing, to be protected from attack or infestation by animal pests, with a pesticidally effective amount of a compound I.

The compounds I are also suitable for use in combating or controlling animal pests. Therefore, the invention also relates to a method of combating or controlling animal pests, which comprises contacting the animal pests, their habitat, breeding ground, or food supply, or the crops, plants, plant propagation materials, e.g. seeds, or soil, or the area, material or environment in which the animal pests are growing or may grow, with a pesticidally effective amount of a compound I.

The compounds I are effective through both contact and ingestion to any and all developmental stages, such as egg, larva, pupa, and adult.

The compounds I can be applied as such or in form of compositions comprising them.

The application can be carried out both before and after the infestation of the crops, plants, plant propagation materials by the pests.

The term “contacting” includes both direct contact (applying the compounds/compositions directly on the animal pest or plant) and indirect contact (applying the compounds/compositions to the locus).

The term “animal pest” includes arthropods, gastropods, and nematodes. Preferred animal pests according to the invention are arthropods, preferably insects and arachnids, in particular insects.

The term “plant” includes cereals, e.g. durum and other wheat, rye, barley, triticale, oats, rice, or maize (fodder maize and sugar maize/sweet and field corn); beet, e.g. sugar beet, or fodder beet; fruits, e.g. pomes, stone fruits, or soft fruits, e.g. apples, pears, plums, peaches, nectarines, almonds, cherries, papayas, strawberries, raspberries, blackberries or gooseberries; leguminous plants, e.g. beans, lentils, peas, alfalfa, or soybeans; oil plants, e.g. rapeseed (oilseed rape), turnip rape, mustard, olives, sunflowers, coconut, cocoa beans, castor oil plants, oil palms, ground nuts, or soybeans; cucurbits, e.g. squashes, pumpkins, cucumber or melons; fiber plants, e.g. cotton, flax, hemp, or jute; citrus fruit, e.g. oranges, lemons, grape-fruits or mandarins; vegetables, e.g. eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, e.g. avocados, cinnamon, or camphor; energy and raw material plants, e.g. corn, soybean, rapeseed, sugar cane or oil palm; tobacco; nuts, e.g. walnuts; pistachios; coffee; tea; bananas; vines; hop; sweet leaf (Stevia); natural rubber plants or ornamental and forestry plants, shrubs, broad-leaved trees or evergreens, eucalyptus; turf; lawn; grass. Preferred plants include potatoes sugar beets, tobacco, wheat, rye, barley, oats, rice, corn, cotton, soybeans, rapeseed, legumes, sunflowers, coffee, or sugar cane; fruits; vines; ornamentals; or vegetables, e.g. cucumbers, tomatoes, beans or squashes.

The term “seed” embraces seeds and plant propagules including true seeds, seed pieces, suckers, corms, bulbs, fruit, tubers, grains, cuttings, cut shoots, and means preferably true seeds.

“Pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions e.g. desired pesticidal effect and duration, weather, target species, locus, mode of application.

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare.

The compounds I are also suitable for use against non-crop insect pests. For use against said non-crop pests, compounds I can be used as bait composition, gel, general insect spray, aero-sol, as ultra-low volume application and bed net (impregnated or surface applied).

The term “non-crop insect pest” refers to pests, which are particularly relevant for non-crop targets, e.g. ants, termites, wasps, flies, ticks, mosquitoes, bed bugs, crickets, or cockroaches, such as: Aedes aegypti, Musca domestica, Tribolium spp.; termites such as Reticulitermes flavipes, Coptotermes formosanus; roaches such as Blatella germanica, Periplaneta Americana; ants such as Solenopsis invicta, Linepithema humile, and Camponotus pennsylvanicus.

The bait can be a liquid, a solid or a semisolid preparation (e.g. a gel). For use in bait compositions, the typical content of active ingredient is from 0.001 wt % to 15 wt %, desirably from 0.001 wt % to 5 wt % of active compound.

The compounds I and its compositions can be used for protecting wooden materials such as trees, board fences, sleepers, frames, artistic artifacts, etc. and buildings, but also construction materials, furniture, leathers, fibers, vinyl articles, electric wires and cables etc. from ants, termites and/or wood or textile destroying beetles, and for controlling ants and termites from doing harm to crops or human beings (e.g. when the pests invade into houses and public facilities or nest in yards, orchards or parks).

Customary application rates in the protection of materials are, e.g., from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 wt %, preferably from 0.1 to 45 wt %, and more preferably from 1 to 25 wt % of at least one repellent and/or insecticide.

The compounds of the invention are especially suitable for efficiently combating animal pests e.g. arthropods, and nematodes including:
insects from the sub-order of Auchenorrhyncha, e.g. Amrasca biguttula, Empoasca spp., Nephotettix virescens, Sogatella furcifera, Mahanarva spp., Laodelphax striatellus, Nilaparvata lugens, Diaphorina citri;
Lepidoptera, e.g. Helicoverpa spp., Heliothis virescens, Lobesia botrana, Ostrinia nubilalis, Plutella xylostella, Pseudoplusia includens, Scirpophaga incertulas, Spodoptera spp., Trichoplusia ni, Tuta absoluta, Cnaphalocrocis medialis, Cydia pomonella, Chilo suppressalis, Anticarsia gemmatalis, Agrotis ipsilon, Chrysodeixis includens;
True bugs, e.g. Lygus spp., Stink bugs such as Euschistus spp., Halyomorpha halys, Nezara viridula, Piezodorus guildinii, Dichelops furcatus;
Thrips, e.g. Frankliniella spp., Thrips spp., Dichromothrips corbettii;
Aphids, e.g. Acyrthosiphon pisum, Aphis spp., Myzus persicae, Rhopalosiphum spp., Schizaphis graminum, Megoura viciae;
Whiteflies, e.g. Trialeurodes vaporariorum, Bemisia spp.;
Coleoptera, e.g. Phyllotreta spp., Melanotus spp., Meligethes aeneus, Leptinotarsa decimlineata, Ceutorhynchus spp., Diabrotica spp., Anthonomus grandis, Atomaria linearia, Agriotes spp., Epilachna spp.;
Flies, e.g. Delia spp., Ceratitis capitate, Bactrocera spp., Liriomyza spp.;
Coccoidea, e.g. Aonidiella aurantia, Ferrisia virgate;
Anthropods of class Arachnida (Mites), e.g. Penthaleus major, Tetranychus spp.;
Nematodes, e.g. Heterodera glycines, Meloidogyne spp., Pratylenchus spp., Caenorhabditis elegans.

The compounds I are suitable for use in treating or protecting animals against infestation or infection by parasites. Therefore, the invention also relates to the use of a compound of the invention for the manufacture of a medicament for the treatment or protection of animals against infestation or infection by parasites. Furthermore, the invention relates to a method of treating or protecting animals against infestation and infection by parasites, which comprises orally, topically or parenterally administering or applying to the animals a parasiticidally effective amount of a compound I.

The invention also relates to the non-therapeutic use of compounds of the invention for treating or protecting animals against infestation and infection by parasites. Moreover, the invention relates to a non-therapeutic method of treating or protecting animals against infestation and infection by parasites, which comprises applying to a locus a parasiticidally effective amount of a compound I.

The compounds of the invention are further suitable for use in combating or controlling parasites in and on animals. Furthermore, the invention relates to a method of combating or controlling parasites in and on animals, which comprises contacting the parasites with a parasitically effective amount of a compound I.

The invention also relates to the non-therapeutic use of compounds I for controlling or combating parasites. Moreover, the invention relates to a non-therapeutic method of combating or controlling parasites, which comprises applying to a locus a parasiticidally effective amount of a compound I.

The compounds I can be effective through both contact (via soil, glass, wall, bed net, carpet, blankets or animal parts) and ingestion (e.g. baits). Furthermore, the compounds I can be applied to any and all developmental stages.

The compounds I can be applied as such or in form of compositions comprising them.

The term “locus” means the habitat, food supply, breeding ground, area, material or environment in which a parasite is growing or may grow outside of the animal.

As used herein, the term “parasites” includes endo- and ectoparasites. In some embodiments of the invention, endoparasites can be preferred. In other embodiments, ectoparasites can be preferred. Infestations in warm-blooded animals and fish include lice, biting lice, ticks, nasal bots, keds, biting flies, muscoid flies, flies, myiasitic fly larvae, chiggers, gnats, mosquitoes and fleas.

The compounds of the invention are especially useful for combating the following parasites: Cimex lectularius, Rhipicephalus sanguineus, and Ctenocephalides felis.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in furbearing animals such as mink, chinchilla and raccoon, birds such as hens, geese, turkeys and ducks and fish such as fresh- and salt-water fish such as trout, carp and eels. Particularly preferred are domestic animals, such as dogs or cats.

The compounds I may be applied in total amounts of 0.5 mg/kg to 100 mg/kg per day, preferably 1 mg/kg to 50 mg/kg per day.

For oral administration to warm-blooded animals, the compounds I may be formulated as animal feeds, animal feed premixes, animal feed concentrates, pills, solutions, pastes, suspensions, drenches, gels, tablets, boluses and capsules. For oral administration, the dosage form chosen should provide the animal with 0.01 mg/kg to 100 mg/kg of animal body weight per day of the compounds I, preferably with 0.5 mg/kg to 100 mg/kg of animal body weight per day.

Alternatively, the compounds I may be administered to animals parenterally, e.g., by intraruminal, intramuscular, intravenous or subcutaneous injection. The compounds I may be dispersed or dissolved in a physiologically acceptable carrier for subcutaneous injection. Alternatively, the compounds I may be formulated into an implant for subcutaneous administration. In addition the compounds I may be transdermally administered to animals. For parenteral administration, the dosage form chosen should provide the animal with 0.01 mg/kg to 100 mg/kg of animal body weight per day of the compounds I.

The compounds I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds I. In addition, the compounds I may be formulated as ear tags for animals, particularly quadrupeds e.g. cattle and sheep.

Oral solutions are administered directly.

Solutions for use on the skin are trickled on, spread on, rubbed in, sprinkled on or sprayed on.

Gels are applied to or spread on the skin or introduced into body cavities.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures.

Emulsions can be administered orally, dermally or as injections.

Suspensions can be administered orally or topically/dermally.

Semi-solid preparations can be administered orally or topically/dermally.

For the production of solid preparations, the active compound is mixed with suitable excipients, if appropriate with addition of auxiliaries, and brought into the desired form.

The compositions which can be used in the invention can comprise generally from about 0.001 to 95% of the compound I.

Ready-to-use preparations contain the compounds acting against parasites, preferably ectoparasites, in concentrations of 10 ppm to 80% by weight, preferably from 0.1 to 65% by weight, more preferably from 1 to 50% by weight, most preferably from 5 to 40% by weight.

Preparations which are diluted before use contain the compounds acting against ectoparasites in concentrations of 0.5 to 90% by weight, preferably of 1 to 50% by weight.

Furthermore, the preparations comprise the compounds of formula I against endoparasites in concentrations of 10 ppm to 2% by weight, preferably of 0.05 to 0.9% by weight, very particularly preferably of 0.005 to 0.25% by weight.

Solid formulations which release compounds of the invention may be applied in total amounts of 10 mg/kg to 300 mg/kg, preferably 20 mg/kg to 200 mg/kg, most preferably 25 mg/kg to 160 mg/kg body weight of the treated animal in the course of three weeks.

### A. PREPARATION EXAMPLES

The compounds were characterized by melting point determination, by NMR spectroscopy or by the mass-to-charge ratio ([m/z]) and retention time (RT; [min]), as determined by mass spectrometry (MS) coupled with HPLC analysis (HPLC-MS=high performance liquid chromatography-coupled mass spectrometry) or LC analysis (LC-MS=liquid chromatography-coupled mass spectrometry).

Method A: LC Agilent 1200 Series G1315D, MS Agilent 6100 Series G6120B; Column: Poroshell 120 SB-C18 4.6×30 mm 2.7 μm; Mobile phase: A: water (0.1% formic acid), B: ACN (0.1% formic acid); Temperature: 60° C.; Gradient: 1% B to 100% B in 1.5 min; 100% B 0.18 min; Flow: 3 mL/min; MS: ESI positive; Mass range (m/z): 83-600.

Method B: Shimadzu LC-30AD MSD: LCMS-2020; Column: Luna-C18 3 μm 2.0×30 mm; Mobile phase: A: water+0.04% TFA; B: ACN+0.02% TFA; Temperature: 40° C.; Gradient: 5% B to 95% B in 1.6 min; 95% B to 100% B in 0.9 min; 100% B to 5% B in 0.02 min; 5% B for 0.48 min; Flow: 0.8 mL/min; MS: ESI positive; Mass range: 50-2000.

Method C: Shimadzu LC-20ADXR MSD: LCMS-2020; Column: Halo C18 5 μm 3.0×30 mm; Mobile phase: A: water+0.04% TFA; B: ACN+0.02% TFA; Temperature: 40° C.; Gradient: 5% B to 95% B in 3.5 min; 95% B for 1.0 min; Flow: 1.0 mL/min; MS: ESI positive; Mass range: 50-2000.

Method D: Shimadzu Nexera UHPLC+Shimadzu LCMS-2020, ESI; Column: Kinetex 1.7p XB-C18 100A, 2.1×50 mm; Mobile phase: A: water+0.1% TFA; B: ACN; Temperature: 60° C.; Gradient: 5% B to 100% B in 1.5 min; 100% B 0.25 min; Flow: 0.8 mL/min to 1.0 mL/min in 1.5 min; MS: ESI positive; Mass range (m/z): 100-700.

### Example 1: Preparation of 3-chloro-N-[1-[6-chloro-3-(triazol-2-yl)-2-pyridyl]ethyl]-5-(trifluoromethyl)benzamide (I-3)

### Step 1: Preparation of 6-chloro-3-(triazol-2-yl)pyridine-2-carboxylic acid

To a solution of 3-bromo-6-chloro-pyridine-2-carboxylic acid (5 g, 21 mmol) in dioxane (80 mL) and H₂O (0.3 mL) were added 2H-1,2,3-triazole (2.92 g, 42.3 mmol), Cs₂CO₃ (13.74 g, 42.3 mmol), and N,N′-dimethylcyclohexane-1,2-diamine (2.92 mg, 42.3 mmol) at 20° C. and the resulting mixture stirred for 10 min. CuI (0.406 mg, 42.3 mmol) was added at 20° C. The mixture was stirred at 80° C. for 16 h, at which time completion was determined by LCMS. The reaction mixture was quenched with H₂O (50 mL), the pH was adjusted to pH=2 with aq. HCl (2N), and the resulting mixture was extracted with EtOAc (15 mL×3). The combined organic layers were washed with brine (50 mL), dried over Na₂SO₄ and concentrated to deliver 6-chloro-3-(triazol-2-yl)pyridine-2-carboxylic acid (3 g, crude) as a yellow solid, which was used in the next step without further purification.

Step 2: Preparation of 6-chloro-N-methoxy-N-methyl-3-(triazol-2-yl)pyridine-2-carboxamide

To a solution of 6-chloro-3-(triazol-2-yl)pyridine-2-carboxylic acid (2 g, 8.93 mmol) in DMF (20 mL) were added N,N-diisopropylethylamine (2.3 g, 35.7 mmol) and (1-[bis(dimethylamino)methylene]-1H-1,2,3-triazolo[4,5-b]pyridinium 3-oxide hexafluorophosphate (3.4 g, 17.85 mmol) at 0° C. The mixture was stirred at 0° C. for 30 min. A solution of N,O-dimethylhydroxylamine hydrochloride (0.653 g, 13.4 mmol) was added at 0° C. The mixture was allowed to warm up to 15° C. and further stirred for 16 h, at which time completion was determined by TLC (EtOAc:EtOH=1:1). The mixture was quenched with H₂O (30 mL), extracted with EtOAc (10 mL×3). The combined organic layers were washed with brine (10 mL×3), dried over Na₂SO₄, filtered, and concentrated. The residue was purified by column chromatography on silica gel (PE/EtOAc=100:0 to 43:56) to furnish 6-chloro-N-methoxy-N-methyl-3-(triazol-2-yl)pyridine-2-carboxamide (1 g, 42% yield) as a yellow solid.

¹H-NMR (400 MHz, CDC₃) 5=8.36 (d, J=8.63 Hz, 1H), 7.82 (s, 2H), 7.45-7.54 (m, 1H), 3.49 (s, 3H), 3.31 (s, 3H).

### Step 3: Preparation of 1-[6-chloro-3-(triazol-2-yl)-2-pyridyl]ethanone

To a solution of 6-chloro-N-methoxy-N-methyl-3-(triazol-2-yl)pyridine-2-carboxamide (0.8 g, 2.99 mmol) in THF (10 mL) at 0° C. was added MeMgBr (3M, 3.9 ml, 11.98 mmol). The mixture was allowed to warm up to 20° C. with stirring over 12 h, at which time completion was determined by LCMS. The reaction mixture was quenched with aq. sat. NH₄Cl (20 mL) and extracted with EtOAc (10 mL×3). The organic layers were washed with brine (20 mL), dried over Na₂SO₄ and concentrated. The residue was purified by column chromatography on silica gel (PE/EtOAc=100:0 to 25:75) to deliver 1-[6-chloro-3-(triazol-2-yl)-2-pyridyl]ethanone (300 mg, 45% yield) as a yellow solid.

¹H-NMR (400 MHz, CDCl₃) δ=8.23 (d, J=8.58 Hz, 1H), 7.86 (s, 2H), 7.56 (d, J=8.46 Hz, 1H), 2.70 (s, 3H).

### Step 4: Preparation of 1-[6-chloro-3-(triazol-2-yl)-2-pyridyl]ethanamine

To a solution of 1-[6-chloro-3-(triazol-2-yl)-2-pyridyl]ethanone (0.168 g, 0.757 mmol) in MeOH (30 mL) was added NH₄OAc (583 mg, 7.57 mmol). The resulting mixture was stirred at 25° C. for 0.5 h. Subsequently, NaBH₃CN (95 mg, 1.514 mmol) was added and the mixture stirred at 25° C. for 0.5 h. The reaction mixture was then stirred at 30° C. for 12 h, at which time completion was determined by LCMS. The mixture was concentrated, brine (30 mL) was added, and the resulting mixture was extracted with DCM/IPA (3:1, 10 mL×3). The organic layers were dried over Na₂SO₄, filtered and concentrated to deliver 1-[6-chloro-3-(triazol-2-yl)-2-pyridyl]ethanamine (0.193 g, crude) as a yellow oil, which was employed in the next step without further purification.

### Step 5: Preparation of 3-chloro-N-[1-[6-chloro-3-(triazol-2-yl)-2-pyridyl]ethyl]-5-(trifluoro-methyl)benzamide (I-3)

To a solution of 1-[6-chloro-3-(triazol-2-yl)-2-pyridyl]ethanamine (0.15 g, 0.67 mmol) in MeCN (3 mL) was added N-methylimidazole (0.11 g, 1.34 mmol), N,N,N′,N′-tetramethylchloroformamidinium hexafluorophosphate (0.188 g, 0.67 mmol) and 3-chloro-5-(trifluoromethyl)benzoic acid (0.1 g, 0.447 mmol) at 20° C., and the resulting mixture stirred at the same temperature for 12 h, until completion was determined by LCMS. The reaction mixture was quenched with H₂O (10 mL) and extracted with EtOAc (3 mL×3). The combined organic layers were concentrated and the residue was purified by preparative HPLC (NH₄HCO₃) to deliver 3-chloro-N-[1-[6-chloro-3-(triazol-2-yl)-2-pyridyl]ethyl]-5-(trifluoromethyl)benzamide (I-3, 78 mg, 39% yield) as a white solid.

¹H-NMR (400 MHz, CDCl₃) δ=8.13 (d, J=8.50 Hz, 1H), 7.93-7.99 (m, 4H), 7.75 (s, 1H), 7.48-7.60 (m, 1H), 7.44 (d, J=8.50 Hz, 1H), 5.96-6.11 (m, 1H), 1.59 (d, J=6.75 Hz, 3H).

### Example 2: Preparation of 3-chloro-N-[1-[6-chloro-3-(triazol-2-yl)-2-pyridyl]ethyl]-N-(cyclopropylmethyl)-5-(trifluoromethyl)benzamide (I-5)

### Step 1: Preparation of 1-[6-chloro-3-(triazol-2-yl)-2-pyridyl]-N-(cyclopropylmethyl)ethanamine

To a solution of 1-[6-chloro-3-(triazol-2-yl)-2-pyridyl]ethanamine (0.5 g, 2.24 mmol) in MeOH (30 mL) were added cyclopropanecarbaldehyde (110 mg, 1.56 mmol) and AcOH (3 drops) at 25° C. and the mixture stirred for 4 h. Subsequently, NaBH₃CN (278 mg, 4.48 mmol) was added at 25° C. and the resulting mixture was stirred at 30° C. for 12 h, at which time completion was determined by LCMS. The reaction mixture was concentrated, quenched with H₂O (30 mL), and extracted with DCM/IPA (3:1, 10 mL×3). The combined organic layers were dried over Na₂SO₄, filtered and concentrated to furnish 1-[6-chloro-3-(triazol-2-yl)-2-pyridyl]-N-(cyclopropyl-methyl)ethanamine (0.2 g, crude) as a yellow oil, which was employed in the next step without further purification.

### Step 2: Preparation of 3-chloro-N-[1-[6-chloro-3-(triazol-2-yl)-2-pyridyl]ethyl]-N-(cyclopropyl-methyl)-5-(trifluoromethyl)benzamide (I-5)

To a solution of 1-[6-chloro-3-(triazol-2-yl)-2-pyridyl]-N-(cyclopropylmethyl)ethanamine (0.2 g, 0.722 mmol) in dichloromethane (DCM, 10 mL) were added 3-chloro-5-(trifluoromethyl)benzoyl chloride (0.263 g, 1.08 mmol) and triethylamine (0.146 g, 1.44 mmol) at 0° C. and the resulting mixture was stirred for 12 h, at which time completion was determined by LCMS. The reaction mixture was quenched with H₂O (10 mL) and extracted with DCM (3 mL×3). The combined organic layers were concentrated and the residue was purified by preparative HPLC (NH₄HCO₃) to deliver 3-chloro-N-[1-[6-chloro-3-(triazol-2-yl)-2-pyridyl]ethyl]-N-(cyclopropyl-methyl)-5-(trifluoromethyl)benzamide (I-5, 100 mg, 28% yield) as a yellow oil.

¹H-NMR (400 MHz, CDCl₃) δ=7.92-8.17 (m, 1H), 7.78 (brs, 1H), 7.55 (brd, J=12.01 Hz, 2H), 6.93-7.44 (m, 3H), 6.24 (br s, 1H), 3.38-3.99 (m, 1H), 2.73-3.31 (m, 1H), 1.74 (d, J=6.88 Hz, 3H), 0.66-1.32 (m, 1H), 0.11-0.62 (m, 3H), −0.56-0.01 (m, 1H).

### Example 3: Preparation of N-(cyclopropylmethyl)-N-[1-[3-[4-(3-pyridyl)triazol-2-yl]pyrazin-2-yl]ethyl]-3,5-bis(trifluoromethyl)benzamide (I-12) and N-(cyclopropylmethyl)-N-[1-[3-[4-(3-pyridyl)triazol-1-yl]pyrazin-2-yl]ethyl]-3,5-bis(trifluoromethyl)benzamide (I-13)

### Step 1: Preparation of 1-(3-chloropyrazin-2-yl)-N-(cyclopropylmethyl)ethanamine

To a solution of 1-(3-chloropyrazin-2-yl)ethanone (13.0 g, 83.0 mmol) in DCE (260 mL) was added cyclopropylmethanamine (11.8 g, 166 mmol) and the mixture was stirred at 25° C. for 0.2 h.

Next, NaBH(OAc)₃ (35.2 g, 166 mmol) and HOAc (9.97 g, 166 mmol, 9.50 mL) were added and the resulting mixture was stirred at 25° C. for 4 h, until completion was determined by TLC (DCM/MeOH 10:1, R_{f, starting material}=0.4, R_{f, product}=0.3). The mixture was concentrated under reduced pressure and the residue was purified by silica gel chromatography (pure DCM to DCM/MeOH 10:1) to afford 1-(3-chloropyrazin-2-yl)-N-(cyclopropylmethyl)ethanamine (17.0 g, 92% yield) as a yellow solid.

### Step 2: Preparation of N-[1-(3-chloropyrazin-2-yl)ethyl]-N-(cyclopropylmethyl)-3,5-bis(trifluoromethyl)benzamide

To a mixture of 1-(3-chloropyrazin-2-yl)-N-(cyclopropylmethyl)ethanamine (17.0 g, 80.3 mmol) and triethylamine (12.1 g, 120 mmol, 10.0 mL) in DCM (204 mL) was added 3,5-bis(trifluoro-methyl)benzoyl chloride (22.2 g, 80.3 mmol, 14.5 mL) dropwise at 0° C. The mixture was heated to 25° C. and stirred for 1 h, at which time completion was determined by TLC (PE/EtOAc 3:1, R_{f, starting material}=0.3). The mixture was concentrated under reduced pressure and the residue was purified by preparative HPLC (column: Welch Xtimate C18 250*70 mm #10 um; mobile phase: water (NH₄HCO₃)/ACN 50%-80%, 20 min) to afford N-[1-(3-chloropyrazin-2-yl)ethyl]-N-(cyclopropylmethyl)-3,5-bis(trifluoromethyl)benzamide (11.7 g, 32% yield) as a white solid.

¹H-NMR (400 MHz, CDCl₃) δ 8.54 (d, J=2.4 Hz, 1H), 8.37 (d, J=2.4 Hz, 1H), 7.95 (s, 1H), 7.91 (s, 2H), 5.98-6.30 (m, 1H), 2.84-3.42 (m, 2H), 1.77 (d, J=6.0 Hz, 3H), 0.51-0.95 (m, 1H), 0.22-0.49 (m, 2H), −0.56-0.10 (m, 2H).

### Step 3: Preparation of N-(cyclopropylmethyl)-N-[1-[3-[4-(3-pyridyl)triazol-2-yl]pyrazin-2-yl]ethyl]-3,5-bis(trifluoromethyl)benzamide (I-12) and N-(cyclopropylmethyl)-N-[1-[3-[4-(3-pyridyl)triazol-1-yl]pyrazin-2-yl]ethyl]-3,5-bis(trifluoromethyl)benzamide (I-13)

A mixture of N-[1-(3-chloropyrazin-2-yl)ethyl]-N-(cyclopropylmethyl)-3,5-bis(trifluoro-methyl)benzamide (300 mg, 0.664 mmol), 3-(2H-triazol-4-yl)pyridine (194 mg, 1.33 mmol), and K₂CO₃ (184 mg, 1.33 mmol) in DMF (9 ml) was stirred at 100° C. for 8 h. The mixture was diluted with water and extracted with DCM. The organic layer was dried over MgSO₄ and the volatiles were removed under reduced pressure. The residue was subjected to column chromatography on silica gel (eluent: cyclohexane/EtOAc 100:0 to 0:100; performed thrice) to deliver compounds I-12 (white solid, 46 mg, 11% yield) and I-13 (white solid, 42 mg, 9% yield).

I-12: ¹H-NMR (500 MHz, toluene-d8, 363.15K) δ 9.01 (d, J=2.4 Hz, 1H), 8.43 (dd, J=4.7, 1.7 Hz, 1H), 8.06 (d, J=2.4 Hz, 1H), 7.93 (d, J=2.4 Hz, 1H), 7.79 (dt, J=7.8, 2.1 Hz, 1H), 7.72 (s, 1H), 7.64 (s, 2H), 7.09 (t, J=1.2 Hz, 1H), 6.83 (ddd, J=7.9, 4.7, 0.9 Hz, 1H), 6.22 (q, J=6.8, 6.3 Hz, 1H), 3.17-3.00 (m, 2H), 1.69 (d, J=6.9 Hz, 3H), 0.65 (s, 1H), 0.26-0.11 (m, 2H), −0.17-−0.34 (m, 2H).

I-13: ¹H NMR (500 MHz, toluene-d8, 363.15K) δ 9.08 (d, J=2.4 Hz, 1H), 8.46 (dd, J=4.9, 1.8 Hz, 1H), 8.22 (s, 1H), 8.09-7.98 (m, 1H), 7.82 (d, J=2.6 Hz, 1H), 7.73 (s, 2H), 7.67 (s, 1H), 7.11-7.06 (m, 1H), 6.89 (dd, J=7.9, 4.7 Hz, 1H), 6.05 (q, J=6.9 Hz, 1H), 3.29 (d, J=6.0 Hz, 2H), 1.76 (d, J=6.9 Hz, 3H), 0.80 (t, J=5.7 Hz, 1H), 0.29 (dt, J=7.8, 2.1 Hz, 2H), −0.05-−0.19 (m, 2H).

### Example 4: Preparation of N-[1-[3-[4-(2-pyridyl)triazol-2-yl]pyrazin-2-yl]ethyl]-3,5-bis(trifluoro-methyl)benzamide (I-14)

A mixture of N-[1-(3-chloropyrazin-2-yl)ethyl]-3,5-bis(trifluoromethyl)benzamide (300 mg, 0.75 mmol), 2-(2H-triazol-4-yl)pyridine (220 mg, 1.50 mmol), and K₂CO₃ (208 mg, 1.50 mmol) in DMF (9 ml) was stirred at 100° C. for 16 h. The mixture was diluted with water and extracted with DCM, the organic layer was dried over MgSO₄ and the volatiles were removed under reduced pressure. The residue was purified by column chromatography on silica gel (eluent: cyclohexane/EtOAc 90:10 to 0:100) to deliver I-14 (white solid, 21 mg, 5% yield).

I-14: ¹H-NMR (500 MHz, CDCl₃) δ 8.73 (d, J=2.4 Hz, 1H), 8.72-8.70 (m, 1H), 8.65 (d, J=2.3 Hz, 1H), 8.63 (s, 1H), 8.26 (s, 2H), 8.17 (d, J=7.9 Hz, 1H), 8.01 (s, 1H), 7.84 (td, J=7.7, 1.8 Hz, 1H), 7.72 (d, J=7.9 Hz, 1H), 7.35 (ddd, J=7.6, 4.9, 1.2 Hz, 1H), 6.35 (dq, J=7.9, 6.7 Hz, 1H), 1.69 (d, J=6.7 Hz, 3H).

### Example 5: Preparation of 2-[3-[1-[[3,5-bis(trifluoromethyl)benzoyl]amino]ethyl]pyrazin-2-yl]-N-(2,4-dichlorophenyl)triazole-4-carboxamide (I-15)

A mixture of N-[1-(3-chloropyrazin-2-yl)ethyl]-3,5-bis(trifluoromethyl)benzamide (300 mg, 0.75 mmol), N-(2,4-dichlorophenyl)-2H-triazole-4-carboxamide (388 mg, 1.50 mmol), and K₂CO₃ (208 mg, 1.50 mmol) in DMF (9 ml) was stirred at 100° C. for 34 h. The mixture was diluted with water and extracted with DCM, the organic layer was dried over MgSO₄ and the volatiles were removed under reduced pressure. The residue was purified by preparative HPLC to deliver I-15 (orange solid, 29 mg, 6% yield).

I-15: ¹H-NMR (400 MHz, CDCl₃) δ 9.23 (s, 1H), 8.81 (d, J=2.3 Hz, 1H), 8.66 (d, J=2.3 Hz, 1H), 8.56 (s, 1H), 8.50 (d, J=8.9 Hz, 1H), 8.23 (s, 2H), 8.02 (s, 1H), 7.50 (d, J=7.9 Hz, 1H), 7.45 (d, J=2.4 Hz, 1H), 7.34 (dd, J=8.9, 2.4 Hz, 1H), 6.11 (dq, J=7.9, 6.7 Hz, 1H), 1.73 (d, J=6.7 Hz, 3H).

### Example 6: Preparation of N-[1-[3-(4-methylsulfonylpyrazol-1-yl)pyrazin-2-yl]ethyl]-3,5-bis(trifluoromethyl)benzamide (I-17)

A mixture of N-[1-(3-chloropyrazin-2-yl)ethyl]-3,5-bis(trifluoromethyl)benzamide (300 mg, 0.75 mmol), 2-(2H-triazol-4-yl)pyridine (220 mg, 1.50 mmol), and K₂CO₃ (208 mg, 1.50 mmol) in DMF (9 ml) was stirred at 130° C. The volatiles were removed under reduced pressure. The residue was purified by preparative HPLC to deliver I-17 (white solid, 80 mg, 31% yield).

I-17: ¹H-NMR (400 MHz, CDCl₃) δ 8.97 (s, 1H), 8.66 (d, J=2.3 Hz, 1H), 8.46 (d, J=2.4 Hz, 1H), 8.23 (s, 2H), 8.17 (s, 1H), 8.00 (s, 1H), 7.60 (d, J=7.9 Hz, 1H), 6.34 (dq, J=7.9, 6.7 Hz, 1H), 3.21 (s, 3H), 1.69 (d, J=6.7 Hz, 3H).

### Example 7: Synthesis of N-[1-[3-(4-methylsulfonyltriazol-2-yl)pyrazin-2-yl]ethyl]-3,5-bis(trifluoromethyl)benzamide (I-20)

To a solution of N-[1-(3-chloropyrazin-2-yl)ethyl]-3,5-bis(trifluoromethyl)benzamide (150 mg, 0.377 mmol) in 1,4-dioxane (10 ml) was added 4-methylsulfonyl-2H-triazole (222 mg, 1.507 mmol), K₃PO₄ (160 mg, 0.754 mmol), 1,1′-bis(diphenylphosphino)ferrocene (125 mg, 0.226 mmol) and palladium(II) acetate (17 mg, 0.075 mmol) at 20° C. under nitrogen. The reaction mixture was heated to 130° C. for 6 h in a microwave. LC-MS showed the reaction was completed. The mixture was cooled to 20° C. filtered through celite and washed with EtOAc (100 ml). The filtrate was concentrated under reduced pressure. The residue was purified by silica gel column (c-hexane:EtOAc=100:0 to 0:100) to give N-[1-[3-(4-methylsulfonyltriazol-2-yl)pyrazin-2-yl]ethyl]-3,5-bis(trifluoromethyl)benzamide (84 mg, 43% yield) as a yellow solid.

¹H-NMR (400 MHz, DMSO-d₆) δ 9.50 (d, J=6.5 Hz, 1H), 9.00 (d, J=2.4 Hz, 1H), 8.84 (s, 1H), 8.74 (d, J=2.3 Hz, 1H), 8.43 (s, 2H), 8.30 (s, 1H), 5.43 (p, J=6.7 Hz, 1H), 3.47 (s, 3H), 1.66 (d, J=6.9 Hz, 3H). LCMS: Desired Mass: m/z=508.4; Observed Mass: m/z=508.9

### Example 8: Synthesis of N-[1-[3-[4-(5-bromo-2-pyridyl)triazol-2-yl]pyrazin-2-yl]ethyl]-3,5-bis(trifluoromethyl)benzamide (I-21)

To a solution of N-[1-(3-chloropyrazin-2-yl)ethyl]-3,5-bis(trifluoromethyl)benzamide (300 mg, 0.754 mmol) in DMF (9 ml) were added 5-bromo-2-(2H-triazol-4-yl)pyridine (255 mg, 1.132 mmol) and potassium carbonate (209 mg, 1.509 mmol) at 20° C. The reaction mixture was heated to 100° C. and stirred for 20 h. LC-MS showed the reaction was completed. The mixture was cooled to 20-25° C., filtered and the filtrate was concentrated under reduced pressure. The residue was dissolved in DCM (300 ml) and washed with water (100 ml) and brine (100 ml). The organic phase was dried over MgSO₄ and concentrated under reduced pressure. The residue was purified by prep.-HPLC (amylose-column) and silica gel column (c-hexane:EtOAc=100:0 to 0:100) to give N-[1-[3-[4-(5-bromo-2-pyridyl)triazol-2-yl]pyrazin-2-yl]ethyl]-3,5-bis(trifluoromethyl)benzamide (36 mg, 8% yield) as a white solid.

¹H-NMR (500 MHz, CDCl₃) δ 8.75 (dd, J=11.0, 2.0 Hz, 2H), 8.64 (d, J=2.2 Hz, 1H), 8.60 (s, 1H), 8.26 (s, 2H), 8.06 (d, J=8.4 Hz, 1H), 8.01 (s, 1H), 7.96 (dd, J=8.4, 2.2 Hz, 1H), 7.71 (d, J=7.9 Hz, 1H), 6.31 (p, J=6.8 Hz, 1H), 1.69 (d, J=6.7 Hz, 3H).

LCMS: Desired Mass: m/z=586.3; Observed Mass: m/z=587.8

### Example 9: Synthesis of N-[1-[3-(4-oxazol-2-yltriazol-2-yl)pyrazin-2-yl]ethyl]-3,5-bis(trifluoromethyl)benzamide (I-24)

Step 1: Synthesis of 1-[3-(4-bromotriazol-2-yl)pyrazin-2-yl]ethenone: To a solution of 1-(3-chloropyrazin-2-yl)ethenone (1.1 g, 7.03 mmol) in ACN (50 ml) was added 4-bromo-2H-triazole (1.04 g, 7.03 mmol) and potassium carbonate (1.94 g, 14.06 mmol) at 20° C. The reaction mixture was heated to 80° C. and stirred for 4 h. LC-MS showed the reaction was completed. The mixture was cooled to 20° C. and filtered and the filtrate was concentrated under reduced pressure. The residue was purified by silica gel column (c-hexane:EtOAc=100:0 to 0:100) to give 1-[3-(4-bromotriazol-2-yl)pyrazin-2-yl]ethenone (1.06 g, 56% yield) as a white solid.

¹H NMR (400 MHz, Chloroform-d) δ 8.70-8.62 (m, 2H), 7.86 (s, 1H), 2.75 (s, 3H).

LCMS: Desired Mass: m/z=268.1; Observed Mass: m/z=269.6

Step 2: Synthesis of 1-[3-(4-bromotriazol-2-yl)pyrazin-2-yl]ethanamine: To a solution of 1-[3-(4-bromotriazol-2-yl)pyrazin-2-yl]ethenone (250 mg, 0.933 mmol) in NH₃ (7N in MeOH, 20 ml) were added ammonium acetate (1.08 g, 13.99 mmol) and NaBH₃CN (176 mg, 2.80 mmol) at 20° C. The reaction mixture was heated to 60° C. and stirred for 5 h. LC-MS showed the reaction was completed. Th mixture was cooled to 20° C., quenched with water (10 ml) and concentrated under reduced pressure. The residue was dissolved in HCl_{aq} (1N, 15 ml) and extracted with DCM (3×30 ml). The water phase was treated with NaOH_{aq} (1N) until pH 12 was reached and extracted with DCM (3×40 ml). This organic phase was dried over MgSO₄ and concentrated under reduced pressure to give 1-[3-(4-bromotriazol-2-yl)pyrazin-2-yl]ethanamine (134 mg, crude). The crude product was used in the next step without further purification. LCMS: Desired Mass: m/z=269.1; Observed Mass: m/z=270.7

Step 3: Synthesis of N-[1-[3-(4-bromotriazol-2-yl)pyrazin-2-yl]ethyl]-3,5-bis(trifluoro-methyl)benzamide: To a solution of 3,5-bis(trifluoromethyl)benzoic acid (164 mg, 0.635 mmol) and 1-[3-(4-bromotriazol-2-yl)pyrazin-2-yl]ethanamine (171 mg, 0.635 mmol) in DMF (7 ml) was added [dimethylamino(triazolo[4,5-b]pyridin-3-yloxy)methylene]-dimethyl-ammonium hexafluorophosphate (314 mg, 0.826 mmol) and N-ethyl-N-isopropyl-propan-2-amine (205 mg, 1.588 mmol) at 20° C. The reaction mixture was stirred for 20 h at 20° C. LC-MS showed the reaction was completed. The mixture was concentrated under reduced pressure and the residue was dissolved in DCM (150 ml). The organic phase was washed with water (60 ml), dried over MgSO₄ and concentrated under reduced pressure. The residue was purified by silica gel column (c-hexane:EtOAc=100:0 to 0:100) to give N-[1-[3-(4-bromotriazol-2-yl)pyrazin-2-yl]ethyl]-3,5-bis(trifluoromethyl)benzamide (291 mg, 90% yield) as a white solid.

LCMS: Desired Mass: m/z=509.2; Observed Mass: m/z=510.9

Step 4: Synthesis of N-[1-[3-(4-oxazol-2-yltriazol-2-yl)pyrazin-2-yl]ethyl]-3,5-bis(trifluoromethyl)benzamide (I-24): To a solution of N-[1-[3-(4-bromotriazol-2-yl)pyrazin-2-yl]ethyl]-3,5-bis(trifluoromethyl)benzamide (70 mg, 0.138 mmol) in toluene (3 ml) was added tributyl(oxazol-2-yl)stannane (74 mg, 0.206 mmol) and tetrakis(triphenylphosphine)palladium(0) (24 mg, 0.021 mmol) at 20° C. under nitrogen. The reaction mixture was heated to 100° C. and stirred for 20 h. LC-MS showed the reaction was completed. The mixture was cooled to 20° C., quenched with KF_{aq} (10%, 5 ml), filtered and washed with EtOAc (50 ml). The organic phase was washed with water (3×20 ml), dried over MgSO₄ and concentrated under reduced pressure. The residue was purified by silica gel column (c-hexane:EtOAc=100:0 to 0:100) to give N-[1-[3-(4-oxazol-2-yltriazol-2-yl)pyrazin-2-yl]ethyl]-3,5-bis(trifluoromethyl)benzamide (36 mg, 53% yield) as a white solid.

¹H-NMR (500 MHz, CDCl₃) δ 8.75 (d, J=2.2 Hz, 1H), 8.64 (d, J=2.2 Hz, 1H), 8.53 (s, 1H), 8.27 (s, 2H), 8.00 (s, 1H), 7.84 (s, 1H), 7.74 (d, J=7.8 Hz, 1H), 7.36 (s, 1H), 6.19 (p, J=6.8 Hz, 1H), 1.68 (d, J=6.7 Hz, 3H). LCMS: Desired Mass: m/z=497.4; Observed Mass: m/z=498.0.

### Example 10: Synthesis of N-[1-[4-chloro-3-(4-methylsulfanylpyrazol-1-yl)-2-pyridyl]ethyl]-3,5-bis(trifluoromethyl)benzamide (I-26)

Step 1: Synthesis of 1-[4-chloro-3-(4-methylsulfanylpyrazol-1-yl)-2-pyridyl]ethenone: To a solution of 1-(4-chloro-3-fluoro-2-pyridyl)ethanone (2 g, 11.5 mmol) in DMF (20 ml) was added 4-methylsulfanyl-1H-pyrazole (1.7 g, 15 mmol) and potassium carbonate (3.2 g, 23 mmol) at 20° C. The reaction mixture was heated to 50° C. and stirred for 16 h. TLC (PE/EtOAc=3:1) showed the reaction was completed. The mixture was quenched with NH₄Cl_{aq} (30 ml) and extracted with EtOAc (2×20 ml). The organic phase was washed with brine (3×20 ml), dried over Na₂SO₄ and concentrated under reduced pressure. The residue was purified by silica gel column (PE:EtOAc=5:1) to give 1-[4-chloro-3-(4-methylsulfanylpyrazol-1-yl)-2-pyridyl]ethenone (2 g, 65% yield) as a yellow solid.

Step 2: Synthesis of 1-[4-chloro-3-(4-methylsulfanylpyrazol-1-yl)-2-pyridyl]ethanamine: To a solution of 1-[4-chloro-3-(4-methylsulfanylpyrazol-1-yl)-2-pyridyl]ethenone in MeOH (100 ml) was added NH₃ (7N in MeOH, 20 ml) and ammonium acetate (3 g, 40 mmol) at 20° C. The reaction mixture was stirred for 0.5 h at 20° C. and then NaBH₃CN (1 g, 16 mmol) was added. The resulting mixture was stirred for 1 h at 20° C. and then for 16 h at 50° C. LC-MS showed the reaction was completed. The reaction mixture was concentrated under reduced pressure and quenched with water (20 ml). The residue was treated with NaOH (aqueous, 1 N) until pH 10 was reached and extracted with i-PrOH/DCM (1:3, 2×20 ml). The organic phase was dried over Na₂SO₄ and concentrated under reduced pressure to give 1-[4-chloro-3-(4-methylsulfanylpyrazol-1-yl)-2-pyridyl]ethanamine (2 g, crude) as a yellow oil. The crude product was used in the next step without further purification.

Step 3: Synthesis of N-[1-[4-chloro-3-(4-methylsulfanylpyrazol-1-yl)-2-pyridyl]ethyl]-3,5-bis(tri-fluoromethyl)benzamide: To a solution of 3,5-bis(trifluoromethyl)benzoic acid (2.9 g, 11.2 mmol) in ACN (30 ml) was added 1-methylimidazole (1.8 g, 22.4 mmol), N,N,N′,N′,-tetramethylchloroformamidinium hexafluorophophate (3.1 g, 11.2 mmol) and 1-[4-chloro-3-(4-methylsulfanylpyrazol-1-yl)-2-pyridyl]ethanamine (2 g crude, −7.5 mmol)) at 20° C. under nitrogen. The reaction mixture was stirred for 16 h. TLC (PE/EtOAc=3:1) showed the reaction was completed. The mixture was quenched with NH₄Cl_{aq} (30 ml) and extracted with EtOAc (2×30 ml). The organic phase was dried over Na₂SO₄ and concentrated under reduced pressure. The residue was purified by silica gel column (PE:EtOAc=3:1) to give N-[1-[4-chloro-3-(4-methylsulfanylpyrazol-1-yl)-2-pyridyl]ethyl]-3,5-bis(trifluoromethyl)benzamide (2 g, 52% yield) as a yellow solid.

¹H-NMR (400 MHz, CDCl₃): δ=8.59 (d, J=5.25 Hz, 1H), 8.24 (s, 2H), 8.01 (s, 1H), 7.83 (s, 1H), 7.72 (s, 1H), 7.54 (br d, J=7.38 Hz, 1H), 7.48 (d, J=5.25 Hz, 1H), 5.14 (t, J=7.07 Hz, 1H), 2.47 (s, 3H), 1.49 (d, J=6.75 Hz, 3H). LCMS: Desired Mass: m/z=508.1; Observed Mass: m/z=509.1.

### Example 11: Synthesis of N-[1-[4-chloro-3-(4-methylsulfonylpyrazol-1-yl)-2-pyridyl]ethyl]-3,5-bis(trifluoromethyl)benzamide (I-27) and N-[1-[4-chloro-3-(4-methylsulfinylpyrazol-1-yl)-2-pyridyl]ethyl]-3,5-bis(trifluoromethyl)benzamide (I-28)

To a solution of N-[1-[4-chloro-3-(4-methylsulfanylpyrazol-1-yl)-2-pyridyl]ethyl]-3,5-bis(tri-fluoromethyl)benzamide (1 g, 2 mmol) in EtOH (15 ml) was added H₂O₂ (446 mg, 4 mmol) and (NH₄)₆Mo₇O₂₄*4H₂O (1.23 g, 1 mmol) at 20° C. The reaction mixture was stirred for 16 h at 20° C. LC-MS showed the starting material was consumed. The mixture was filtered and the filtrate was concentrated under reduced pressure. The residue was purified by prep.-HPLC (NH₄HCO₃) to give N-[1-[4-chloro-3-(4-methylsulfonylpyrazol-1-yl)-2-pyridyl]ethyl]-3,5-bis(trifluoromethyl)benzamide (700 mg, 66% yield) as a white solid and N-[1-[4-chloro-3-(4-methylsulfinylpyrazol-1-yl)-2-pyridyl]ethyl]-3,5-bis(trifluoromethyl) benzamide (100 mg, 9% yield) as a white solid.

I-27: ¹H-NMR (400 MHz, CDCl₃): δ=8.65 (d, J=5.25 Hz, 1H), 8.32 (s, 1H), 8.20 (d, J=4.88 Hz, 3H), 8.02 (s, 1H), 7.51 (d, J=5.25 Hz, 1H), 7.29 (br s, 1H), 4.97 (br t, J=7.07 Hz, 1H), 3.27 (s, 3H), 1.55 (d, J=6.75 Hz, 3H). LCMS: Desired Mass: m/z=540.0; Observed Mass: m/z=540.0

I-28: ¹H-NMR (400 MHz, CDCl₃): δ=8.63 (dd, J=5.25, 0.88 Hz, 1H), 8.22 (s, 2H), 8.13-8.21 (m, 2H), 8.02 (s, 1H), 7.50 (dd, J=5.25, 2.25 Hz, 1H) 7.38 (br dd, J=15.38, 7.38 Hz, 1H), 4.99 (td, J=6.97, 3.56 Hz, 1H), 3.02 (d, J=3.25 Hz, 3H), 1.53 (dd, J=6.63, 5.50 Hz, 3H).

LCMS: Desired Mass: m/z=524.1; Observed Mass: m/z=525.1

### Example 12: Synthesis of N-[1-[3-[4-(1-methylpyrazol-3-yl)triazol-2-yl]pyrazin-2-yl]ethyl]-3,5-bis(trifluoromethyl)benzamide (I-29)

To a solution of N-[1-[3-(4-bromotriazol-2-yl)pyrazin-2-yl]ethyl]-3,5-bis(trifluoromethyl)benzamide (80 mg, 0.157 mmol) in toluene (4 ml) was added tributyl-(1-methylpyrazol-3-yl)stannane (87 mg, 0.236 mmol) and tetrakis(triphenylphosphine)palladium(0) (27 mg, 0.024 mmol) at 20° C. under nitrogen. The reaction mixture was heated to 100° C. and stirred for 20 h. LC-MS showed the reaction was completed. The mixture was cooled to 20° C., quenched with KF_{aq} (10%, 5 ml), filtered and washed with EtOAc (50 ml). The organic phase was washed with water (3×20 ml), dried over MgSO₄ and concentrated under reduced pressure. The residue was purified by silica gel column (c-hexane:EtOAc=100:0 to 0:100) to give N-[1-[3-[4-(1-methylpyrazol-3-yl)triazol-2-yl]pyrazin-2-yl]ethyl]-3,5-bis(trifluoromethyl)benzamide (76 mg, 95% yield) as a white solid.

¹H-NMR (400 MHz, CDCl₃) δ 8.67 (d, J=2.4 Hz, 1H), 8.60 (d, J=2.4 Hz, 1H), 8.36 (s, 1H), 8.28 (s, 2H), 7.99 (s, 1H), 7.89 (d, J=7.8 Hz, 1H), 7.45 (d, J=2.3 Hz, 1H), 6.83 (d, J=2.3 Hz, 1H), 6.33 (p, J=6.7 Hz, 1H), 4.00 (s, 3H), 1.65 (d, J=6.7 Hz, 3H).

LCMS: Desired Mass: m/z=510.4; Observed Mass: m/z=511.0.

### Example 13: Synthesis of N-[1-[3-(4-isopropylsulfanylpyrazol-1-yl)pyrazin-2-yl]ethyl]-3,5-bis(trifluoromethyl)benzamide (I-48)

Step 1: Synthesis of tert-butyl N-(1H-pyrazol-4-yl)carbamate: To a solution of 1H-pyrazol-4-amine (30 g, 361.4 mmol) in THF (600 ml) was added tert-butoxycarbonyl tert-butyl carbonate (86.6 g, 397.6 mmol) and a solution of sodium hydrogen carbonate (75.9 g, 903.5 mmol) in water (60 ml) at 0° C. The cooling bath was removed and the resulting mixture was stirred for 12 h to warm up to 25° C. TLC (PE:EtOAc=1:1) showed the reaction was completed. The mixture was poured into water (1 L) and extracted with EtOAc (2×600 ml). The organic phase was washed with brine (500 ml), dried over Na₂SO₄ and concentrated under reduced pressure. The residue was purified by silica gel column (PE:EtOAc=100:0 to 50:50) to give tert-butyl N-(1H-pyrazol-4-yl)carbamate (55.6 g, crude) as a purple solid.

¹H-NMR (400 MHz, DMSO-d6): δ=12.44 (br s, 1H), 9.06 (br s, 1H), 7.58 (s, 1H), 7.34 (s, 1H), 1.44 (s, 9H).

Step 2: Synthesis of tert-butyl N-[1-(3-acetylpyrazin-2-yl)pyrazol-4-yl]carbamate: 2 batches: To a solution of tert-butyl N-(1H-pyrazol-4-yl)carbamate (5 g, 27.32 mmol) in ACN/DMF (100 ml/30 ml) was added 1-(3-chloropyrazin-2-yl)ethanone (5.1 g, 32.78 mmol) and K₂CO₃ (9.4 g, 68.3 mmol) at 25° C. The reaction mixture was heated and stirred for 16 h at 80° C. and 16 h at 90° C. TLC (PE:EtOAc=3:1) showed the reaction was completed. Two batches were combined, poured into water (300 ml) and extracted with EtOAc (2×300 ml). The organic phase was washed with brine (200 ml), dried over Na₂SO₄ and concentrated under reduced pressure. The residue was purified by silica gel column (PE:EtOAc=100:0 to 70:30) to give tert-butyl N-[1-(3-acetylpyrazin-2-yl)pyrazol-4-yl]carbamate (11 g, 66% yield) as a yellow solid.

¹H-NMR (400 MHz, CDCl₃): δ=8.59 (br s, 1H), 8.39-8.45 (m, 2H), 7.63 (s, 1H), 6.44 (s, 1H), 2.66 (s, 3H), 1.53 (s, 9H).

Step 3: Synthesis of tert-butyl N-[1-[3-(1-aminoethyl)pyrazin-2-yl]pyrazol-4-yl]carbamate: 3 batches: To a solution of tert-butyl N-[1-(3-acetylpyrazin-2-yl)pyrazol-4-yl]carbamate (10 g, 33.0 mmol) in EtOH (500 ml) was added NH₃ (7N in MeOH, 100 ml) and ammonium acetate (25.4 g, 330.0 mmol) at 25° C. The reaction mixture was stirred for 1 h and then NaBH₃CN (6.2 g, 99.0 mmol) was added. The mixture was heated to 50° C. and stirred for 12 h. LC-MS showed the reaction was completed. Three batches were combined and quenched with water (130 ml). The resulting mixture was concentrated and extracted with EtOAc (3×100 ml). The organic phase was washed with brine (100 ml), dried over Na₂SO₄ and concentrated under reduced pressure to give tert-butyl N-[1-[3-(1-aminoethyl)pyrazin-2-yl]pyrazol-4-yl]carbamate (30 g, crude) as a yellow oil. The crude product was used in the next step without further purification.

¹H-NMR (400 MHz, DMSO-d6): δ=9.59 (br s, 1H), 8.71 (d, J=2.50 Hz, 1H), 8.62 (d, J=2.50 Hz, 1H), 8.49 (s, 1H), 7.85 (s, 1H), 5.44 (q, J=6.50 Hz, 1H), 1.46-1.51 (m, 12H)

Step 4: Synthesis of tert-butyl N-[1-[3-[1-[[3,5-bis(trifluoromethyl)benzoyl]amino]ethyl]pyrazin-2-yl]pyrazol-4-yl]carbamate: 2 batches: To a solution of tert-butyl N-[1-[3-(1-aminoethyl)pyrazin-2-yl]pyrazol-4-yl]carbamate (15 g, 49.3 mmol) in DCM (300 ml) was added triethylamine (13.7 ml, 98.6 mmol) and 3,5-bis(trifluoromethyl)benzoyl chloride (13.6 g, 49.3 mmol) at 0° C. under nitrogen. The cooling bath was removed and the resulting mixture was stirred for 3 h to warm up to 25° C. TLC (PE:EtOAc=3:1) showed the reaction was completed. Two batches were combined, poured into water (300 ml) and extracted with DCM (2×200 ml). The organic phase was washed with brine (100 ml), dried over Na₂SO₄ and concentrated under reduced pressure. The residue was purified by silica gel column (PE:EtOAc=100:0 to 80:20) to give tert-butyl N-[1-[3-[1-[[3,5-bis(trifluoromethyl)benzoyl]amino]ethyl]pyrazin-2-yl]pyrazol-4-yl]carbamate (31 g, 58% yield) as a white solid.

¹H-NMR (400 MHz, CDCl₃): δ=8.69 (br s, 1H), 8.49 (d, J=2.25 Hz, 1H), 8.38 (d, J=2.25 Hz, 1H), 8.28 (s, 2H), 8.09 (br d, J=7.00 Hz, 1H), 8.01 (s, 1H), 7.82 (s, 1H), 6.69 (br s, 1H), 6.51 (quin, J=7.00 Hz, 1H), 1.62 (br d, J=6.63 Hz, 3H), 1.52 (br s, 9H)

Step 5: Synthesis of [1-[3-[1-[[3,5-bis(trifluoromethyl)benzoyl]amino]ethyl]pyrazin-2-yl]pyrazol-4-yl]ammonium chloride: A solution of tert-butyl N-[1-[3-[1-[[3,5-bis(trifluoromethyl)benzoyl]amino]ethyl]pyrazin-2-yl]pyrazol-4-yl]carbamate (31 g, 57.0 mmol) in HCl (EtOAc, 350 ml) was stirred for 12 h at 25° C. TLC (PE:EtOAc=1:1) showed the reaction was completed. The reaction mixture was concentrated under reduced pressure to give [1-[3-[1-[[3,5-bis(trifluoro-methyl)benzoyl]amino]ethyl]pyrazin-2-yl]pyrazol-4-yl]ammonium chloride (25 g, 93% yield) as a white solid.

¹H-NMR (400 MHz, methanol-d4): δ=8.73 (s, 1H), 8.69 (d, J=2.38 Hz, 1H), 8.47 (d, J=2.38 Hz, 1H), 8.40 (s, 2H), 8.14 (s, 1H), 8.01 (s, 1H), 6.00 (q, J=6.88 Hz, 1H), 1.73 (d, J=6.88 Hz, 3H)

Step 6: Synthesis of [1-[3-[1-[[3,5-bis(trifluoromethyl)benzoyl]amino]ethyl]pyrazin-2-yl]pyrazol-4-yl]thiocyanate: 2 batches: To a mixture of [1-[3-[1-[[3,5-bis(trifluoromethyl)benzoyl]amino]ethyl]pyrazin-2-yl]pyrazol-4-yl]ammonium chloride (5 g, 11.26 mmol), copper(I) thiocyanate (2.72 g, 22.52 mmol) and potassium thiocyanate (2.18 g, 22.52 mmol) in ACN (150 ml) was added tert-butyl nitrite (2.32 g, 22.52 mmol) during 3 h at 70° C. The resulting mixture was stirred for 1 h at 70° C. TLC (PE:EtOAc=3:1) showed the reaction was completed. Two batches were combined and filtered. The filtrate was diluted with water (200 ml) and extracted with EtOAc (3×100 ml). The organic phase was dried over Na₂SO₄ and concentrated under reduced pressure. The residue was purified by silica gel column (PE:EtOAc=100:0 to 78:22) to give [1-[3-[1-[[3,5-bis(trifluoromethyl)benzoyl]amino]ethyl]pyrazin-2-yl]pyrazol-4-yl]thiocyanate (3.7 g, 34% yield) as a yellow solid.

¹H-NMR (400 MHz, CDCl₃): δ=8.79 (s, 1H), 8.64 (d, J=2.38 Hz, 1H), 8.45 (d, J=2.25 Hz, 1H), 8.24 (s, 2H), 8.00-8.05 (m, 2H), 7.63 (br d, J=7.63 Hz, 1H), 6.37 (quin, J=6.97 Hz, 1H), 1.69 (d, J=6.63 Hz, 3H). LCMS: Desired Mass: m/z=486.1; Observed Mass: m/z=487.1.

Step 7: Synthesis of N-[1-[3-(4-isopropylsulfanylpyrazol-1-yl)pyrazin-2-yl]ethyl]-3,5-bis(tri-fluoromethyl)benzamide: 7 batches: To a solution of [1-[3-[1-[[3,5-bis(trifluoromethyl)benzoyl]amino]ethyl]pyrazin-2-yl]pyrazol-4-yl]thiocyanate (100 mg, 0.206 mmol) in THF (5 ml) was added copper(I) iodide (47 mg, 0.25 mmol) at 25° C. under nitrogen. The reaction mixture was stirred for 0.5 h and cooled down to 0° C. Then i-PrMgCl (2M, 0.31 ml, 0.62 mmol) was added and stirred for 1 h at 0° C. TLC (PE:EtOAc=5:1) showed the reaction was completed. Seven batches were combined and poured into NH₄Cl_{aq} (50 ml) and extracted with EtOAc (2×30 ml). The organic phase was washed with brine (30 ml), dried over Na₂SO₄ and concentrated under reduced pressure. The residue was purified by silica gel column (PE:EtOAc=100:0 to 84:16) to give N-[1-[3-(4-isopropylsulfanylpyrazol-1-yl)pyrazin-2-yl]ethyl]-3,5-bis(trifluoromethyl)benzamide (200 mg, 27.5% yield) as a yellow solid.

¹H-NMR (400 MHz, CDCl₃): δ=8.56 (d, J=2.38 Hz, 1H), 8.49 (s, 1H), 8.42 (d, J=2.38 Hz, 1H), 8.27 (s, 2H), 8.01 (s, 1H), 7.95 (br d, J=8.25 Hz, 1H), 7.84 (s, 1H), 6.40-6.50 (m, 1H), 3.12 (dt, J=13.38, 6.69 Hz, 1H), 1.65 (d, J=6.63 Hz, 3H), 1.30 (d, J=6.63 Hz, 6H).

LCMS: Desired Mass: m/z=503.1; Observed Mass: m/z=504.1.

### Example 14: Synthesis of N-[1-[4-fluoro-3-(4-methylsulfonylpyrazol-1-yl)-2-pyridyl]ethyl]-3,5-bis(trifluoromethyl)benzamide (I-53) and N-[1-[4-hydroxy-3-(4-methylsulfonylpyrazol-1-yl)-2-pyridyl]ethyl]-3,5-bis(trifluoromethyl)benzamide (I-59)

To a solution of N-[1-[4-chloro-3-(4-methylsulfonylpyrazol-1-yl)-2-pyridyl]ethyl]-3,5-bis(tri-fluoromethyl)benzamide (300 mg, 0.55 mmol) in DMSO (10 ml) was added potassium fluoride (164 mg, 2.8 mmol) under nitrogen. The reaction mixture was heated to 140° C. and stirred for 2 h. LC-MS showed the reaction was completed. The mixture was quenched with NH₄Cl_{aq} (20 ml) and extracted with EtOAc (2×20 ml). The organic phase was dried over Na₂SO₄ and concentrated under reduced pressure. The residue was purified by prep.-HPLC (NH₄HCO₃) to give N-[1-[4-fluoro-3-(4-methylsulfonylpyrazol-1-yl)-2-pyridyl]ethyl]-3,5-bis(trifluoromethyl)benzamide (100 mg, 26% yield) as a white solid and N-[1-[4-hydroxy-3-(4-methylsulfonylpyrazol-1-yl)-2-pyridyl]ethyl]-3,5-bis(trifluoromethyl)benzamide (35 mg, 9% yield) as a white solid.

I-53: ¹H-NMR (400 MHz, CDCl₃): δ=8.72 (dd, J=7.09, 5.58 Hz, 1H), 8.35 (s, 1H), 8.21 (d, J=11.29 Hz, 3H), 8.02 (s, 1H), 7.40 (br d, J=7.53 Hz, 1H), 7.23-7.27 (m, 1H), 5.21 (t, J=7.03 Hz, 1H), 3.26 (s, 3H), 1.57 (d, J=6.78 Hz, 3H).

LCMS: Desired Mass: m/z=524.0; Observed Mass: m/z=525.0.

I-59: ¹H-NMR (400 MHz, CDCl₃): δ=8.47-8.57 (m, 1H), 8.36 (s, 2H), 8.32 (s, 1H), 7.99 (s, 1H), 7.96 (s, 1H), 7.62 (br d, J=6.53 Hz, 1H), 6.58 (br d, J=7.03 Hz, 1H), 4.63-4.97 (m, 1H), 3.18 (s, 3H), 1.55 (br d, J=7.15 Hz, 3H).

LCMS: Desired Mass: m/z=522.1; Observed Mass: m/z=523.1

### Example 15: Synthesis of N-[1-[3-(4-bromotriazol-2-yl)-4-chloro-2-pyridyl]ethyl]-3,5-bis(tri-fluoromethyl)benzamide (I-54)

Step 1: Synthesis of 1-(4-chloro-3-fluoro-2-pyridyl)ethanone: A mixture of 1,4-diazabicyclo-[2,2,2]-octane (25.8 g, 229.2 mmol) in MTBE (200 ml) was cooled to −78° C. under nitrogen. n-butyllithium (92 ml, 229.2 mmol) was added dropwise and the mixture was stirred for 0.5 h. Then a solution of 4-chloro-3-fluoro-pyridine (20 g, 152.8 mmol) in MTBE (50 ml) was added dropwise and the mixture was stirred for 30 min at −78° C. And then a solution of N-methoxy-N-methyl-acetamide (23.6 g, 229.2 mmol) in MTBE (50 ml) and the mixture was stirred for 16 h at −78° C. HPLC showed the reaction was completed. The reaction mixture was allowed to warm up to 25° C. and was quenched with ammonium acetate (aqueous, 300 ml) and extracted with EtOAc (2×300 ml). The organic phase was dried over Na₂SO₄ and concentrated under reduced pressure. The residue was purified by silica gel column (PE:EtOAc=5:1) to give 1-(4-chloro-3-fluoro-2-pyridyl)ethanone (16 g, 61% yield) as a yellow solid.

¹H-NMR (400 MHz, CDCl₃): δ=8.39 (d, J=5.02 Hz, 1H), 7.57 (t, J=5.02 Hz, 1H), 2.71 (d, J=0.75 Hz, 3H).

Step 2: Synthesis of 1-[3-(4-bromotriazol-2-yl)-4-chloro-2-pyridyl]ethenone: To a solution of 1-(4-chloro-3-fluoro-2-pyridyl)ethanone (1 g, 5.8 mmol) in DMF (20 ml) was added 4-bromo-2H-triazole (858 mg, 5.8 mmol) and potassium carbonate (1.6 g, 11.6 mmol) at 20° C. The reaction mixture was stirred for 16 h at 20° C. TLC (PE/EtOAc=5:1) showed the reaction was completed. The mixture was quenched with NH₄Cl_{aq} (30 ml) and extracted with EtOAc (2×20 ml). The organic phase was washed with brine (3×20 ml), dried over Na₂SO₄ and concentrated under reduced pressure. The residue was purified by silica gel column (PE:EtOAc=7:1) to give 1-[3-(4-bromotriazol-2-yl)-4-chloro-2-pyridyl]ethenone (1.2 g, 69% yield) as a white solid.

¹H-NMR (400 MHz, CDCl₃): δ=8.70 (d, J=5.27 Hz, 1H), 7.86 (s, 1H), 7.70 (d, J=5.27 Hz, 1H), 2.65 (s, 3H).

Step 3: Synthesis of 1-[3-(4-bromotriazol-2-yl)-4-chloro-2-pyridyl]ethanamine: To a solution of 1-[3-(4-bromotriazol-2-yl)-4-chloro-2-pyridyl]ethenone (1.3 g, 4 mmol) in MeOH (100 ml) was added NH₃ (7N in MeOH, 20 ml) and ammonium acetate (3 g, 40 mmol) at 20° C. The reaction mixture was stirred for 0.5 h at 20° C. and then sodium cyanoborohydride (504 mg, 8 mmol) was added. The resulting mixture was stirred for 1 h at 20° C. and then for 16 h at 50° C. LC-MS showed the reaction was completed. The reaction mixture was concentrated under reduced pressure and quenched with water (20 ml). The residue was treated with NaOH_{aq} (1 N) until pH was reached and extracted with i-PrOH/DCM (1:3, 2×20 ml). The organic phase was dried over Na₂SO₄ and concentrated under reduced pressure to give 1-[3-(4-bromotriazol-2-yl)-4-chloro-2-pyridyl]ethanamine (1.3 g crude) as a yellow oil. The crude product was used in the next step without further purification.

Step 4: Synthesis of N-[1-[3-(4-bromotriazol-2-yl)-4-chloro-2-pyridyl]ethyl]-3,5-bis(trifluoro-methyl)benzamide: To a solution of 3,5-bis(trifluoromethyl)benzoic acid (1.3 g, 5 mmol) in ACN (15 ml) was added 1-methylimidazole (820 mg, 10 mmol), N,N,N′,N′,-tetramethylchloroformamidinium hexafluorophophate (1.4 g, 5 mmol) and 1-[3-(4-bromotriazol-2-yl)-4-chloro-2-pyridyl]ethanamine (1 g crude, ~3.3 mmol) at 20° C. under nitrogen. The reaction mixture was stirred for 16 h. TLC (PE/EtOAc=3:1) showed the reaction was completed. The mixture was quenched with NH₄Cl (aqueous, 20 ml) and extracted with EtOAc (2×20 ml). The organic phase was washed with brine (3×20 ml), dried over Na₂SO₄ and concentrated under reduced pressure. The residue was purified by silica gel column (PE:EtOAc=3:1) to give N-[1-[3-(4-bromotriazol-2-yl)-4-chloro-2-pyridyl]ethyl]-3,5-bis(trifluoromethyl)benzamide (1.1 g, 62% yield) as a yellow solid.

¹H-NMR (400 MHz, CDCl₃): δ=8.69 (d, J=5.27 Hz, 1H), 8.25 (s, 2H), 8.02 (s, 1H), 7.97 (s, 1H), 7.66 (br d, J=7.28 Hz, 1H), 7.54 (d, J=5.27 Hz, 1H), 5.06-5.16 (m, 1H), 1.48 (d, J=6.78 Hz, 3H).

LCMS: Desired Mass: m/z=541.0; Observed Mass: m/z=542.0.

### Example 16: Synthesis of N-[1-[4-chloro-3-[4-(2-pyridyl)triazol-2-yl]-2-pyridyl]ethyl]-3,5-bis(tri-fluoromethyl)benzamide (I-57) and N-[1-[4-chloro-3-(triazol-2-yl)-2-pyridyl]ethyl]-3,5-bis(trifluoro-methyl)benzamide (I-68)

To a solution of N-[1-[3-(4-bromotriazol-2-yl)-4-chloro-2-pyridyl]ethyl]-3,5-bis(trifluoro-methyl)benzamide (I-54, 700 mg, 1.3 mmol) in toluene (10 ml) was added bis(triphenylphosphine)palladium(II)dichloride (100 mg cat.), CuI (100 mg, cat.) and tributyl(2-pyridyl)stannane (716 mg, 1.9 mmol) at 20° C. The reaction mixture was heated to 120° C. and stirred for 16 h. LC-MS showed the reaction was completed. The mixture was quenched with NH₄Cl_{aq} (20 ml) and extracted with EtOAc (2×20 ml). The organic phase was dried over Na₂SO₄ and concentrated under reduced pressure. The residue was purified by prep.-HPLC (NH₄HCO₃) to give N-[1-[4-chloro-3-[4-(2-pyridyl)triazol-2-yl]-2-pyridyl]ethyl]-3,5-bis(trifluoromethyl)benzamide (200 mg, 28% yield) as a yellow solid and N-[1-[4-chloro-3-(triazol-2-yl)-2-pyridyl]ethyl]-3,5-bis(trifluoro-methyl)benzamide (50 mg, 7% yield).

I-57: ¹H-NMR (400 MHz, methanol-d4): δ=8.74 (d, J=5.38 Hz, 1H), 8.58 (dt, J=4.82, 1.28 Hz, 1H), 8.51 (s, 1H), 8.32 (s, 2H), 8.02 (s, 1H), 7.78-7.86 (m, 2H), 7.72 (d, J=5.38 Hz, 1H), 7.39 (ddd, J=7.00, 5.13, 1.63 Hz, 1H), 5.17 (q, J=7.00 Hz, 1H), 1.62 (d, J=7.00 Hz, 3H).

LCMS: Desired Mass: m/z=540.1; Observed Mass: m/z=541.1.

I-68: ¹H-NMR (400 MHz, methanol-d4): δ=8.72 (d, J=5.25 Hz, 1H), 8.40 (s, 2H), 8.15 (s, 1H), 8.07 (s, 2H), 7.70 (d, J=5.25 Hz, 1H), 4.96 (d, J=6.88 Hz, 1H), 1.54 (d, J=7.00 Hz, 3H).

LCMS: Desired Mass: m/z=463.1; Observed Mass: m/z=464.1.

### Example 17: Synthesis of 3,5-bis(trifluoromethyl)-N-[1-[3-[4-(trifluoromethylsulfonyl)pyrazol-1-yl]pyrazin-2-yl]ethyl]benzamide (I-55) and 3,5-bis(trifluoromethyl)-N-[1-[3-[4-(trifluoromethyl-sulfanyl)pyrazol-1-yl]pyrazin-2-yl]ethyl]benzamide (I-60)

Step 1: Synthesis of 3,5-bis(trifluoromethyl)-N-[1-[3-[4-(trifluoromethylsulfanyl)pyrazol-1-yl]pyrazin-2-yl]ethyl]benzamide: To a solution of [1-[3-[1-[[3,5-bis(trifluoromethyl)benzoyl]amino]ethyl]pyrazin-2-yl]pyrazol-4-yl]thiocyanate (500 mg, 1.03 mmol) in THF (15 ml) was added trimethyl(trifluoromethyl)silane (438 mg, 3.09 mmol) under nitrogen. The reaction mixture was cooled to 0° C. and tetra n-butylammonium fluoride (0.31 ml, 0.31 mmol) was added dropwise. The mixture was stirred for 1 h at 0° C. TLC (PE:EtOAc=5:1) showed the reaction was completed. The reaction mixture was poured into water (20 ml) and extracted with EtOAc (2×20 ml). The organic phase was washed with brine (1×20 ml), dried over Na₂SO₄ and concentrated under reduced pressure. The residue was purified by silica gel column (PE:EtOAc=100:0 to 85:15) to give 3,5-bis(trifluoromethyl)-N-[1-[3-[4-(trifluoromethylsulfanyl)pyrazol-1-yl]pyrazin-2-yl]ethyl]benzamide (290 mg, 53% yield) as a white solid.

¹H-NMR (400 MHz, CDCl₃): δ=8.75 (s, 1H), 8.62 (d, J=1.63 Hz, 1H), 8.45 (d, J=1.38 Hz, 1H), 8.25 (s, 2H), 8.01 (br d, J=7.13 Hz, 2H), 7.72 (br d, J=7.63 Hz, 1H), 6.42 (quin, J=6.91 Hz, 1H), 1.68 (d, J=6.63 Hz, 3H). LCMS: Desired Mass: m/z=529.1; Observed Mass: m/z=530.1.

Step 2: Synthesis of 3,5-bis(trifluoromethyl)-N-[1-[3-[4-(trifluoromethylsulfonyl)pyrazol-1-yl]pyrazin-2-yl]ethyl]benzamide: To a solution of 3,5-bis(trifluoromethyl)-N-[1-[3-[4-(trifluoro-methylsulfanyl)pyrazol-1-yl]pyrazin-2-yl]ethyl]benzamide (100 mg, 0.189 mmol) in ACN/water (4 ml/1 ml) was added sodium periodate (30 mg, cat.) and ruthenium chloride (5 mg, cat.) at 25° C. The reaction mixture was stirred for 5 h at this temperature. TLC (PE:EtOAc=3:1) showed the reaction was completed. The mixture was diluted with water (5 ml) and EtOAc (10 ml) and filtered through celite. The filtrate was extracted with EtOAc (2×10 ml). The organic phase was washed with brine (10 ml), dried over Na₂SO₄ and concentrated under reduced pressure. The residue was purified by prep.-TLC (PE:EtOAc=3:1) to give 3,5-bis(trifluoro-methyl)-N-[1-[3-[4-(trifluoromethylsulfonyl)pyrazol-1-yl]pyrazin-2-yl]ethyl]benzamide (105 mg, 99% yield) as a white solid.

¹H-NMR (400 MHz, CDCl₃): δ=9.16 (s, 1H), 8.74 (d, J=2.25 Hz, 1H), 8.51 (d, J=2.25 Hz, 1H), 8.29 (s, 1H), 8.23 (s, 2H), 8.03 (s, 1H), 7.49 (br d, J=7.75 Hz, 1H), 6.33 (quin, J=7.00 Hz, 1H), 1.72 (d, J=6.63 Hz, 3H). LCMS: Desired Mass: m/z=561.1; Observed Mass: m/z=562.0.

### Example 18: Synthesis of N-[1-[3-(4-cyclopropylsulfonylpyrazol-1-yl)pyrazin-2-yl]ethyl]-3,5-bis(trifluoromethyl)benzamide (I-65)

Step 1: Synthesis of N-[1-[3-(4-cyclopropylsulfanylpyrazol-1-yl)pyrazin-2-yl]ethyl]-3,5-bis(trifluoromethyl)benzamide: 7 batches: To a solution of [1-[3-[1-[[3,5-bis(trifluoromethyl)benzoyl]amino]ethyl]pyrazin-2-yl]pyrazol-4-yl]thiocyanate in THF (5 ml) was added CuI (47 mg, 0.247 mmol) at 25° C. under nitrogen. The reaction mixture was stirred for 0.5 h and cooled down to 0° C. Then cyclopropylmagnesium bromide (1M, 1.23 ml, 1.23 mmol) was added and stirred for 1 h at 0° C. TLC (PE:EtOAc=5:1) showed the reaction was completed. Seven batches were combined and poured into NH₄Cl_{aq} (50 ml) and extracted with EtOAc (2×30 ml). The organic phase was washed with brine (30 ml), dried over Na₂SO₄ and concentrated under reduced pressure. The residue was purified by silica gel column (PE:EtOAc=100:0 to 85:15) to give N-[1-[3-(4-cyclopropylsulfanylpyrazol-1-yl)pyrazin-2-yl]ethyl]-3,5-bis(trifluoromethyl)benzamide (230 mg, 31.8% yield) as a yellow solid.

¹H-NMR (400 MHz, CDCl₃): δ=8.54 (d, J=2.50 Hz, 1H), 8.46 (d, J=0.63 Hz, 1H), 8.42 (d, J=2.50 Hz, 1H), 8.27 (s, 2H), 8.01 (s, 1H), 7.97 (br d, J=8.25 Hz, 1H), 7.87 (s, 1H), 6.42-6.51 (m, 1H), 2.17-2.26 (m, 1H), 1.66 (d, J=6.63 Hz, 3H), 0.94-1.00 (m, 2H), 0.71-0.77 (m, 2H).

LCMS: Desired Mass: m/z=501.1; Observed Mass: m/z=502.1.

Step 2: Synthesis of N-[1-[3-(4-cyclopropylsulfonylpyrazol-1-yl)pyrazin-2-yl]ethyl]-3,5-bis(trifluoromethyl)benzamide: To a solution of N-[1-[3-(4-cyclopropylsulfanylpyrazol-1-yl)pyrazin-2-yl]ethyl]-3,5-bis(trifluoromethyl)benzamide (180 mg, 0.359 mmol) in EtOH (8 ml) was added (NH₄)₆Mo₇O₂₄*4H₂O (209 mg, 0.18 mmol) and hydrogen peroxide (30%, 122 mg, 1.077 mmol) at 25° C. The reaction mixture was stirred for 12 h. TLC (PE:EtOAc=1:1) showed the reaction was completed. The reaction mixture was poured into water (20 ml) and extracted with EtOAc (2×20 ml). The organic phase was washed with brine (20 ml), dried over Na₂SO₄ and concentrated under reduced pressure. The residue was purified by prep.-TLC (PE:EtOAc=1:1) to give N-[1-[3-(4-cyclopropylsulfonylpyrazol-1-yl)pyrazin-2-yl]ethyl]-3,5-bis(trifluoromethyl)benzamide (99 mg, 52% yield) as a white solid.

¹H-NMR (400 MHz, CDCl₃): δ=8.95 (s, 1H), 8.66 (d, J=2.38 Hz, 1H), 8.47 (d, J=2.25 Hz, 1H), 8.25 (s, 2H), 8.15 (s, 1H), 8.03 (s, 1H), 7.61 (br d, J=8.00 Hz, 1H), 6.35-6.46 (m, 1H), 2.59-2.68 (m, 1H), 1.70 (d, J=6.63 Hz, 3H), 1.41-1.46 (m, 2H), 1.10-1.18 (m, 2H).

LCMS: Desired Mass: m/z=533.1; Observed Mass: m/z=534.1.

### Example 19: Synthesis of N-[1-[4-fluoro-3-[4-(2-pyridyl)triazol-2-yl]-2-pyridyl]ethyl]-3,5-bis(tri-fluoromethyl)benzamide (I-66)

Synthesis of N-[1-[4-fluoro-3-[4-(2-pyridyl)triazol-2-yl]-2-pyridyl]ethyl]-3,5-bis(trifluoro-methyl)benzamide: To a solution of N-[1-[4-chloro-3-[4-(2-pyridyl)triazol-2-yl]-2-pyridyl]ethyl]-3,5-bis(trifluoromethyl)benzamide (100 mg, 0.18 mmol) in DMSO (5 ml) was added KF (56 mg, 0.96 mmol) at 140° C. under nitrogen. The reaction mixture was stirred for 2 h at 140° C. LC-MS showed the reaction was completed. The mixture was quenched with NH₄Cl_{aq} (20 ml) and extracted with EtOAc (2×20 ml). The organic phase was dried over Na₂SO₄ and concentrated under reduced pressure. The residue was purified by prep.-HPLC (NH₄HCO₃) to give N-[1-[4-fluoro-3-[4-(2-pyridyl)triazol-2-yl]-2-pyridyl]ethyl]-3,5-bis(trifluoromethyl)benzamide (30 mg, 32% yield) as a yellow solid.

¹H-NMR (400 MHz, methanol-d4): δ=8.82 (dd, J=7.19, 5.69 Hz, 1H), 8.61 (brd, J=4.88 Hz, 1H), 8.53 (s, 1H), 8.33 (s, 2H), 8.04 (s, 1H), 7.83-7.94 (m, 2H), 7.50 (dd, J=9.13, 5.63 Hz, 1H), 7.40-7.46 (m, 1H), 5.33 (q, J=7.09 Hz, 1H), 1.67 (d, J=7.00 Hz, 3H).

LCMS: Desired Mass: m/z=524.1; Observed Mass: m/z=525.1.

### Example 20: Synthesis of N-[1-[3-(4-isopropylsulfonylpyrazol-1-yl)pyrazin-2-yl]ethyl]-3,5-bis(tri-fluoromethyl)benzamide (I-67) and N-[1-[3-(4-isopropylsulfinylpyrazol-1-yl)pyrazin-2-yl]ethyl]-3,5-bis(trifluoromethyl)benzamide (I-69)

To a solution of N-[1-[3-(4-isopropylsulfanylpyrazol-1-yl)pyrazin-2-yl]ethyl]-3,5-bis(trifluoro-methyl)benzamide (160 mg, 0.318 mmol) in EtOH (5 ml) was added (NH₄)₆Mo₇O₂₄*4H₂O (185 mg, 0.159 mmol) and H₂O₂ (30%, 108 mg, 0.954 mmol) at 25° C. The reaction mixture was stirred for 12 h. TLC (PE:EtOAc=1:1) showed the reaction was completed. The reaction mixture was poured into water (20 ml) and extracted with EtOAc (2×20 ml). The organic phase was washed with brine (20 ml), dried over Na₂SO₄ and concentrated under reduced pressure. The residue was purified by prep.-TLC (PE:EtOAc=1:1) to give N-[1-[3-(4-isopropylsulfonylpyrazol-1-yl)pyrazin-2-yl]ethyl]-3,5-bis(trifluoromethyl)benzamide (92 mg, 54% yield) as a yellow solid and N-[1-[3-(4-isopropylsulfinylpyrazol-1-yl)pyrazin-2-yl]ethyl]-3,5-bis(trifluoromethyl)benzamide (43 mg, 26% yield) as a white solid.

I-67: ¹H-NMR (400 MHz, CDCl₃): δ=8.94 (s, 1H), 8.67 (d, J=2.25 Hz, 1H), 8.47 (d, J=2.25 Hz, 1H), 8.24 (s, 2H), 8.14 (s, 1H), 8.02 (s, 1H), 7.61 (br d, J=7.88 Hz, 1H), 6.40 (quin, J=7.04 Hz, 1H), 3.27 (dt, J=13.73, 6.83 Hz, 1H), 1.69 (d, J=6.63 Hz, 3H), 1.42 (d, J=6.88 Hz, 6H).

LCMS: Desired Mass: m/z=535.1; Observed Mass: m/z=536.1.

I-69: ¹H-NMR (400 MHz, CDCl₃): δ=8.78-8.95 (m, 1H), 8.61-8.69 (m, 1H), 8.43-8.49 (m, 1H), 8.26 (s, 2H), 8.04-8.15 (m, 1H), 8.02 (s, 1H), 7.74 (br dd, J=7.50, 4.00 Hz, 1H), 6.35-6.51 (m, 1H), 3.05-3.33 (m, 1H), 1.67 (dd, J=6.63, 3.00 Hz, 3H), 1.35 (dd, J=6.82, 3.44 Hz, 3H), 1.29 (dd, J=6.88, 2.25 Hz, 3H). LCMS: Desired Mass: m/z=519.1; Observed Mass: m/z=520.1.

TABLE I
Compounds of formula I

| | |
|---|---|
| 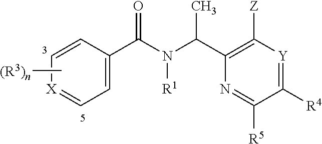 | I* |

| | | | | | | | | | | phys. data | | |
|---|---|---|---|---|---|---|---|---|---|---|---|---|
| | | | | | | | | | | | HPLC | M + H |
| No. | R¹ | R³ | X | Y | R⁴ | R⁵ | Z | R^{6a} | R^{6b} | Method | RT [min] | [m/z] |
| I-1 | H | 3-Cl,5-CF₃ | CH | CH | H | H | Q1 | H | H | A | 1.313 | 396.0 |
| I-2 | H | 3-Cl,5-CF₃ | CH | CH | H | H | Q2 | CN | H | A | 1.213 | 420.0 |
| I-3 | H | 3-Cl,5-CF₃ | CH | CH | H | Cl | Q1 | H | H | B | 1.893 | 430.0 |
| I-4 | H | 3,5-(CF₃)₂ | CH | CH | H | Cl | Q1 | H | H | C | 2.173 | 464.1 |
| I-5 | CH₂—cC₃H₅ | 3-Cl, 5-CF₃ | CH | CH | H | Cl | Q1 | H | H | B | 2.059 | 484.0 |
| I-6 | H | 3-Cl, 5-CF₃ | CH | CH | H | CH₃ | Q1 | H | H | B | 1.846 | 410.0 |
| I-7 | H | 3-Cl, 5-CF₃ | CH | CH | H | cC₃H₅ | Q1 | H | H | B | 1.975 | 436.0 |
| I-8 | H | 3,5-(CF₃)₂ | CH | N | H | H | Q1 | pyridyl-3 | H | D | 1.023 | 508.0 |
| I-9 | H | 3,5-(CF₃)₂ | CH | N | H | H | Q4 | pyridyl-3 | H | D | 0.995 | 508.0 |
| I-10 | H | 3,5-(CF₃)₂ | CH | N | H | H | Q3 | pyridyl-3 | H | D | 1.010 | 508.0 |
| I-11 | H | 3,5-(CF₃)₂ | CH | N | H | H | Q2 | pyridyl-3 | H | D | 1.005 | 507.0 |
| I-12 | CH₂—cC₃H₅ | 3,5-(CF₃)₂ | CH | N | H | H | Q1 | pyridyl-3 | H | D | 1.135 | 562.1 |
| I-13 | CH₂—cC₃H₅ | 3,5-(CF₃)₂ | CH | N | H | H | Q4 | pyridyl-3 | H | D | 1.114 | 562.1 |
| I-14 | H | 3,5-(CF₃)₂ | CH | N | H | H | Q1 | pyridyl-2 | H | D | 1.179 | 508.0 |
| I-15 | H | 3,5-(CF₃)₂ | CH | N | H | H | Q1 | C(O)NH- | H | D | 1.410 | 618.1 |
| | | | | | | | | (2,4-Cl₂—C₆H₃) | | | | |
| I-16 | H | 3,5-(CF₃)₂ | CH | N | H | H | Q1 | C₆H₅ | H | D | 1.323 | 508.0 |
| I-17 | H | 3,5-(CF₃)₂ | CH | N | H | H | Q2 | S(O)₂CH₃ | H | D | 1.148 | 508.0 |
| I-18 | H | 3,5-(CF₃)₂ | CH | N | H | H | Q1 | pyridyl-4 | H | D | 0.955 | 508.1 |
| I-19 | H | 3,5-(CF₃)₂ | CH | N | H | H | Q4 | pyridyl-4 | H | D | 0.950 | 508.1 |
| I-20 | H | 3,5-(CF₃)₂ | CH | N | H | H | Q1 | S(O)₂CH₃ | H | D | 1.169 | 508.9 |
| I-21 | H | 3,5-(CF₃)₂ | CH | N | H | H | Q1 | 5-Br-pyridyl-2 | H | D | 1.382 | 587.8 |
| I-22 | H | 3,5-(CF₃)₂ | CH | N | H | H | Q4 | 5-Br-pyridyl-2 | H | D | 1.382 | 587.8 |
| I-23 | CH₂—cC₃H₅ | 3,5-(CF₃)₂ | CH | N | H | H | Q2 | S(O)₂CH₃ | H | D | 1.281 | 562 |
| I-24 | H | 3,5-(CF₃)₂ | CH | N | H | H | Q1 | oxazolyl-2 | H | D | 1.171 | 498 |
| I-25 | H | 3-Cl, 5-SO₂CH₃ | CH | N | H | H | Q4 | pyridyl-2 | H | D | 0.867 | 484 |
| I-26 | H | 3,5-(CF₃)₂ | CH | C—Cl | H | H | Q2 | SCH₃ | H | B | 1.911 | 509.1 |
| I-27 | H | 3,5-(CF₃)₂ | CH | C—Cl | H | H | Q2 | S(O)₂CH₃ | H | B | 1.699 | 541 |
| I-28 | H | 3,5-(CF₃)₂ | CH | C—Cl | H | H | Q2 | S(O)CH3 | H | B | 1.562 | 525 |
| I-29 | H | 3,5-(CF₃)₂ | CH | N | H | H | Q1 | 1-CH₃-pyrazolyl-3 | | D | 1.166 | 511 |
| I-30 | H | 3,5-Cl₂ | CH | N | H | H | Q4 | pyridyl-2 | | D | 1.051 | 440 |
| I-31 | H | 3,5-Cl₂ | CH | N | H | H | Q2 | S(O)₂CH₃ | H | D | 1.088 | 440 |
| I-32 | H | 3-Br, 5-SO₂CF₃ | CH | N | H | H | Q2 | S(O)₂CH₃ | H | D | 1.145 | 583.9 |
| I-33 | H | 3-Cl, 5-OCF₃ | N | N | H | H | Q2 | S(O)₂CH₃ | H | D | 1.109 | 491 |
| I-34 | H | 3-Br, | CH | N | H | H | Q2 | S(O)₂CH₃ | H | D | 1.068 | 518.8 |
| | | 5-C(CH₃)₂CN | | | | | | | | | | |
| I-35 | H | 3-Cl, | N | N | H | H | Q2 | S(O)₂CH₃ | H | D | 1.024 | 472 |
| | | 5-(1-CN—cC₃H₄) | | | | | | | | | | |
| I-36 | H | 3-(1-CN—cC₃H₄), | CH | N | H | H | Q2 | S(O)₂CH₃ | H | D | 1.022 | 503 |
| | | 5-OCF₂H | | | | | | | | | | |
| I-37 | H | 3-Cl, 5-OCF₂H | CH | N | H | H | Q2 | S(O)₂CH₃ | H | D | 1.07 | 471.9 |
| I-38 | H | 3-Cl, 5-OCF₃ | CH | N | H | H | Q2 | S(O)₂CH₃ | H | D | 1.151 | 490 |
| I-39 | H | 3-CN, 5-OCF₃ | CH | N | H | H | Q2 | S(O)₂CH₃ | H | D | 1.052 | 481 |
| I-40 | H | 3-Br, | CH | N | H | H | Q2 | S(O)₂CH₃ | H | D | 1.041 | 516.9 |
| | | 5-(1-CN—cC₃H₄) | | | | | | | | | | |
| I-41 | H | 3,5-(CF₃)₂ | CH | N | H | H | Q2 | 2-F—C₆H₄ | H | D | 1.434 | 524 |
| I-42 | H | 3,5-(CF₃)₂ | CH | N | H | H | Q2 | pyrimidinyl-4 | H | D | 1.18 | 508.4 |
| I-43 | H | 3,5-(CF₃)₂ | CH | N | H | H | Q2 | 3-Cl-pyridyl-2 | H | D | 1.371 | 514 |
| I-44 | H | 3,5-(CF₃)₂ | CH | N | H | H | Q2 | pyrazinyl-2 | H | D | 1.041 | 507 |
| I-45 | H | 3,5-(CF₃)₂ | CH | N | H | H | Q2 | 3-OCH₃, | H | D | 1.453 | 570.2 |
| | | | | | | | | 4-Cl—C₆H₃ | | | | |
| I-46 | H | 3,5-(CF₃)₂ | CH | N | H | H | Q2 | pyridyl-2 | H | D | 1.037 | 507.3 |
| I-47 | H | 3,5-(CF₃)₂ | CH | N | H | H | Q2 | morpholinyl-4 | H | D | 1.245 | 515.1 |
| I-48 | H | 3,5-(CF₃)₂ | CH | N | H | H | Q2 | SCH(CH₃)₂ | | D | 1.445 | 504 |
| I-49 | H | 3,5-(CF₃)₂ | CH | N | H | H | Q2 | 4-C(O)OC₂H₅- | H | D | 1.321 | 585 |
| | | | | | | | | thiazolyl-2 | | | | |
| I-50 | H | 3,5-(CF₃)₂ | CH | N | H | H | Q2 | NHS(O)₂CH₂C₆H₅ | H | D | 1.278 | 599 |
| I-51 | H | 3,5-(CF₃)₂ | CH | N | H | H | Q2 | thiazolyl-2 | H | D | 1.279 | 513.3 |
| I-52 | H | 3,5-(CF₃)₂ | CH | N | H | H | Q2 | NH-pyridyl-2 | H | D | 1.01 | 522 |
| I-53 | H | 3,5-(CF₃)₂ | CH | C—F | H | H | Q2 | S(O)₂CH₃ | H | B | 1.658 | 525 |
| I-54 | H | 3,5-(CF₃)₂ | CH | C—Cl | H | H | Q1 | Br | H | B | 1.923 | 544 |
| I-55 | H | 3,5-(CF₃)₂ | CH | N | H | H | Q2 | S(O)₂CF₃ | H | B | 1.937 | 562 |
| I-56 | H | 3,5-(CF₃)₂ | CH | N | H | H | Q2 | S(O)₂C₂H₅ | H | B | 1.719 | 522.1 |
| I-57 | H | 3,5-(CF₃)₂ | CH | C—Cl | H | H | Q1 | pyridyl-2 | H | B | 1.809 | 541.1 |
| I-58 | H | 3,5-(CF₃)₂ | CH | N | H | H | Q2 | S(O)C₂H₅ | H | B | 1.6 | 506.1 |
| I-59 | H | 3,5-(CF₃)₂ | CH | C—OH | H | H | Q2 | S(O)₂CH₃ | H | B | 1.404 | 523.1 |
| I-60 | H | 3,5-(CF₃)₂ | CH | N | H | H | Q2 | SCF₃ | H | B | 2.045 | 530.1 |
| I-61 | H | 3,5-(CF₃)₂ | CH | N | H | H | Q2 | SC₂H₅ | H | B | 1.993 | 490.1 |
| I-62 | H | 3,5-(CF₃)₂ | CH | N | H | H | Q4 | pyridyl-2 | H | D | 1.133 | 508 |
| I-63 | H | 3,5-(CF₃)₂ | CH | N | H | H | Q2 | S—cC₃H₅ | H | B | 2.018 | 502.1 |
| I-64 | H | 3,5-(CF₃)₂ | CH | N | H | H | Q2 | S(O)—cC₃H₅ | H | B | 1.639 | 518.1 |
| I-65 | H | 3,5-(CF₃)₂ | CH | N | H | H | Q2 | S(O)₂—cC₃H₅ | H | B | 1.752 | 534.1 |
| I-66 | H | 3,5-(CF₃)₂ | CH | C—F | H | H | Q1 | pyridyl-2 | H | B | 1.771 | 525.1 |
| I-67 | H | 3,5-(CF₃)₂ | CH | N | H | H | Q2 | S(O)₂CH(CH₃)₂ | H | B | 1.771 | 536.1 |
| I-68 | H | 3,5-(CF₃)₂ | CH | C—Cl | H | H | Q1 | H | H | B | 1.816 | 464.1 |
| I-69 | H | 3,5-(CF₃)₂ | CH | N | H | H | Q2 | S(O)CH(CH₃)₂ | H | B | 1.668 | 520.1 |
| I-70 | H | 3,5-(CF₃)₂ | CH | N | H | H | Q2 | 6-Cl-pyridyl-2 | H | D | 1.39 | 541.1 |
| I-71 | H | 3,5-(CF₃)₂ | CH | N | H | H | Q2 | thiophenyl-2 | H | D | 1.413 | 512.1 |
| I-72 | H | 3,5-(CF₃)₂ | CH | N | H | H | Q2 | S(O)₂C(CH₃)₃ | H | B | 1.826 | 550.1 |
| I-73 | H | 3-Cl, 5-SO₂CH₃ | CH | N | H | H | Q1 | pyridyl-2 | H | D | 0.92 | 483.9 |
| I-74 | H | 3,5-(CF₃)₂ | CH | N | H | H | Q2 | 2-SCH₃-pyrimidi- | H | D | 1.347 | 554 |
| | | | | | | | | nyl-4 | | | | |
| I-75 | H | 3-Cl, | CH | N | H | H | Q1 | pyridyl-2 | H | D | 0.878 | 531 |
| | | 5-S(O)-pyridinyl-4 | | | | | | | | | | |
| I-76 | H | 3-Cl, 5- | CH | N | H | H | Q1 | pyridyl-2 | H | D | 0.907 | 506 |
| | | C[N(CH₃)₂]═NOCH₃ | | | | | | | | | | |
| I-77 | H | 3-(1-CN-cC₃H₄), | N | N | H | H | Q1 | pyridyl-2 | H | D | 1.146 | 522 |
| | | 5-OC(CH₃)₃ | | | | | | | | | | |
| I-78 | H | 3-Cl, 5- | CH | N | H | H | Q1 | pyridyl-2 | H | D | 1.167 | 503 |
| | | C(cC₃H₅)═NOCH₃ | | | | | | | | | | |
| I-79 | H | 3-Cl,5-SO₂CC₃H₅ | CH | N | H | H | Q1 | pyridyl-2 | H | D | 0.998 | 510 |
| I-80 | H | 3-Cl, | CH | N | H | H | Q1 | pyridyl-2 | H | D | 1.126 | 563.9 |
| | | 5-SO₂(4-F—C₆H₄) | | | | | | | | | | |
| I-81 | H | 3-(1-CN—cC₃H₄), | CH | N | H | H | Q1 | pyridyl-2 | H | D | 1.053 | 503 |
| | | 5-OCHF₂ | | | | | | | | | | |
| I-82 | H | 3-Cl, | CH | N | H | H | Q1 | pyridyl-2 | H | D | 1.243 | 472 |
| | | 5-C(cC₃H₅)═CH₂ | | | | | | | | | | |
| I-83 | H | 3-Cl, | CH | N | H | H | Q1 | pyridyl-2 | H | D | 0.869 | 515 |
| | | 5-S-pyridyl-4 | | | | | | | | | | |
| I-84 | H | 3-Cl, | CH | N | H | H | Q1 | pyridyl-2 | H | D | 1.014 | 547 |
| | | 5-SO₂-pyridyl-4 | | | | | | | | | | |

### BIOLOGICAL EXAMPLES

If not otherwise specified, the test solutions were prepared as follow: The active compound was dissolved at the desired concentration in a mixture of 1:1 (vol:vol) distilled water:acetone. The test solution was prepared on the day of use.

The activity of the compounds of formula I of the present invention can be demonstrated and evaluated by the following biological tests.

B.1 Tobacco Budworm (Heliothis virescens)

For evaluating control of tobacco budworm (Heliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom-built micro atomizer, at two replications.

After application, microtiter plates were incubated at about 28±1° C. and about 80±5% relative humidity for 5 days. Egg and larval mortality was then visually assessed.

In this test, compounds I-1, I-3, I-4, I-7, I-8, I-9, I-11, I-14, I-16, I-17, I-18, I-19, I-20, I-21, I-22, I-23, I-26, I-29, I-31, I-32, I-33, I-34, I-35, I-36, I-37, I-38, I-39, I-40, I-41, I-42, I-43, I-44, I-46, I-47, I-48, I-50, I-51, I-53, I-55, I-56, I-57, I-58, I-60, I-61, I-63, I-64, I-65, I-67, I-68, I-69, I-70, I-72, and I-73, resp., at 2500 ppm showed at least 75% mortality in comparison with untreated controls.

B.2 Boll Weevil (Anthonomus grandis)

For evaluating control of boll weevil (Anthonomus grandis) the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom-built micro atomizer, at two replications.

After application, microtiter plates were incubated at about 25±1° C. and about 75±5% relative humidity for 5 days. Egg and larval mortality was then visually assessed.

In this test, compounds I-1, I-2, I-3, I-4, I-8, I-9, I-10, I-11, I-12, I-13, I-14, I-16, I-17, I-18, I-19, I-20, I-21, I-23, I-24, I-25, I-26, I-28, I-29, I-30, I-31, I-32, I-33, I-34, I-35, I-36, I-37, I-38, I-39, I-40, I-41, I-42, I-43, I-44, I-46, I-47, I-48, I-50, I-51, I-53, I-55, I-56, I-57, I-58, I-60, I-61, I-63, I-64, I-65, I-67, I-68, I-69, I-70, I-71, and I-73, resp., at 2500 ppm showed at least 75% mortality in comparison with untreated controls.

B.3 Southern Armyworm (Spodoptera eridania), 2nd Instar Larvae

The active compounds were formulated by a Tecan liquid handler in 100% cyclohexanone as a 10,000-ppm solution supplied in tubes. The 10,000-ppm solution was serially diluted in 100% cyclohexanone to make interim solutions. These served as stock solutions for which final dilutions were made by the Tecan in 50% acetone:50% water (v/v) into 10 or 20 ml glass vials. A non-ionic surfactant (Kinetic@) was included in the solution at a volume of 0.01% (v/v). The vials were then inserted into an automated electrostatic sprayer equipped with an atomizing nozzle for application to plants/insects. Lima bean plants (variety Sieva) were grown 2 plants to a pot and selected for treatment at the 1st true leaf stage. Test solutions were sprayed onto the foliage by an auto-mated electrostatic plant sprayer equipped with an atomizing spray nozzle. The plants were dried in the sprayer fume hood and then removed from the sprayer. Each pot was placed into perforated plastic bags with a zip closure. Ten to 11 armyworm larvae were placed into the bag and the bags zipped closed. Test plants were maintained in a growth room at about 25° C. and about 20-40% relative humidity for 4 days, avoiding direct exposure to fluorescent light (14:10 light:dark photoperiod) to prevent trapping of heat inside the bags. Mortality and reduced feeding were assessed 4 days after treatment, compared to untreated control plants.

In this test, compounds I-1, I-3, I-4, I-5, I-8, I-9, I-12, I-14, I-16, I-17, I-18, I-19, I-20, I-21, I-23, I-24, I-26, I-28, I-30, I-31, I-32, I-33, I-34, I-35, I-36, I-37, I-38, I-39, I-40, I-42, I-44, I-46, I-47, I-48, I-50, I-51, I-56, I-57, I-60, and I-61, resp., at 300 ppm showed at least 75% mortality in comparison with untreated controls.

B.4 Orchid Thrips (Dichromothrips corbetti)

Dichromothrips corbetti adults used for bioassay were obtained from a colony maintained continuously under laboratory conditions. For testing purposes, the test compound is diluted in a 1:1 mixture of acetone:water (vol:vol), plus Kinetic HV at a rate of 0.01% v/v.

Thrips potency of each compound was evaluated by using a floral-immersion technique. All petals of individual, intact orchid flowers were dipped into treatment solution and allowed to dry in Petri dishes. Treated petals were placed into individual re-sealable plastic along with about adult thrips. All test arenas were held under continuous light and a temperature of about 28° C. for duration of the assay. After 3 days, the numbers of live thrips were counted on each petal. The percent mortality was recorded 72 hours after treatment.

In this test, compounds I-1, I-12, I-18, I-19, I-23, I-24, I-30, I-32, and I-39, resp., at 300 ppm showed at least 75% mortality in comparison with untreated controls.

B.5 Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae.

The active compounds were formulated using a solution containing 75% (v/v) water and 25% (v/v) DMSO. Different concentrations of formulated compounds or mixtures were sprayed onto the insect diet at 2.5 μl, using a custom-built micro atomizer, at two replications.

After application, microtiter plates were incubated at 281° C., 80±5% RH for 2 days. Larval mortality was then visually assessed.

In this test, compounds I-1, I-2, I-3, I-4, I-5, I-6, I-7, I-8, I-9, I-10, I-11, I-12, I-14, I-16, I-17, I-18, I-19, I-20, I-21, I-23, I-24, I-25, I-26, I-29, I-31, I-32, I-33, I-34, I-35, I-36, I-37, I-38, I-39, I-40, I-42, I-44, I-46, I-47, I-50, I-51, I-53, I-55, I-56, I-57, I-58, I-60, I-61, I-63, I-64, I-65, I-67, I-68, I-69, I-70, and I-73, resp., at 2500 ppm showed at least 75% mortality in comparison with untreated controls.

B.6 Green Peach Aphid (Myzus persicae)

For evaluating control of green peach aphid (Myzus persicae) through systemic means, the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, compounds I-8, I-9, I-11, I-12, I-13, I-14, I-17, I-18, I-20, I-21, I-23, I-24, I-25, I-28, I-29, I-30, I-31, I-32, I-34, I-35, I-36, I-37, I-38, I-39, I-40, I-42, I-44, I-46, I-51, I-56, I-57, I-58, I-61, I-64, I-65, I-67, I-69, and I-73, resp., at 2500 ppm showed at least 75% mortality in comparison with untreated controls.

The beneficial activity of the triazolone compounds according to the invention over structurally close compounds known from prior art with N-bonded triazoles was demonstrated by the following comparative experiments:

### C.1

| Structure | Example | B.1, 90 ppm | B.6, 30 ppm |
|---|---|---|---|
| 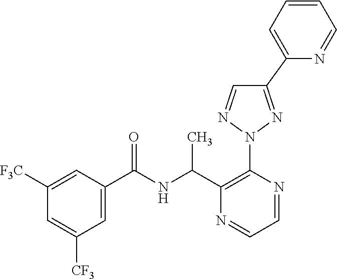 [SMILES: CC(NC(=O)c1cc(C(F)(F)F)cc(C(F)(F)F)c1)c1nccnc1-n1ncc(-c2ccccn2)n1] | I-14 | 100% | 100% |
| 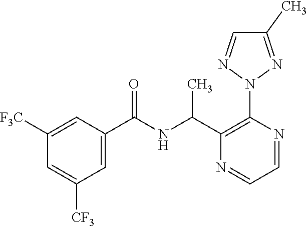 [SMILES: Cc1cnn(-c2nccnc2C(C)NC(=O)c2cc(C(F)(F)F)cc(C(F)(F)F)c2)n1] | WO2021/037614 P1 | 25% | 25% |

### C.2

| Structure | Example | B.6, 30 ppm |
|---|---|---|
| 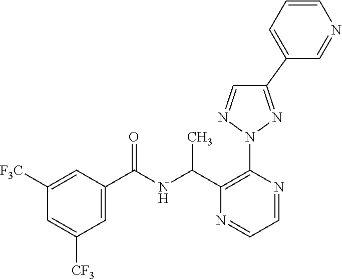 [SMILES: CC(NC(=O)c1cc(C(F)(F)F)cc(C(F)(F)F)c1)c1nccnc1-n1ncc(-c2cccnc2)n1] | I-8 | 100% |
| 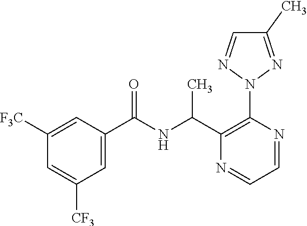 [SMILES: Cc1cnn(-c2nccnc2C(C)NC(=O)c2cc(C(F)(F)F)cc(C(F)(F)F)c2)n1] | WO2021/037614 P1 | 25% |

### C.3

| Structure | Example | B.2, 30 ppm | B.6, 90 ppm |
|---|---|---|---|
| 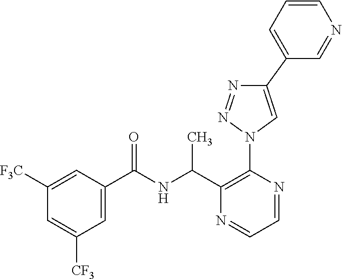 [SMILES: CC(NC(=O)c1cc(C(F)(F)F)cc(C(F)(F)F)c1)c1nccnc1-n1cc(-c2cccnc2)nn1] | I-9 | 100% | 100% |
| 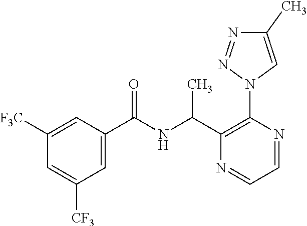 [SMILES: Cc1cn(-c2nccnc2C(C)NC(=O)c2cc(C(F)(F)F)cc(C(F)(F)F)c2)nn1] | WO2021/037614 P41 | 0% | 0% |

### C.4

| | | B.5, | B.2, | B.6, |
|---|---|---|---|---|
| Structure | Example | 90 ppm | 90 ppm | 30 ppm |
| 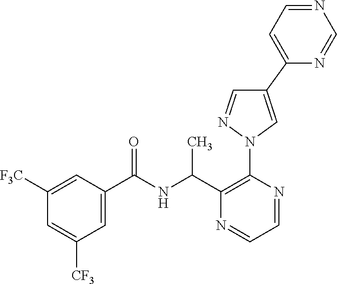 [SMILES: CC(NC(=O)c1cc(C(F)(F)F)cc(C(F)(F)F)c1)c1nccnc1-n1cc(-c2ccncn2)cn1] | I-42 | 100% | 100% | 100% |
| 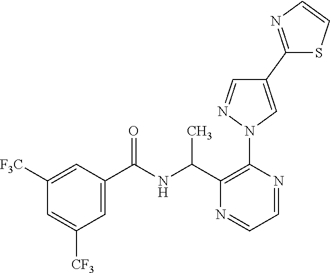 [SMILES: CC(NC(=O)c1cc(C(F)(F)F)cc(C(F)(F)F)c1)c1nccnc1-n1cc(-c2nccs2)cn1] | I-51 | 100% | 100% | 100% |
| 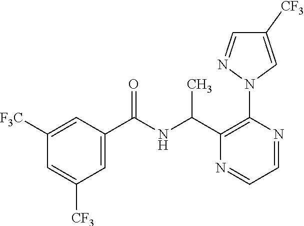 [SMILES: CC(NC(=O)c1cc(C(F)(F)F)cc(C(F)(F)F)c1)c1nccnc1-n1cc(C(F)(F)F)cn1] | WO2021/069575 I-92 | 0% | 0% | 0% |
| 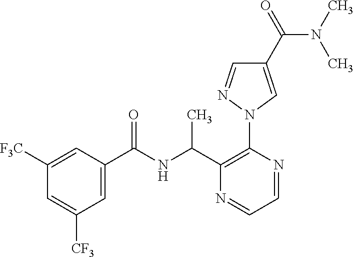 [SMILES: CC(NC(=O)c1cc(C(F)(F)F)cc(C(F)(F)F)c1)c1nccnc1-n1cc(C(=O)N(C)C)cn1] | WO2021/069575 I-88 | 0% | 0% | 50% |

### C.5

| | | B.2, | B.5, | B.6, |
|---|---|---|---|---|
| Structure | Example | 90 ppm | 80 ppm | 2500 ppm |
| 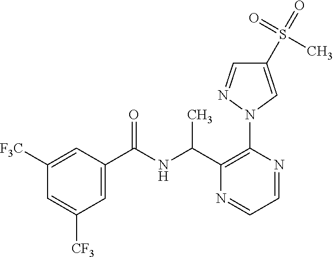 [SMILES: CC(NC(=O)c1cc(C(F)(F)F)cc(C(F)(F)F)c1)c1nccnc1-n1cc(S(C)(=O)=O)cn1] | I-17 | 100% | 100% | 100% |
| 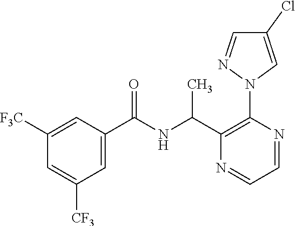 [SMILES: CC(NC(=O)c1cc(C(F)(F)F)cc(C(F)(F)F)c1)c1nccnc1-n1cc(Cl)cn1] | WO2021/069575 I-96 | 50% | 0% | 0% |

### C.6

| | | B.1, | B.2, |
|---|---|---|---|
| Structure | Example | 100 ppm | 90 ppm |
| 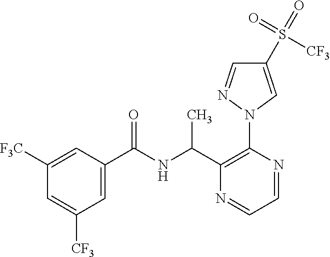 [SMILES: CC(NC(=O)c1cc(C(F)(F)F)cc(C(F)(F)F)c1)c1nccnc1-n1cc(S(=O)(=O)C(F)(F)F)cn1] | I-55 | 100% | 100% |
| 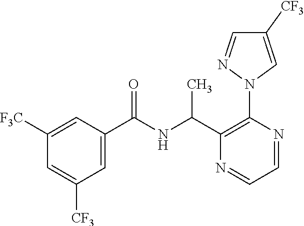 [SMILES: CC(NC(=O)c1cc(C(F)(F)F)cc(C(F)(F)F)c1)c1nccnc1-n1cc(C(F)(F)F)cn1] | WO2021/069575 I-92 | 50% | 0% |

### C.7

| | | B.1, | B.2, |
|---|---|---|---|
| Structure | Example | 90 ppm | 90 ppm |
| 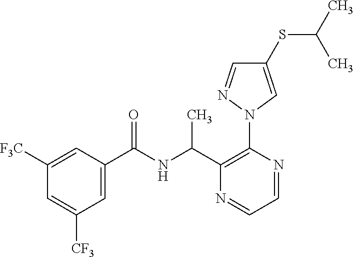 [SMILES: CC(C)Sc1cnn(-c2nccnc2C(C)NC(=O)c2cc(C(F)(F)F)cc(C(F)(F)F)c2)c1] | I-48 | 100% | 100% |
| 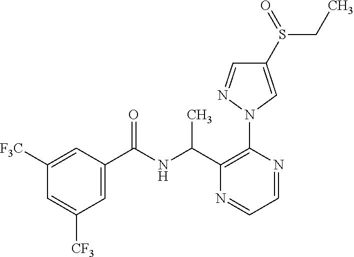 [SMILES: CCS(=O)c1cnn(-c2nccnc2C(C)NC(=O)c2cc(C(F)(F)F)cc(C(F)(F)F)c2)c1] | I-58 | 100% | 100% |
| 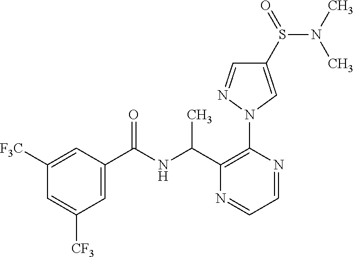 [SMILES: CC(NC(=O)c1cc(C(F)(F)F)cc(C(F)(F)F)c1)c1nccnc1-n1cc(S(=O)N(C)C)cn1] | WO2021/069575 I-88 | 0% | 0% |

### C.8

| | | B.2, | B.6, |
|---|---|---|---|
| Structure | Example | 90 ppm | 2500 ppm |
| 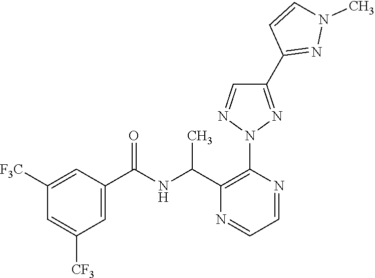 [SMILES: CC(NC(=O)c1cc(C(F)(F)F)cc(C(F)(F)F)c1)c1nccnc1-n1ncc(-c2ccn(C)n2)n1] | I-29 | 100% | 100% |
| 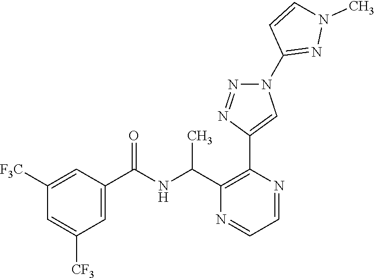 [SMILES: CC(NC(=O)c1cc(C(F)(F)F)cc(C(F)(F)F)c1)c1nccnc1-c1cn(-c2ccn(C)n2)nn1] | WO2021/259997 I-12 | 0% | 0% |

### C.9

| | | B.1, | B.2, |
|---|---|---|---|
| Structure | Example | 274 ppm | 9.9 ppm |
| 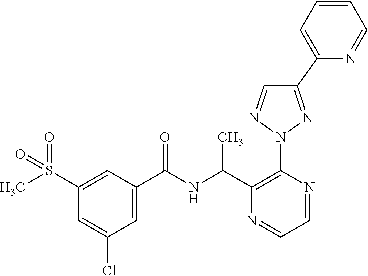 [SMILES: CC(NC(=O)c1cc(Cl)cc(S(C)(=O)=O)c1)c1nccnc1-n1ncc(-c2ccccn2)n1] | I-73 | 100% | 100% |
| 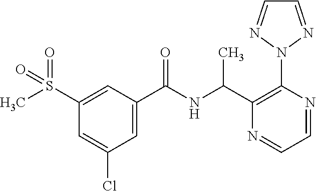 [SMILES: CC(NC(=O)c1cc(Cl)cc(S(C)(=O)=O)c1)c1nccnc1-n1nccn1] | WO2021/259997 I-1 | 0% | 0% |

1. A compound of formula I 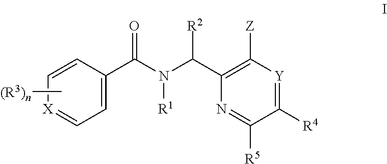
wherein
R¹ is H, OR¹⁰, C₁-C₆-alkyl, C₁-C₆-haloalkyl, C₃-C₆-cycloalkyl, C₃-C₆-halocycloalkyl, C₁-C₅-alkoxy, C₁-C₄-alkyl-C₃-C₆-cycloalkyl, C₁-C₄-alkyl-C₃-C₆-halocycloalkyl, which groups are unsubstituted or partially or fully substituted with R¹¹;
or C(═N—R¹¹)R¹², C(O)R^{11a};
R¹⁰ is H, C₁-C₄-alkyl, C₁-C₄-haloalkyl, C₃-C₆-cycloalkyl, C₃-C₆-halocycloalkyl, C₃-C₄-cycloalkyl-C₁-C₂-alkyl, C₃-C₄-halocycloalkyl-C₁-C₂-alkyl, C(O)—C₁-C₄-alkyl, C(O)—C₁-C₄-haloalkyl, C(O)—C₃-C₄-cycloalkyl, C(O)—C₃-C₄-halocycloalkyl, or phenyl which is unsubstituted or partially or fully substituted with R^{3a};
R¹¹ is halogen, CN, NO₂, NR¹²R¹³, C(O)NH₂, C(S)NH₂, C(O)OH, OR¹⁰, Si(CH₃)₃; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, 5- or 6-membered hetaryl, or phenyl, which rings are unsubstituted or substituted with halogen, C₁-C₃-haloalkyl, and/or CN;
R^{11a} is NR¹²R¹³, C(O)NH₂, C(S)NH₂, C(O)OH, OR¹⁰, Si(CH₃)₃; C₁-C₆-haloalkyl; C₂-C₆-alkenyl; C₂-C₆-haloalkenyl; C₂-C₆-alkynyl; C₂-C₆-haloalkynyl; C₃-C₄-cycloalkyl-C₁-C₂-alkyl, which ring is unsubstituted or substituted with 1 or 2 halogen; 3- to 6-membered heterocyclyl, which rings are unsubstituted or substituted with halogen, C₁-C₃-haloalkyl, and/or CN;
R¹², R¹³ are independently from each other H, C₁-C₄-alkyl, C₁-C₄-alkoxy, C₁-C₄-haloalkoxy, C₁-C₄-haloalkyl, C₃-C₆-cycloalkyl, C(O)—C₁-C₄-alkyl, C(O)—C₁-C₄-haloalkyl, C(O)—C₃-C₄-cycloalkyl, C(O)—C₃-C₄-halocycloalkyl, C(O)NH—C₁-C₄-alkyl, C(O)NH—C₁-C₄-haloalkyl, C(O)N(C₁-C₄-alkyl)-C₁-C₄-alkyl, C(O)N(C₁-C₄-haloalkyl)-C₁-C₄-alkyl, C(O)N(C₁-C₄-haloalkyl)-C₁-C₄-haloalkyl, C(O)NH—C₁-C₄-alkoxy, C(O)NH—C₁-C₄-haloalkoxy, C(O)NH—C₁-C₄-alkoxy-C₁-C₄-alkyl, C(O)NH—C₁-C₄-alkoxy-C₁-C₄-haloalkyl; C(O)NH-phenyl, C(O)NH-3-6-membered heterocyclyl or 5- or 6-membered hetaryl, C(O)NH—C₁-C₄-alkyl-phenyl, C(O)NH—C₁-C₄-alkyl-3-6-membered heterocyclyl or 5- or 6-membered hetaryl which rings are unsubstituted or substituted with halogen, C₁-C₃-haloalkyl, and/or CN; S(O)ₘ—C₁-C₄-haloalkyl, S(O)ₘ—C₃-C₄-cycloalkyl, S(O)ₘ—C₃-C₄-halocycloalkyl; 3- to 6-membered heterocyclyl, 5- or 6-membered hetaryl, or phenyl, which rings are unsubstituted or substituted with halogen, C₁-C₃-haloalkyl, and/or CN; or
or R¹² and R¹³ together with the nitrogen atom to which they are bound, form a 3-, 4-, 5-, 6-, or 7-membered saturated, partially or fully unsaturated heterocycle, which heterocycle may additionally contain 1 or 2 heteroatoms or heteroatom-containing groups selected from N, O, S(O)ₘ, and C(O) as ring members, and which heterocycle is unsubstituted or substituted with one or more R^{3a};
R¹⁴ is as defined for R¹⁰;
m is 0, 1, or 2;
R² is H, CN, C₁-C₃-alkyl, C₁-C₃-haloalkyl, C₂-C₃-alkynyl;
R³ is halogen, CN, NO₂;
C₁-C₄-alkyl, C₃-C₆-cycloalkyl, C₁-C₆-haloalkyl, C₁-C₆-halocycloalkyl, C₁-C₆-alkenyl, C₁-C₆-alkynyl, C₃-C₆-cycloalkyl-C₁-C₆-alkyl, C₁-C₆-alkyl-C₃-C₆-cycloalkyl, which are unsubstituted or substituted with R^{3a};
OR¹⁴, NR¹²R¹³, C(O)NR¹²R¹³, C(O)OR¹⁴, C(O)R¹⁵, S(O)ₘ—R¹⁵, C(═N—OR¹⁰)R¹⁴,
C(═N—OR¹⁰)NR¹²R¹³, or C(═N—NR¹²R¹³)R¹⁴;
R^{3a} is halogen, CN, NO₂, OH, C₁-C₄-alkyl, C₁-C₄-haloalkyl, C₁-C₄-alkoxy-C₁-C₄-alkyl, C₁-C₄-alkoxy, C₁-C₄-haloalkoxy, C₃-C₄-cycloalkyl, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl;
R¹⁵ is H, C₁-C₄-alkyl, or C₁-C₄-haloalkyl, C₃-C₆-cycloalkyl, C₁-C₆-halocycloalkyl, which carbon chains are unsubstituted or partially or fully substituted with R¹¹; or 3- to 6-membered heterocyclyl, 5- or 6-membered hetaryl, or phenyl, which rings are unsubstituted or substituted with R^{3a};
n is 0, 1, 2, or 3;
R⁴ and R⁵ are, independently from each other, H, halogen, CN, C₁-C₆-alkyl, C₁-C₃-haloalkyl, C₃-C₆-cycloalkyl, C₃-C₆-cyclohaloalkyl, OR¹⁰, NR¹²R¹³, 3- to 6-membered heterocyclyl, 5- or 6-membered hetaryl, or phenyl, which rings are unsubstituted or partially or fully substituted with R^{3a};
X, Y are independently from each other N, CH, or CR^{3b};
R^{3b} is as defined for R³;
Z is Q1, Q2, Q3, or Q4, wherein # indicates the bond to the adjacent 6-membered ring 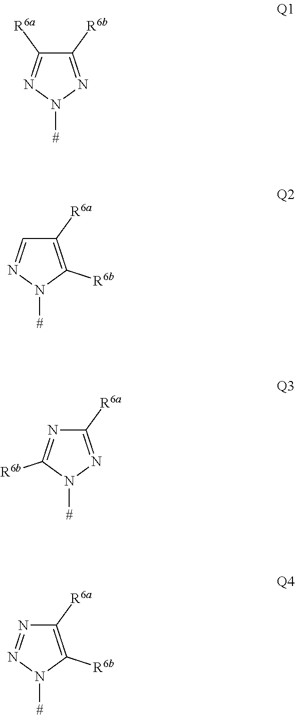
R^{6a} is S(O)ₘ—C₁-C₄-alkyl, S(O)ₘ—C₁-C₄-haloalkyl, S(O)ₘ—C₃-C₆-cycloalkyl, S(O)ₘ—C₃-C₄-halocycloalkyl, NR¹²R¹³, C(O)NR¹²R¹³, C(O)OR¹⁵, 3- to 6-membered heterocyclyl, 5- or 6-membered hetaryl, or phenyl, which rings are unsubstituted or substituted with R³;
R^{6b} is H, halogen, CN, C₁-C₃-alkyl, C₁-C₃-haloalkyl, C₃-C₄-cycloalkyl, C₃-C₄-halocycloalkyl, OR¹⁰, which rings are unsubstituted or substituted with R³;
or, provided that Y is not N,
R^{6a}, R^{6b} are, independently from each other, H, halogen, CN, C₁-C₃-alkyl, C₁-C₃-haloalkyl, C₃-C₄-cycloalkyl, C₃-C₄-halocycloalkyl, OR¹⁰, S(O)ₘ—C₁-C₄-alkyl, S(O)ₘ—C₁-C₄-haloalkyl, S(O)ₘ—C₃-C₆-cycloalkyl, S(O)ₘ—C₃-C₄-halocycloalkyl, NR¹²R¹³, C(O)NR¹²R¹³, C(O)OR¹⁵, 3- to 6-membered heterocyclyl, 5- or 6-membered hetaryl, or phenyl, which rings are unsubstituted or substituted with R³;
and the N-oxides, stereoisomers, and agriculturally or veterinarily acceptable salts thereof.

2. The compound of formula I according to claim 1, wherein
R³ is halogen, CN, NO₂;
C₁-C₄-alkyl, C₃-C₆-cycloalkyl, C₁-C₆-haloalkyl, C₁-C₆-halocycloalkyl, C₁-C₆-alkenyl, C₁-C₆-alkynyl, C₃-C₆-cycloalkyl-C₁-C₆-alkyl, C₁-C₆-alkyl-C₃-C₆-cycloalkyl, which are unsubstituted or substituted with R^{3a};
OR¹⁴, NR¹²R¹³, C(O)NR¹²R¹³, C(O)OR¹⁴, C(O)R¹⁵, S(O)ₘ—R¹⁵;

3. The compound of formula I according to claim 1, wherein R¹ is H or CH₂-cC₃H₅.

4. The compound of formula I according to claim 1, wherein R² is CH₃.

5. The compound of formula I according to claim 1, wherein R³ is halogen, CN, C₁-C₄-haloalkyl, C₁-C₄-haloalkoxy, C₃-C₄-cycloalkyl unsubstituted or substituted with one or more CN, C₃-C₄-halocycloalkyl, S(O)ₘ—C₁-C₄-alkyl, S(O)ₘ—C₁-C₄-haloalkyl, S(O)ₘ—C₃-C₄-cycloalkyl, S(O)ₘ—C₃-C₄-halocycloalkyl, or S(O)ₘ—R¹⁴, wherein R¹⁴ is phenyl, which is partially substituted with R¹¹.

6. The compound of formula I according to claim 1, wherein n is 2 and R³ is in positions 3 and 5.

7. The compound of formula I according to claim 1, wherein X is CH.

8. The compound of formula I according to claim 1, wherein X is N.

9. The compound of formula I according to claim 1, wherein R⁴ is H, F, Cl, or CH₃, and R⁵ is H, halogen, C₁-C₄-alkyl, or C₃-C₆-cycloalkyl.

10. The compound of formula I according to claim 1, wherein R^{6a} is 2-, 3-, or 4-pyridine.

11. The compound of formula I according to claim 1, which corresponds to formula I.AQ1

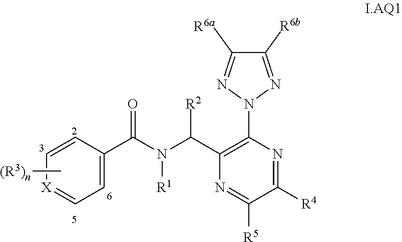

12. The compound of formula I according to claim 11, wherein R^{6a} is 2-pyridine or 3-pyridine, and R^{6b} is H.

13. The compound of formula I according to claim 1, which corresponds to formula I.B

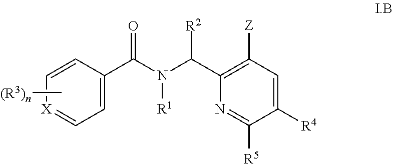

14. The compound of formula I according to claim 13, which corresponds to formula I.BQ1

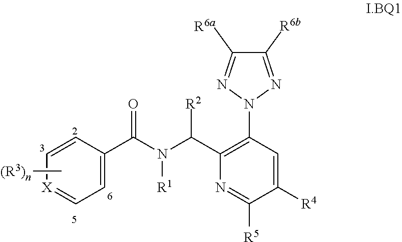

15. The compound of formula I according to claim 1, which corresponds to formula I.C

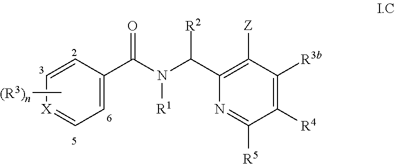

16. The compound of formula I according to claim 15, which corresponds to formula I.CQ1

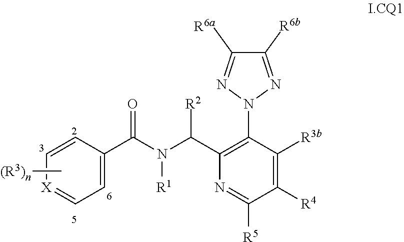

17. The compound of formula I according to claim 1, comprising mainly of isomer I.S.

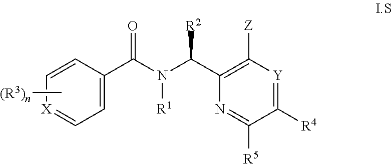

18. An agricultural or veterinary composition comprising at least one compound according to claim 1 and/or at least one agriculturally or veterinarily acceptable salt thereof, and at least one inert liquid and/or solid agriculturally or veterinarily acceptable carrier.

19. An agricultural composition for combating animal pests comprising at least one compound as defined in claim 1 and at least one inert liquid and/or solid acceptable carrier and, if optionally, at least one surfactant.

20. A method for combating or controlling invertebrate pests, comprising contacting said pest or its food supply, habitat or breeding grounds with a pesticidally effective amount of at least one compound as defined in claim 1.

21. A method for protecting growing plants from attack or infestation by invertebrate pests, comprising contacting a plant, or soil or water in which the plant is growing, with a pesticidally effective amount of at least one compound as defined in claim 1.

22. A seed comprising a compound as defined in claim 1, or an enantiomer, diastereomer, or salt thereof, in an amount of from 0.1 g to 10 kg per 100 kg of seed.

23. A method for treating or protecting an animal from infestation or infection by invertebrate pests comprising bringing the animal in contact with a pesticidally effective amount of at least one compound of the formula I as defined in claim 1, a stereoisomer thereof, and/or at least one veterinarily acceptable salt thereof.
